SEQUENCE LISTING (101 sequences)

SEQ ID NO: 1 - protein, 462 aa, Caenorhabditis elegans
MSLGQLSYTPVTDVGIGAIELYFPQNFVDQNDLEKFNNVSSGKYTIGLGQQQMGFCSDNEDIVSISLTVTRKLIETYKISTDSIGCLVVGTETMIDKSKSVKTALMDLFPGNSDIEGVDIKNACFGGAQALLHAIDWVTVNHPLDKKNAIVVVADIAIYEEGPARCTGGAGAIAFLICPDASIPIDRQFSACHMKNTWDFFKPITPIPSEYPVVDGSLSLSSYLEAVRMTYTYFISKVNRHTTGIDGLNSFDGVFLHSPFTKMVQKGLAVMNYTDSQLRHKQLNGNGVDHKLDENDRAGLAKMIELSAQVWKEKTDPYLVFNRRIGNMYTPSLFAQLLAYLAADDCVTGEKSILFFAYGSGLASAIFPGRVRQTSNLDKIRQVAIRAIKRLDDRIQFTPEEFTETLQKREVFLRSKEIPKSPSETSLFPNTYFLDNMDKLYRRSYTLHEEPNGVQNGNGIHH

SEQ ID NO: 2 - protein, 447 aa, Schizosaccharomyces pombe
MSFDRKDIGIKGLVLYTPNQYVEQAALEAHDGVSTGKYTIGLGLTKMAFVDDREDIYSFGLTALSQLIKRYQIDISKIGRLEVGTETIIDKSKSVKSVLMQLFGDNHNVEGIDCVNACYGGVNALFNTIDWIESSAWDGRDGIVVAGDIALYAKGNARPTGGAGCVALLVGPNAPIVFEPGLRGTYMQHAYDFYKPDLTSEYPYVDGHFSLECYVKALDGAYANYNVRDVAKNGKSQGLGLDRFDYCIFHAPTCKQVQKAYARLLYTDSAAEPSNPELEGVRELLSTLDAKKSLTDKALEKGLMAITKERFNKRVSPSVYAPTNCGNMYTASIFSCLTALLSRVPADELKGKRVGAYSYGSGLAASFFSFVVKGDVSEIAKKTNLVNDLDNRHCLTPTQYEEAIELRHQAHLKKNFTPKGSIERLRSGTYYLTGIDDMFRRSYSVKP

SEQ ID NO: 3 - protein, 491 aa, Saccharomyces cerevisiae
MKLSTKLCWCGIKGRLRPQKQQQLHNTNLQMTELKKQKTAEQKTRPQNVGIKGIQIYIPTQCVNQSELEKFDGVSQGKYTIGLGQTNMSFVNDREDIYSMSLTVLSKLIKSYNIDTNKIGRLEVGTETLIDKSKSVKSVLMQLFGENTDVEGIDTLNACYGGTNALFNSLNWIESNAWDGRDAIVVCGDIAIYDKGAARPTGGAGTVAMWIGPDAPIVFDSVRASYMEHAYDFYKPDFTSEYPYVDGHFSLTCYVKALDQVYKSYSKKAISKGLVSDPAGSDALNVLKYFDYNVFHVPTCKLVTKSYGRLLYNDFRANPQLFPEVDAELATRDYDESLTDKNIEKTFVNVAKPFHKERVAQSLIVPTNTGNMYTASVYAAFASLLNYVGSDDLQGKRVGLFSYGSGLAASLYSCKIVGDVQHIIKELDITNKLAKRITETPKDYEAAIELRENAHLKKNFKPQGSIEHLQSGVYYLTNIDDKFRRSYDVKK

SEQ ID NO: 4 - protein, 461 aa, Arabidopsis thaliana
MAKNVGILAMDIYFPPTCVQQEALEAHDGASKGKYTIGLGQDCLAFCTELEDVISMSFNAVTSLFEKYKIDPNQIGRLEVGSETVIDKSKSIKTFLMQLFEKCGNTDVEGVDSTNACYGGTAALLNCVNWVESNSWDGRYGLVICTDSAVYAEGPARPTGGAAAIAMLIGPDAPIVFESKLRASHMAHVYDFYKPNLASEYPVVDGKLSQTCYLMALDSCYKHLCNKFEKIEGKEFSINDADYIVFHSPYNKLVQKSFARLLYNDFLRNASSIDEAAKEKFTPYSSLTLDESYQSRDLEKVSQQISKPFYDAKVQPTTLIPKEVGNMYTASLYAAFASLIHNKHNDLAGKRVVMFSYGSGSTATMFSLRLNDNKPPFSISNIASVMDVGGKLKARHEYAPEKFVETMKLMEHRYGAKDFVTTKEGIIDLLAPGTYYLKEVDSLYRRFYGKKGEDGSVANGH

SEQ ID NO: 5 - protein, 482 aa, Dictyostelium discoideum
MTKPENIGIHGIEVYFPSTYVAQEDLEKFDGVSQGKYTLGLGQTNMAFCGDREDIYSLSLNAVNNLMDKFNVDPNSIGRLEVGTETVIDKSKSVKTVLMDLFAKHGNTSIDGIDTINACYGGTSALHNALQWMESSYWDGRNAIVVAGDIAVYEKGPARPTGGAGVVAMLIGPNAPITFESGLRGVHMENVYDFYKPDMDSEYPRVDGKLSISCYFRAIDNCYNRYAKAFEKKYGKSFSLDQVDFALFHSPYNKLVQKSFGRMLYNDFLNNPNDSRYASLEAYKNVKPEDTYFDSVLEKALSAITKNDYATKVAPTTLLAKQLGNTYCGSTYSGLLSLLDEKSNDLVGKRVLTFSYGSGLAASAFSFKVEKPINHIVEKVDLKNRLAKRVRVEPEIFTEKLSLRETRHNLKNYVPSDETTNMFPGSFYLSSVDNAGIRKYDRTYSTSAVLGAFQRRQQISQSTIKSLNLFRATKSVLSILKK

SEQ ID NO: 6 - protein, 453 aa, Blattella germanica
MWPSDVGIVALELIFPSQYVDQVDLEVYDNVSAGKYTVGLGQARMGFCTDREDINSLCLTVVSRLMERWSIPYSQIGRLEVGTETLLDKSKSVKTVLMQLFKDNTDIEGVDTVNACYGGTSALFNAISWVESSSWDGRYALVVAGDIAVYAKGSARPTGGAGAVAMLVGANAPLVFDRGVRSSHMQHAYDFYKPDLSSLYPTVDGKLSIQCYLSALDHCYQLYCSKIQKQLGEKFDIERLDAVLFHAPYCKLVQKSLARLVLNDFVRASEEERTTKYSSLEALKGVKLEDTYFDREVEKAVMTYSKNMFEEKTKPSLLLANQVGNMYTPSLYGGLVSLLVSKSAQELAGKRVALFSYGSGLASSMFSLRISSDASAKSSLQRLVSNLSHIKPQLDLRHKVSPEEFAQTMETREHNHHKAPYTPEGSIDVLFPGTWYLESVDSLYRRSYKQVPG

SEQ ID NO: 7 - protein, 522 aa, Gallus gallus
MPGSLPVNTESCWPKDVGIVALEIYFPSQYVDQTELEKYDGVDAGKYTIGLGQSKMGFCSDREDINSLCLTVVQKLMERNSLSYDCIGRLEVGTETIIDKSKSVKTVLMQLFEESGNTDVEGIDTTNACYGGTAALFNAINWIESSSWDGRYALVVAGDIAVYATGNARPTGGAGAVAMLVGSNAPLIFERGLRGTHMQHAYDFYKPDMVSEYPVVDGKLSIQCYLSALDRCYSVYRNKIHAQWQKEGTDRGFTLNDFGFMIFHSPYCKLVQKSVARLLLNDFLSDQNAETANGVFSGLEAFRDVKLEDTYFDRDVEKAFMKASAELFNQKTKASLLVSNQNGNMYTPSVYGCLASLLAQYSPEHLAGQRISEFSYGSGFAATLYSIRVTQDATPGSALDKITASLSDLKARLDSRKCIAPDVFAENMKIRQETHHLANYIPQCSVEDLFEGTWYLVRVDEKHRRTYARRPVMGDGPLEAGVEVVHPGIVHEHIPSPAKKVPRIPATTESEGVTVAISNGVH

SEQ ID NO: 8 - protein, 520 aa, Homo sapiens
MPGSLPLNAEACWPKDVGIVALEIYFPSQYVDQAELEKYDGVDAGKYTIGLGQAKMGFCTDREDINSLCMTVVQNLMERNNLSYDCIGRLEVGTETIIDKSKSVKTNLMQLFEESGNTDIEGIDTTNACYGGTAAVFNAVNWIESSSWDGRYALVVAGDIAVYATGNARPTGGVGAVALLIGPNAPLIFERGLRGTHMQHAYDFYKPDMLSEYPIVDGKLSIQCYLSALDRCYSVYCKKIHAQWQKEGNDKDFTLNDFGFMIFHSPYCKLVQKSLARMLLNDFLNDQNRDKNSIYSGLEAFGDVKLEDTYFDRDVEKAFMKASSELFSQKTKASLLVSNQNGNMYTSSVYGSLASVLAQYSPQQLAGKRIGVFSYGSGLAATLYSLKVTQDATPGSALDKITASLCDLKSRLDSRTGVAPDVFAENMKLREDTHHLVNYIPQGSIDSLFEGTWYLVRVDEKHRRTYARRPTPNDDTLDEGVGLVHSNIATEHIPSPAKKVPRLPATAAEPEAAVISNGVW

SEQ ID NO: 9 - protein, 508 aa, Homo sapiens
MQRLLTPVKRILQLTRAVQETSLTPARLLPVAHQRFSTASAVPLAKTDTWPKDVGILALEVYFPAQYVDQTDLEKYNNVEAGKYTVGLGQTRMGFCSVQEDINSLCLTVVQRLMERIQLPWDSVGRLEVGTETIIDKSKAVKTVLMELFQDSGNTDIEGIDTTNACYGGTASLFNAANWMESSSWDGRYAMVVCGDIAVYPSGNARPTGGAGAVAMLIGPKAPLALERGLRGTHMENVYDFYKPNLASEYPIVDGKLSIQCYLRALDRCYTSYRKKIQNQWKQAGSDRPFTLDDLQYMIFHTPFCKMVQKSLARLMFNDFLSASSDTQTSLYKGLEAFGGLKLEDTYTNKDLDKALLKASQDMFDKKTKASLYLSTHNGNMYTSSLYGCLASLLSHHSAQELAGSRIGAFSYGSGLAASFFSFRVSQDAAPGSPLDKLVSSTSDLPKRLASRKCVSPEEFTEIMNQREQFYHKVNFSPPGDTNSLFPGTWYLERVDEQHRRKYARRPV

SEQ ID NO: 10 - protein, 468 aa, Dictyostelium discoideum
MKKTKDIGICAIDIYFPQTYVNQSELKKYDKVSNGKYTIGLGQTNMSFVGDREDIVSMAMTSVKMMMSKYSIDYQSIGRLEVGTETIIDKSKSVKSSIMSLFQEYGNTSLEGVDTLNACYGGTNALFNSLQWIESSYWDGRYALVVTGDIAVYSKGAARPTGGAGVVTMLIGPNATLIFDQSLRGTHMENVNDFYKPDLSSEYPYVDGKLSIECYLRALDKCYLEYKKKFESINDDNKFSMDSFDYVCFHSPYNRLVQKSYARLIYNDFLQNPNNPKYQDLLPFKDLSTGKDSYINSKLDQITLKLSLDDFKTKVNPSTLLSKECGNSYCGSVYSGILSLLSNVNDLNNKKVLVFSYGSGLAASLFSFRINNNKNRNNNNNNNNCFFKTTNDIGKISNIKERLSNRVKVSPEEFTRILDIREKSHQMVGARTPIDTLDYISAGTFYLEKIDEKLIRHYKSKPIISSKL

SEQ ID NO: 11 - protein, 388 aa, Staphylococcus epidermidis
MNIGIDKISFYVPKYYVDMAKLAEARQVDPNKFLIGIGQTEMTVSPVNQDIVSMGANAAKDIITEEDKKNIGMVIVATESAIDNAKAAAVQIHHLLGIQPFARCFEMKEACYAATPAIQLAKDYLAQRPNEKVLVIASDTARYGIHSGGEPTQGAGAVAMMISHDPSILKLNDDAVAYTEDVYDFWRPTGHQYPLVAGALSKDAYIKSFQESWNEYARRHNKTLADFASLCFHVPFTKMGQKALDSIINHADETTQDRLNSSYQDAVDYNRYVGNIYTGSLYLSLISLLETRDLKGGQTIGLFSYGSGSVGEFFSGTLVDGFKEQLDVERHKSLLNNRIEVSVDEYEHFFKRFDQLELNHELEKSNADRDIFYLKSIDNNIREYHIAE

SEQ ID NO: 12 - protein, 389 aa, Lactobacillus fermentum
MKIGIDKLAFATTPYYLAMEDLAQGRNVDPNKYLIGIGQSKQAVVPPTQDVVTLAAAAADKLLDPVERDQVSTVIVATESGIDNSKAAAVYVKHLLKLSDFTRAVEVKEACYSATAALQFARGLVALNPQEKILVIASDIARYGLETGGEVTQGAGAVAMLITANPRVLAIEPTSVAYTKDVMDFWRPLYAEEALVNGKYSTNVYIDFFKQCWTRYQQLAGYGLEDFAALAFHLPFTKMGKKALEAELGDRDDQVATRLRANLTAGQEACRQVGNLYTGSLYLGLMSLLTEGDVKPGERIGLFSYGSGAEGEFFAGILQPGYQEGLGDLNEQLAARTQVSLAEYEDLFNQQLGLKEEDVTFKTPAAGQRFVLVGQKDHQRQYRDLAERD

SEQ ID NO: 13 - protein, 351 aa, Hyperthermus butylicus
MPRGSGIVGWGGYVPRYRIKAAEIVRVWGWEPSVPAGLGVKEKAVENVDEDSVTMGYEAARNAIARANVDPREIKAVFFGTESKPYAVKPSATIIAEALGITPETMASDLEFACRAASEGLRASLALVEAGYMKYALVVASDTAQANPGDVLEFTAASGAAAFVVGPASESVAVLEGVYTYVTDTPDFWRGQHSRYPMHGEAFTGEPAYFHHIESAVKGLMEKLGLKPEDFDYAVFHQPNGKFPLRVGARLGFPKEKILPGLLTPIIGNTYNASALLGFARILDQAKPGQRILVAPFGSGAGSDAYSFIVTDRIEEARNRAPKVDDYVNWKRYIDYAMHARMRKLYDRRPV

SEQ ID NO: 14 - protein, 349 aa, Chloroflexus aggregans
MMKPNQPVGIIGYGVYIPRYRIAAREIARIWTDGQNGVPVEAKSVPGPDEDTITMAIEAARNALVRADIPASALGAVWIGSESHPYSVKPSGTVVADALGAGPWVSAADWEFACKAGSEALTAAMALVGSGMQRYALAIGADTAQGRPGDALEYTASAGAAALIVGPATEALATIDATVSYVTDTPDFYRRADRPYPVHGNRFTGEPAYFHQIQSAASELLRQLNRTAADFTYAVFHQPNAKFPQTVAKRLGFTDAQIAPGLLSPQIGNTYSGAALLGLCAILDVAKPGDTIFVTSYGSGAGSDAYALTVTEAIVERRERAPLTAAYLARKVMIDYAMYAKWRGKLVMG

SEQ ID NO: 15 - protein, 387 aa, Lactobacillus delbrueckii
MDIGIDQIGFYTPNKFVDMVDLANARNQDPNKFLIGIGQDRMAVADKTQDAVSMGINATAEYLDQVDLEQLGLLIFATESGIDQSKSASLFVKEALNLPARIRTFEIKEACFALTASLQVARDYVRAHPHHSAMIIGSDIARYGLATAGEVTQGAGAISMLIKENPAIIALEDGHTSHSENINDFWRPNNLATAVVDGHYSRDVYLDFFKSTFKPFLAEKQLQVSDFAGICYHLPYTKMGYKAHKIAIEGQDDETVKRLSDNFQLSAKYSRQVGNIYTASLYMSVLSLLENGDLEAGDRIGFFSYGSGAMAEFFSGKVVAGYQKRLRPALHARMLKERIRLGVGQYEDIFTEGLEALPENVEFTSDANHGTWYLAGQEGYVRQYKQK

SEQ ID NO: 16 - protein, 388 aa, Staphylococcus haemolyticus
MSIGIDKINFYVPKYYVDMAKLAEARQVDPNKFLIGIGQTQMAVSPVSQDIVSMGANAAKDIITDDDKKHIGMVIVATESAIDNAKAAAVQIHNLLGVQPFARCFEMKEACYAATPAIQLAKDYIEKRPNEKVLVIASDTARYGIQSGGEPTQGAGAVAMLISNNPSILELNDDAVAYTEDVYDFWRPTGHKYPLVAGALSKDAYIKSFQESWNEYARREDKTLSDFESLCFHVPFTKMGKKALDSIINDADETTQERLTSGYEDAVYYNRYVGNIYTGSLYLSLISLLENRSLKGGQTIGLFSYGSGSVGEFFSATLVEGYEKQLDIEGHKALLNERQEVSVEDYESFFKRFDDLEFDHATEQTDDDKSIYYLENIQDDIRQYHIPK

SEQ ID NO: 17 - protein, 420 aa, Bacillus subtilis
MVSAGIEAMNVFGGTAYLDVMELAKYRHLDTARFENLLMKEKAVALPYEDPVTFGVNAAKPIIDALSEAEKDRIELLITCSESGIDFGKSLSTYIHEYLGLNRNCRLFEVKQACYSGTAGFQMAVNFILSQTSPGAKALVIASDISRFLIAEGGDALSEDWSYAEPSAGAGAVAVLVGENPEVFQIDPGANGYYGYEVMDTCRPIPDSEAGDSDLSLMSYLDCCEQTFLEYQKRVPGANYQDTFQYLAYHTPFGGMVKGAHRTMMRKVAKVKTSGIETDFLTRVKPGLNYCQRVGNIMGAALFLALASTIDQGRFDTPKRIGCFSYGSGCCSEFYSGITTPQGQERQRTFGIEKHLDRRYQLSMEEYELLFKGSGMVRFGTRNVKLDFEMIPGIMQSTQEKPRLFLEEISEFHRKYRWIS

SEQ ID NO: 18 - protein, 388 aa, Mycobacterium marinum
MVSIGIHDLSIATAHYVLDHATLAEHHGVDVNKYLIGLGQQQMSIVAPDEDIVTLAAAAADPIIKRHGSQKIRTIVIGTETGVDQSKSAGIWVSSLLGLPSSARVLEVKQACYGATGALQLALALVHRDPTQQVLVIAADVARYDLDSPGEPTQGAAAAAMLVSADPALLRLEEPTGIYTADIMDFWRPNYRSTALVDGKASVTAYMEAASGAWKDYTERGGRAFGEFAAFCYHQPFTKMAYKAHKQLAAEAGEDASGAAVQAAVGNTVEYNRRIGNSYTASLYLALAALLDQADDLSDQPIAMLSYGSGCVAELFAGTVTPGYQQHLRTDQHRAALETRIPLSYEHYRRLHNLTLPTNGNHHSLPVETSRPFRLTAISEHKRMYGAV

SEQ ID NO: 19 - protein, 435 aa, Zea mays
MLAASTKVGSRLASPHASLSAGAAAAALASSPVLGSGMLPGAGFGETGNHHAADAPPPLPCSSSGDSREYYQWKRLVNQRQSTLHVGEVPAALGHHVFGAGCSSRKQHIYRYFSSSSHQGSIWARSKILHDLPGYVKIVEVGPRDGLQNEKDIVPTPVKVELIRRLATSGLPVVEATSFVSPKWVPQLADAKDVMEAVRTIGGVRFPVLTPNLKGFEAAIAAGAKEIAIFASASEGFSKSNINCTIKESIARYNDVALAAKEKEIPVRGYVSCVVGCPVDGPVPPSNVAYVAKELYDMGCYEVSLGDTIGVGTPGTVVPMLEAAISVVPVEKLAVHFHDTYGQSLSNILISLQMGVSVVDSSVAGLGGCPYAKGASGNVATEDVVYMLNGLGVKTGVDLGKVMAAGEFICRHLGRQSGSKAATALSKVTANASKL

SEQ ID NO: 20 - protein, 335 aa, Danio rerio
  REGION 1..335: note = Danio rerio (Brachydanio rerio)
MGNVSSAVKHCLSYETFLRDYPWLPRLLWEEKCSELPKLPVYVKIVEVGPRDGLQNEKEIVPTEVKIQLIDLLSQTGLPVIEATSFVSSKWVAQMADHTAVLKGIKRSPDVRYPVLTPNIQGFQAAVAAGANEVAVFGSASETFSRKNINCSIEESLQRFEQVVSAAKQEGIPVRGYVSCALGCPYEGQVKPSQVTKVAKRLFELGCYEVSLGDTIGVGTAGSMAEMLSDVLTEVPAGALAVHCHDTYGQALPNILIALQMGVSVVDASVAGLGGCPFAKGASGNVSTEDLLYMLHGLGIETGVDLLKVMEAGDFICKALNRKTNSKVSQATRNN

SEQ ID NO: 21 - protein, 325 aa, Bos taurus
MATVKKVLPRRLVGLATLRAVSTSSVGTFPKQVKIVEVGPRDGLQNEKNIVPTPVKIKLIDMLSEAGLPVVEATSFVSPKWVPQMADHAEVLKGIQKFPGVNYPVLTPNFKGFQAAVAAGAKEVAIFGAASELFTKKNINCSIDESLQRFDEILKAARAAGISVRGYVSCVLGCPYEGKISPAKVAEVTKKLYSMGCYEISLGDTIGVGTPGAMKDMLSAVLQEVPVTALAVHCHDTYGQALANTLTALQMGVSVMDSSVAGLGGCPYAQGASGNLATEDLVYMLAGLGIHTGVNLQKLLEAGAFICQALNRRTNSKVAQATCKL

SEQ ID NO: 22 - protein, 325 aa, Homo sapiens
MAAMRKALPRRLVGLASLRAVSTSSMGTLPKRVKIVEVGPRDGLQNEKNIVSTPVKIKLIDMLSEAGLSVIETTSFVSPKWVPQMGDHTEVLKGIQKFPGINYPVLTPNLKGFEAAVAAGAKEVVIFGAASELFTKKNINCSIEESFQRFDAILKAAQSANISVRGYVSCALGCPYEGKISPAKVAEVTKKFYSMGCYEISLGDTIGVGTPGIMKDMLSAVMQEVPLAALAVHCHDTYGQALTNTLMALQMGVSVVDSSVAGLGGCPYAQGASGNLATEDLVYMLEGLGIHTGVNLQKLLEAGNFICQALNRKTSSKVAQATCKL

SEQ ID NO: 23 - protein, 299 aa, Pseudomonas putida
  REGION 1..299: note = Pseudomonas putida Q88H25
MSLPKHVRLVEVGPRDGLQNEAQPISVADKVRLVNDLTEAGLAYIEVGSFVSPKWVPQMAGSAEVFAGIQQRPGVTYAALAPNLRGFEDALAAGVKEVAVFAAASEAFSQRNINCSISESLKRFEPIMDAARSHGMRVRGYVSCVLGCPYEGKVSAEQVAPVARALHDMGCYEVSLGDTIGTGTAGDTRRLFEVVSAQVPREQLAGHFHDTYGQALANVYASLLEGISVFDSSVAGLGGCPYAKGATGNIASEDVVYLLQGLGIETGIDLGLLIAAGQRISGVLGRDNGSRVARACSAQ

SEQ ID NO: 24 - protein, 312 aa, Acinetobacter baumannii
  REGION 1..312: note = Acinetobacter baumannii B7H4C6
MTAFSDLLVVQEVSPRDGLQIEPTWVPTDKKIDLINQLSTMGFSRIEAGSFVSPKAIPNLRDGEEVFTGITRHKDIIYVGLIPNLKGALRAVEANANELNLVLSASQTHNLANMRMTKAQSFAGFTEIVEQLQGKTQFNGTVATTFGCPFEGKISEREVFSLVEHYLKLGIHNITLADTTGMANPVQVKRIVSHVLSLISPEQLTLHFHNTRGLGLTNVLAAYEVGARRFDAALGGLGGCPFAPGASGNICTEDLVNMCEEIGIPTTIDLDALIQLSRTLPALLGHDTPSQLAKAGRNTDLHPIPDYIKSLN

SEQ ID NO: 25 - protein, 286 aa, Thermus thermophilus
  REGION 1..286: note = Thermus thermophilus Q72IH0
MKASVRWVECPRDAWQGFSRFIPTEEKVAFLNELLEAGFAHLDLTSFVSPKWVPQMQDAEEVLKALPPPNGRTYLAIVANEKGLERALAAPNLTHVGYPFSLSETFQQRNTNRSIEASWPLVGAMVERTEGRLGLVVYLSMAFGNPYGDPWSVEAVLEALARLKEMGVREIALADTYGVAEPERIHEVLKAAVARFGPEGLGAHLHARPEGALAKVEAVLAAGVTWLEGALAGVGGCPFAGDELVGNLPTEVVLPHLEKRGLATGVDLSRLPLLAEEAARLKALYA

SEQ ID NO: 26 - protein, 301 aa, Clostridium acetobutylicum
  REGION 1..301: note = Phosphate butyryltransferase
  REGION 1..301: note = Clostridium acetobutylicum ATCC 824
MIKSFNEIIMKVKSKEMKKVAVAVAQDEPVLEAVRDAKKNGIADAILVGDHDEIVSIALKIGMDVNDFEIVNEPNVKKAALKAVELVSTGKADMVMKGLVNTATFLRSVLNKEVGLRTGKTMSHVAVFETEKFDRLLFLTDVAFNTYPELKEKIDIVNNSVKVAHAIGIENPKVAPICAVEVINPKMPSTLDAAMLSKMSDRGQIKGCVVDGPLALDIALSEEAAHHKGVTGEVAGKADIFLMPNIETGNVMYKTLTYTTDSKNGGILVGTSAPVVLTSRADSHETKMNSIALAALVAGNK

SEQ ID NO: 27 - protein, 329 aa, Corynebacterium glutamicum
  REGION 1..329: note = Corynebacterium glutamicum ATCC 13032
  REGION 1..329: note = Phosphate acetyltransferase
MSAELFENWLLKRARAEHSHIVLPEGDDDRILMAAHQLLDQDICDITILGDPVKIKERATELGLHLNTAYLVNPLTDPRLEEFAEQFAELRKSKSVTIDEAREIMKDISYFGTMMVHNGDADGMVSGAANTTAHTIKPSFQIIKTVPEASVVSSIFLMVLRGRLWAFGDCAVNPNPTAEQLGEIAVVSAKTAAQFGIDPRVAILSYSTGNSGGGSDVDRAIDALAEARRLNPELCVDGPLQFDAAVDPGVARKKMPDSDVAGQANVFIFPDLEAGNIGYKTAQRTGHALAVGPILQGLNKPVNDLSRGATVPDIVNTVAITAIQAGGRS

SEQ ID NO: 28 - protein, 402 aa, Salmonella enterica
  REGION 1..402: note = Salty propionate kinase
  REGION 1..402: note = subsp. enterica serovar Typhimurium str. LT2
MNEFPVVLVINCGSSSIKFSVLDVATCDVLMAGIADGMNTENAFLSINGDKPINLAHSNYEDALKAIAFELEKRDLTDSVALIGHRIAHGGELFTQSVIITDEIIDNIRRVSPLAPLHNYANLSGIDAARHLFPAVRQVAVFDTSFHQTLAPEAYLYGLPWEYFSSLGVRRYGFHGTSHRYVSRRAYELLDLDEKDSGLIVAHLGNGASICAVRNGQSVDTSMGMTPLEGLMMGTRSGDVDFGAMAWIAKETGQTLSDLERVVNKESGLLGISGLSSDLRVLEKAWHEGHERARLAIKTFVHRIARHIAGHAASLHRLDGIIFTGGIGENSVLIRQLVIEHLGVLGLTLDVEMNKQPNSHGERIISANPSQVICAVIPTNEEKMIALDAIHLGNVKAPVEFA

SEQ ID NO: 29 - protein, 402 aa, Escherichia coli
  REGION 1..402: note = Propionate kinase
  REGION 1..402: note = Escherichia coli K-12
MNEFPVVLVINCGSSSIKFSVLDASDCEVLMSGIADGINSENAFLSVNGGEPAPLAHHSYEGALKAIAFELEKRNLNDSVALIGHRIAHGGSIFTESAIITDEVIDNIRRVSPLAPLHNYANLSGIESAQQLFPGVTQVAVFDTSFHQTMAPEAYLYGLPWKYYEELGVRRYGFHGTSHRYVSQRAHSLLNLAEDDSGLVVAHLGNGASICAVRNGQSVDTSMGMTPLEGLMMGTRSGDVDFGAMSWVASQTNQSLGDLERVVNKESGLLGISGLSSDLRVLEKAWHEGHERAQLAIKTFVHRIARHIAGHAASLRRLDGIIFTGGIGENSSLIRRLVMEHLAVLGLEIDTEMNNRSNSCGERIVSSENARVICAVIPTNEEKMIALDAIHLGKVNAPAEFA

SEQ ID NO: 30 - protein, 154 aa, Haemophilus influenzae
  REGION 1..154: note = Haemophilus influenzae R2866
  REGION 1..154: note = Acyl-CoA thioesterase YciA
MSANFTDKNGRQSKGVLLLRTLAMPSDTNANGDIFGGWIMSQMDMGGAILAKEIAHGRVVTVAVESMNFIKPISVGDVVCCYGECLKVGRSSIKIKVEVWVKKVASEPIGERYCVTDAVFTFVAVDNNGRSRTIPRENNQELEKALALISEQPL

SEQ ID NO: 31 - protein, 140 aa, Homo sapiens
  REGION 1..140: note = Acyl-coenzyme A thioesterase
MTSMTQSLREVIKAMTKARNFERVLGKITLVSAAPGKVICEMKVEEEHTNAIGTLHGGLTATLVDNISTMALLCTERGAPGVSVDMNITYMSPAKLGEDIVITAHVLKQGKTLAFTSVDLTNKATGKLIAQGRHTKHLGN

SEQ ID NO: 32 - protein, 132 aa, Escherichia coli
  REGION 1..132: note = Escherichia coli K-12
MSTTHNVPQGDLVLRTLAMPADTNANGDIFGGWLMSQMDIGGAILAKEIAHGRVVTVRVEGMTFLRPVAVGDVVCCYARCVQKGTTSVSINIEVWVKKVASEPIGQRYKATEALFKYVAVDPEGKPRALPVE

SEQ ID NO: 33 - protein, 286 aa, Escherichia coli
  REGION 1..286: note = Escherichia coli K-12
MSQALKNLLTLLNLEKIEEGLFRGQSEDLGLRQVFGGQVVGQALYAAKETVPEERLVHSFHSYFLRPGDSKKPIIYDVETLRDGNSFSARRVAAIQNGKPIFYMTASFQAPEAGFEHQKTMPSAPAPDGLPSETQIAQSLAHLLPPVLKDKFICDRPLEVRPVEFHNPLKGHVAEPHRQVWIRANGSVPDDLRVHQYLLGYASDLNFLPVALQPHGIGFLEPGIQIATIDHSMWFHRPFNLNEWLLYSVESTSASSARGFVRGEFYTQDGVLVASTVQEGVMRNHN

SEQ ID NO: 34 - protein, 289 aa, Pseudomonas putida
MSHVLDDLVDLLSLESIEENLFRGRSQDLGFRQLYGGQVLGQSLSAASQTVEDARHVHSLHGYFLRPGDASLPVVYSVDRVRDGGSFSTRRVTAIQKGQTIFTCSASFQYDEEGFEHQAQMPDVVGPENLPTEVELAHAMADQLPERIRDKVLCAKPIEIRPVTERDPFNPKPGDPVKYAWFRADGNLPDVPALHKYMLAYASDFGLLTTALLPHGKSVWQRDMQIASLDHSLWFHGNLRADQWLLYATDSPWAGNSRGFCRGSIFNQAGQLVASSSQEGLIRHRKDWA

SEQ ID NO: 35 - protein, 258 aa, unidentified
  REGION 1..258: note = Myxococcus species
MPEFKVDARGPIEIWTIDGESRRNAISRAMLQELGEMVTRVSSSREVRAVVITGAGDKAFCAGADLKERATMAEDEVRAFLDGLRRTFRALEKSDCVFIAAINGAAFGGGTELALACDLRVAAPAAELGLTEVKLGIIPGGGGTQRLTRLVGPGRAKDLILTARRINAAEAFSVGLVNRLAPEGHLLAVAYGLAESVVENAPIAVATAKHAIDEGTGLELDDALALELRKYEEILKTEDRLEGLRAFAEKRAPVYKGR

SEQ ID NO: 36 - protein, 447 aa, Schizosaccharomyces pombe; identical to SEQ ID NO: 2
  REGION 1..447: note = Schizosaccharomyces pombe (strain 972 / ATCC 24843)

SEQ ID NO: 37 - protein, 392 aa, Clostridium acetobutylicum
  REGION 1..392: note = Clostridium acetobutylicum ATCC 824
MKEVVIASAVRTAIGSYGKSLKDVPAVDLGATAIKEAVKKAGIKPEDVNEVILGNVLQAGLGQNPARQASFKAGLPVEIPAMTINKVCGSGLRTVSLAAQIIKAGDADVIIAGGMENMSRAPYLANNARWGYRMGNAKFVDEMITDGLWDAFNDYHMGITAENIAERWNISREEQDEFALASQKKAEEAIKSGQFKDEIVPVVIKGRKGETVVDTDEHPRFGSTIEGLAKLKPAFKKDGTVTAGNASGLNDCAAVLVIMSAEKAKELGVKPLAKIVSYGSAGVDPAIMGYGPFYATKAAIEKAGWTVDELDLIESNEAFAAQSLAVAKDLKFDMNKVNVNGGAIALGHPIGASGARILVTLVHAMQKRDAKKGLATLCIGGGQGTAILLEKC

SEQ ID NO: 38 - protein, 258 aa, Bacillus subtilis
  REGION 1..258: note = subsp. subtilis str. RO-NN-1
MNAISLAVDQFVAVLTIHNPPANALSSRILEELSSCLDQCETDAGVRSIIIHGEGRFFSAGADIKEFTSLKGNEDFSLLAERGQQLMERIESFPKPIIAAIHGAALGGGLELAMACHIRIAADDAKLGLPELNLGIIPGFAGTQRLPRYVGTAKALELIGSGEPISGKEALDLGLVSIGAKDEAEVIEKAKALAAKFAEKSPQTLASLLELLYSNKVYSYEGSLKLEAKRFGEAFESEDAKEGIQAFLEKRKPQFKGE

SEQ ID NO: 39 - protein, 263 aa, Bacillus anthracis
MKNERLVICSKKGSSAVITIQNPPVNALSLEVVQQLINVLEEIEMDDDIAVVIITGIGGKAFVAGGDIKEFPGWIGKGEKYAEMKSIELQRPLNQLENLSKPTIAAINGLALGGGCELALACDLRVIEEQALIGLPEITLGLFPGAGGTQRLPRLIGEGKAKEMMFTGKPITAKEAKEINLVNYITSRGEALNKAKEIAKDISEFSLPALSYMKLAIREGLAVPLQEGLQIEARYFGKVFQTEDVKEGVKAFIEKRVPRFTNK

SEQ ID NO: 40 - protein, 229 aa, Candida albicans
  REGION 1..229: note = Candida albicans (strain SC5314 / ATCC MYA-2876)
MIARVCLRRSNVLPIFQIPSRKYSINYEKVNNSIYNNVIKPKRIVLAITGATGTQIGVRLLEILKELGVETHLVMSKWGIATLKYETDYQVDYVTSLATKTYSARDVTAPISSGSFVHDGMIVAPCSMKSLSAIRTGFTEDLIVRAADVSLKERRKLLLVARETPLSDIHLDNMLYLSRMGVTIFPPVPAFYTKPKTIDDIVEQTCGRILDNFGINIDTFERWDGINHR

SEQ ID NO: 41 - protein, 227 aa, Aspergillus niger
MFNSLLSGTTTPNSGRASPPASEMPIDNDHVAVARPAPRRRRIVVAMTGATGAMLGIKVLIALRRLNVETHLVMSKWAEATIKYETDYHPSNVRALADYVHNINDMAAPVSSGSFRADGMIVVPCSMKTLAAIHSGFCDDLISRTADVMLKERRRLVLVARETPLSEIHLRNMLEVTRAGAVIFPPVPAFYIKAGSIEDLIDQSVGRMLDLFDLDTGDFERWNGWEK

SEQ ID NO: 42 - protein, 242 aa, Saccharomyces cerevisiae
  REGION 1..242: note = Saccharomyces cerevisiae S288c
MLLFPRRTNIAFFKTTGIFANFPLLGRTITTSPSFLTHKLSKEVTRASTSPPRPKRIVVAITGATGVALGIRLLQVLKELSVETHLVISKWGAATMKYETDWEPHDVAALATKTYSVRDVSACISSGSFQHDGMIVVPCSMKSLAAIRIGFTEDLITRAADVSIKENRKLLLVTRETPLSSIHLENMLSLCRAGVIIFPPVPAFYTRPKSLHDLLEQSVGRILDCFGIHADTFPRWEGIKSK

SEQ ID NO: 43 - protein, 180 aa, Cryptococcus gattii
  REGION 1..180: note = Cryptococcus gattii WM276
  REGION 1..180: note = serotype B
MRRKRYVVAVTGATGATLAIRLLQALRALDIETHLIISKWAVKTLKYETDMIERELKDLADYSYSNSDLAAPPSSGSFIHDGMFIIPCSMKTLAAVRIGLGDELISRSADVCLKEGRKLMLVVRETPLNDIHLENMLFLRRAGAIIFPPVPAYYIRPQTIDDLTNQTVGRILDSSKCSQK

SEQ ID NO: 44 - protein, 189 aa, Escherichia coli
  REGION 1..189: note = Escherichia coli K-12
MKRLIVGISGASGAIYGVRLLQVLRDVTDIETHLVMSQAARQTLSLETDFSLREVQALADVTHDARDIAASISSGSFQTLGMVILPCSIKTLSGIVHSYTDGLLTRAADVVLKERRPLVLCVRETPLHLGHLRLMTQAAEIGAVIMPPVPAFYHRPQSLDDVINQTVNRVLDQFAITLPEDLFARWQGA

SEQ ID NO: 45 - protein, 192 aa, Bacillus subtilis
MKLVIGMTGATGAIFGIRLLEYLKAAEIETHLVVSPWANVTITHETDYTLKDVEKLASYTYSHKDQAAAISSGSFETDGMIIAPCSMKSLASIRTGMADNLLTRAADVILKERKKLVLLTRETPLSQIHLENMLALTKMGSVILPPMPAFYNKPADMDELIDHIVFRTLDQFGIRLPEAKRWYGIEKQKGGI

SEQ ID NO: 46 - protein, 209 aa, Pseudomonas aeruginosa
MSGPERITLAMTGASGAQYGLRLLDCLVQEEREVHFLISKAAQLVMATETDVALPAKPQAMQAFLTEYCGAAAGQIRVFGQNDWMAPPASGSSAPNAMVICPCSTGTLSAVATGACNNLIERAADVALKERRPLVLVPREAPFSSIHLENMLKLSNLGAVILPAAPGFYHQPQSVEDLVDFVVARILNTLGIPQDMLPRWGEQHLVSDE

SEQ ID NO: 47 - protein, 219 aa, unidentified
  REGION 1..219: note = Enterobacter species DC4
MLRQVRANALTCNSPQNPAQSALKSVRAKIMKRLIVGLSGASGAIYGVRLLQVLRNVAEVETHLVMSQAARQTLSLETDLSLRDVQALADVVHDARDIAASISSGSFKTAGMVILPCSIKTLSGIVNSYTDTLVTRAADVVLKERRPLVLCVRETPLHLGHLRLMTQAAELGAIIMPPVPAFYHRPTSLDDVINQTVNRVLDQFDIDLPEDLFTRWQGA

SEQ ID NO: 48 - protein, 503 aa, Saccharomyces cerevisiae
  REGION 1..503: note = Saccharomyces cerevisiae S288c
MRKLNPALEFRDFIQVLKDEDDLIEITEEIDPNLEVGAIMRKAYESHLPAPLFKNLKGASKDLFSILGCPAGLRSKEKGDHGRIAHHLGLDPKTTIKEIIDYLLECKEKEPLPPITVPVSSAPCKTHILSEEKIHLQSLPTPYLHVSDGGKYLQTYGMWILQTPDKKWTNWSIARGMVVDDKHITGLVIKPQHIRQIADSWAAIGKANEIPFALCFGVPPAAILVSSMPIPEGVSESDYVGAILGESVPVVKCETNDLMVPATSEMVFEGTLSLTDTHLEGPFGEMHGYVFKSQGHPCPLYTVKAMSYRDNAILPVSNPGLCTDETHTLIGSLVATEAKELAIESGLPILDAFMPYEAQALWLILKVDLKGLQALKTTPEEFCKKVGDIYFRTKVGFIVHEIILVADDIDIFNFKEVIWAYVTRHTPVADQMAFDDVTSFPLAPFVSQSSRSKTMKGGKCVTNCIFRQQYERSFDYITCNFEKGYPKGLVDKVNENWKRYGYK

SEQ ID NO: 49 - protein, 168 aa, unidentified
  REGION 1..168: note = Enterobacter species MGH 24
MSTFDKHDLSGFVGKHLVYTYDNGWNYEIYVKNETTLDYRIHSGLVANRWVKDQQAYIVRVGESIYKISWTEPTGTDVSLIVNLGDKLFHGTIFFPRWVMNNPEKTVCFQNDHIPLMNSYRDAGPAYPTEVIDEFATITFVRDCGANNESVIACAASELPNDFPANLN

SEQ ID NO: 50 - protein, 161 aa, Bacillus pumilus
MDQFVGLHMIYTYENGWEYEIYIKNDHTIDYRIHSGMVGGRWVRDQEVNIVKLTKGVYKVSWTEPTGTDVSLNFMPEEKRMHGVIFFPKWVHERPDITVCYQNDYIDLMKESREKYETYPKYVVPEFADITYIHHAGVNDETIIAEAPYEGMTDEIRAGRK

SEQ ID NO: 51 - protein, 534 aa, Aspergillus niger
  REGION 1..534: note = Aspergillus niger CBS 513.88
MLRMLRPGRRIPTHPSRSFSTTPHRSNDSPALNFRSLLSALRAQDDLVDITQPASPDLEIAALTRRVYESHSPAPLFHNVTDTDPETGLFKILGAPVGLRADPATRFGRLAIQLGLPQNATPLDILEKLIAAKHSTPLPPTPVPASSAPCKENILHGSQIDMTKWPIPRLHPLDGGNYLATYGFHILQSPDKAWTSWSISRTMHVANTPRTIVAPIMPGQHIAQVHQMWADQGAKDTPWALVLGGPPAAAFVGGMPLPAFVSEDGYIGALCGEAMDVVKCETNDLYVPANAEIVLEGRISTTEKVGEGPMGEYHGYMFQDKAVPEPRIEVDCVTYRRDPVVPICVAGLAPDETHTVWGAAISAEILDALRGAELPVKMAWMPYEAQCCWVVVSVDVERLGRMGIKKEELSRRVGEVVFGTHAGWEAPKVFVVGDDVDVTDIGQFVWALATRYRPGADELVFEEADGLPMIPYMTRASRREVPNPGKGGKSVVNLLLPSEFEGKRPWVPGSFEGLYSEELKQKVLGRWGELFEKK

SEQ ID NO: 52 - protein, 513 aa, Candida dubliniensis
  REGION 1..513: note = Candida dubliniensis CD36
MSLNPALKFRDFIQVLKNEGDLIEIDTEVDPNLEVGAITRKAYENKLAAPLFNNLKQDPENIDPKNLFRILGCPGGLRGFGNDHARIALHLGLDSQTPMKEIIDFLVANRNPKKYIPPVLVPNDQSPHKKHHLTKEQIDLTKLPVPLLHHGDGGKFIQTYGMWVLQTPDKSWTNWSIARGMVHDSKSITGLVINPQHVKQVSDAWVAAGKGDKIPFALCFGVPPAAILVSSMPIPDGATEAEYIGGLCNQAVPVVKCETNDLEVPADCEMVFEGYLDRDTLVREGPFGEMHGYCFPKDHHTQPLYRVNHISYRDQAIMPISNPGLCTDETHTLIGGLVSAETKYLISQHPVLSKIVEDVFTPYEAQALWLAVKINTHELVKLKTNAKELSNLVGDFLFRSKECYKVCSILHEIILVGDDIDIFDFKQLIWAYTTRHTPVQDQLYFDDVKPFALAPFASQGPLIKTRQGGKCVTTCIFPKQFTDPDFEFVTCNFNGYPEEVKNKISQNWDKYYK

SEQ ID NO: 53 - protein, 497 aa, Escherichia coli
  REGION 1..497: note = Escherichia coli K-12
MDAMKYNDLRDFLTLLEQQGELKRITLPVDPHLEITEIADRTLRAGGPALLFENPKGYSMPVLCNLFGTPKRVAMGMGQEDVSALREVGKLLAFLKEPEPPKGFRDLFDKLPQFKQVLNMPTKRLRGAPCQQKIVSGDDVDLNRIPIMTCWPEDAAPLITWGLTVTRGPHKERQNLGIYRQQLIGKNKLIMRWLSHRGGALDYQEWCAAHPGERFPVSVALGADPATILGAVTPVPDTLSEYAFAGLLRGTKTEVVKCISNDLEVPASAEIVLEGYIEQGETAPEGPYGDHTGYYNEVDSFPVFTVTHITQREDAIYHSTYTGRPPDEPAVLGVALNEVFVPILQKQFPEIVDFYLPPEGCSYRLAVVTIKKQYAGHAKRVMMGVWSFLRQFMYTKFVIVCDDDVNARDWNDVIWAITTRMDPARDTVLVENTPIDYLDFASPVSGLGSKMGLDATNKWPGETQREWGRPIKKDPDVVAHIDAIWDELAIFNNGKSA

SEQ ID NO: 54 - protein, 473 aa, Bacillus megaterium
  REGION 1..473: note = Bacillus megaterium QM B1551
MAYKDFRDFLNTLHKEGQLLTVTDEVQPDPDLGSAGQAISNLGDQTPGLLFTNIYGYNNAKVALNVMGSWSNHALMMGLPKSTPVKEQFFEFARRYEKFPVKVKREETAPFHECEIKDDINLFDLLPLFRLNQGDGGYYLDKACVISRDQHDKENFGKQNVGIYRMQVKGKDRLGIQPVPQHDIAIHLKQAEEKGENLPVSIALGCEPAIVTAAATPLHYDQSEYEMAGAIQGEPYRIVKSQLSDLDVPWGAEVILEGEILAGEREYEGPFGEFTGHYSGGRSMPVIKINRVYHRKDPIFESLYIGMPWTETDYLIGINTSVPLYQQLKEAYPEEIEAVNAMYTHGLVAIVSTKSRYGGFAKAVGMRALTTPHGLGYCKLVILVDEDVDPFNLPQVMWALSTKMHPKHDVITVPNLSVLPLDPGSEPAGITDKMILDATTPVAPETRGHYSQPLDTPLETEKWEKILTNMMQK

SEQ ID NO: 55 - protein, 423 aa, unidentified
  REGION 1..423: note = Methanothermobacter sp. CaT2
MRNFLDKIGEEALVVEDEVSTSFEAASILREHPRDLVILKNLKESDIPVISGLCNTREKIALSLNCRVHEITHRIVEAMENPTPISSVGGLDGYRSGRADLSELPILRHYRRDGGPYITAGVIFARDPDTGVRNASIHRMMVIGDDRLAVRIVPRHLYTYLQKAEERGEDLEIAIAIGMDPATLLATTTSIPIDADEMEVANTFHEGELELVRCEGVDMEVPPAEIILEGRILCGVREREGPFVDLTDTYDVVRDEPVISLERMHIRKDAMYHAILPAGFEHRLLQGLPQEPRIYRAVKNTVPTVRNVVLTEGGCCWLHAAVSIKKQTEGDGKNVIMAALAAHPSLKHVVVVDEDIDVLDPEEIEYAIATRVKGDDDILIVPGARGSSLDPAALPDGTTTKVGVDATAPLASAEKFQRVSRSE

SEQ ID NO: 56 - protein, 494 aa, Mycobacterium chelonae
  REGION 1..494: note = Mycobacterium chelonae 1518
MAFNDLRRYLADLEAHGELRTIKTPVSTEIQLGAIARLACETYGPAALFENLVGYPTFRGLAAFETYSGNPDNRAWRLARALGLSDDTTGDQIVDFLAGFRDTAGVAPVLVETGPVHENIVRDRGELLDYLPIPHLHPGDGGPYVNTIGFFVLESPDRSWVNWAVARCMKLDGDRMVGMTAVMQHIGMLRREWDKIGTSVPFALVLGADPITTLISGGPLAKFGASEGDIIGAIRGEPLEVVECVTSSLRVPAHAEIVIEGYIDLTESADEGPMAEYHGYIDKAKNTTGAPDFGVYHITAVTHRNDAIYPSTCAGKPVDEDHTITGPGVAAASLNALRAASLPVEKAWMVPESASHVLAVTVSDGWSGEFPDANELCRKIGNAVKTMDHSAYWVQRILVTDNDIDPTSPSDLWWAYATRCRPGDDSIILEDVPIMALSPIVNTREERTKTRGRVEVLNCLIPPYADDLSVTSAALRQAYPHDAIAFAERTYLGE

SEQ ID NO: 57 - protein, 512 aa, Hypocrea atroviridis
  REGION 1..512: note = Hypocrea atroviridis (strain ATCC 20476)
MSSTTYKSEAFDPEPPHLSFRSFVEALRQDNDLVDINEPVDPDLEAAAITRLVCETDDKAPLFNNVIGAKDGLWRILGAPASLRSSPKERFGRLARHLALPPTASAKDILDKMLSANSIPPIEPVIVPTGPVKENSIEGENIDLEALPAPMVHQSDGGKYIQTYGMHVIQSPDGCWTNWSIARAMVSGKRTLAGLVISPQHIRKIQDQWRAIGQEEIPWALAFGVPPTAIMASSMPIPDGVSEAGYVGAIAGEPIKLVKCDTNNLYVPANSEIVLEGTLSTTKMAPEGPFGEMHGYVYPGESHPGPVYTVNKITYRNNAILPMSACGRLTDETQTMIGTLAAAEIRQLCQDAGLPITDAFAPFVGQATWVALKVDTKRLRAMKTNGKAFAKRVGDVVFTQKPGFTIHRLILVGDDIDVYDDKDVMWAFTTRCRPGTDEVFFDDVVGFQLIPYMSHGNAEAIKGGKVVSDALLTAEYTTGKDWESADFKNSYPKSIQDKVLNSWERLGFKKLD

SEQ ID NO: 58 - protein, 508 aa, Sphaerulina musiva
  REGION 1..508: note = Sphaerulina musiva (strain SO2202)
MSSSKQQHLSHANQELPHLNFRSFVQALKDDGDLIEIDDEIDPHLEAGAIIRRACETDGKAPLLNNLKGAKDGLWRILGAPASLRSDPSQKYGRVARHLALPPTATMKDILDKMLSAAHAEPIPPNIVESGPVKENKLVDGEFDLSTLPAPWLHQADGGKYIQTYGMHIVQSPDGKWTNWSIARAMVHDKNHLTGLVIEPQHIWQIHQQWKKVGKDVPWALAFGVPPAAIMAASMPIPDGVTEAGYIGAMTGSALDVVKCETNGMYVPANAEIVLEGTLSITETAPEGPFGEMHGYVFPGDTHPWPKYKVDAITYRNGAILPVSNCGRITDETHTLIGPLAAAQIRQLCQDAGLPITDAFAPFHTQVTWVALKVDIEKLGKMNTTPEAFRKQVGDLVFNHKAGYTIHRLVLCGSDIDVYEWDDIAFAFSTRCRPNKDETFYEDCQGFPLIPYMSHGTGSPIKGGKVISDALMPSEYRGQQDWQQASFKHSYPESLQKSVIERWASWGF

SEQ ID NO: 59 - protein, 498 aa, Penicillium roqueforti
MANIEPHLCFRSFVEALKADNDLVEIDTPIDPNLEAAAITRLVCETNDKAPLFNNIIGTEKGLFRILGAPASLRNSSKDRYGRLARHLALPPTASMRDILDKMLSAGTPIPPNIVSTGPCKENFLEESQIDLTKLPAPLIHQADGGKYIQTYGMHIVQSPDGSWTNWSIARAMVSDDKHLTGLVIEPQHLWQIHQMWKKEGRDAPWALAFGVPPAAIMASSMPIPDGVSEAGYVGSMTGSALDLVKCDTNDLYVPATSEIVFEGTLSITEKGPEGPFGEMHGYVFPGDVHLCPKYKVNRITYRNDPIMPMSSCGRLTDETHTMIGSLAAAVIRKICQQAGLPVNDAFAPFESQVTWVALRIDTAKLREMKTTPKEFSKKVGELIFNSKAGYTIHRLVLCGDDIDVYNGKDVMWAFSTRCRPNLDEIFFEDVPGFPLIPYMSHGNGSPVKGGKVVSDALLPCEYTTGKNWEAADFESSYPEAVKQKVLANWTKMGFREE

SEQ ID NO: 60 - protein, 503 aa, Fusarium oxysporum
  REGION 1..503: note = Fusarium oxysporum f. sp. lycopersici
MPSKTLPHMDFRSYVEALEADGDLVSITEECDPHLEVGAIIRKVVENNDKAPLFNKLKGQDENGFWRILGAPNSLRSDPKQRYGRLARHLGLPTDSSMKVILDKMIAAKTTPPIPPTVVETGPCKEHILTPDQFDLTKLPAPLLHQSDGGKYIQTYGMHIVQSPDGKWTNWSIARAMVYDRNHLAGLVIKPQHLYQIHEMWKKEGRDMPWALAFGVPPAAIMASSMPLPDGLSEAEYIGSLVGSSLDVIKCETNGLYVPANSEIVFEGTCSITETAPEGPFGEMHGYVFPGDAHPWPKYTVDLITHRKDAILPVSNCGRLTDETHTMIGPLAAAEIGFLLKSKGLPIKEAFSPFESQVTWVALQVDTQKLREMKTTSEKFCREIGDIIFNHKVGYTIHRLVIVGDDINVYDFKDVIWAFCTRCRPGTDEYFFEDVAGFPLIPYMSHGNGAPNRGGKVVSDSLLPVEYTTGKNWEAADFENSFPEEIKDRVCSRWQTLGFSSAK

SEQ ID NO: 61 - protein, 503 aa, Saccharomyces kudriavzevii
  REGION 1..503: note = Saccharomyces kudriavzevii (strain ATCC MYA-4449)
MSALNPALQFRDFIQVLKDEDDLIEITKEVDPNLEVGAIMRKAYESKLPAPFFKNIKGASKDLFNILGCPAGLRNKKKGDHGRIAHHLGLDPKTTIKEIIDYLLECKNKKPLPPSSISASSAPCKAHVLSEEEIHLESLPTPYLHTSDGGNYLQTYGMWILQTPDKKWTNWSIARGMVVDDKHITGLVIKPQHIRQIADAWGAIGKGNKIPFALCFGVPPAAILVSSMPIPEGVSESDYVGAILGKPVPVVKCETNDLMVPATSEIVFEGTLSLTDTHAEGPFGEMHGYVFGGQGHPCPLYTVKAMTHRDNAILPVSNPGLCTDETHTLIGSLVATEAKELAIKSGLPVLDAFTPYEAQALWLVLKVDLKRLQALKTTPEEFSKKVGDIYFRTKVGFIIHEIVLVADDIDIFNFKEVFWAYVTRHTPVADQTAFDDVTSFPLAPFVSQSPRSKTMKGGKCVTNCIFRQQYERDFDYVTCSFEKGYSKELVDRINENWREYGYK

SEQ ID NO: 62 - protein, 503 aa, Saccharomyces cerevisiae; identical to SEQ ID NO: 48
  REGION 1..503: note = Saccharomyces cerevisiae S288c

SEQ ID NO: 63 - protein, 503 aa, Aspergillus parasiticus
MAAISEVDHSFRAFVEALKADDDLVEINTEIDSNLEAAAITRLVCETDDKAPLFNNLKGMGKNGLFRILGAPGSLRKSKRDRYGRLARHLALPPTASMKEILDKMLSASQLPPIDPKIVETGPVKENSLEGDEIDLTALPVPMVHKSDGGKYLQTYGMHIVQSPDGKWTNWSIARAMVKDKNHLTGLVIEPQHIWQIHQMWKKEGKDVPWALCFGVPPAAIMASSMPIPDGVTEAGYVGAMTGRALELVKCDTNHLYVPANAEIVLEGTLSITETADEGPFGEMHGYVFPGDSHKCPVYKVNKITYRTDAILPMSACGRLTDETHTMIGSLAAAEIRKICQLAGLPITDTFSPFEAQVTWVALKVDTAKLRQMNLTPKELQKWVGDVVFNHKAGYTIHRLVLVGDDIDPYEWKDVMWAFATRCRPNADEMFFEDVRGFPLIPYMGHGTGSPTKGGKVVSDALMPTEYTTGADWEAADFEHSYPEEIKAKVRAQWQALGFRKQE

SEQ ID NO: 64 - protein, 513 aa, Candida albicans
MSLNPALKFRDFIQVLKNEGDLVEIDTEVDPNLEVGAITRKAYENKLAAPLFNNLKQDPGNVDPKNLFRILGCPGGLRGFGNDHARIALHLGLDSQTPMKEIVDFLVANRNPKKFIPPVLVPNEKSPHKKHHLTHEQIDLTKLPVPLLHHGDGGKFIQTYGMWVLQTPDKSWTNWSIARGMVHDSKSITGLVINPQHVKQVSDAWVAAGKGDKIPFALCFGVPPAAILVSSMPIPDGATEAEYIGGLCNQAVPVVKCETNDLEVPADCEMVFEGYLDRDTLVTEGPFGEMHGYCFPQDHHTQPLYRVNHISYRDEAIMPISNPGLCTDETHTLIGGLVSAETKYLISQHLVLSKIVEDVFTPYEAQALWLAVKINIQELIKLKTNAKELSNLVGDFLFKSKECYKVCSILHEVILVGDDIDIFDFKQLIWAYTTRHTPVQDQVYFDDVKPFPLAPFISQGSLIKTRQGGKCVTSCIFPKQFTDPDFKFVTCNFDGYPEEVKNKVSQNWEKYYK

SEQ ID NO: 65 - protein, 500 aa, Grosmannia clavigera
  REGION 1..500: note = Grosmannia clavigera kw1407
MASSQDLPHMSFRAFVDELRADGDIVEINDECDADLEVGAIIRLACETDAKAPLFNKLKGMDGNGLWRILGAPNSLRADPAQRFGRLARHINLPPTASMKEILDKMGAAKSTPPIPPKTVPTGSCKEVKLTPDQFDLTTLPSPQLHKSDGGKYVQTYGMHIVQTPDGKWTNWSIARAMVHDRNHLVGLVIPPQHIWKVQQEWKKIGKDMPWALVFGVPPAAIMAASMPLPDGLSEAEYIGSLVGTALEVTKCDTNDLLVPANSEIVFEGFMSSTETAPEGPFGEMHGYVFPGDAHPQPLYTVNMITHRKDAILPVSNCGRLTDETHTMIGPLVAVEINVMLKAAGLPITDAYTPFESQVTWCAVKVDTAKLRELKTTPKEFCRKIGDLIFNTKVGSTIHRIAVVGDDIDIFNFKDVIWAFCTRCRPGMDEYLFEDVPGFPLIPYMSHGNGPANRGGKVVSDCLLPKEYTTGKNWEAASFKESIPESVQAKVLGNWKAWGF

SEQ ID NO: 66 - protein, 490 aa, Aspergillus terreus
MTKQSADSNAKSGVTSEICHWASNLATDDIPSDVLERAKYLILDGIACAWVGARVPWSEKYVQATMSFEPPGACRVIGYGQKLGPVAAAMTNSAFIQATELDDYHSEAPLHSASIVLPAVFAASEVLAEQGKTISGIDVILAAIVGFESGPRIGKAIYGSDLLNNGWHCGAVYGAPAGALATGKLLGLTPDSMEDALGIACTQACGLMSAQYGGMVKRVQHGFAARNGLLGGLLAHGGYEAMKGVLERSYGGFLKMFTKGNGREPPYKEEEVVAGLGSFWHTFTIRIKLYACCGLVHGPVEAIENLQGRYPELLNRANLSNIRHVHVQLSTASNSHCGWIPEERPISSIAGQMSVAYILAVQLVDQQCLLSQFSEFDDNLERPEVWDLARKVTSSQSEEFDQDGNCLSAGRVRIEFNDGSSITESVEKPLGVKEPMPNERILHKYRTLAGSVTDESRVKEIEDLVLGLDRLTDISPLLELLNCPVKSPLV

SEQ ID NO: 67 - protein, 649 aa, Pseudomonas amygdali
  REGION 1..649: note = Pseudomonas amygdali pv. tabaci str. ATCC 11528
MSTPELTTLLIANRGEIACRIMRTAKTMGLTTVAVHSAIDRDARHSREADIRVDLGGSKAAESYLAIDKLIDAARASGAQAIHPGYGFLSENADFARAIEEAGLIFLGPPASAIDAMGSKSAAKSLMEQAGVPLVPGYHGDAQDIETFRSAAERIGYPVLLKATAGGGGKGMKVVEHSGELAEALASAQREALSSFGDARMLVEKYVLTPRHVEIQVFADRHGHCLYLNERDCSIQRRHQKVVEEAPAPGLTPELRKAMGEAAVKAAQAIGYVGAGTVEFLLDARGEFFFMEMNTRLQVEHPVTEYITGLDLVEWQIRVARGEPLPITQEQVPLIGHAIEVRLYAEDPANDFLPATGTLELYRESASGPGKRVDSGVSEGDNISPFYDPMLGKLIAWGENREQARLRLLAMLDEFAVGGVRTNLAFLRRIIAHPAFAAAELDTGFIPRYQDKLLPQTGELCEELWQAAAEAFSQSEPARVDQADLHSPWAVTAGFRAGLPAERDLRLSCNGQTRTVYLRNSSDSPFKLSNEHLTVEHNGVRRSHLAIRRGGTLYLKWQGDLHTITRLDPIAQADVSDSQHGGLTAPMNGSIVRVLVEVGQAVESGAQLMVLEAMKMEHSIRAASAGVVTALYCHEGEMVNEGAVLVELT

SEQ ID NO: 68 - protein, 325 aa, Aspergillus clavatus
MAKIDVHHHFYPQAMREALERAGGDPSGWYIPPWTLDLDKEISRVLKVQTTILSVTAPGPGIETDPGKAAALARLCNEEAAAIRDAHPLQYGFFASVPSLFDTAAVLAEIEHAFTNLHADGVTLYTRYGAGHSYLGDERFRPVWAELSKRRAVVFIHPTHAVDTQLINSWMPQPMFDYPHETGRTAMDLLTRGVIRDYPGCKIILSHAGGTLPYLIHRAATMLPFMPRNLGMSREEIVEAARTLYFDTAISANPVTLKALLEFAKPGHVLFGSDFPNAPRGAITHFTSFLEGYDNMSEETRRLVEREAALELFPRLRGQSTRACL

SEQ ID NO: 69 - protein, 267 aa, Geobacillus stearothermophilus
MALTVKVSLYRKFAELLNEAEREKREVARITEEVPDLSAEEAYKIQEELIKIKTNSGHRIIGPKMGLTSQAKMAQMKVKEPIYGYLFDYMFVPSGGAIHMSELIHPKVEVEIAFILGEDLEGPHVTSTQVLSATKYVAPALEIIDSRYQDFTFTLPDVIADNASSSRVVIGNTMTPIHSLKTDLDLIGAALYINGELKACGAGAAVFNHPANSVAVLANMLARKGERLKAGDIILTGGITEAIQLSAGDTVIGQLDQLGDVSLSVKE

SEQ ID NO: 70 - protein, 429 aa, Salmonella enterica
  REGION 1..429: note = Salmonella enterica subsp. enterica serovar Dublin
MKGTVFAVALNHRSQLDAWQEAFSQPPYNAPPKTAVWFIKPRNTVIRHGEPILYPQGEKVLSGATVALIVGKTASRKRSEAAAEYIAGYALANEVSLPEESFYRPAIKAKCRDGFCPLGEMAPLSDVDNLTIITEINGREADHWNTADLQRSAAQLLSALSEFATLNPGDAILLGTPQNRVALRPGDRVRILAKGLPALENPVVAEDEFARNQTFTWPLSATGTLFALGLNYADHASELAFTPPKEPLVFIKAPNTFTEHHQTSVRPNNVEYMHYEAELVVVIGKTARKVSEAEAMEYVAGYTVCNDYAIRDYLENYYRPNLRVKSRDGLTPIGPWIVDKEAVSDPHNLTLRTFVNGELRQEGTTADLIFSIPFLISYLSEFMTLQPGDMIATGTPKGLSDVVPGDEVVLEIKGVGRLVNQIVCEESAN

SEQ ID NO: 71 - protein, 891 aa, Escherichia coli
  REGION 1..891: note = Escherichia coli K-12
MSSTLREASKDTLQAKDKTYHYYSLPLAAKSLGDITRLPKSLKVLLENLLRWQDGNSVTEEDIHALAGWLKNAHADREIAYRPARVLMQDFTGVPAVVDLAAMREAVKRLGGDTAKVNPLSPVDLVIDHSVTVDRFGDDEAFEENVRLEMERNHERYVFLKWGKQAFSRFSVVPPGTGICHQVNLEYLGKAVWSELQDGEWIAYPDTLVGTDSHTTMINGLGVLGWGVGGIEAEAAMLGQPVSMLIPDVVGFKLTGKLREGITATDLVLTVTQMLRKHGVVGKFVEFYGDGLDSLPLADRATIANMSPEYGATCGFFPIDAVTLDYMRLSGRSEDQVELVEKYAKAQGMWRNPGDEPIFTSTLELDMNDVEASLAGPKRPQDRVALPDVPKAFAASNELEVNATHKDRQPVDYVMNGHQYQLPDGAVVIAAITSCTNTSNPSVLMAAGLLAKKAVTLGLKRQPWVKASLAPGSKVVSDYLAKAKLTPYLDELGFNLVGYGCTTCIGNSGPLPDPIETAIKKSDLTVGAVLSGNRNFEGRIHPLVKTNWLASPPLVVAYALAGNMNINLASEPIGHDRKGDPVYLKDIWPSAQEIARAVEQVSTEMFRKEYAEVFEGTAEWKGINVTRSDTYGWQEDSTYIRLSPFFDEMQATPAPVEDIHGARILAMLGDSVTTDHISPAGSIKPDSPAGRYLQGRGVERKDFNSYGSRRGNHEVMMRGTFANIRIRNEMVPGVEGGMTRHLPDSDVVSIYDAAMRYKQEQTPLAVIAGKEYGSGSSRDWAAKGPRLLGIRVVIAESFERIHRSNLIGMGILPLEFPQGVTRKTLGLTGEEKIDIGDLQNLQPGATVPVTLTRADGSQEVVPCRCRIDTATELTYYQNDGILHYVIRNMLK

SEQ ID NO: 72 - protein, 467 aa, Escherichia coli
  REGION 1..467: note = Escherichia coli K-12
MNTVRSEKDSMGAIDVPADKLWGAQTQRSLEHFRISTEKMPTSLIHALALTKRAAAKVNEDLGLLSEEKASAIRQAADEVLAGQHDDEFPLAIWQTGSGTQSNMNMNEVLANRASELLGGVRGMERKVHPNDDVNKSQSSNDVFPTAMHVAALLALRKQLIPQLKTLTQTLNEKSRAFADIVKIGRTHLQDATPLTLGQEISGWVAMLEHNLKHIEYSLPHVAELALGGTAVGTGLNTHPEYARRVADELAVITCAPFVTAPNKFEALATCDALVQAHGALKGLAASLMKIANDVRWLASGPRCGIGEISIPENEPGSSIMPGKVNPTQCEALTMLCCQVMGNDVAINMGGASGNFELNVFRPMVIHNFLQSVRLLADGMESFNKHCAVGIEPNRERINQLLNESLMLVTALNTHIGYDKAAEIAKKAHKEGLTLKAAALALGYLSEAEFDSWVRPEQMVGSMKAGR

SEQ ID NO: 73 - protein, 299 aa, Bacillus subtilis
  REGION 1..299: note = Bacillus subtilis subsp. subtilis str. 168
MKLKDLIGKASIHKNKTIAVAHAEDEEVIRAVKLAAEHLSARFLLTGDSKKLNELTSSMQGHQVEIVHANTPEESAKLAVRAVHHKTADVLMKGNVPTSVLLKAVLNRQEGLRSASVLSHVAVFDIPDFDRLMFVTDSAMNIAPSLEELRQILQNAVHVAHAVGNNMPKAAALAAVETVNPKMEATVNAAALAQMYKRGQIKGCIVDGPLALDNAVSQIAAAQKKISGDVAGNADILLVPTIEAGNILYKSLIYFAKASVAAVITGAKAPIALTSRADSAENKLYSIALAICASEEYTH

SEQ ID NO: 74 - protein, 273 aa, Enterococcus faecalis
MITVSIAGGSQPEILQLVKKALKEAEQPLQFIVFDTNENLDTENLWKYVHCSDEATVAQEAVSLVATGQAQILLKGIIQTHTLLKEMLKSEHQLKNKPILSHVAMVELPAGKTFLLTDCAMNIAPTQATLIEIVENAKEVAQKLGLHHPKIALLSAAENFNPKMPSSVLAKEVTAHFNGQQEATVFGPLSLDLATSEEAVAHKRYSGPIMGDADILVVPTIDVGNCLYKSLTLFGHAKVGGTIVGTKVPVVLTSRSDSTESKFHSLRFAMRQV

SEQ ID NO: 75 - protein, 384 aa, Lactobacillus casei
  REGION 1..384: note = Lactobacillus casei W56
MRDCTTERRCLMTMHPKRDVVIVINPGSTSSKIALFKAGKMVAERTLNHSLAELSQFDSVIAQKDFRMQAIQEFLADQDFSASEVLAVAGRGGLLKPIPGGTYAVNEAMLDDLTAAKRNEHASNLGAGLAQQVADQYGVKAYVVDPPVVDELQPLARISGLKGIERHSAAHVLNQKAMARQVLATMGKTYATSRVIVAHIGGGLSIHAHENGRMIDGNNGIDGEGPYSPERAGSLPLVDFVAKVLAERLTLDQVKKLLASQSGLRSYLNDISIKNIVTRIAEGDETAKFYLDGMIYQIKKQIAEMAGVLNGQVDVIILTGGAAYATAVTVPLQHDLAWIAPVVVRPGEMEMQALYEGVMRVLNHEEPVRVYQSDASTIKGGTGR

SEQ ID NO: 76 - protein, 370 aa, unidentified
  REGION 1..370: note = Geobacillus sp. GHH01
MEEQKFRILTINPGSTSTKIGVFENERPLLEKTIRHEADVLRQYKTIADQYEFRKQTILQALDEEGINLSKLSAVCGRGGLLRPIEGGTYRVNEAMLEDLRRGYSGQHASNLGGILAHEIASALNIPAFIVDPVVVDELDPIARISGFPLIERRSIFHALNQKAVARRVAKQLGKRYDELNLIVAHMGGGITVGAHKQGRVVDVNNGLDGEGPFSPERAGTVPAGDLVALCFSGEYYREEIMNMLVGGGGLVGYLGTNDAVKVENMIEAGDEKAKLVYEAMAYQVAKEIGAASAVLSGKVDAIILTGGLAYGKSFVEQITRRVQWIADVIVHPGENELQALAEGALRVLRGEEEEKVYPGEAVSPIPARR

SEQ ID NO: 77 - protein, 556 aa, Methanobacterium formicicum
  REGION 1..556: note = Methanobacterium formicicum DSM 3637
MSSLLEKFVSQVNFESYPDFKDNFRIKIPENFNFAYDVVDEYARLYPEKVAMVWCNDDTDRIFTFKTLKEYSDRAANFFAQQGIKKGDRVMLTLKSRYEFWFCILALHKLGAITIPATHMLKTRDIVYRIKNAGIKMVVCIAEDGVPGYFDEAHLQLDDAPFVKALVGDEDREGWFNFRKELENASPELQRPSGEEGTQNDDVALIYFSSGTTGLPKMIMHDYTYPLGHIITAKYWQNVVEDGLHYTVADTGWAKAMWGQIYGQWISGTAIFVYDYERFDAAKMLDKASHHGVTTFCAPPTIYRFLIKEDLSQYDFSTLKYAVTAGEPLNPEVYNKFYEFTGLRLREGFGQTECVVCIANFPWIEPRPGSMGKSAPEYDIQIMDKEGKQCDVGEEGEIVIKTADGKPPGLFCGYYKEDNKTEAAWFDGYYHTGDTAWKDEDGYLWFVGRNDDMIKSSGYRIGPFEVESAVISHQAVLECAITGVPHPVRGQVIKATIVLTGDYEPSPELAKEIQNHVKQVTAPYKYPRVVEFVDELPKTISGKIRRVEIREKDEKE

SEQ ID NO: 78 - protein, 502 aa, Klebsiella pneumoniae
MTAPIQDLRDAIALLQQHDNQYLETDHPVDPNAELAGVYRHIGAGGTVKRPTRIGPAMMFNNIKGYPHSRILVGMHASRQRAALLLGCEASQLALEVGKAVKKPVAPVVVPASSAPCQEQIFLADDPDFDLRTLLPAPTNTPIDAGPFFCLGLALASDPVDASLTDVTIHRLCVQGRDELSMFLAAGRHIEVFRQKAEAAGKPLPITINMGLDPAIYIGACFEAPTTPFGYNELGVAGALRQRPVELVQGVSVPEKAIARAEIVIEGELLPGVRVREDQHTNSGHAMPEFPGYCGGANPSLPVIKVKAVTMRNNAILQTLVGPGEEHTTLAGLPTEASIWNAVEAAIPGFLQNVYAHTAGGGKFLGILQVKKRQPADEGRQGQAALLALATYSELKNIILVDEDVDIFDSDDILWAMTTRMQGDVSITTIPGIRGHQLDPSQTPEYSPSIRGNGISCKTIFDCTVPWALKSHFERAPFADVDPRPFAPEYFARLEKNQGSAK

SEQ ID NO: 79 - protein, 496 aa, Enterobacter cloacae
MENQINDLRSAIALLQRHEGQYIETDRPVDPNAELAGVYRHIGAGGTVKRPTRTGPAMMFNSIKGYPHSRILVGMHASRERAALLLGCEPSELAKHVGQAVKKPVAPVVVPASQAPCQEQVFYADDPDFDLRKLLPAPTNTPIDAGPFFCLGLVLASDPEDSSLTDVTIHRLCVQERDELSMFLAAGRHIEVFRKKAEDAGKPLPVTINMGLDPAIYIGACFEAPTTPFGYNELGVAGALRQQPVELVQGVAVKEKAIARAEIIIEGELLPGVRVREDQHTNTGHAMPEFPGYCGEANPSLPVIKVKAVTMRNHAILQTLVGPGEEHTTLAGLPTEASIRNAVEEAIPGFLQNVYAHTAGGGKFLGILQVKKRQPSDEGRQGQAALIALATYSELKNIILVDEDVDIFDSDDILWAMTTRMQGDVSITNIPGIRGHQLDPSQSPDYSTSIRGNGISCKTIFDCTVPWALKDRFERAPFMEVDPRPWAPELFADNTK

SEQ ID NO: 80 - protein, 510 aa, unidentified
  REGION 1..510: note = Leptolyngbya species
MLIDQEQAKTDHPLGWNVPDINDLRAAIAHLKKFKGQYIETDHPVDPIAELAGVYRYIGAGGTVMRPTRIGPAMTFNNVKGYPNSRVLVGMMASRERVSILLGAPTRELGMQMGKAVKTIVPPATIDAKDAPCQEEIYRADDPTFDLRKLLPAPTNTEEDAGPYFCMGLVLGSDPDDETNTDVTIHRLCVQSRDEMSIFFAPGRHIDAYRQKAEAAGKPLPISVNMGLDPAIHIGACFEAPTTPFGFDELCVAGGLRGKPVELVNCVTVQQKAIARAEIVIEGEVLPNVRVAEDQNTHTGYAMPEFPGYTGPANPSLPVIKVTAVTMRHNAILQTLVGPGEEHVNLAGIPTEASIYNAVELALPGLLQNVYSHSSGGGKFLAILQIKKRVAGDDGSARQAALIALAVYREVKNIILVDEDVDLFDSDDVLWAMQTRYQGDTGTIVVPGITGHVLDPSQIPEYSPSIHTKGSTCKTIFDCTVPFALKEHFKRAQFRELDPRPFAPELFNEP

SEQ ID NO: 81 - protein, 492 aa, unidentified
  REGION 1..492: note = Phascolarctobacterium species
MTTKINDLRSALDYLRTIPGQLVETNVEADPRAEISGIYRYVGAKGTVKRPTRLGPAMIFNNVKGHPGAKVAIGVLSSRARVGYLLGCEPEKLGFLLKDSVSTPIAPVVVSADQAPCQEVVHLATEEGFDIRKLIPAPTNTEEDAGPYVTMGLCYGTDPETGDTDITIHRLCLQGKDEISMYFVPGRHLDVFRQKYEKAGKPMPISISIGVDPAIEIAACFEPPTTPLGFNELSIAGSIRGEGVQMVQCKTINEKAIARAEYVIEGELLPDVRVREDQNSNTGKAMPEFPGYTGAMKPAIPLIKVKAVTHRRDPIMQSCIGPSEEHVNMAGIPTEASILGMTEKALPGNVKNVYAHCSGGGKYMAVIQFVKKAPPDEGRQRQAALLAFSAFSELKHVILVDDDVDLFDTDDVLWALNTRFQGDVDVITIPGVRCHPLDPSQSPEFSPSIRDVGISCKTIFDCTVPFGLKEHFQRSKFKEVNPAKWVPELFKK

SEQ ID NO: 82 - protein, 136 aa, Escherichia coli
  REGION 1..136: note = Escherichia coli K-12
MIWKRKITLEALNAMGEGNMVGFLDIRFEHIGDDTLEATMPVDSRTKQPFGLLHGGASVVLAESIGSVAGYLCTEGEQKVVGLEINANHVRSAREGRVRGVCKPLHLGSRHQVWQIEIFDEKGRLCCSSRLTTAIL

SEQ ID NO: 83 - protein, 136 aa, Salmonella enterica
MIWKREVTLDALNAMGEGNMVGLLDIRFERIGDDTLEATMPVDHRTKQPFGLLHGGASVVLAESIGSVAGYLCTQGEQKVVGLEVNANHVRSARQGRVRGVCKALHTGARHQVWQIEIFDEQGRLCCSSRLTTAIV

SEQ ID NO: 84 - protein, 517 aa, Megasphaera sp.
MRKVEIITAEQAAQLVKDNDTITSIGFVSSAHPEALTKALEKRFLDTNTPQNLTYIYAGSQGKRDGRAAEHLAHTGLLKRAIIGHWQTVPAIGKLAVENKIEAYNFSQGTLVHWFRALAGHKLGVFTDIGLETFLDPRQLGGKLNDVTKEDLVKLIEVDGHEQLFYPTFPVNVAFLRGTYADESGNITMDEEIGPFESTSVAQAVHNCGGKVVVQVKDVVAHGSLDPRMVKIPGIYVDYVVVAAPEDHQQTYDCEYDPSLSGEHRAPEGAADAALPMSAKKIIGRRGALELTENAVVNLGVGAPEYVASVAGEEGIADTITLTVEGGAIGGVPQGGARFGSSRNADAIIDHTYQFDFYDGGGLDIAYLGLAQCDGSGNINVSKFGTNVAGCGGFPNISQQTPNVYFCGTFTAGGLKIAVEDGKVKILQEGKAKKFIKAVDQITFNGSYAARNGKHVLYITERCVFELTKEGLKLIEVAPGIDIEKDILAHMDFKPIIDNPKLMDARLFQDGPMGLKK

SEQ ID NO: 85 - protein, 265 aa, unidentified
  REGION 1..265: note = Pseudomonas species
MNNLPVCQTLLLELHNGVLHVTLNRPECRNAMSSQMVAELRSVLAAVRDKPGVRALVIGGVGGHFCAGGDIKDMANARAQGPTAHRDLNRVFGALLQEVQHAPQVVITVLQGAVLGGGLGLACVSDIALADHQAQFGLPETSLGLLPAQIAPFVVQRIGLTEARRLALTAARFDGHQARRMGLVHFVEHDPQALAERLDEVLAHVLCCAPGANAATKKLLLASAGQPSDELLDQAAEWFSEAVTGAEGVEGTMAFVQKRKPGWAS

SEQ ID NO: 86 - protein, 277 aa, Acinetobacter baumannii
MTLSASLHIDIDDSIQLEQDGSILYLWLNRPESRNAMNLNMVNAIQQVFTAIRDDLSIRAVIIRGEGGTFCAGGDIKDMAALRVEATNVGSLQPYTNFNRRFGAMLEQVEAAPQTVVVILESAVLGGGFGLACVSDVAISRDNAQFGLPETGLGVIPAQIAPFVVKRIGLTQARRLALLGMRFEGHTALSVGVVHQIAHNEIELEQALQETIQQIKRAAPQASRVTKALLHRTLNEPLNQLLDDAAQQFAQAVGSAEGQEGTMAFIQKRLPNWADET

SEQ ID NO: 87 - protein, 264 aa, Pseudomonas aeruginosa
MSLPHCETLLLEPIEGVLRITLNRPQSRNAMSLAMVGELRAVLAAVRDDRSVRALVLRGADGHFCAGGDIKDMAGARAAGAEAYRTLNRAFGSLLEEAQAAPQLLVALVEGAVLGGGFGLACVSDVAIAAADAQFGLPETSLGILPAQIAPFVVRRIGLTQARRLALTAARFDGREALRLGLVHFCEADADALEQRLEETLEQLRRCAPNANAATKALLLASESGELGALLDDAARQFAEAVGGAEGSEGTLAFVQKRKPVWAQ

SEQ ID NO: 88 - protein, 273 aa, Marinobacter santoriniensis
MEQLPHCETLLLEKHGPALFLTINRPDVRNAMSLQMVAELSTIFNQIEQDNTIRAVVIRGKDGHFCAGGDIKDMAGARGQKADEGQHDPFYKLNRAFGHMIQQVNESSKVVIAVTEGAVMGGGFGLACVSDVAIAGPTARFGMPETSLGVIPAQIAPFVVERIGLTQARRLALLGLRIDAREACALGIVHQAADSETQLEELLQATLERVRLCAPNATAETKALLHRVGHEPMNKLLDSAAETFAEAIRGPEGAEGTMAFMQKREPKWADDSN

SEQ ID NO: 89 - protein, 265 aa, Pseudomonas knackmussii
MSELPNCETLLLERDGGVLHVTLNRPDSRNAMSLAMVGELRAVLAAVRDDRAVRAIVLRGAGGHFCAGGDIKDMAGARAAGTDAYAKLNRAFGSLLEEAQAQPQVLVAVLEGAVLGGGFGLACVSDIAIAADGAQFGLPETTLGILPAQIAPFVAKRVGLTQARRLALTAARFDGREALRLGLVHFSEADADALGQRLADCLEQVRRCAPGANAATKALLLATEREELGSLLDGAARQFAEAVTGSEGAEGTMAFVQKRKPNWAQ

SEQ ID NO: 90 - protein, 264 aa, Pseudomonas pseudoalcaligenes
MELPKTETLLLEHADGLLRITLNRPESRNAMSLAMVEELRAVLAAARRAPEVRVLALRGAGGHFCAGGDIKDMASARATGGEAYQRLNRAFGRLLEEAQAQPQVVIAVLEGAVLGGGFGLACVSDIALAAESAQFGLPETSLGILPAQIAPFVVKRVGLTQARRLALTAARFDGTEALRLGLVHFTEADDAALDARLAATLDQVRRCAPGANARTKALLLATEERELGPLLDDAAAWFAEAVTSAEGTEGTLAFVQKRKPTWAQ

SEQ ID NO: 91 - protein, 264 aa, Pseudomonas flexibilis
MADLPHCDTLLLNLDAGVLHITLNRPDSRNAMSLAMVHELRAVLESVRNDPAVRALVLRGAGGHFCAGGDIKDMAGARAKGHDAYRDLNRAFGALLEEAQAAPQVVVAVLEGAVLGGGFGLACVSDIAIAAEGCKFGLPETTLGILPAQIAPFVVKRVGLTQARRLALTAARFDGAEALRLGLVHYCEAADRLDSRLAEVIQQVRQCAPQANAQTKALLLASETEAMNSLLDRAAEQFAAAVTGAEGVEGTMAFVQKRAPKWAQ

SEQ ID NO: 92 - protein, 271 aa, Alcanivorax dieselolei
MTLPETETITLHRDGTTLSVTLNRPQSRNAMSLIMVDELMAVFDWVEANPDVRAVVLRGAGGHFCAGGDIKDMAGARQQAAAGDDQAFFTLNRRFGAMVSRAERLPAVLVCVLEGAVLGGGFGLACVSDVALAAGDARFGLPETGLGVIPAQIAPFVVRRIGLTQARRLALTGGRFDGHGAQALGVVHEVADSTEELEQRLRQVLEQIRRCAPHANRVTKQLVLSVDEQPLDAVLDQAARDFANAVTSEEGQEGTLAFVQKRAPSWSTDKE

SEQ ID NO: 93 - DNA, 3343 bp, synthetic construct
  source 1..3343: mol_type = other DNA
  misc_feature 1..3343: note = Plasmid name pGB 5796
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcggccgcggggttaattaatttctcctctttaataaagcaaataaattttttatgatttgtttaaacctaggcatgcctctagattattatgcgccctgccagcgggcaaagagatcttcaggaagggttatcgcaaactggtcaagaacacgattaaccgtctgatttatcacatcatcaagggattgcgggcgatgataaaacgccggaacgggaggcataatcaccgcaccgatttctgccgcctgagtcattaaacgcagatggcctaagtgcaatggtgtttcacgcacgcagagcaccaacgggcgacgctctttcagcaccacatctgccgcacgggtcagtaagccatcagtatagctatggacaatgccggaaagggttttgattgaacagggtaaaatcaccatccccagcgtctggaaagaaccggaagagatgctggcggcaatatcgcgcgcatcgtgcgtgacatcggctaatgcctgcacttcgcgcagagaaaaatccgtttcgagggataaggtctggcgcgctgcctggctcatcaccagatgcgtttcgatatctgtgacatcgcgcagaacctgtaataagcgcacgccataaatcgcgccgctggcaccgctgatgcctacaatgagtcgtttcataaaaaaaatgtatatctccttcggtaccgagctcgaacctgcaggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 94 - DNA, 12214 bp, synthetic construct
  source 1..12214: mol_type = other DNA
  misc_feature 1..12214: note = Plasmid name pGB 5771
ctcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgcttctcagggcgttttatggcgggtctgctatgtggtgctatctgactttttgctgttcagcagttcctgccctctgattttccagtctgaccctagtcaaggccttaagtgagtcgtattacggactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcccggggaactatagtttaaacttttcaatgaattcatttaagcggccgcatcaattctagaatttaaatagtcaaaagcctccgaccggaggcttttgactgacctattgacaattaaaggctaaaatgctataattccactaatagaaataattttgtttaactttaggtctctatcgtaagaaggagatatatgaaagaagtggtgattgccagcgcagttcgtaccgcaattggtagctatggtaaaagcctgaaagatgttccggcagttgatctgggtgcaaccgcaattaaagaagcagttaaaaaagccggtattaaaccggaagatgtgaacgaagttattctgggtaatgttctgcaagcaggtctgggtcagaatccggcacgtcaggcctcgtttaaagcaggtctgccggttgaaattccggcaatgaccattaacaaagtttgtggtagcggtctgcgtaccgttagcctggcagcacagattatcaaagccggtgatgcagatgttattattgccggtggtatggaaaatatgagccgtgcaccgtatctggcaaataatgcacgttggggttatcgtatgggtaatgccaaatttgtggatgagatgattaccgatggtctgtgggatgcctttaatgattatcacatgggtattaccgcagagaatattgcagaacgttggaatattagccgtgaagaacaggatgaatttgcactggcaagccagaaaaaagcagaagaagcaattaaaagcggtcagttcaaagatgaaattgtgccggttgttatcaaaggtcgtaaaggtgaaaccgttgttgataccgatgaacatccgcgttttggtagcaccattgaaggtctggcaaaactgaaaccggcattcaaaaaagatggcaccgttaccgcaggtaatgcaagcggtctgaatgattgtgcagcagttctggttattatgagcgcagaaaaagcaaaagaactgggtgttaaaccgctggcaaaaattgtgagctatggtagtgccggtgttgatccggcaattatgggttatggtccgttttatgcaaccaaagcagcaattgaaaaagcaggttggaccgttgatgaactggatctgattgaaagcaatgaagcatttgcagcacagagcctggcagttgcaaaagacctgaaattcgatatgaataaagtgaatgtgaatggcggtgcaattgccctgggtcatccgattggtgcaagcggtgcacgtattctggttaccctggttcatgcaatgcagaaacgtgatgcaaaaaaaggtctggccaccctgtgtattggtggtggtcagggcaccgcaattctgctggaaaaatgctaataagcttgaaggagatataatgaccattggtattgataaaatcagctttttcgtgcctccgtactatattgatatgaccgcactggccgaagcacgtaatgttgatccgggtaaatttcatattggtattggtcaggatcagatggccgttaatccgattagccaggatattgttacctttgcagcaaatgcagcagaagcaattctgaccaaagaagataaagaggccattgatatggttattgttggcaccgaaagcagcattgatgaaagcaaagcagcagcagttgttctgcatcgtctgatgggtattcagccgtttgcacgtagctttgaaattaaagaagcatgttacggagcaaccgcaggtctgcaactggcaaaaaatcatgttgcactgcatccggataaaaaagttctggttgttgcagcagatattgccaaatatggtctgaatagcggtggtgaaccgacccagggtgccggtgcagttgcaatgctggttgcaagcgaaccgcgtattctggcactgaaagaagataatgttatgctgacccaggatatttatgatttttggcgtccgaccggtcatccgtatccgatggttgatggtccgctgagcaatgaaacctatattcagagctttgcacaggtgtgggatgaacataaaaaacgtaccggtctggatttcgcagattatgatgcactggcatttcatatcccgtataccaaaatgggtaaaaaagcactgctggccaaaattagcgatcagaccgaagccgaacaagaacgcattctggcacgttatgaagaaagcattgtttatagccgtcgtgtgggtaatctgtataccggtagcctgtatctgggtctgattagcctgctggaaaatgcaaccaccctgaccgcaggtaatcagattggtctgtttagctatggtagcggtgccgttgcagaatttttcacaggtgaactggttgcaggttatcagaatcatctgcaaaaagaaacccatctggcactgctggataatcgtaccgaactgagcattgcagaatatgaagcaatgtttgcagaaaccctggataccgatattgatcagaccctggaagatgaactgaaatatagcattagcgccattaataacaccgtgcgtagctatcgtaactaataaggtagaaggagatatacatatgagtcaggcgctaaaaaatttactgacattgttaaatctggaaaaaattgaggaaggactctttcgcggccagagtgaagatttaggtttacgccaggtgtttggcggccaggtcgtgggtcaggccttgtatgctgcaaaagagacggtccctgaagaacggctggtacattcgtttcacagctactttcttcgccctggcgatagtaagaagccgattatttatgatgtcgaaacgctgcgtgacggtaacagcttcagcgcccgccgggttgctgctattcaaaacggcaaaccgattttttatatgactgcctctttccaggcaccagaagcgggtttcgaacatcaaaaaacaatgccgtccgcgccagcgcctgatggcctcccttcggaaacgcaaatcgcccaatcgctggcgcacctgctgccgccagtgctgaaagataaattcatctgcgatcgtccgctggaagtccgtccggtggagtttcataacccactgaaaggtcacgtcgcagaaccacatcgtcaggtgtggattcgcgcaaatggtagcgtgccggatgacctgcgcgttcatcagtatctgctcggttacgcttctgatcttaacttcctgccggtagctctacagccgcacggcatcggttttctcgaaccggggattcagattgccaccattgaccattccatgtggttccatcgcccgtttaatttgaatgaatggctgctgtatagcgtggagagcacctcggcgtccagcgcacgtggctttgtgcgcggtgagttttatacccaagacggcgtactggttgcctcgaccgttcaggaaggggtgatgcgtaatcacaattaataagaacgaaggagatataatgaaaaccgcacgttggtgtagcctggaagaagcagttgcaagcattccggatggtgcaagcctggcaaccggtggttttatgctgggtcgtgcaccgatggcactggttatggaactgattgcacagggtaaacgtgatctgggtctgattagcctgccgaatccgctgccagcagaatttctggttgccggtggttgtctggctcgtctggaaattgcatttggtgcactgagtctgcaaggtcgtgttcgtccgatgccgtgtctgaaacgtgcaatggaacagggcaccctggcatggcgtgaacatgatggttatcgtgttgttcagcgtctgcgtgcagcaagcatgggtctgccgtttattccggcaccggatgcagatgttagcggtctggcacgtaccgaaccgcctccgaccgttgaagatccgtttaccggtctgcgtgttgcagttgaaccggcattttatccggatgttgcactgctgcacgcacgtgcagccgatgaacgtggtaatctgtatatggaagatccgaccaccgatctgctggttgcgggtgcagcaaaacgtgttattgcaaccgttgaagaacgtgttgcaaaactgcctcgtgcaaccctgcctggttttcaggttgatcgtattgttctggcaccgggtggtgcactgccgaccggttgtgcaggtctgtatccgcatgatgatgaaatgctggcacgttatctgagcctggcagaaaccggtcgtgaagccgaatttctggaaaccctgctgacccgtcgtgcagcataatgaggatccgaaggagatatacatatgagcgcaaccctggatattacaccggcagaaaccgttgttagcctgctggcacgtcagattgatgatggtggtgttgttgcaaccggtgttgcaagtccgctggcaattctggccattgcagttgcacgtgccacccatgcaccggatctgacctatctggcatgtgttggtagcctggacccggaaattccgaccctgctgccgagcagcgaagacctgggttatctggatggtcgtagcgcagaaattaccattccggacctgtttgatcatgcacgtcgtggtcgtgttgataccgttttttttggtgcagccgaagttgatgccgaaggtcgtaccaatatgaccgcaagcggtagtctggataaaccgcgtaccaaatttccgggtgttgccggtgcagccaccctgcgtcagtgggttcgtcgtccggttctgctggttccgcgtcagagccgtcgtaatctggttccggaagttcaggttgcaaccacccgtgatccgcgtcgtccggtgaccctgattagcgatctgggtgtttttgaactgggtgcaagcggtgcacgtctgctggcacgccatccgtgggcaagcgaagaacatattgcagaacgtaccggttttgcatttcaggttagcgaagcactgagcgttaccagcctgccggatgcacgtaccgttgcagcaattcgtgcaattgatccgcatggctatcgtgatgcactggttggtgcataattagtcagaaggagatatacatatgagcctgccgcattgtgaaaccctgctgctggaaccgattgaaggtgttctgcgtattaccctgaatcgtccgcagagccgtaatgcaatgagcctggcaatggttggtgaactgcgtgcagttctggcagcagttcgtgatgatcgtagcgttcgtgcactggttctgcgtggtgcagatggtcatttttgtgccggtggtgatattaaagatatggcaggcgcacgtgcagccggtgcagaagcatatcgtacactgaatcgtgcatttggtagcctgctggaagaagcacaggcagcaccgcagctgctggttgcactggttgaaggtgccgttctgggtggtggttttggtctggcatgtgttagtgatgttgcaattgcagcagcagatgcacagtttggtctgccggaaaccagcctgggtattctgcctgcacagattgcaccgtttgttgttcgtcgtattggtctgacccaggcacgtcgtctggcactgaccgcagcacgttttgatggtcgtgaagcactgcgtctgggtctggttcatttttgtgaagcagatgcagatgcactggaacagcgtctggaagaaaccctggaacagctgcgtcgttgtgcaccgaatgcaaatgcagcaaccaaagcactgctgctggcaagcgaaagcggtgaactgggtgcactgctggatgatgcagcacgtcagtttgccgaagcagttggtggtgcagaaggtagcgaaggcaccctggcatttgttcagaaacgtaaaccggtttgggcacagtaataatgaaagagaccagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtcaccccatgcgagagtagggaactgccaggcatcaaataaaacgaaaggctcagtcgaaagactgggcctttcgttttatctgttgtttgtcggtgaactactagaatttaaatagtcaaaagcctccgaccggaggcttttgactgacctattgacaattaaaggctaaaatgctataattccactaatagaaataattttgtttaactttaggtctctatcgaccataattaattaactttaagaaggagatatacatatgagcagcaccacctataaaagcgaagcatttgatccggaaccgcctcatctgagctttcgtagctttgttgaagcactgcgtcaggataatgatctggtggatattaatgaaccggttgatccggatctggaagcagcagcaattacccgtctggtttgtgaaaccgatgataaagcaccgctgtttaataacgtgattggtgcaaaagatggtctgtggcgtattctgggtgcaccggcaagcctgcgtagcagcccgaaagaacgttttggtcgtctggcacgtcatctggcactgcctccgaccgcaagcgcaaaagatattctggataaaatgctgagcgccaatagcattccgcctattgaaccggttattgttccgaccggtccggttaaagaaaatagcattgaaggcgaaaacattgatctggaagccctgcctgcaccgatggttcatcagagtgatggtggcaagtatatccagacctatggtatgcatgttatccagagtccggatggttgttggaccaattggagcattgcccgtgcaatggttagcggtaaacgtaccctggcaggtctggttattagtccgcagcatattcgtaaaattcaggatcagtggcgtgcaattggtcaagaagaaattccttgggcactggcatttggtgttccgcctaccgcaattatggcaagcagtatgccgattccggatggtgttagcgaagcaggttatgttggtgcaattgccggtgaaccgattaaactggttaaatgcgataccaacaatctgtatgttccggcaaatagcgaaattgttctggaaggcaccctgagcaccaccaaaatggcaccggaaggtccgtttggtgaaatgcatggttatgtttatccgggtgaaagccatccgggtccggtttataccgttaacaaaattacctatcgcaacaatgcaattctgccgatgagcgcatgtggtcgtctgaccgatgaaacccagaccatgattggcaccctggcagcagcagaaattcgtcagctgtgtcaggatgcaggtctgccgattaccgatgcatttgcaccgtttgttggtcaggcaacctgggttgcactgaaagttgataccaaacgtctgcgtgcaatgaaaaccaatggtaaagcatttgcaaaacgtgttggtgatgttgtgtttacccagaaaccgggttttaccattcatcgtctgattctggttggtgatgatattgatgtgtatgacgataaagatgtgatgtgggcatttaccacccgttgtcgtccgggtacagatgaagttttttttgatgatgttgtgggctttcagctgatcccgtatatgagtcatggtaatgccgaagcaattaaaggtggtaaagttgttagtgatgcactgctgaccgcagaatataccaccggtaaagattgggaaagcgcagatttcaaaaacagctatccgaaaagcatccaggataaagttctgaatagctgggaacgcctgggtttcaaaaaactggattaataaccatggttataagagagaccagcctgactcctgttgatagatccagtaatgacctcagaactccatctggatttgttcagaacgctcggttgccgccgggcgttttttattggtgagaataactactagttggcgggcggccgcttagctctgcagatgagaaattcttgaagacgaaagggcctcgtgatacgcctatttttataggttaatgtcatgataataatggtttaagcttcttagaatagctcttctatgaggtggcacttttcggggaaagatatccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatcggtacctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccacgagctcctgtcagaccaagtttacgagctcgcttggactcctgttgatagatccagtaatgacctcagaactccatctggatttgttcagaacgctcggttgccgccgggcgttttttattggtgagaatccaagcactagggacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggacatgtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccataagcacagatgaaaacggtgtaaaaaagatagatacatcagagcttttacgagtttttggtgcatttaaagctgttcaccatgaacagatcgacaatgtaacgcatgcaccgagcgcagcgagtcagtgagcgaggaagcggaacagcgcctg

SEQ ID NO: 95 - protein, 274 aa, Ustilago maydis
MTASALAYLEPDSSAELTGVYHLVLDRPEARNAISRSLLQDVLQCLQVLVCKITQPKQDEPLPRVLILRANGPCFCAGADLKERREMSEAEVIEFLQDLRHMLEQVEKLPIPTLAAIDGPALGGGLELALACDFRIAAETVSKIGFPEVKLGIIPGAGGTQRAPRIIGMQRAKELIYTGTQLNATQAKDLGLIDHVAPGSTCLKLCQELAQQMMPSAPLALRAAKMAISMGANVELARGLDLEWACYEPLLESKDRREALDAFQQKRKPIFTGK

SEQ ID NO: 96 - protein, 262 aa, unidentified
  REGION 1..262: note = Bacillus sp. GeD10
MLQLQNISVDYVTPHVVKISLYRERQANSLSLALLEELQNILTQISEESNTRVVILTGAGEKAFCAGADLKERAGMNEEQVRHAVSMIRTTMEMVEQLPQPVIAAINGIALGGGTELSLACDFRIAAESASLGLTETTLAIIPGAGGTQRLPRLIGVGRAKELIYTGRRISAQEAKEYGLVEFVVPANLLEEKAIEIAEKIASNGPIAVRLAKEAISNGIQVDLHTGLQMEKQAYEGVIHTKDRLEGLQAFKEKRTPTYKGE

SEQ ID NO: 97 - protein, 263 aa, Labilithrix luteola
MADESFPVEVEQRGNVVIWTIDRESRMNSLSRATLFALGKLTREAVSNPSVRAIVITGRGEKAFCAGADLKERQGMTENDIRVQVELYRSELGPLDRSPKPVIAALNGVAFGGGLELALVCDMRVAASHALIGLPETTLGIIPGAGGTQRLPRIVGEARAKEMILLGRKLSATEAHAWGLVNRVTPEGANVVEDTLAFIDPIANGAPIAQAAALEAIDRSFDTTLELGLELEKVSYDKVLVSEDRREALAAFAEKRKPQFKGR

SEQ ID NO: 98 - protein, 258 aa, Myxococcus xanthus
MPEFKVDARGPIEIWTIDGESRRNAISRAMLKELGELVTRVSSSRDVRAVVITGAGDKAFCAGADLKERATMAEDEVRAFLDGLRRTFRAIEKSDCVFIAAINGAALGGGTELALACDLRVAAPAAELGLTEVKLGIIPGGGGTQRLARLVGPGRAKDLILTARRINAAEAFSVGLANRLAPEGHLLAVAYGLAESVVENAPIAVATAKHAIDEGTGLELDDALALELRKYEEILKTEDRLEGLRAFAEKRAPVYKGR

SEQ ID NO: 99 - protein, 383 aa, Enterococcus faecalis
MTIGIDKISFFVPPYYIDMTALAEARNVDPGKFHIGIGQDQMAVNPISQDIVTFAANAAEAILTKEDKEAIDMVIVGTESSIDESKAAAVVLHRLMGIQPFARSFEIKEACYGATAGLQLAKNHVALHPDKKVLVVAADIAKYGLNSGGEPTQGAGAVAMLVASEPRILALKEDNVMLTQDIYDFWRPTGHPYPMVDGPLSNETYIQSFAQVWDEHKKRTGLDFADYDALAFHIPYTKMGKKALLAKISDQTEAEQERILARYEESIIYSRRVGNLYTGSLYLGLISLLENATTLTAGNQIGLFSYGSGAVAEFFTGELVAGYQNHLQKETHLALLDNRTELSIAEYEAMFAETLDTDIDQTLEDELKYSISAINNTVRSYRN

SEQ ID NO: 100 - protein, 265 aa, Myxococcus xanthus
MKTARWCSLEEAVASIPDGASLATGGFMLGRAPMALVMELIAQGKRDLGLISLPNPLPAEFLVAGGCLARLEIAFGALSLQGRVRPMPCLKRAMEQGTLAWREHDGYRVVQRLRAASMGLPFIPAPDADVSGLARTEPPPTVEDPFTGLRVAVEPAFYPDVALLHARAADERGNLYMEDPTTDLLVAGAAKRVIATVEERVAKLPRATLPGFQVDRIVLAPGGALPTGCAGLYPHDDEMLARYLSLAETGREAEFLETLLTRRAA

SEQ ID NO: 101 - protein, 246 aa, Myxococcus xanthus
MSATLDITPAETVVSLLARQIDDGGVVATGVASPLAILAIAVARATHAPDLTYLACVGSLDPEIPTLLPSSEDLGYLDGRSAEITIPDLFDHARRGRVDTVFFGAAEVDAEGRTNMTASGSLDKPRTKFPGVAGAATLRQWVRRPVLLVPRQSRRNLVPEVQVATTRDPRRPVTLISDLGVFELGASGARLLARHPWASEEHIAERTGFAFQVSEALSVTSLPDARTVAAIRAIDPHGYRDALVGA

(19) United States
(12) Patent Application Publication
(10) Pub. No.: US 2026/0209804 A1
(43) Pub. Date: Jul. 23, 2026

(54) METHODS FOR PRODUCING ISOBUTENE FROM 3-METHYLCROTONIC ACID

(71) Applicants: Global Bioenergies, Evry Cedex (FR); Scientist of Fortune, S.A., Luxembourg (LU)

(72) Inventors: Mathieu Allard, Rigny-la-Nonneuse (FR); Maria Anissimova, Nozay (FR); Philippe Marlière, Luxembourg (LU)

(73) Assignees: Global Bioenergies, Evry Cedex (FR); Scientist of Fortune, S.A., Luxembourg (LU)

(21) Appl. No.: 19/311,993

(22) Filed: Aug. 27, 2025

Related U.S. Application Data
(62) Division of application No. 17/362,950, filed on Jun. 29, 2021.
Application No. 17/362,950 is a division of application No. 15/776,495, filed on Nov. 17, 2016, now Pat. No. 11,124,806.

(30) Foreign Application Priority Data
Nov. 17, 2015 (EP) 15194984.9

(51) Int. Cl. C12P 5/02; C12N 9/00; C12N 9/10; C12N 9/12; C12N 9/16; C12N 9/88; C12N 15/52; C12P 7/40

(52) U.S. Cl. CPC C12P 5/026; C12N 9/1029; C12N 9/1085; C12N 9/1217; C12N 9/13; C12N 9/16; C12N 9/88; C12N 9/93; C12N 15/52; C12P 7/40; C12Y 205/01; C12Y 207/02007; C12Y 207/02014; C12Y 207/02015; C12Y 208/03008; C12Y 301/02018; C12Y 301/0202; C12Y 401/01006; C12Y 401/01063; C12Y 604/01004; C12Y 604/01005; C12Y 208/03001; C12Y 301/02001

(57) ABSTRACT

Described are methods for the production of isobutene comprising the enzymatic conversion of 3-methylcrotonic acid into isobutene wherein said 3-methylcrotonic acid is obtained by the enzymatic conversion of 3-methylcrotonyl-CoA into 3-methylcrotonic acid or wherein said 3-methylcrotonic acid is obtained by the enzymatic conversion of 3-hydroxyisovalerate (HIV) into 3-methylcrotonic acid. It is described that the enzymatic conversion of 3-methylcrotonic acid into isobutene can, e.g., be achieved by making use of a 3-methylcrotonic acid decarboxylase, preferably an FMN-dependent decarboxylase associated with an FMN prenyl transferase, an aconitate decarboxylase (EC 4.1.1.6), a methylcrotonyl-CoA carboxylase (EC 6.4.1.4), or a geranoyl-CoA carboxylase (EC 6.4.1.5).

### CROSS-REFERENCE TO RELATED APPLICATIONS

This application is a Divisional Application of U.S. Ser. No. 17/362,950 filed on Jun. 29, 2021, which is a Divisional Application of U.S. Ser. No. 15/776,495 filed on May 16, 2018, issuing as U.S. Pat. No. 11,124,806 on Sep. 21, 2021, which is a § 371 National Stage Application of PCT/EP2016/077956 filed on Nov. 17, 2016, which claims priority to EP 15194984.9 filed on Nov. 17, 2015, all of which are hereby incorporated by reference in their entirety.

### REFERENCE TO ELECTRONIC SEQUENCE LISTING

The application contains a Sequence Listing which has been submitted electronically in.XML format and is hereby incorporated by reference in its entirety. Said .XML copy, created on Jul. 22, 2025, is named “GB-29-US-D2.xml” and is 11,074,658 bytes in size. The sequence listing contained in this .XML file is part of the specification and is hereby incorporated by reference herein in its entirety.

The present invention relates to methods for the production of isobutene comprising the enzymatic conversion of 3-methylcrotonic acid into isobutene wherein said 3-methylcrotonic acid is obtained by the enzymatic conversion of 3-methylcrotonyl-CoA into 3-methylcrotonic acid or wherein said 3-methylcrotonic acid is obtained by the enzymatic conversion of 3-hydroxyisovalerate (HIV) into 3-methylcrotonic acid. The enzymatic conversion of 3-methylcrotonic acid into isobutene can, e.g., be achieved by making use of a 3-methylcrotonic acid decarboxylase, preferably an FMN-dependent decarboxylase associated with an FMN prenyl transferase, an aconitate decarboxylase (EC 4.1.1.6), a methylcrotonyl-CoA carboxylase (EC 6.4.1.4), or a geranoyl-CoA carboxylase (EC 6.4.1.5). Further, said 3-methylcrotonyl-CoA can be obtained by the enzymatic conversion of 3-methylglutaconyl-CoA into 3-methylcrotonyl-CoA.

A large number of chemical compounds are currently derived from petrochemicals. Alkenes (such as ethylene, propylene, the different butenes, or else the pentenes, for example) are used in the plastics industry, for example for producing polypropylene or polyethylene, and in other areas of the chemical industry and that of fuels.

Butylene exists in four forms, one of which, isobutene (also referred to as isobutylene), enters into the composition of methyl-tert-butyl-ether (MTBE), an anti-knock additive for automobile fuel. Isobutene can also be used to produce isooctene, which in turn can be reduced to isooctane (2,2,4-trimethylpentane); the very high octane rating of isooctane makes it the best fuel for so-called “gasoline” engines. Alkenes such as isobutene are currently produced by catalytic cracking of petroleum products (or by a derivative of the Fischer-Tropsch process in the case of hexene, from coal or gas). The production costs are therefore tightly linked to the price of oil. Moreover, catalytic cracking is sometimes associated with considerable technical difficulties which increase process complexity and production costs.

The production by a biological pathway of alkenes such as isobutene is called for in the context of a sustainable industrial operation in harmony with geochemical cycles. The first generation of biofuels consisted in the fermentative production of ethanol, as fermentation and distillation processes already existed in the food processing industry. The production of second generation biofuels is in an exploratory phase, encompassing in particular the production of long chain alcohols (butanol and pentanol), terpenes, linear alkanes and fatty acids. Two recent reviews provide a general overview of research in this field: Ladygina et al. (Process Biochemistry 41 (2006), 1001) and Wackett (Current Opinions in Chemical Biology 21 (2008), 187).

The conversion of isovalerate to isobutene by the yeast Rhodotorula minuta has been described (Fujii et al. (Appl. Environ. Microbiol. 54 (1988), 583)), but the efficiency of this reaction, less than 1 millionth per minute, or about 1 for 1000 per day, is far from permitting an industrial application. The reaction mechanism was elucidated by Fukuda et al. (BBRC 201 (1994), 516) and involves a cytochrome P450 enzyme which decarboxylates isovalerate by reduction of an oxoferryl group Fe^{v}═O. Large-scale biosynthesis of isobutene by this pathway seems highly unfavourable, since it would require the synthesis and degradation of one molecule of leucine to form one molecule of isobutene. Also, the enzyme catalyzing the reaction uses heme as cofactor, poorly lending itself to recombinant expression in bacteria and to improvement of enzyme parameters. For all these reasons, it appears very unlikely that this pathway can serve as a basis for industrial exploitation. Other microorganisms have been described as being marginally capable of naturally producing isobutene from isovalerate; the yields obtained are even lower than those obtained with Rhodotorula minuta (Fukuda et al. (Agric. Biol. Chem. 48 (1984), 1679)).

Gogerty et al. (Appl. Environm. Microbiol. 76 (2010), 8004-8010) and van Leeuwen et al. (Appl. Microbiol. Biotechnol. 93 (2012), 1377-1387) describe the production of isobutene from acetoacetyl-CoA by enzymatic conversions wherein the last step of the proposed pathway is the conversion of 3-hydroxy-3-methylbutyric acid (also referred to as 3-hydroxyisovalerate (HIV)) by making use of a mevalonate diphosphate decarboxylase. This reaction for the production of isobutene from 3-hydroxy-3-methylbutyric acid is also described in WO2010/001078. In Gogerty et al. (loc. cit.) and in van Leeuwen et al. (loc. cit.) the production of 3-hydroxy-3-methylbutyric acid is proposed to be achieved by the conversion of 3-methylcrotonyl-CoA via 3-hydroxy-3-methylbutyryl-CoA. In order to further improve the efficiency and variability of methods for producing isobutene from renewable resources, there is a need for alternative routes for the provision of isobutene and its precursors.

The present invention meets this demand by providing a method for the production of isobutene comprising the enzymatic conversion of 3-methylcrotonic acid (also termed 3-methyl-2-butenoic acid) into isubutene.

The enzymatic conversion of 3-methylcrotonic acid into isobutene is a decarboxylation reaction. A decarboxylation is a chemical reaction that removes a carboxyl group and releases carbon dioxide (CO₂).

The decarboxylation of 3-methylcrotonic acid has already been suggested in US-A1-2009/0092975 while there is no experimental evidence for this conversion. In US-A1-2009/0092975, a nucleic acid sequence called PAD1 derived from Saccharomyces cerevisiae is described and is disclosed to encode a decarboxylation enzyme. This enzyme is suggested to be useful as a selectable marker in a recombinant organism while it is described that a “weak acid” may be used as the selecting agent. 3-methylcrotonic acid is mentioned, among many others, as a potential “weak acid”. However, it was only later found that the above PAD1, in reality, does not provide for the decarboxylase activity.

In fact, the bacterial ubiD and ubiX or the homologous eukaryotic fdc1 and pad1 genes have been implicated in the non-oxidative reversible decarboxylation. The combined action of phenylacrylic acid decarboxylase (PAD) and ferulic acid decarboxylase (FDC) is considered to be essential for the decarboxylation of phenylacrylic acid in Saccharomyces cerevisiae (J. Biosci. Bioeng. 109, (2010), 564-569; AMB Express, 5:12 (2015) 1-5; ACS Chem. Biol. 10 (2015), 1137-1144).

Recently, the above enzyme family described as phenylacrylic acid decarboxylase (PAD) was characterized as an FMN prenyl-transferase and no longer as a decarboxylase. It has been shown that Fdc1 (but not PAD) is solely responsible for the reversible decarboxylase activity and that it requires a new type of cofactor, namely a prenylated flavin synthesized by the associated UbiX (or Pad1) protein. Thus, the real enzymatic activity of this PAD enzyme has been identified as the transformation of a flavin mononucleotide (FMN) cofactor with a prenyl moiety (from di-methyl-allyl-phosphate or pyrophosphate called DMAP or DMAPP).

Accordingly, in contrast to the prior art's belief, the real decarboxylase is the ferulic acid decarboxylase (FDC) in association with the modified FMN (prenylated-FMN). This mechanism of the ferulic acid decarboxylase (FDC) in association with the modified FMN (prenylated-FMN) (the latter provided by the PAD enzyme) was recently described and involves a surprising enzymatic mechanism, i.e., an α,β-unsaturated acid decarboxylation via a 1,3-dipolar cyclo-addition. Moreover, the structure of this FDC decarboxylase has recently been elucidated (Nature 522 (2015), 497-501; Nature, 522 (2015), 502-505; Appl. Environ. Microbiol. 81 (2015), 4216-4223).

The use of the above family of enzymes has previously been described for the conversion of α-β unsaturated carboxylic acid into terminal alkenes in US-A1-2009/0092975 as mentioned above while WO2012/018624 is directed to microorganisms and methods for the biosynthesis of aromatics, 2,4-pentadienoate and 1,3-butadiene and WO2013/028519 is directed to microorganisms and methods for producing 2,4-pentadienoate, butadiene, propylene, 1,3-butanediol and related alcohols.

Moreover, WO2013/186215 describes a method for preparing a mono-unsaturated alkene comprising contacting an aliphatic mono-unsaturated carboxylic acid with an Fdc1 polypeptide and a Pad1 polypeptide. However, in WO2013/186215, both, the Fdc1 polypeptide and the Pad1 polypeptide are classified as enzymes having a decarboxylase activity.

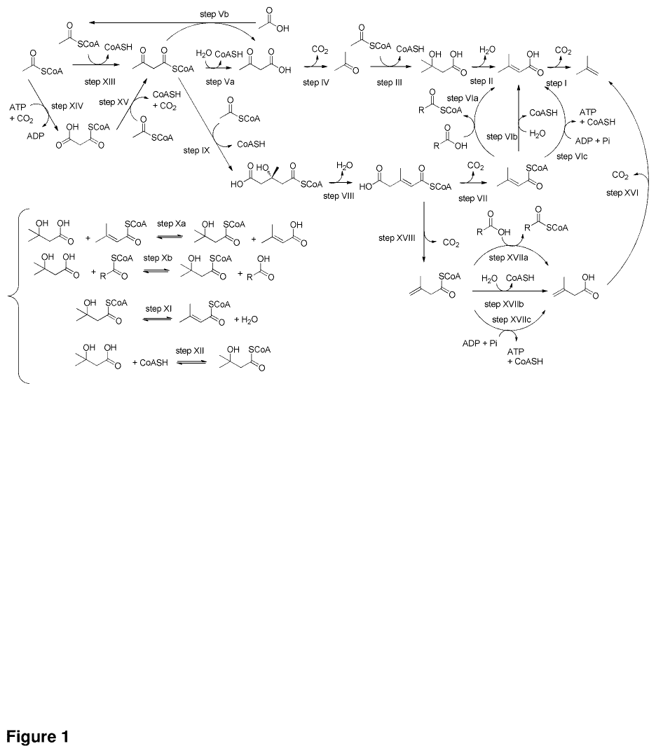

In contrast, in the present invention, the above enzymes are artificially implemented in a pathway which ultimately leads to the production of isobutene. Thus, in a main aspect, the present invention relates to a method for the production of isobutene comprising the enzymatic conversion of 3-methylcrotonic acid into isobutene (step I as shown in FIG. 1),

wherein said method further comprises
(a) providing the 3-methylcrotonic acid by the enzymatic conversion of 3-methylcrotonyl-CoA into 3-methylcrotonic acid (steps VIa, VIb or VIc as shown in FIG. 1), or
(b) providing the 3-methylcrotonic acid by the enzymatic conversion of 3-hydroxyisovalerate (HIV) into 3-methylcrotonic acid (step II as shown in FIG. 1).

Preferably, the enzymatic conversion of 3-methylcrotonic acid into isobutene is achieved by making use of a 3-methylcrotonic acid decarboxylase.

The method for the production of isobutene from 3-methylcrotonyl-CoA via 3-methylcrotonic acid or from 3-hydroxyisovalerate (HIV) via 3-methylcrotonic acid may be embedded in a pathway for the production of isobutene starting from acetyl-CoA which is a central component and an important key molecule in metabolism used in many biochemical reactions. The corresponding reactions are schematically shown in FIG. 1.

Therefore, the present invention also relates to pathways starting from acetyl-CoA and leading to 3-methylcrotonic acid (which is then ultimately converted into isobutene) via two alternative pathways which are schematically shown in FIG. 1 and will be explained in more detail further below.

The routes for the enzymatic conversion from acetyl-CoA into isobutene via acetoacetyl-CoA and 3-methylcrotonic acid

The enzymatic conversion of 3-methylcrotonic acid into isobutene: step I as shown in FIG. 1

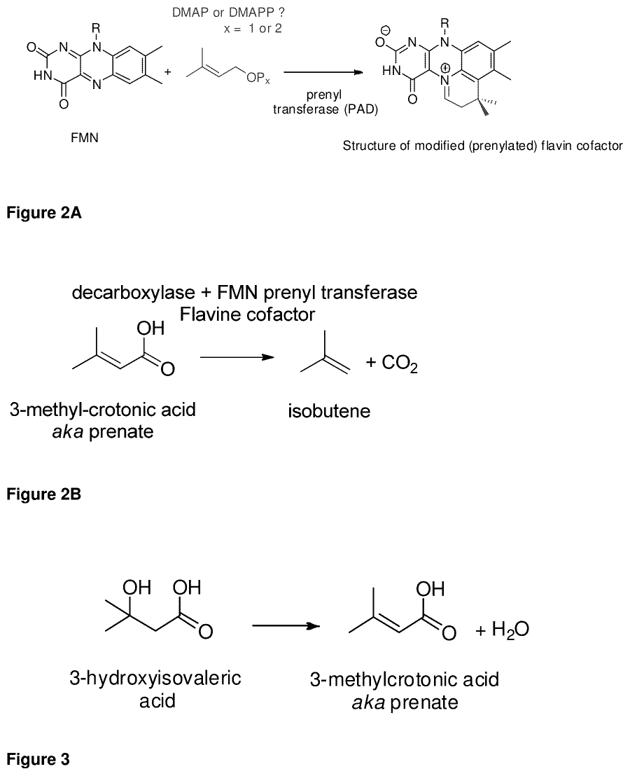

The enzymatic conversion of 3-methylcrotonic acid into isobutene is schematically shown in FIG. 2B.

According to the present invention, the enzymatic conversion of 3-methylcrotonic acid (also termed 3-methyl-2-butenoic acid or 3,3-dimethyl-acrylic acid) into isobutene (also termed isobutylene or 2-methyl-propene) can be achieved by a decarboxylation. “Decarboxylation” is generally a chemical reaction that removes a carboxyl group and releases carbon dioxide (CO₂).

The enzymatic conversion of 3-methylcrotonic acid into isobutene can preferably be achieved by making use of a 3-methylcrotonic acid decarboxylase. In accordance with the present invention, a 3-methylcrotonic acid decarboxylase is an enzyme which is capable of converting 3-methylcrotonic acid into isobutene in a decarboxylation reaction.

In preferred embodiments, the 3-methylcrotonic acid decarboxylase is selected from the group consisting of:
(i) an FMN-dependent decarboxylase associated with an FMN prenyl transferase; or
(ii) an aconitate decarboxylase (EC 4.1.1.6); or
(iii) a methylcrotonyl-CoA carboxylase (EC 6.4.1.4); or
(iv) a geranoyl-CoA carboxylase (EC 6.4.1.5).

Thus, according to one aspect, the enzymatic conversion of 3-methylcrotonic acid into isobutene can preferably be achieved by making use of a 3-methylcrotonic acid decarboxylase, wherein said 3-methylcrotonic acid decarboxylase is an FMN-dependent decarboxylase associated with an FMN prenyl transferase.

The enzymatic conversion of 3-methylcrotonic acid into isobutene utilizing an FMN-dependent decarboxylase associated with an FMN prenyl transferase relies on a reaction of two consecutive steps catalyzed by the two enzymes, i.e., the FMN-dependent decarboxylase (catalyzing the actual decarboxylation of 3-methylcrotonic acid into isobutene) with an associated FMN prenyl transferase which provides the modified flavin cofactor. The flavin cofactor may preferably be FMN or FAD. FMN (flavin mononucleotide; also termed riboflavin-5′-phosphate) is a biomolecule produced from riboflavin (vitamin B2) by the enzyme riboflavin kinase and functions as prosthetic group of various reactions. FAD (flavin adenine dinucleotide) is a redox cofactor, more specifically a prosthetic group, involved in several important reactions in metabolism.

Thus, in the conversion of 3-methylcrotonic acid into isobutene, in a first step, a flavin cofactor (FMN or FAD) is modified into a (modified) flavin-derived cofactor. This modification is catalyzed by said FMN prenyl transferase. FMN prenyl transferase prenylates the flavin ring of the flavin cofactor (FMN or FAD) into a (modified) prenylated flavin cofactor. This reaction is schematically illustrated in FIG. 2A.

In a second step, the actual conversion of 3-methylcrotonic acid into isobutene is catalyzed by said FMN-dependent decarboxylase via a 1,3-dipolar cycloaddition based mechanism wherein said FMN-dependent decarboxylase uses the prenylated flavin cofactor (FMN or FAD) provided by the associated FMN prenyl transferase. This reaction is schematically illustrated in FIG. 2B.

In a preferred embodiment, said FMN prenyl transferase which modifies the flavin cofactor (FMN or FAD) into a (modified) flavin-derived cofactor is a phenylacrylic acid decarboxylase (PAD)-type protein, or the closely related prokaryotic enzyme UbiX, an enzyme which is involved in ubiquinone biosynthesis in prokaryotes.

In Escherichia coli, the protein UbiX (also termed 3-octaprenyl-4-hydroxybenzoate carboxy-lyase) has been shown to be involved in the third step of ubiquinone biosynthesis.

It catalyses the reaction 3-octaprenyl-4-hydroxybenzoate [symbol] 2-octaprenylphenol+CO₂.

Moreover, the knockout of the homologous protein in yeast (Pad1) has been shown to confer sensitivity to phenylacrylic acid, showing that this enzyme functions as a phenylacrylic acid decarboxylase. E. coli strains also contain, in addition to UbiX, a second paralogue named Pad1. Its amino acid sequence shows 52% identity to UbiX and slightly higher sequence identity to Saccharomyces cerevisiae phenylacrylic acid decarboxylase Pad1. Despite its higher sequence similarity with yeast Pad1, E. coli Pad1 does not seem to have phenylacrylic acid decarboxylase activity. Its function is unknown, Pad1 may remove the carboxylate group from derivatives of benzoic acid but not from substituted phenolic acids.

Thus, in a preferred embodiment, the modification of a flavin cofactor (FMN or FAD) into the corresponding (modified) flavin-derived cofactor is catalyzed by the FMN-containing protein phenylacrylic acid decarboxylase (PAD). The enzymes involved in the modification of the flavin cofactor (FMN or FAD) into the corresponding modified flavin-derived cofactor were initially annotated as decarboxylases (EC 4.1.1.-). Some phenylacrylic acid decarboxylases (PAD) are now annotated as flavin prenyl transferases as EC 2.5.1.-.

In a more preferred embodiment, the conversion of 3-methylcrotonic acid into isobutene makes use of a phenylacrylic acid decarboxylase (PAD)-type protein as the FMN prenyl transferase which modifies a flavin cofactor (FMN or FAD) into the corresponding (modified) flavin-derived cofactor wherein said phenylacrylic acid decarboxylase (PAD)-type protein is derived from Candida albicans (Uniprot accession number Q5A8L8), Aspergillus niger (Uniprot accession number A3F715), Saccharomyces cerevisiae (Uniprot accession number P33751) or Cryptococcus gattii (Uniprot accession number E6R9Z0).

In a preferred embodiment, the phenylacrylic acid decarboxylase (PAD)-type protein employed in the method of the present invention is a phenylacrylic acid decarboxylase (PAD)-type protein derived from Candida albicans (Uniprot accession number Q5A8L8; SEQ ID NO:40), Aspergillus niger (Uniprot accession number A3F715; SEQ ID NO:41), Saccharomyces cerevisiae (Uniprot accession number P33751; SEQ ID NO:42) or Cryptococcus gattii (Uniprot accession number E6R9Z0; SEQ ID NO:43) having the amino acid sequence as shown in SEQ ID NO:40, SEQ ID NO:41, SEQ ID NO:42 and SEQ ID NO:43, respectively.

In a preferred embodiment of the present invention the phenylacrylic acid decarboxylase (PAD)-type protein is an enzyme comprising an amino acid sequence selected from the group consisting of SEQ ID NOs: 40 to 43 or a sequence which is at least n % identical to any of SEQ ID NOs: 40 to 43 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of modifying a flavin cofactor (FMN or FAD) into the corresponding (modified) flavin-derived cofactor.

As regards the determination of sequence identity, the following should apply: When the sequences which are compared do not have the same length, the degree of identity either refers to the percentage of amino acid residues in the shorter sequence which are identical to amino acid residues in the longer sequence or to the percentage of amino acid residues in the longer sequence which are identical to amino acid residues in the shorter sequence. Preferably, it refers to the percentage of amino acid residues in the shorter sequence which are identical to amino acid residues in the longer sequence. The degree of sequence identity can be determined according to methods well known in the art using preferably suitable computer algorithms such as CLUSTAL.

When using the Clustal analysis method to determine whether a particular sequence is, for instance, at least 60% identical to a reference sequence default settings may be used or the settings are preferably as follows: Matrix: blosum 30; Open gap penalty: 10.0; Extend gap penalty: 0.05; Delay divergent: 40; Gap separation distance: 8 for comparisons of amino acid sequences. For nucleotide sequence comparisons, the Extend gap penalty is preferably set to 5.0.

In a preferred embodiment ClustalW2 is used for the comparison of amino acid sequences. In the case of pairwise comparisons/alignments, the following settings are preferably chosen: Protein weight matrix: BLOSUM 62; gap open: 10; gap extension: 0.1. In the case of multiple comparisons/alignments, the following settings are preferably chosen: Protein weight matrix: BLOSUM 62; gap open: 10; gap extension: 0.2; gap distance: 5; no end gap.

Preferably, the degree of identity is calculated over the complete length of the sequence.

Amino acid residues located at a position corresponding to a position as indicated herein-below in the amino acid sequence shown in any one of SEQ ID NOs:40 to 43 can be identified by the skilled person by methods known in the art. For example, such amino acid residues can be identified by aligning the sequence in question with the sequence shown in any one of SEQ ID NOs:40 to 43 and by identifying the positions which correspond to the above indicated positions of any one of SEQ ID NOs:40 to 43. The alignment can be done with means and methods known to the skilled person, e.g. by using a known computer algorithm such as the Lipman-Pearson method (Science 227 (1985), 1435) or the CLUSTAL algorithm. It is preferred that in such an alignment maximum homology is assigned to conserved amino acid residues present in the amino acid sequences.

In a preferred embodiment ClustalW2 is used for the comparison of amino acid sequences. In the case of pairwise comparisons/alignments, the following settings are preferably chosen: Protein weight matrix: BLOSUM 62; gap open: 10; gap extension: 0.1. In the case of multiple comparisons/alignments, the following settings are preferably chosen: Protein weight matrix: BLOSUM 62; gap open: 10; gap extension: 0.2; gap distance: 5; no end gap.

Preferably, the degree of identity is calculated over the complete length of the sequence.

In another preferred embodiment, the modification of a flavin cofactor (FMN or FAD) into the corresponding (modified) flavin-derived cofactor is catalyzed by the FMN-containing protein 3-octaprenyl-4-hydroxybenzoate carboxy-lyase also termed UbiX (initially annotated EC 4.1.1.-). As mentioned above, the enzymes involved in the modification of the flavin cofactor (FMN or FAD) into the corresponding modified flavin-derived cofactor were initially annotated as decarboxylases. Some phenylacrylic acid decarboxylases (PAD) are now annotated as flavin prenyl transferases as EC 2.5.1.-.

In a more preferred embodiment, the conversion of 3-methylcrotonic acid into isobutene makes use of a 3-octaprenyl-4-hydroxybenzoate carboxy-lyase (also termed UbiX) as the FMN prenyl transferase which modifies the flavin cofactor (FMN or FAD) into the corresponding (modified) flavin-derived cofactor wherein said 3-octaprenyl-4-hydroxybenzoate carboxy-lyase (also termed UbiX) is derived from Escherichia coli (Uniprot accession number P0AG03), Bacillus subtilis (Uniprot accession, number A0A086WXG4), Pseudomonas aeruginosa (Uniprot accession number A0A072ZCW8) or Enterobacter sp. DC4 (Uniprot accession number W7P6B1).

In an even more preferred embodiment, the 3-octaprenyl-4-hydroxybenzoate carboxy-lyase (also termed UbiX) employed in the method of the present invention is a 3-octaprenyl-4-hydroxybenzoate carboxy-lyase (also termed UbiX) derived from Escherichia coli (Uniprot accession number P0AG03; SEQ ID NO:44), Bacillus subtilis (Uniprot accession, number A0A086WXG4; SEQ ID NO:45), Pseudomonas aeruginosa (Uniprot accession number A0A072ZCW8; SEQ ID NO:46) or Enterobacter sp. DC4 (Uniprot accession number W7P6B1; SEQ ID NO:47) having the amino acid sequence as shown in SEQ ID NO:44, SEQ ID NO:45, SEQ ID NO:46 and SEQ ID NO:47, respectively.

In a preferred embodiment of the present invention the 3-octaprenyl-4-hydroxybenzoate carboxy-lyase is an enzyme comprising an amino acid sequence selected from the group consisting of SEQ ID NOs: 44 to 47 or a sequence which is at least n % identical to any of SEQ ID NOs: 44 to 47 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of modifying a flavin cofactor (FMN or FAD) into the corresponding (modified) flavin-derived cofactor. As regards the determination of the sequence identity, the same applies as has been set forth above.

In another preferred embodiment, the modification of a flavin cofactor (FMN or FAD) into the corresponding (modified) flavin-derived cofactor is catalyzed by a flavin prenyl transferase.

As mentioned above, the actual decarboxylation, i.e., the conversion of 3-methylcrotonic acid into isobutene is catalyzed by an FMN-dependent decarboxylase via a 1,3-dipolar cycloaddition based mechanism wherein said FMN-dependent decarboxylase uses the prenylated flavin cofactor (FMN or FAD) provided by any of the above described associated FMN prenyl transferases.

In a preferred embodiment, said FMN-dependent decarboxylase catalyzing the decarboxylation of 3-methylcrotonic acid into isobutene is catalyzed by a ferulic acid decarboxylase (FDC). Ferulic acid decarboxylases (FDC) belong to the enzyme class EC 4.1.1.-.

In an even more preferred embodiment, the conversion of 3-methylcrotonic acid into isobutene makes use of a ferulic acid decarboxylases (FDC) which is derived from Saccharomyces cerevisiae (Uniprot accession number Q03034), Enterobacter sp. (Uniprot accession number V3P7U0), Bacillus pumilus (Uniprot accession number Q45361), Aspergillus niger (Uniprot accession number A2R0P7) or Candida dubliniensis (Uniprot accession number B9WJ66).

In a preferred embodiment, the ferulic acid decarboxylases (FDC) employed in the method of the present invention is a ferulic acid decarboxylases (FDC) derived from Saccharomyces cerevisiae (Uniprot accession number Q03034; SEQ ID NO:48), Enterobacter sp. (Uniprot accession number V3P7U0; SEQ ID NO:49), Bacillus pumilus (Uniprot accession number Q45361; SEQ ID NO:50), Aspergillus niger (Uniprot accession number A2R0P7; SEQ ID NO:51) or Candida dubliniensis (Uniprot accession number B9WJ66; SEQ ID NO:52) having the amino acid sequence as shown in SEQ ID NO:48, SEQ ID NO:49, SEQ ID NO:50, SEQ ID NO:51 and SEQ ID NO:52, respectively.

In another more preferred embodiment, the conversion of 3-methylcrotonic acid into isobutene makes use of a protocatechuate decarboxylase (EC 4.1.1.63).

Thus, in one preferred embodiment, the conversion of 3-methylcrotonic acid into isobutene is catalyzed by a protocatechuate (PCA) decarboxylase (EC 4.1.1.63). PCA decarboxylases (also termed AroY) are known to catalyze the following reaction, i.e., the enzymatic conversion of protocatechuate (PCA) into catechol (Johnson et al., Metabolic Engineering Communications 3 (2016), 111):

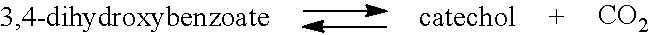

This enzyme occurs in a variety of organisms and has, e.g., been described in Enterobacter aerogenes, Enterobacter cloacae, Rhodopseudomonas sp. and Sedimentibacter hydroxybenzoicus.

In a preferred embodiment of the present invention, the PCA decarboxylase employed in the method of the present invention is a PCA decarboxylase which is derived from Klebsiella pneumoniae (Uniprot accession number B9AM6), Leptolyngbya sp. (Uniprot accession number A0A0S3U6D8), or Phascolarctobacterium sp. (Uniprot accession number R6IIV6).

In a preferred embodiment, the PCA decarboxylase employed in the method of the present invention is an enzyme derived from Klebsiella pneumonia (SEQ ID NO:78), Leptolyngbya sp. (SEQ ID NO:80), or Phascolarctobacterium sp. (SEQ ID NO:81).

In a preferred embodiment of the present invention the PCA decarboxylase is an enzyme comprising an amino acid sequence selected from the group consisting of SEQ ID NOs: 78, 80 and 81 or a sequence which is at least n % identical to any of SEQ ID NOs: 78, 80 and 81 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylcrotonic acid into isobutene. As regards the determination of the sequence identity, the same applies as has been set forth above.

In a preferred embodiment of the present invention the ferulic acid decarboxylase (FDC) is an enzyme comprising an amino acid sequence selected from the group consisting of SEQ ID NOs: 48 to 52 or a sequence which is at least n % identical to any of SEQ ID NOs: 48 to 52 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylcrotonic acid into isobutene. As regards the determination of the sequence identity, the same applies as has been set forth above.

In another preferred embodiment, said FMN-dependent decarboxylase catalyzing the decarboxylation of 3-methylcrotonic acid into isobutene is an enzyme which is closely related to the above ferulic acid decarboxylase (FDC), namely a 3-polyprenyl-4-hydroxybenzoate decarboxylase (also termed UbiD). 3-polyprenyl-4-hydroxybenzoate decarboxylase belongs to the UbiD decarboxylase family classified as EC:4.1.1.-.

In a more preferred embodiment, the conversion of 3-methylcrotonic acid into isobutene makes use of a 3-polyprenyl-4-hydroxybenzoate decarboxylase (UbiD) which is derived from Hypocrea atroviridis (UniProt Accession number G9NLP8), Sphaerulina musiva (UniProt Accession number M3DF95), Penecillinum requeforti (UniProt Accession number W6QKP7), Fusarium oxysporum f. sp. lycopersici (UniProt Accession number W9LTH3), Saccharomyces kudriavzevii (UniProt Accession number J8TRN5), Saccaromyces cerevisiae, Aspergillus parasiticus, Candida albicans, Grosmannia clavigera, Escherichia coli (Uniprot accession number P0AAB4), Bacillus megaterium (Uniprot accession number D5DTL4), Methanothermobacter sp. CaT2 (Uniprot accession number T2GKK5), Mycobacterium chelonae 1518 (Uniprot accession number X8EX86) or Enterobacter cloacae (Uniprot accessin number V3DX94).

In an even more preferred embodiment, the 3-polyprenyl-4-hydroxybenzoate decarboxylase (UbiD) employed in the method of the present invention is a 3-polyprenyl-4-hydroxybenzoate decarboxylase (UbiD) derived from Escherichia coli (Uniprot accession number P0AAB4; SEQ ID NO:53), Bacillus megaterium (Uniprot accession number D5DTL4; SEQ ID NO:54), Methanothermobacter sp. CaT2 (Uniprot accession number T2GKK5; SEQ ID NO:55) Mycobacterium chelonae 1518 (Uniprot accession number X8EX86; SEQ ID NO:56), Hypocrea atroviridis (SEQ ID NO:57), Sphaerulina musiva (SEQ ID NO:58), Penecillinum requeforti (SEQ ID NO:59), Fusarium oxysporum f. sp. lycopersici (SEQ ID NO:60), Saccharomyces kudriavzevii (SEQ ID NO:61), Saccaromyces cerevisiae (SEQ ID NO:62), Aspergillus parasiticus (SEQ ID NO:63), Candida albicans (SEQ ID NO:64), Grosmannia clavigera (SEQ ID NO:65) or Enterobacter cloacae (SEQ ID NO:79) having the amino acid sequence as shown in SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, and SEQ ID NO:79, respectively.

In a preferred embodiment of the present invention the 3-polyprenyl-4-hydroxybenzoate decarboxylase (UbiD) is an enzyme comprising an amino acid sequence selected from the group consisting of SEQ ID NOs: 53 to 65 or a sequence which is at least n % identical to any of SEQ ID NOs: 53 to 65 and SEQ ID NO:79 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylcrotonic acid into isobutene. As regards the determination of the sequence identity, the same applies as has been set forth above.

As mentioned above, in another aspect, the 3-methylcrotonic acid decarboxylase may preferably be an aconitate decarboxylase (EC 4.1.1.6). This decarboxylase does not require the association with an FMN prenyl transferase as it has been described for the above decarboxylases and, accordingly, does not require the provision of a prenylated cofactor.

Thus, in one preferred embodiment, the conversion of 3-methylcrotonic acid into isobutene is catalyzed by an aconitate decarboxylase (EC 4.1.1.6). Aconitate decarboxylases (EC 4.1.1.6) have been described to catalyze the following reaction:

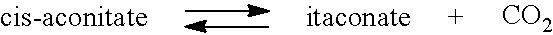

This enzyme occurs in a variety of organisms, and has, e.g., been described in Aspergillus itaconicus, Aspergillus terreus, Homo sapiens and Mus musculus. In a preferred embodiment, the aconitate decarboxylase (EC 4.1.1.6) employed in the method of the present invention in the conversion of 3-methylcrotonic acid into isobutene is the aconitase decarboxylase derived from Aspergillus terreus (UniProt accession number B3IUN8), Homo sapiens (UniProt accession number A6NK06) or Mus musculus (UniProt accession number P54987).

In a preferred embodiment, the aconitate decarboxylase (EC 4.1.1.6) employed in the method of the present invention in the conversion of 3-methylcrotonic acid into isobutene is a aconitate decarboxylase derived from Aspergillus terreus (SEQ ID NO:66).

In a preferred embodiment of the present invention the aconitate decarboxylase is an enzyme comprising the amino acid sequence of SEQ ID NO: 66 or a sequence which is at least n % identical to SEQ ID NO: 66 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylcrotonic acid into isobutene. As regards the determination of the sequence identity, the same applies as has been set forth above.

As mentioned above, in another aspect, the 3-methylcrotonic acid decarboxylase may preferably be a methylcrotonyl-CoA carboxylase (EC 6.4.1.4). This decarboxylase does not require the association with an FMN prenyl transferase as it has been described for the above decarboxylases and, accordingly, does not require the provision of a prenylated cofactor.

Thus, in one preferred embodiment, the conversion of 3-methylcrotonic acid into isobutene is catalyzed by a methylcrotonyl-CoA carboxylase (EC 6.4.1.4). Methylcrotonyl-CoA carboxylases have been described to catalyze the following reaction:

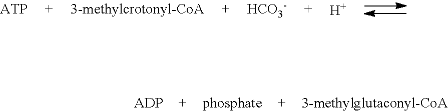

i.e. the carboxylation, but they can also be used to catalyze the reaction of decarboxylation. Methylcrotonyl-CoA carboxylases occur in a variety of organisms, including eukaryotic and prokaryotic organisms, such as plants, animals, fungi and bacteria. The enzyme has, e.g., been described in Daucus carota, Glycine max, Hordeum vulgare, Pisum sativum, Solanum lycopersicum, Solanum tuberosum, Zea mays, Arabidopsis sp., Lens culinaris, Homo sapiens, Bos taurus, Rattus norvegicus, Mus musculus, Pagrus major, Emericella nidulans, Pseudomonas aeruginosa, Pseudomonas citronellolis, Pseudomonas amygdali, Acidaminococcus fermentans, Escherichia coli, Mycobacterium sp. and Achromobacter sp.

In a preferred embodiment, the methylcrotonyl-CoA carboxylase (EC 6.4.1.4) employed in the method of the present invention in the conversion of 3-methylcrotonic acid into isobutene is a methylcrotonyl-CoA carboxylase derived from Pseudomonas amygdali (SEQ ID NO:67).

In a preferred embodiment of the present invention the methylcrotonyl-CoA carboxylase is an enzyme comprising the amino acid sequence of SEQ ID NO: 67 or a sequence which is at least n % identical to SEQ ID NO: 67 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylcrotonic acid into isobutene. As regards the determination of the sequence identity, the same applies as has been set forth above.

In another preferred embodiment, the methylcrotonyl-CoA carboxylase (EC 6.4.1.4) employed in the method of the present invention in the conversion of 3-methylcrotonic acid into isobutene is a methylcrotonyl-CoA carboxylase derived from Myxcoxoccus xanthus. In Myxococcus xanthus, the the IiuB gene codes for an enzyme having the two subunits AibA and AibB (Li et al., Angew. Chem. Int. Ed. 52 (2013), 1304-1308). The methylcrotonyl-CoA carboxylase derived from Myxcoxoccus xanthus is a hetero-dimeric enzyme which are annotated as glutaconyl-CoA transferase subunits A and B (SEQ ID NOs: 100 and 101).

In a preferred embodiment of the present invention the methylcrotonyl-CoA carboxylase is an enzyme comprising the amino acid sequence of SEQ ID NO: 100 or 101 a sequence which is at least n % identical to SEQ ID NO: 100 or 101 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylcrotonic acid into isobutene. As regards the determination of the sequence identity, the same applies as has been set forth above.

As mentioned above, in another aspect, the 3-methylcrotonic acid decarboxylase may preferably be a geranoyl-CoA carboxylase (EC 6.4.1.5). This decarboxylase does not require the association with an FMN prenyl transferase as it has been described for the above decarboxylases and, accordingly, does not require the provision of a prenylated cofactor.

Thus, in another preferred embodiment, the conversion of 3-methylcrotonic acid via decarboxylasion into isobutene is catalyzed by a geranoyl-CoA carboxylase (EC 6.4.1.5). Geranoyl-CoA carboxylases naturally catalyze the following reaction:

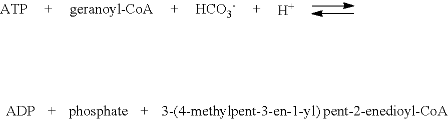

The enzyme occurs in eukaryotes and prokaryotes, such as plants and bacteria. The enzyme has, e.g., been described in Daucus carota, Glycine max, Zea mays, Pseudomonas sp., Pseudomonas aeruginosa, Pseudomonas citronellolis and Pseudomonas mendocina.

In another aspect, the 3-methylcrotonic acid decarboxylase may preferably be a 6-methylsalicylate decarboxylase (EC 4.1.1.52).

Thus, in another preferred embodiment, the conversion of 3-methylcrotonic acid via decarboxylasion into isobutene is catalyzed by a 6-methylsalicylate decarboxylase (EC 4.1.1.52). 6-methylsalicylate decarboxylases (EC 4.1.1.52) naturally catalyze the following reaction:

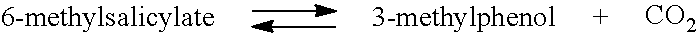

The enzyme occurs in a variety of organisms, in particular in eucaryotes and prokaryotes, such as bacteria and fungi. The enzyme has, e.g., been described in Aspergillus clavatus (UniProt Accession number T1PRE6), Penicillium griseofulvum and Valsa friesii.

In a preferred embodiment, the 6-methylsalicylate decarboxylase (EC 4.1.1.52) employed in the method of the present invention in the conversion 3-methylcrotonic acid via decarboxylasion into isobutene is a 6-methylsalicylate decarboxylase derived from Aspergillus clavatus (SEQ ID NO:68).

In a preferred embodiment of the present invention the 6-methylsalicylate decarboxylase is an enzyme comprising the amino acid sequence of SEQ ID NO: 68 or a sequence which is at least n % identical to SEQ ID NO: 68 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylcrotonic acid via decarboxylasion into isobutene. As regards the determination of the sequence identity, the same applies as has been set forth above.

In another aspect, the 3-methylcrotonic acid decarboxylase may preferably be a 2-oxo-3-hexenedioate decarboxylase (EC 4.1.1.77).

Thus, in another preferred embodiment, the conversion of 3-methylcrotonic acid via decarboxylasion into isobutene is catalyzed by a 2-oxo-3-hexenedioate decarboxylase (EC 4.1.1.77). 2-oxo-3-hexenedioate decarboxylases (EC 4.1.1.77) naturally catalyze the following reaction:

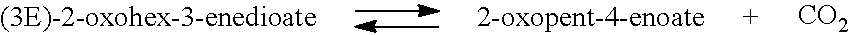

The enzyme occurs in a variety of organisms, in particular in prokaryotes, such as bacteria. The enzyme has, e.g., been described in Bordetella sp., Cupriavidus nexator, Geobacillus stearothermophilus (UniProt Accession number B0VXM8), Pseudomonas putida and Ralstonia pickettii.

In a preferred embodiment, the 2-oxo-3-hexenedioate decarboxylase (EC 4.1.1.77) employed in the method of the present invention in the conversion 3-methylcrotonic acid via decarboxylasion into isobutene is a 2-oxo-3-hexenedioate decarboxylase derived from Geobacillus stearothermophilus (SEQ ID NO:69).

In a preferred embodiment of the present invention the 2-oxo-3-hexenedioate decarboxylase is an enzyme comprising the amino acid sequence of SEQ ID NO: 69 or a sequence which is at least n % identical to SEQ ID NO: 69 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylcrotonic acid via decarboxylasion into isobutene. As regards the determination of the sequence identity, the same applies as has been set forth above.

In another possibility, the 3-methylcrotonic acid decarboxylase may preferably be a 5-oxopent-3-ene-1,2,5-tricarboxylate decarboxylase (EC 4.1.1.68).

Thus, in another preferred embodiment, the conversion of 3-methylcrotonic acid via decarboxylasion into isobutene is catalyzed by a 5-oxopent-3-ene-1,2,5-tricarboxylate decarboxylase (EC 4.1.1.68). 5-oxopent-3-ene-1,2,5-tricarboxylate decarboxylases (EC 4.1.1.68) naturally catalyze the following reaction:

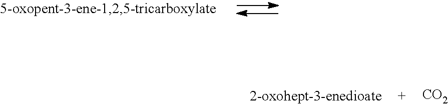

The enzyme has been described to occur in prokaryotes such as bacteria. The enzyme has, e.g., been described in E. coli and Salmonella dublin.

In a preferred embodiment, the 5-oxopent-3-ene-1,2,5-tricarboxylate decarboxylase (EC 4.1.1.68) employed in the method of the present invention in the conversion 3-methylcrotonic acid via decarboxylasion into isobutene is a 5-oxopent-3-ene-1,2,5-tricarboxylate decarboxylase derived from Salmonella dublin (SEQ ID NO:70).

In a preferred embodiment of the present invention the 5-oxopent-3-ene-1,2,5-tricarboxylate decarboxylase is an enzyme comprising the amino acid sequence of SEQ ID NO: 70 or a sequence which is at least n % identical to SEQ ID NO: 70 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylcrotonic acid via decarboxylasion into isobutene. As regards the determination of the sequence identity, the same applies as has been set forth above.

The enzymatic conversion of 3-hydroxyisovalerate (HIV) into 3-methylcrotonic acid: step II as shown in FIG. 1

The 3-methylcrotonic acid which is converted according to the method of the present invention into isobutene may itself be provided by an enzymatic reaction.

According to the present invention, the 3-methylcrotonic acid can be provided via different routes which are schematically shown in FIG. 1.

Thus, according to one option, the 3-methylcrotonic acid may itself be provided by the enzymatic conversion of 3-hydroxyisovalerate (HIV) into 3-methylcrotonic acid. The enzymatic conversion of 3-hydroxyisovalerate (HIV) into 3-methylcrotonic acid (step II as shown in FIG. 1) is schematically illustrated in FIG. 3.

According to the present invention, the enzymatic conversion of 3-hydroxyisovalerate (HIV) into said 3-methylcrotonic acid preferably makes use of an enzyme catalyzing the dehydration of a R-hydroxy acid (i.e., e.g., 3-hydroxyisovalerate (HIV)) into an α,β-unsaturated acid (i.e., e.g., 3-methylcrotonic acid). The term “dehydration” generally refers to a reaction involving the removal of H₂O. Enzymes catalyzing 3-hydroxyisovalerate (HIV) dehydration are enzymes which catalyze the reaction as shown in FIG. 3. Preferably, such an enzyme belongs to the family of hydro-lyases (EC 4.2.-.-).

Preferred examples of such enzymes which are classified as EC 4.2.-.- (i.e., hydro-lyases) are:
aconitase (EC 4.2.1.3);
fumarase (EC 4.2.1.2); and
enoyl-CoA hydratase/dehydratease (EC 4.2.1.17).

Thus, in one preferred embodiment, the enzymatic conversion of 3-hydroxyisovalerate (HIV) into 3-methylcrotonic acid is achieved by the use of an aconitase (EC 4.2.1.3). Aconitases (EC 4.2.1.3) (also termed aconitase hydratases) are enzymes which catalyze the following reaction:

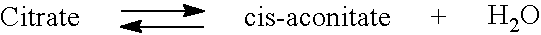

The enzyme is known from a variety of organisms, including eukaryotic and prokaryotic organisms, such as plants, animals, fungi and bacteria. The enzyme has, e.g., been described in Acer pseudoplatanus, Advenella kashmirensis, Arabidopsis thaliana, Aspergillus niger, Bacillus cereus, Bacillus subtilis, Bacterioides fragilis, Bos taurus, Caenorhabditis elegans, Citrus elementina, Canis lupus familiaris, Corynebacterium glutamicum, Drosophila melanogaster, E. coli, Glycine max, Helobacter pylori, Homo sapiens, Mus musculus, Mycobacterium tuberculosis, Nicotiana benthamiana, Plasmodium falciparum, Pseudomonas aeruginosa, Rattus norvegicus, Rattus rattus, Saccharomyces cerevisiae, Saccharomycopsis lipolytica, Salmonella enterica, Sinapis alba, Sinorhizobium meliloti, Solanum tuberosum, Streptomyces aureus, Streptomyces viridochromogenes, Sulfolobus acidocaldarius, Sulfolobus solfataricus, Sus scorfa, Trametes sanguinea, Trypanosoma brucei, Xanthomonas campestris, Xanthomonas euvesicatoria, Yarrowia lipolytica and Zea mays.

In a preferred embodiment, the aconitase (EC 4.2.1.3) is from Advenella kashmirensis (TrEMBL accession number B3TZE0), Bacterioides fragilis (SwissProt accession number Q8RP87), Caenorhabditis elegans (SwissProt accession number Q23500), Citrus elementina (UniProt accession number D3GQL0, D3GQL1, or D3GQL2), Drosophila melanogaster (SwissProt accession number Q9NFX3 or Q9NFX2), E. coli (SwissProt accession number P36683 or UniProt accession number P25516), Homo sapiens (UniProt accession number P21399 or Q99798), Mus musculus (UniProt accession number P28271), Rattus norvegicus (UniProt accession number Q9ER34 or Q63270), Sus scorfa (UniProt accession number P16276) or Trypanosoma brucei (SwissProt accession number Q9NJQ8 or Q9NJQ9).

In a preferred embodiment, the aconitase (EC 4.2.1.3) employed in the method of the present invention in the conversion of 3-hydroxyisovalerate (HIV) into 3-methylcrotonic acid is an aconitase derived from E. coli (SEQ ID NO:71).

In a preferred embodiment of the present invention the aconitase is an enzyme comprising the amino acid sequence of SEQ ID NO: 71 or a sequence which is at least n % identical to SEQ ID NO: 71 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-hydroxyisovalerate (HIV) into 3-methylcrotonic acid. As regards the determination of the sequence identity, the same applies as has been set forth above.

In another preferred embodiment, the enzymatic conversion of 3-hydroxyisovalerate (HIV) into 3-methylcrotonic acid is achieved by the use of a fumarase (EC 4.2.1.2). Fumarases (EC 4.2.1.2) (also termed fumarase hydratases) are enzymes which catalyze the following reaction:

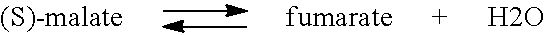

The enzyme is known from a variety of organisms, including eukaryotic and prokaryotic organisms, such as plants, animals, fungi and bacteria. The enzyme has, e.g., been described in Arabidopsis thaliana, Ascaris suum, Azotobacter vinelandii, Brevibacterium flavum, Campylobacter coli, Campylobacter fetus, Campylobacter jejuni, Corynebacterium ammoniagenes, Corynebacterium glutamicum, Erwinia sp., E. coli, Euglena gracilis, Geobacillus stearothermophilus, Gluconacetobacter diazotrophicus, Heliobacter pylori, Homo sapiens, Leishmania major, Mesembryanthemum crystallinum, Mycobacterium tuberculosis, Pelotomaculum thermopropionicum, Pisum sativum, Pseudomonas aeruginosa, Pseudomonas fluorescens, Pycobaculum neutrophilum, Rattus novegicus, Rhizopus oryzae, Rickettsia prowazekii, Saccharomyces bayanus, Sacchoromyces cerevisiae, Solanum lycopersicum, Solanum tuberosum, Streptomyces coelicolor, Streptomyces lividans, Streptomyces thermovulgaris, Sulfolobus solfataricus, Sus scrofa, Thermus sp., Thermus thermophilus and Zea mays.

In a preferred embodiment, the fumarase (EC 4.2.1.2) is from Arabidopsis thaliana (UniProt accession number P93033 or Q9F153), Ascaris suum (SwissProt accession number Q8NRN8), Corynebacterium glutamicum (UniProt accession number P28271), E. coli (P05042), Homo sapiens (SwissProt accession number P07954), Mycobacterium tuberculosis (P9WN93), Pycobaculum neutrophilum (UniProt accession number B1Y931 or B1Y932), Rhizopus oryzae (UniProt accession number P55250), Rickettsia prowazekii (UniProt accession number Q9ZCQ4), Sacchoromyces cerevisiae (SwissProt accession number P08417), Streptomyces thermovulgaris (SwissProt accession number A5Y6J1) or Sulfolobus solfataricus (UniProt accession number P39461).

In a preferred embodiment, the fumarase (EC 4.2.1.2) employed in the method of the present invention in the conversion of 3-hydroxyisovalerate (HIV) into 3-methylcrotonic acid is a fumarase derived from E. coli (SEQ ID NO:72).

In a preferred embodiment of the present invention the fumarase is an enzyme comprising the amino acid sequence of SEQ ID NO: 72 or a sequence which is at least n % identical to SEQ ID NO: 72 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-hydroxyisovalerate (HIV) into 3-methylcrotonic acid. As regards the determination of the sequence identity, the same applies as has been set forth above.

In another preferred embodiment, the enzymatic conversion of 3-hydroxyisovalerate (HIV) into 3-methylcrotonic acid is achieved by the use of an enoyl-CoA hydratase/dehydratase (EC 4.2.1.17). Enoyl-CoA hydratases/dehydratases (EC 4.2.1.17) catalyze the following reaction:

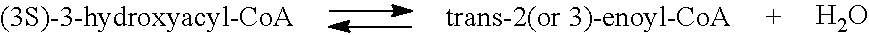

Enoyl-CoA hydratase is an enzyme that normally hydrates the double bond between the second and third carbon atoms on acyl-CoA. However, it can also be employed to catalyze the reaction in the reverse direction.

Enoyl-CoA hydratases/dehydratases (EC 4.2.1.17) are also termed 3-hydroxyacyl-CoA dehydratases and enoyl-CoA hydratases. Both enzymes catalyze the same reaction while the name of one of these enzymes denotes one direction of the corresponding reaction while the other name denotes the reverse reaction. As the reaction is reversible, both enzyme names can be used.

This enzyme, also known as crotonase, is naturally involved in metabolizing fatty acids to produce both acetyl-CoA and energy. Enzymes belonging to this class have been described to occur, e.g. in rat (Rattus norvegicus), humans (Homo sapiens), mouse (Mus musculus), wild boar (Sus scrofa), Bos taurus, E. coli, Clostridium acetobutylicum and Clostridium aminobutyricum. Nucleotide and/or amino acid sequences for such enzymes have been determined, e.g. for rat, humans and Bacillus subtilis and Bacillus anthracis. In principle, any enoyl-CoA hydratase (EC 4.2.1.17) which can catalyze the conversion of 3-hydroxyisovalerate (HIV) into 3-methylcrotonic acid can be used in the context of the present invention. In a preferred embodiment the enoyl-CoA hydratase is an enoyl-CoA hydratase of Galactomyces reessii (Dhar et al., J. Ind. Microbiol. Biotechnol. 28 (2002), 81-87), an enoyl-CoA hydratase of Bacillus subtilis (Uniprot G4PBC3; SEQ ID NO: 38) or an enoyl-CoA hydratase of Bacillus anthracis (Uniprot Q81YG6; SEQ ID NO: 39).

In a preferred embodiment, the enoyl-CoA hydratase employed in the method of the invention has an amino acid sequence as shown in any one of SEQ ID NOs: 38 or 39 or shows an amino acid sequence which is at least x % homologous to any one of SEQ ID NOs: 38 or 39 and has the activity of an enoyl-CoA hydratase with x being an integer between 30 and 100, preferably 35, 40, 45, 50, 55, 60, 65, 70, 75, 80, 85, 90, 91, 92, 93, 94, 95, 96, 97, 98 or 99 wherein such an enzyme is capable of converting 3-hydroxyisovalerate (HIV) into 3-methylcrotonic acid as set forth herein above. As regards the determination of the degree of identity, the same applies as has been set forth herein above.

The enzymatic condensation of acetone and acetyl-CoA into 3-hydroxyisovalerate (HIV): step III as shown in FIG. 1

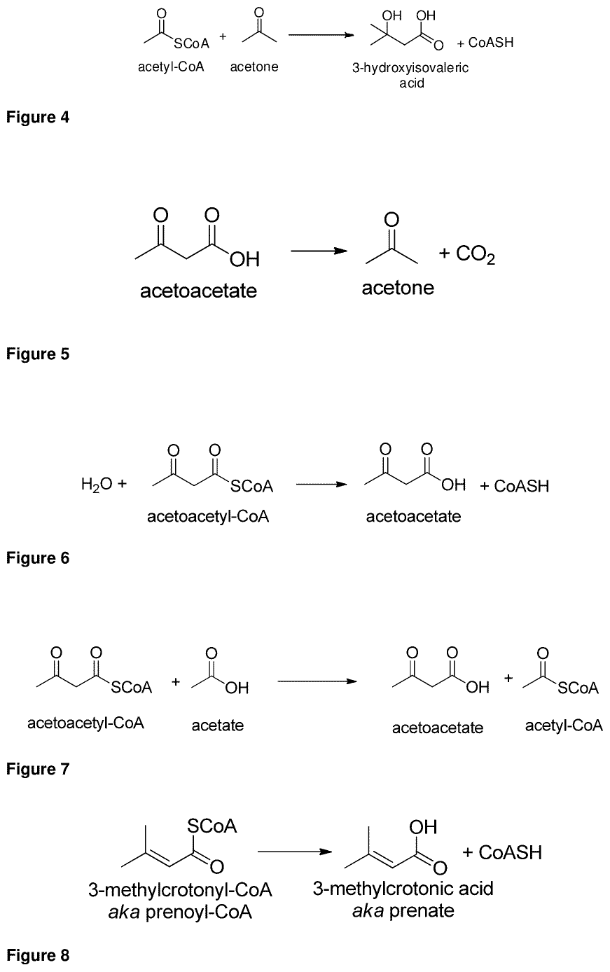

The 3-hydroxyisovalerate (HIV) which is converted according to the method of the present invention into 3-methylcrotonic acid may itself be provided by an enzymatic reaction, namely the enzymatic condensation of acetone and acetyl-CoA into said 3-hydroxyisovalerate (HIV). The condensation of acetone and acetyl-CoA into said 3-hydroxyisovalerate (HIV) (step III as shown in FIG. 1) is schematically illustrated in FIG. 4.

Thus, the present invention also relates to a method for producing isobutene from acetone in which acetone is first condensed with acetyl-CoA into 3-hydroxyisovalerate (HIV) which is then converted into 3-methylcrotonic acid. Further, 3-methylcrotonic acid is then converted into isobutene as described herein above.

According to the present invention, the condensation of acetone and acetyl-CoA into 3-hydroxyisovalerate (HIV) preferably makes use of an enzyme which is capable of catalyzing the formation of a covalent bond between the carbon atom of the oxo (i.e., the C═O) group of acetone and acetyl-CoA, in particular the methyl group of acetyl-CoA. According to this reaction scheme, the oxo group of acetone reacts as an electrophile and the methyl group of acetyl-CoA reacts as a nucleophile. The general reaction of the conversion of acetone and acetyl-CoA is shown in FIG. 4. Enzymes which are capable of enzymatically condensing acetone and acetyl-CoA into 3-hydroxyisovalerate (HIV) are known in the art and have, e.g., been described in WO 2011/032934.

Preferably, the enzyme employed in the enzymatic condensation of acetone and acetyl-CoA into 3-hydroxyisovalerate (HIV) is an enzyme with the activity of a HMG CoA synthase (EC 2.3.3.10) and/or a PksG protein and/or an enzyme with the activity of a C—C bond cleavage/condensation lyase, such as a HMG CoA lyase (EC 4.1.3.4). HMG CoA synthase has been described for various organisms.

Examples of HMG CoA synthases from different organisms are given in SEQ ID NO: 1 to 16. SEQ ID NO: 1 shows the sequence of the cytoplasmic HMG CoA synthase of Caenorhabditis elegans (P54871, gene bank F25B4.6), SEQ ID NO: 2 shows the sequence of the cytoplasmic HMG CoA synthase of Schizosaccharomyces pombe (fission yeast; P54874), SEQ ID NO: 3 shows the sequence of the cytoplasmic HMG CoA synthase of Saccharomyces cerevisiae (baker's yeast; P54839, gene bank CAA65437.1), SEQ ID NO: 4 shows the sequence of the cytoplasmic HMG CoA synthase of Arabidopsis thaliana (Mouse-ear cress; P54873), SEQ ID NO: 5 shows the sequence of the cytoplasmic HMG CoA synthase of Dictyostelium discoideum (Slime mold; P54872, gene bank L2114), SEQ ID NO: 6 shows the sequence of the cytoplasmic HMG CoA synthase of Blattella germanica (German cockroach; P54961, gene bank X73679), SEQ ID NO: 7 shows the sequence of the cytoplasmic HMG CoA synthase of Gallus gallus (Chicken; P23228, gene bank CHKHMGCOAS), SEQ ID NO: 8 shows the sequence of the cytoplasmic HMG CoA synthase of Homo sapiens (Human; 001581, gene bank X66435), SEQ ID NO: 9 shows the sequence of the mitochondrial HMG CoA synthase of Homo sapiens (Human; P54868, gene bank X83618), SEQ ID NO: 10 shows the sequence of the mitochondrial HMG CoA synthase of Dictyostelium discoideum (Slime mold; Q86HL5, gene bank XM_638984), SEQ ID NO: 11 shows the sequence of the HMG CoA synthase of Staphylococcus epidermidis (Q9FD76), SEQ ID NO: 12 shows the sequence of the HMG CoA synthase of Lactobacillus fermentum (B2GBL1), SEQ ID NO: 13 shows the sequence of the HMG CoA synthase of Hyperthermus butylicus (A2BMY8), SEQ ID NO: 14 shows the sequence of the HMG CoA synthase of Chloroflexus aggregans (B8G795), SEQ ID NO: 15 shows the sequence of the HMG CoA synthase of Lactobacillus delbrueckii (Q1GAH5) and SEQ ID NO: 16 shows the sequence of the HMG CoA synthase of Staphylococcus haemolyticus Q4L958 (I98>V difference compared to wild type protein).

In a preferred embodiment of the present invention the HMG CoA synthase is an enzyme comprising an amino acid sequence selected from the group consisting of SEQ ID NOs: 1 to 16 or a sequence which is at least n % identical to any of SEQ ID NOs: 1 to 16 and having the activity of a HMG CoA synthase with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99.

As regards the determination of sequence identity, the same applies as has been set forth above.

Another example for a protein which can be used in the condensation of acetone and acetyl-CoA into 3-hydroxyisovalerate is a PksG protein. In the context of the present application the term “PksG protein” or “a protein/enzyme having the activity of a PksG protein” refers to any enzyme which is able to catalyze the reaction which is naturally catalyzed by the PksG protein, i.e., the transfer of —CH₂COO— from acetyl-S-AcpK (Ac-S-AcpK) to a β-ketothioester polyketide intermediate linked to one of the thiolation domains of the PksL protein. This is a reaction which is analogous to that catalyzed by HMG CoA synthase with the difference that the acetyl-thioester of the phosphopantetheyl moiety is attached to a carrier protein rather than to part of Coenzyme A. Although the PksG protein in the reaction which it naturally catalyzes transfers the acetyl group from acetyl-S-AcpK to an acceptor, it has been shown previously that the PksG protein can also effect the reaction which is normally catalyzed by HMG CoA synthase, i.e. the synthesis of HMG CoA starting from acetoacetyl CoA and acetyl CoA.

Examples of PksG proteins are given in SEQ ID NO: 17 and 18. Preferably, the PksG protein is an enzyme comprising an amino acid sequence which is at least n % identical to SEQ ID NO: 17 or 18 and having the activity of a PksG protein with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99.

SEQ ID NO: 17 shows the amino acid sequence of the PksG protein of Bacillus subtilis (P40830) and SEQ ID NO: 18 shows the amino acid sequence of the PksG protein of Mycobacterium marinum (B2HGT6).

As regards the determination of the degree of sequence identity the same applies as has been set forth above in connection with HMG CoA synthase.

Examples of “C—C bond cleavage/condensation lyases” in particular include enzymes which are classified as isopropylmalate synthase (EC 2.3.3.13), as homocitrate synthase (EC 2.3.3.14) or as 4-hydroxy-2-ketovalerate aldolase (EC 4.1.3.39). Isopropylmalate synthase catalyzes the following reaction: acetyl-CoA+3-methyl-2-oxobutanoate+H₂O [symbol] (2S)-2-isopropylmalate+CoA. Examples for such enzymes are the corresponding enzyme from Brucella abortus (strain 2308; Q2YRT1) and the corresponding enzyme from Hahella chejuensis (strain KCTC 2396; Q2SFA7).

A homocitrate synthase (EC 2.3.3.14) is an enzyme that catalyzes the chemical reaction acetyl-CoA+H₂O+2-oxoglutarate [symbol] (R)-2-hydroxybutane-1,2,4-tricarboxylate+CoA. The 4-hydroxy-2-ketovalerate aldolase catalyzes the chemical reaction 4-hydroxy-2-oxopentanoate [symbol] acetaldehyde+pyruvate.

Examples for enzymes classified as “HMG CoA lyase” or “a protein/enzyme having the activity of a HMG CoA lyase” in the EC number EC 4.1.3.4, are given in SEQ ID NOs: 19 to 25. SEQ ID NO: 19 shows the sequence of the HMG CoA lyase of Zea mays (Accession number B6U7B9, gene bank ACG45252), SEQ ID NO: 20 shows the sequence of the HMG CoA lyase of Danio rerio (Brachydanio rerio; A8WG57, gene bank BC154587), SEQ ID NO: 21 shows the sequence of the HMG CoA lyase of Bos taurus (Uniprot accession number Q29448) and SEQ ID NO: 22 shows the sequence of the HMG CoA lyase of Homo sapiens (mitochondrial, Uniprot accession number P35914, gene bank HUMHYMEGLA), SEQ ID NO: 23 shows the sequence of the HMG CoA lyase of Pseudomonas putida (Q88H25), SEQ ID NO: 24 shows the sequence of the HMG CoA lyase of Acinetobacter baumannii (B7H4C6) and SEQ ID NO: 25 shows the sequence of the HMG CoA lyase of Thermus thermophilus (Q721H0).

In a preferred embodiment of the present invention the HMG CoA lyase is an enzyme comprising an amino acid sequence selected from the group consisting of SEQ ID NOs: 19 to 25 or a sequence which is at least n % identical to any of SEQ ID NOs: 19 to 25 and having the activity of a HMG CoA lyase with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99.

As regards the determination of the degree of sequence identity the same applies as has been set forth above in connection with HMG CoA synthase.

The enzymatic conversion of acetoacetate into acetone: step IV as shown in FIG. 1

The acetone which is converted according to the method of the present invention into 3-hydroxyisovalerate (HIV) may itself be provided by an enzymatic reaction, namely the enzymatic conversion of acetoacetate into acetone. The conversion of acetoacetate into acetone (step IV as shown in FIG. 1) is schematically illustrated in FIG. 5. This reaction is a decarboxylation reaction and is a natural occurring reaction in organisms capable of producing acetone, i.e., organisms of the genus Clostridia.

Thus, the present invention also relates to a method for producing isobutene from acetoacetate in which acetoacetate is first converted into acetone which is then condensed with acetyl-CoA into 3-hydroxyisovalerate (HIV) which is then converted into 3-methylcrotonic acid as described herein above. Further, said 3-methylcrotonic acid is then converted into isobutene as described herein above.

According to the present invention, the conversion of acetoacetate into said acetone preferably makes use of an acetoacetate decarboxylase (EC 4.1.1.4). Nucleotide sequences from several organisms encoding this enzyme are known in the art, e.g. the adc gene from Clostridium acetobutylicum (Uniprot accession numbers P23670 and P23673), Clostridium beijerinckii (Clostridium MP; Q9RPK1), Clostridium pasteurianum (Uniprot accession number P81336), Bradyrhizobium sp. (strain BTAi1/ATCC BAA-1182; Uniprot accession number A5EBU7), Burkholderia mallei (ATCC 10399 A9LBS0), Burkholderia mallei (Uniprot accession number A3MAE3), Burkholderia mallei FMH A5XJB2, Burkholderia cenocepacia (Uniprot accession number A0B471), Burkholderia ambifaria (Uniprot accession number Q0b5P1), Burkholderia phytofirmans (Uniprot accession number B2T319), Burkholderia spec. (Uniprot accession number Q38ZU0), Clostridium botulinum (Uniprot accession number B2TLN8), Ralstonia pickettii (Uniprot accession number B2UIG7), Streptomyces nogalater (Uniprot accession number Q9EYI7), Streptomyces avermitilis (Uniprot accession number Q82NF4), Legionella pneumophila (Uniprot accession number Q5ZXQ9), Lactobacillus salivarius (Uniprot accession number Q1WVG5), Rhodococcus spec. (Uniprot accession number Q0S7W4), Lactobacillus plantarum (Uniprot accession number Q890GO), Rhizobium leguminosarum (Uniprot accession number Q1 M911), Lactobacillus casei (Uniprot accession number Q03B66), Francisella tularensis (Uniprot accession number Q0BLC9), Saccharopolyspora erythreae (Uniprot accession number A4FKR9), Korarchaeum cryptofilum (Uniprot accession number B1 L3N6), Bacillus amyloliquefaciens (Uniprot accession number A7Z8K8), Cochliobolus heterostrophus (Uniprot accession number Q8NJQ3), Sulfolobus islandicus (Uniprot accession number C3ML22) and Francisella tularensis subsp. holarctica (strain OSU18).

In a preferred embodiment, the acetoacetate decarboxylase employed in the method of the present invention in the conversion of acetoacetate into acetone is an acetoacetate decarboxylase (EC 4.1.1.4) derived from Clostridium acetobutylicum (Uniprot accession numbers P23670 and P23673).

The enzymatic conversion of acetoacetyl-CoA into acetoacetate: step Va and step Vb as shown in FIG. 1

The acetoacetate which is converted according to the method of the present invention into acetone may itself be provided by an enzymatic reaction, namely the enzymatic conversion of acetoacetyl-CoA into acetoacetate. The conversion of acetoacetyl-CoA into acetoacetate can be achieved by two different routes. One possibility is the conversion of acetoacetyl-CoA into acetoacetate by hydrolysing the CoA thioester of acetoacetyl-CoA into acetoacetate. This reaction (step Va as shown in FIG. 1) is schematically illustrated in FIG. 6. In another, more preferred, aspect the CoA group of acetoacetyl-CoA is transferred on acetate, resulting in the formation of acetoacetate and acetyl-CoA. This reaction (step Vb as shown in FIG. 1) is schematically illustrated in FIG. 7.

Thus, the present invention also relates to a method for producing isobutene from acetoacetyl-CoA in which acetoacetyl-CoA is first converted into acetoacetate which is then converted into acetone which is then condensed with acetyl-CoA into 3-hydroxyisovalerate (HIV) which is then converted into 3-methylcrotonic acid as described herein above. Further, said 3-methylcrotonic acid is then converted into isobutene as described herein above.

As mentioned, in one aspect, the CoA thioester of acetoacetyl-CoA is hydrolyzed to result in acetoacetate. According to this aspect of the present invention, the enzymatic conversion of acetoacetyl-CoA into acetoacetate is achieved by preferably making use of an acetoacetyl-CoA hydrolase (EC 3.1.2.11) which naturally catalyzes this reaction.

Acetoacetyl-CoA hydrolases (EC 3.1.2.11) catalyse the following reaction:

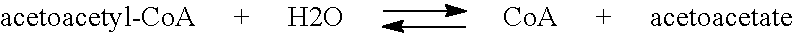

This enzyme is known from various organisms and has, e.g., been described in eukaryotic organisms. The enzyme has, e.g., been described in Bos taurus, Columba livia, Gallus gallus, Homo sapiens, Mus musculus, Oncorhynchus mykiss, Oryctolagus cuniculus, or Rattus norvegicus. Thus, in a preferred embodiment, the enzyme is from the genus selected from the group consisting of Bos, Columba, Gallus, Mus, Oncorhynchus, Oryctolagus, and Rattus. In a more preferred embodiment, the enzyme is from the species selected from the group consisting of Bos taurus, Columba livia, Gallus gallus, Homo sapiens, Mus musculus, Oncorhynchus mykiss, Oryctolagus cuniculus, or Rattus norvegicus. Bos taurus, Columba livia, Gallus gallus, Homo sapiens, Mus musculus, Oncorhynchus mykiss, Oryctolagus cuniculus, and Rattus norvegicus.

As mentioned, in another, more preferred, possibility, the CoA group of acetoacetyl-CoA is transferred on acetate, resulting in the formation of acetoacetate and acetyl-CoA. According to this possibility of the present invention, the enzymatic conversion of acetoacetyl-CoA into acetoacetate is achieved by preferably making use of an enzyme which is capable of transferring the CoA group of acetoacetyl-CoA on acetate.

Preferably, such an enzyme capable of transferring the CoA group of acetoacetyl-CoA on acetate belongs to the family of CoA transferases (EC 2.8.3.-).

Thus, the present invention relates to a method for the enzymatic conversion of acetoacetyl-CoA into acetoacetate by making use of an enzyme capable of transferring the CoA group of acetoacetyl-CoA on acetate, preferably a CoA transferase (EC 2.8.3.-). A preferred example of an enzyme catalysing the conversion of acetoacetyl-CoA into acetoacetate which can be employed in the method of the present invention is an enzyme classified as an acetate CoA transferase (EC 2.8.3.8).

Acetate CoA transferases (EC 2.8.3.8) catalyse the following reaction:

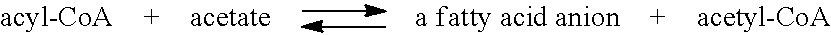

Acetate CoA transferases (EC 2.8.3.8) are known from various organisms, e.g., from E. coli in which it is encoded by the atoD gene atoA genes (UniProt accession numbers P76458 and P76459). An acetate CoA transferase is also known from Clostrtidium acetobutylicum in which it is encoded by the ctfAB gene. Thus, in a preferred embodiment, of the invention, an acetate CoA transferase (EC 2.8.3.8) is used for the conversion of acetoacetyl-CoA into acetoacetate which is derived from E. coli and which it is encoded by the atoD gene atoA genes (UniProt accession numbers P76458 and P76459) or which is derived from Clostrtidium acetobutylicum and which it is encoded by the ctfAB gene.

The enzymatic conversion of 3-methylcrotonyl-CoA into 3-methylcrotonic acid: step VI as shown in FIG. 1

The 3-methylcrotonic acid can be provided by another possible route which is described in the following.

Thus, in another embodiment, the 3-methylcrotonic acid which is converted into isobutene may itself be provided by another enzymatic reaction, namely the enzymatic conversion of 3-methylcrotonyl-CoA into 3-methylcrotonic acid. The conversion of 3-methylcrotonyl-CoA into 3-methylcrotonic acid (step VI as shown in FIG. 1) is schematically illustrated in FIG. 8.

The conversion of 3-methylcrotonyl-CoA into 3-methylcrotonic acid can, e.g., be achieved in different ways, e.g., by three alternative enzymatic routes described in the following and as shown in FIG. 1 (step VIa, step VIb or step VIc as shown in FIG. 1).

Thus, the enzymatic conversion of 3-methylcrotonyl-CoA into 3-methylcrotonic acid may be achieved by
(a) a single enzymatic reaction in which 3-methylcrotonyl-CoA is directly converted into 3-methylcrotonic acid, preferably by making use of a CoA transferase (EC 2.8.3.-), preferably a propionate:acetate-CoA transferase (EC 2.8.3.1), an acetate CoA-transferase (EC 2.8.3.8) or a succinyl-CoA:acetate CoA-transferase (EC 2.8.3.18) (step VIa as shown in FIG. 1);
(b) a single enzymatic reaction in which 3-methylcrotonyl-CoA is directly converted into 3-methylcrotonic acid, preferably by making use of a thioester hydrolase (EC 3.1.2.-), preferably an acetyl-CoA hydrolase (EC 3.1.2.1), an ADP-dependent short-chain-acyl-CoA hydrolase (EC 3.1.2.18) or an acyl-CoA hydrolase (EC 3.1.2.20) (step VIb as shown in FIG. 1); or
(c) two enzymatic steps comprising
   (i) first enzymatically converting 3-methylcrotonyl-CoA into 3-methylcrotonyl phosphate; and
   (ii) then enzymatically converting the thus obtained 3-methylcrotonyl phosphate into said 3-methylcrotonic acid (step VIc as shown in FIG. 1).

Thus, one possibility is a two-step conversion from 3-methylcrotonyl-CoA via 3-methylcrotonyl phosphate into 3-methylcrotonic acid. Two other options involve a direct conversion of 3-methylcrotonyl-CoA into 3-methylcrotonic acid. These three options will be discussed in the following.

Accordingly, in one embodiment, the enzymatic conversion of 3-methylcrotonyl-CoA into 3-methylcrotonic acid is achieved by two enzymatic steps comprising (i) first enzymatically converting 3-methylcrotonyl-CoA into 3-methylcrotonyl phosphate; and (ii) then enzymatically converting the thus obtained 3-methylcrotonyl phosphate into said 3-methylcrotonic acid (as shown in step VIc of FIG. 1). The corresponding reaction is schematically shown in FIG. 11.

The conversion of 3-methylcrotonyl-CoA into 3-methylcrotonyl phosphate can, e.g., be achieved by the use of a phosphate butyryltransferase (EC 2.3.1.19) or a phosphate acetyltransferase (EC 2.3.1.8).

Phosphate butyryltransferase (EC 2.3.1.19) naturally catalyzes the following reaction

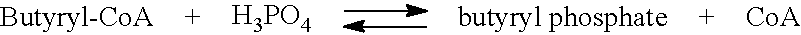

It has been described by Wiesenborn et al. (Appl. Environ. Microbiol. 55 (1989), 317-322) and by Ward et al. (J. Bacteriol. 181 (1999), 5433-5442) that phosphate butyryltransferases (EC 2.3.1.19) can use a number of substrates in addition to butyryl coenzyme A (butyryl-CoA), in particular acetyl-CoA, propionyl-CoA, isobutyryl-CoA, valeryl-CoA and isovaleryl-CoA.

The enzyme has been described to occur in a number of organisms, in particular in bacteria and in protozoae. In one embodiment the enzyme is from the protozoae Dasytricha ruminantium. In a preferred embodiment the phosphate butyryltransferase is a phosphate butyryltransferase from a bacterium, preferably from a bacterium of the genus Bacillus, Butyrivibrio, Enterococcus or Clostridium, more preferably Enterococcus or Clostridium, and even more preferably from Bacillus megaterium, Bacillus subtilis, Butyrivibrio fibrisolvens, Clostridium acetobutylicum, Clostridium beijerinckii, Clostridium butyricum, Clostridium kluyveri, Clostridium saccharoacetobutylicum, Clostridium sprorogenes or Enterococcus faecalis. Most preferably, the enzyme is from Clostridium acetobutylicum, in particular the enzyme encoded by the ptb gene (Uniprot Accession number F0K6W0; Wiesenborn et al. (Appl. Environ. Microbiol. 55 (1989), 317-322)) or from Enterococcus faecalis (Uniprot Accession number K4YRE8; Ward et al. (J. Bacteriol. 181 (1999), 5433-5442)).

In a preferred embodiment, the conversion of 3-methylcrotonyl-CoA into 3-methylcrotonyl phosphate is achieved by making use of a phosphate butyryltransferase from Clostridium acetobutylicum, preferably from Clostridium acetobutylicum strain ATCC 824. The amino acid sequence of said protein is shown in SEQ ID NO: 26.

It is, of course, not only possible to use an enzyme exactly showing this amino acid of SEQ ID NO:26. It is also possible to use an enzyme which comprises a sequence which is at least 60% identical to the amino acid sequence shown in SEQ ID NO: 26.

Preferably, the sequence identity is at least 70%, more preferably at least 80%, 85% or 90%, even more preferably 91%, 92%, 93,%, 94%, 95%, 96%, 97%, 98% and particularly preferred at least 99% to SEQ ID NO:26 and the enzyme has the enzymatic activity of converting 3-methylcrotonyl-CoA into 3-methylcrotonyl phosphate. As regards the determination of the sequence identity, the same applies as has been set forth above.

In another preferred embodiment, the conversion of 3-methylcrotonyl-CoA into 3-methylcrotonyl phosphate is achieved by making use of a phosphate butyryltransferase from Bacillus subtilis, preferably from Bacillus subtilis having the UniProt Accession number P54530. The amino acid sequence of said protein is shown in SEQ ID NO: 73.

In a preferred embodiment of the present invention the phosphate butyryltransferase is an enzyme comprising the amino acid sequence of SEQ ID NO: 73 or a sequence which is at least n % identical to SEQ ID NO: 73 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylcrotonyl-CoA into 3-methylcrotonyl phosphate. As regards the determination of the sequence identity, the same applies as has been set forth above.

In another preferred embodiment, the conversion of 3-methylcrotonyl-CoA into 3-methylcrotonyl phosphate is achieved by making use of a phosphate butyryltransferase from Enterococcus faecalis, preferably from Enterococcus faecalis having the UniProt Accession number S4BZL5. The amino acid sequence of said protein is shown in SEQ ID NO: 74.

In a preferred embodiment of the present invention the phosphate butyryltransferase is an enzyme comprising the amino acid sequence of SEQ ID NO: 74 or a sequence which is at least n % identical to SEQ ID NO: 74 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylcrotonyl-CoA into 3-methylcrotonyl phosphate. As regards the determination of the sequence identity, the same applies as has been set forth above.

Phosphate acetyltransferase (EC 2.3.1.8) naturally catalyzes the following reaction

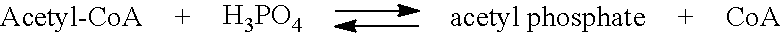

It has been described by Veit et al. (J. Biotechnol. 140 (2009), 75-83) that phosphate acetyltransferase can also use as a substrate butyryl-CoA or propionyl-CoA.

The accession numbers for this enzyme family in InterPro database are IPR012147 and IPR002505, “http://www.ebi.ac.uk/interpro/entry/IPR002505” (http://www.ebi.ac.uk/interpro/entry/IPR012147 http://www.ebi.ac.uk/interpro/entry/IPR002505) See also http://pfam.sanger.ac.uk/family/PF01515

The enzyme has been described in a variety of organisms, in particular bacteria and fungi. Thus, in one preferred embodiment the enzyme is an enzyme from a bacterium, preferably of the genus Escherichia, Chlorogonium, Clostridium, Veillonella, Methanosarcina, Corynebacterium, Ruegeria, Salmonella, Azotobacter, Bradorhizobium, Lactobacillus, Moorella, Rhodopseudomonas, Sinorhizobium, Streptococcus, Thermotoga or Bacillus, more preferably of the species Escherichia coli, Chlorogonium elongatum, Clostridium kluyveri, Clostridium acetobutylicum, Clostridium acidurici, Veillonella parvula, Methanosarcina thermophila, Corynebacterium glutamicum, Ruegeria pomeroyi, Salmonella enterica, Azotobacter vinelandii, Bradyrhizobium japonicum, Lactobacillus fermentum, Lactobacillus sanfranciscensis, Moorella thermoacetica, Rhodopseudomonas palustris, Sinorhizobium meliloti, Streptococcus pyogenes, Thermotoga maritima or Bacillus subtilis. In another preferred embodiment the enzyme is an enzyme from a fungus, preferably from the genus Saccharomyces, more preferably of the species Saccharomyces cerevisiae.

In a preferred embodiment, the conversion of 3-methylcrotonyl-CoA into 3-methylcrotonyl phosphate is achieved by making use a phosphate acetyltransferase from Corynebacterium glutamicum, preferably from Corynebacterium glutamicum strain ATCC 13032. The amino acid sequence of said protein is shown in SEQ ID NO: 27.

It is, of course, not only possible to use an enzyme exactly showing this amino acid of SEQ ID NO:27. It is also possible to use an enzyme which comprises a sequence which is at least 60% identical to the amino acid sequence shown in SEQ ID NO: 27. Preferably, the sequence identity is at least 70%, more preferably at least 80%, 85% or 90%, even more preferably 91%, 92%, 93,%, 94%, 95%, 96%, 97%, 98% and particularly preferred at least 99% to SEQ ID NO:27 and the enzyme has the enzymatic activity of converting 3-methylcrotonyl-CoA into 3-methylcrotonyl phosphate. As regards the determination of the sequence identity, the same applies as has been set forth above.

The conversion of 3-methylcrotonyl phosphate into 3-methylcrotonic acid can, e.g., be achieved by making use of an enzyme which is classified as EC 2.7.2.-, i.e., a phosphotransferase. Such enzymes use a carboxy group as acceptor. Thus, the conversion of 3-methylcrotonyl phosphate into 3-methylcrotonic acid can, e.g., be achieved by making use of an enzyme with a carboxy group as acceptor (EC 2.7.2.-). In a preferred embodiment, the conversion of 3-methylcrotonyl phosphate into 3-methylcrotonic acid is achieved by the use of a propionate kinase (EC 2.7.2.15), an acetate kinase (EC 2.7.2.1), a butyrate kinase (EC 2.7.2.7) or a branched-chain-fatty-acid kinase (EC 2.7.2.14).

Butyrate kinases (EC 2.7.2.7) naturally catalyze the following reaction

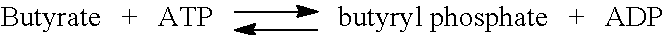

It has been described, e.g. by Hartmanis (J. Biol. Chem. 262 (1987), 617-621) that butyrate kinase can use a number of substrates in addition to butyrate, e.g. valerate, isobutyrate, isovalerate and vinyl acetate. The enzyme has been described in a variety of organisms, in particular bacteria. In one preferred embodiment the enzyme is from a bacterium, preferably from a bacterium of the genus Clostridium, Butyrivibrio, Thermotoga or Enterococcus. Preferred is Clostridium. More preferably the enzyme is from a bacterium of the species Clostridium acetobutylicum, Clostridium proteoclasticum, Clostridium tyrobutyricum, Clostridium butyricum, Clostridium pasteurianum, Clostridium tetanomorphum, Butyrivibrio firbrosolvens, Butyrivibrio hungatei, Thermotoga maritime or Enterococcus durans. Preferred is Clostridium acetobutylicum. For this organism two butyrate kinases have been described: butyrate kinase 1 (Uniprot Accession number: Q45829) and butyrate kinase II (Uniprot Accession number: Q971119).

In another preferred embodiment, the conversion of 3-methylcrotonyl phosphate into 3-methylcrotonic acid is achieved by making use of a butyrate kinase from Lactobacillus, preferably from Lactobacillus casei (UniProt Accession number KON529) or a butyrate kinase from Geobacillus, preferably from Geobacillus sp. (UniProt Accession number L8A0E1). The amino acid sequence of these proteins are shown in SEQ ID NO:75 and SEQ ID NO:76, respectively.

In a preferred embodiment of the present invention the butyrate kinase is an enzyme comprising the amino acid sequence of SEQ ID NO: 75 or 76 or a sequence which is at least n % identical to SEQ ID NO: 75 or 76 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylcrotonyl phosphate into 3-methylcrotonic acid. As regards the determination of the sequence identity, the same applies as has been set forth above.

Branched-chain-fatty-acid kinases (EC 2.7.2.14) naturally catalyze the following reaction

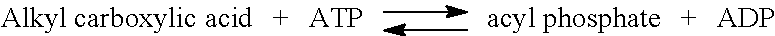

wherein “alkyl” may be 2-methylbutanoate, butanoate, isobutanoate, pentanoate or propionate. The latter reaction with propionate has been described for a branched-chain fatty acid kinase from a spirochaete (J. Bacteriol. 152 (1982), 246-54).

This enzyme has been described to occur in a number of bacteria. Thus, in one preferred embodiment the enzyme is an enzyme from a bacterium, preferably of the genus Spirochaeta or Thermotoga, more preferably Thermotoga maritime.

Propionate kinases (EC 2.7.2.15) naturally catalyze the following reactions

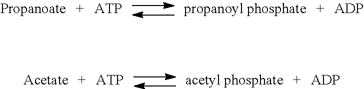

This enzyme has been described to occur in a number of bacteria, in particular Enterobacteriacea. Thus, in one preferred embodiment the enzyme is an enzyme from a bacterium, preferably of the genus Salmonella or Escherichia, more preferably of the species Salmonella enterica, Salmonella typhimurium or Escherichia coli.

In a preferred embodiment, the conversion of 3-methylcrotonyl phosphate into 3-methylcrotonic acid is achieved by making use of a propionate kinase from Salmonella typhimurium, preferably from Salmonella typhimurium strain ATCC 700720. The amino acid sequence of said protein is shown in SEQ ID NO: 28.

It is, of course, not only possible to use an enzyme exactly showing this amino acid of SEQ ID NO:28. It is also possible to use an enzyme which comprises a sequence which is at least 60% identical to the amino acid sequence shown in SEQ ID NO: 28. Preferably, the sequence identity is at least 70%, more preferably at least 80%, 85% or 90%, even more preferably 91%, 92%, 93,%, 94%, 95%, 96%, 97%, 98% and particularly preferred at least 99% to SEQ ID NO:28 and the enzyme has the enzymatic activity of converting 3-methylcrotonyl phosphate into 3-methylcrotonic acid. As regards the determination of the sequence identity, the same applies as has been set forth above.

In another preferred embodiment, the conversion of 3-methylcrotonyl phosphate into 3-methylcrotonic acid is achieved by making use of a propionate kinase from Escherichia coli, preferably from Escherichia coli strain K12. The amino acid sequence of said protein is shown in SEQ ID NO: 29.

It is, of course, not only possible to use an enzyme exactly showing this amino acid of SEQ ID NO:29. It is also possible to use an enzyme which comprises a sequence which is at least 60% identical to the amino acid sequence shown in SEQ ID NO: 29. Preferably, the sequence identity is at least 70%, more preferably at least 80%, 85% or 90%, even more preferably 91%, 92%, 93,%, 94%, 95%, 96%, 97%, 98% and particularly preferred at least 99% to SEQ ID NO:29 and the enzyme has the enzymatic activity of converting 3-methylcrotonyl phosphate into 3-methylcrotonic acid. As regards the determination of the sequence identity, the same applies as has been set forth above.

Acetate kinases (EC 2.7.2.1) naturally catalyze the following reaction

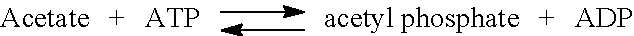

This enzyme has been described to occur in a number of organisms, in particular bacteria and eukaryotes. In one preferred embodiment the enzyme is from a bacterium, preferably from a bacterium of the genus Methanosarcina, Cryptococcus, Ethanoligenens, Propionibacterium, Roseovarius, Streptococcus, Salmonella, Acholeplasma, Acinetobacter, Ajellomyces, Bacillus, Borrelia, Chaetomium, Clostridium, Coccidioides, Coprinopsis, Cryptococcus, Cupriavidus, Desulfovibrio, Enterococcus, Escherichia, Ethanoligenes, Geobacillus, Helicobacter, Lactobacillus, Lactococcus, Listeria, Mesoplasma, Moorella, Mycoplasma, Oceanobacillus, Propionibacterium, Rhodospeudomonas, Roseovarius, Salmonella, Staphylococcus, Thermotoga or Veillonella, more preferably from a bacterium of the species Methanosarcina thermophila, Cryptococcus neoformans, Ethanoligenens harbinense, Propionibacterium acidipropionici, Streptococcus pneumoniae, Streptococcus enterica, Streptococcus pyogenes, Acholeplasma laidlawii, Acinetobacter calcoaceticus, Ajellomyces capsulatus, Bacillus subtilis, Borrelia burgdorferi, Chaetomium globosum, Clostridium acetobutylicum, Clostridium thermocellum, Coccidioides immitis, Coprinopsis cinerea, Cryptococcus neoformans, Cupriavidus necator, Desulfovibrio vulgaris, Enterococcus faecalis, Escherichia coli, Ethanoligenes harbinense, Geobacillus stearothermophilus, Helicobacter pylori, Lactobacillus delbrueckii, Lactobacillus acidophilus, Lactobacillus sanfranciscensis, Lactococcus lactis, Listeria monocytogenes, Mesoplasma florum, Methanosarcina acetivorans, Methanosarcina mazei, Moorella thermoacetica, Mycoplasma pneumoniae, Oceanobacillus iheyensis, Propionibacterium freudenreichii, Propionibacterium acidipropionici, Rhodospeudomonas palustris, Salmonella enteric, Staphylococcus aureus, Thermotoga maritime or Veillonella parvula.

In another preferred embodiment the enzyme is an enzyme from a fungus, preferably from a fungus of the genus Aspergillus, Gibberella, Hypocrea, Magnaporthe, Phaeosphaeria, Phanerochaete, Phytophthora, Sclerotinia, Uncinocarpus, Ustilago or Neurospora even more preferably from a fungus of the species Aspergillus fumigates, Aspergillus nidulans, Gibberella zeae, Hypocrea jecorina, Magnaporthe grisea, Phaeosphaeria nodorum, Phanerochaete chrysosporium, Phytophthora ramorum, Phytophthora sojae, Sclerotinia sclerotiorum, Uncinocarpus reesii, Ustilago maydis or Neurospora crassa.

In a further preferred embodiment the enzyme is an enzyme from a plant or an algae, preferably from the genus Chlamydomonas, even more preferably from the species Chlamydomonas reinhardtii.

In another embodiment the enzyme is from an organism of the genus Entamoeba, more preferably of the species Entamoeba histolytica.

The above mentioned enzyme families suitable for the conversion of 3-methylcrotonyl-CoA into 3-methylcrotonyl phosphate have been shown to be evolutionary related and contain common sequence signatures. Theses signatures are referenced and described in Prosite database: http://prosite.expasy.org/cgi-bin/prosite/nicedoc.pl?PS01075 Gao et al. (FEMS Microbiol. Lett. 213 (2002), 59-65) already described genetically modified E. coli cells which have been transformed, inter alia, with the ptb gene and the buk gene from Clostridium acetobutylicum encoding a phosphate butyryltransferase (EC 2.3.1.19) and a butyrate kinase (EC 2.7.2.7), respectively. These E. coli cells have been shown to be able to produce D-(−)-3-hydroxybutyric acid (3HB).

As mentioned above, the conversion of 3-methylcrotonyl-CoA into 3-methylcrotonic acid can also be achieved by two alternative conversions wherein 3-methylcrotonyl-CoA is directly converted into 3-methylcrotonic acid.

Preferably, in one embodiment, 3-methylcrotonyl-CoA is directly converted into 3-methylcrotonic acid by hydrolyzing the thioester bond of 3-methylcrotonyl-CoA into 3-methylcrotonic acid by making use of an enzyme which belongs to the family of thioester hydrolases (in the following referred to as thioesterases (EC 3.1.2.-)). This reaction is schematically shown in FIG. 10.

Examples for preferred thioester hydrolases (EC 3.1.2.-) are an acetyl-CoA hydrolase (EC 3.1.2.1), an ADP-dependent short-chain-acyl-CoA hydrolase (EC 3.1.2.18) and an acyl-CoA hydrolase (EC 3.1.2.20) (step VIb as shown in FIG. 1).

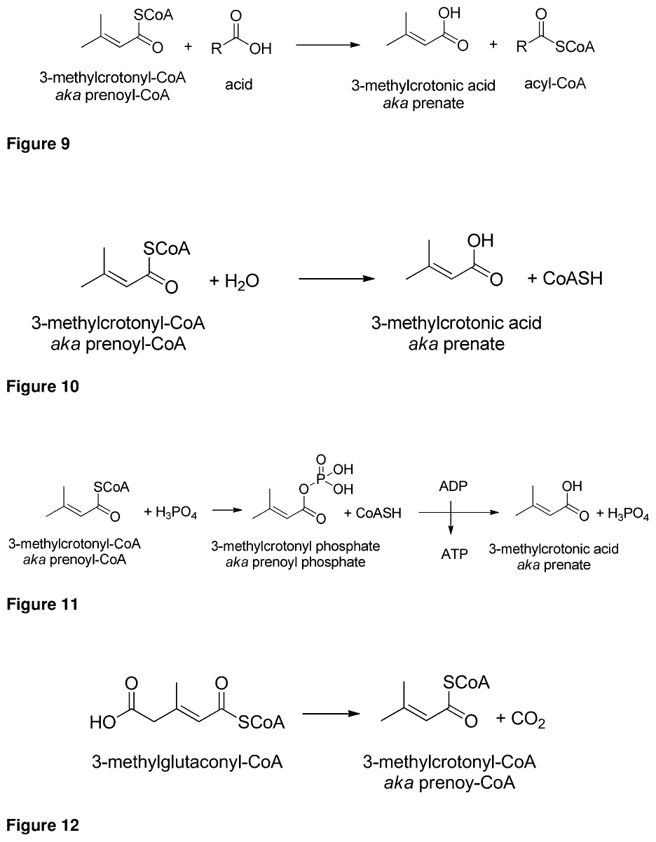

In an alternative embodiment, 3-methylcrotonyl-CoA is directly converted into 3-methylcrotonic acid, preferably by making use of an enzyme which belongs to the family of CoA-transferases (EC 2.8.3.-). This reaction is schematically shown in FIG. 9.

Examples for preferred CoA transferases (EC 2.8.3.-) are a propionate:acetate-CoA transferase (EC 2.8.3.1), an acetate CoA-transferase (EC 2.8.3.8) and a succinyl-CoA:acetate CoA-transferase (EC 2.8.3.18) (step VIa as shown in FIG. 1).

Thioesterases (TEs; also referred to as thioester hydrolases) are enzymes which are classified as EC 3.1.2. Presently thioesterases are classified as EC 3.1.2.1 through EC 3.1.2.30 while TEs which are not yet classified/unclassified are grouped as enzymes belonging to EC 3.1.2.-. Cantu et al. (Protein Science 19 (2010), 1281-1295) describe that there are 23 families of thioesterases which are unrelated to each other as regards the primary structure. However, it is assumed that all members of the same family have essentially the same tertiary structure. Thioesterases hydrolyze the thioester bond between a carbonyl group and a sulfur atom.

In a preferred embodiment, a thioesterase employed in a method according to the present invention for converting 3-methylcrotonyl-CoA into 3-methylcrotonic acid is selected from the group consisting of:
acetyl-CoA hydrolase (EC 3.1.2.1);
palmitoyl-CoA hydrolase (EC 3.1.2.2);
3-hydroxyisobutyryl-CoA hydrolase (EC 3.1.2.4);
oleoyl-[acyl-carrier-protein]hydrolase (EC 3.1.2.14);
ADP-dependent short-chain-acyl-CoA hydrolase (EC 3.1.2.18);
ADP-dependent medium-chain-acyl-CoA hydrolase (EC 3.1.2.19); and
acyl-CoA hydrolase (EC 3.1.2.20).

Thus, in one preferred embodiment the direct conversion of 3-methylcrotonyl-CoA into 3-methylcrotonic acid is achieved by making use of an acetyl-CoA hydrolase (EC 3.1.2.1). Acetyl-CoA hydrolases are enzymes which catalyze the following reaction:

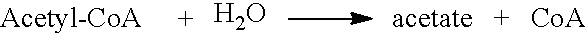

This enzyme occurs in a variety of organism, including eukaryotic and prokaryotic organisms, such as plants, animals, fungi and bacteria. The enzyme has, e.g., been described in Rattus norvegicus (Uniprot Accession number: Q99NB7), Mus musculus, Sus scrofa, Bos taurus, Gallus gallus, Platyrrhini, Ovis aries, Mesocricetus auratus, Ascaris suum, Homo sapiens (Uniprot Accession number: Q8WYK0), Pisum sativum, Cucumis sativus, Panicus sp., Ricinus communis, Solanum tuberosum, Spinacia oleracea, Zea mays, Glycine max, Saccharomyces cerevisiae, Neurospora crassa, Candida albicans, Trypanosoma brucei brucei, Trypanosoma cruzi, Trypanosoma dionisii, Trypanosoma vespertilionis, Crithidia fasciculate, Clostridium aminovalericum, Acidaminococcus fermaentans, Bradyrhizobium japonicum and Methanosarcina barkeri.

In another preferred embodiment the direct conversion of 3-methylcrotonyl-CoA into 3-methylcrotonic acid is achieved by making use of a palmitoyl-CoA hydrolase (EC 3.1.2.2). Palmitoyl-CoA hydrolases are enzymes which catalyze the following reaction:

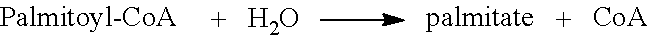

This enzyme occurs in a variety of organism, including eukaryotic and prokaryotic organisms, such as plants, animals, fungi and bacteria. The enzyme has, e.g., been described in Arabidopsis thaliana (Uniprot Accession number: Q8GYW7), Pisum sativum, Spinacia oleracea, Bumilleriopsis filiformis, Eremosphaera viridis, Mougeotia scalaris, Euglena gracilis, Rhodotorula aurantiaca, Saccharaomyces cerevisiae, Candida rugosa, Caenorhabditis elegans, Mus musculus (Uniprot Accession number: P58137), Homo sapiens, Platyrrhini, Bos taurus, Canis lupus familiaris, Sus scrofa, Cavia procellus, Columba sp., Cricetulus griseus, Mesocricetus auratus, Drosophila melanogaster, Rattus norvegicus, Oryctolagus cuniculus, Gallus gallus, Anas platyrhynchos, Mycobacterium tuberculosis, Mycobacterium phlei, Mycobacterium smegmatis, Acinetobacter colcaceticus, Haemophilus influenza, Helicobacter pylori, Bacillus subtilis, Pseudomonas aeruginosa, Rhodobacter shpaeroides, Streptomyces coelicolor, Streptomyces venezuelae and E. coli.

In a further preferred embodiment the direct conversion of 3-methylcrotonyl-CoA into 3-methylcrotonic acid is achieved by making use of a 3-hydroxyisobutyryl-CoA hydrolase (EC 3.1.2.4). 3-hydroxyisobutyryl-CoA hydrolases are enzymes which catalyze the following reaction:

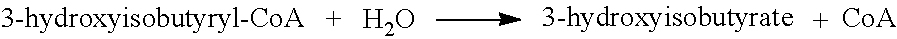

This enzyme occurs in a variety of organism, including eukaryotic and prokaryotic organisms, such as plants, animals, fungi and bacteria. The enzyme has, e.g., been described in Arabidopsis thaliana, Homo sapiens, Canus lupus familiaris, Rattus norvegicus, Bacillus cereus, Pseudomonas fluorescens and Pseudomonas aeruginosa.

In yet another preferred embodiment the direct conversion of 3-methylcrotonyl-CoA into 3-methylcrotonic acid is achieved by making use of an oleoyl-[acyl-carrier-protein]hydrolase (EC 3.1.2.14). Oleoyl-[acyl-carrier-protein]hydrolases are enzymes which catalyze the following reaction:

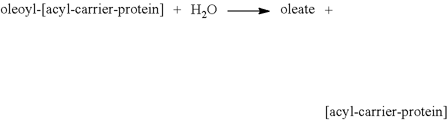

This enzyme occurs in a variety of plants and bacteria. The enzyme has, e.g., been described in Arabidopsis thaliana, Allium ampeloprasum, Curcurbita moschata, Cuphea calophylla, Cuphea hookeriana, Cuphea lanceolata, Cuphea wrightii, Umbellularia californica, Coriandrum sativum, Spinacia oleracea, Elaeis sp., Elaeis guineensis, Glycine max, Persea americana, Pisum sativum, Sinapis alba, Ulmus americana, Zea mays, Brassica juncea, Brassica napus, Brassica rapa subsp. campestris, Jatropha curcas, Ricinus communis, Cinnamomum camphorum, Macadamia tetraphylla, Magnifera indica, Madhuca longifolia, Populus tomentosa, Chimonanthus praecox, Gossypium hirsutum, Diploknema butyracea, Helianthus annuus and Streptococcus pyogenes.

In yet another preferred embodiment the direct conversion of 3-methylcrotonyl-CoA into 3-methylcrotonic acid is achieved by making use of an ADP-dependent short-chain-acyl-CoA hydrolase (EC 3.1.2.18). ADP-dependent short-chain-acyl-CoA hydrolases are enzymes which catalyze the following reaction:

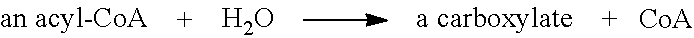

This enzyme occurs in a variety of animals and has, e.g., been described in Mus musculus, Rattus norvegicus and Mesocricetus auratus.

In yet another preferred embodiment the direct conversion of 3-methylcrotonyl-CoA into 3-methylcrotonic acid is achieved by making use of an ADP-dependent medium-chain-acyl-CoA hydrolase (EC 3.1.2.19). ADP-dependent medium-chain-acyl-CoA hydrolases are enzymes which catalyze the following reaction:

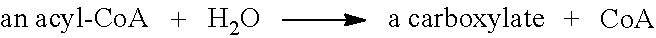

This enzyme occurs in a variety of animals and has, e.g., been described in Rattus norvegicus and Mesocricetus auratus.

In a further preferred embodiment the direct conversion of 3-methylcrotonyl-CoA into 3-methylcrotonic acid is achieved by making use of an acyl-CoA hydrolase (EC 3.1.2.20). Acyl-CoA hydrolases are enzymes which catalyze the following reaction:

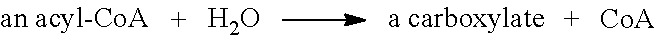

This enzyme occurs in a variety of organism, including eukaryotic and prokaryotic organisms, such as plants, animals, fungi and bacteria. The enzyme has, e.g., been described in Arabidopsis thaliana, Rhodotorula aurantiaca, Bumilleriopsis filiformis, Eremosphaera viridis, Euglena gracilis, Mus musculus, Rattus norvegicus, Homo sapiens, Sus, scrofa, Bos taurus, Cais lupus familiaris, Cavia porcellus, Cricetus griseus, Drosophila melanogaster, Anas platyrhynchos, Gallus gallus, Caenorhabditis elegans, Saccharomyces cerevisia, Candida rugosa, Escherichia coli, Haemophilus influenzae, Xanthomonas campestris, Streptomyces sp., Streptomyces coelicolor, Alcaligenes faecalis, Pseudomonas aeruginosa, Pseudomonas putida, Amycolatopsis mediterranei, Acinetobacter calcoaceticus, Helicobacter pylori, Rhodobacter spaeroides and Mycobacterium phlei. In a preferred embodiment the acyl-CoA hydrolase is an enzyme from Escherichia coli, from Pseudomonas putida or from Haemophilus influenza, more preferably the YciA enzyme from E. coli or its closely related homolog H10827 from Haemophilus influenza (Zhuang et al., Biochemistry 47 (2008), 2789-2796). The YciA enzyme from Haemophilus influenza is described to catalyze the hydrolysis of propionyl-CoA into propionic acid (Zhuang et al., Biochemistry 47 (2008), 2789-2796). In another preferred embodiment the acetyl-CoA hydrolase is an enzyme from Homo sapiens (UniProt: Q9NPJ3) which is described to hydrolyze propionyl-CoA (Cao et al., Biochemistry 48 (2009), 1293-1304).

Particularly preferred enzymes are the above-described acyl-CoA hydrolase YciA enzyme from Haemophilus influenza strain R2866 (SEQ ID NO: 30) and the acetyl-CoA hydrolase enzyme from Homo sapiens (UniProt: Q9NPJ3; SEQ ID NO:31). Particularly preferred are also the enzymes acyl-CoA thioester hydrolase from E. coli (Uniprot P0A8Z0; SEQ ID NO: 32), acyl-CoA thioesterase 2 from E. coli (Uniprot P0AGG2; SEQ ID NO: 33) and acyl-CoA thioesterase II from Pseudomonas putida (Uniprot Q88DR1; SEQ ID NO: 34). Particularly preferred is the thioesterase TesB from E. coli K12 (uniprot:P0AGG2), as this enzyme is already described to efficiently catalyze this reaction in E. coli for the biosynthesis of propionic acid (Tseng and Prather, P.N.A.S. 2012, 109(44),p17925-17930).

In another preferred embodiment, the acyl-CoA hydrolase is an enzyme derived from the family of 1,4-dihydroxy-2-naphthoyl-CoA hydrolases. Enzymes of this family of 1,4-dihydroxy-2-naphthoyl-CoA hydrolases are known to catalyze the following reaction:

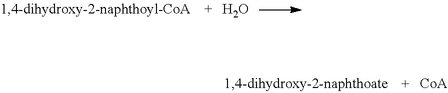

These enzymes are also often referred to as YdiI thioesterases. Enzymes of this family occur in a variety of organisms and have, e.g., been described in Escherichia coli and Salmonella enterica.

Thus, particularly preferred acyl-CoA hydrolases for the enzymatic conversion of 3-methylcrotonyl-CoA into 3-methylcrotonic acid of the present invention are enzymes which belong to the family of 1,4-dihydroxy-2-naphthoyl-CoA hydrolases, more preferably the 1,4-dihydroxy-2-naphthoyl-CoA hydrolase derived from Escherichia coli (SEQ ID NO:82) or the 1,4-dihydroxy-2-naphthoyl-CoA hydrolase derived from Salmonella enterica (SEQ ID NO:83).

In a particularly preferred embodiment, the acyl-CoA hydrolase employed in the method of the invention has an amino acid sequence as shown in any one of SEQ ID NOs: 30 to 34 and SEQ ID NOs:82 and 83 or shows an amino acid sequence which is at least x % homologous to any one of SEQ ID NOs: 30 to 34 and SEQ ID NOs:82 and 83 and has the activity of an acyl-CoA hydrolase with x being an integer between 30 and 100, preferably 35, 40, 45, 50, 55, 60, 65, 70, 75, 80, 85, 90, 91, 92, 93, 94, 95, 96, 97, 98 or 99 wherein such an enzyme is capable of catalyzing the conversion of 3-methylcrotonyl-CoA into 3-methylcrotonic acid. As regards the determination of the sequence identity, the same applies as has been set forth above.

As described above, the direct conversion of 3-methylcrotonyl-CoA into 3-methylcrotonic acid can also be achieved by making use of an enzyme which is classified as a CoA-transferase (EC 2.8.3.-) capable of transferring the CoA group of 3-methylcrotonyl-CoA to a carboxylic acid.

CoA-transferases are found in organisms from all lines of descent. Most of the CoA-transferases belong to two well-known enzyme families (referred to in the following as families I and II) and there exists a third family which had been identified in anaerobic metabolic pathways of bacteria. A review describing the different families can be found in Heider (FEBS Letters 509 (2001), 345-349).

Family I contains, e.g., the following CoA-transferases:

For 3-oxo acids: enzymes classified in EC 2.8.3.5 or EC 2.8.3.6;

For short chain fatty acids: enzymes classified in EC 2.8.3.8 or EC 2.8.3.9;

For succinate: succinyl-CoA:acetate CoA-transferases, i.e. enzymes classified in EC 2.8.3.18 (see also Mullins et al., Biochemistry 51(2012), 8422-34; Mullins et al., J. Bacteriol. 190 (2006), 4933-4940).

Most enzymes of family I naturally use succinyl-CoA or acetyl-CoA as CoA donors. These enzymes contain two dissimilar subunits in different aggregation states. Two conserved amino acid sequence motives have been identified:
Prosites entry PS01273 (http://prosite.expasy.org/cgi-bin/prosite/prosite-search-ac?PDOC00980)
COA_TRANSF_1, PS01273; Coenzyme A transferases signature 1 (PATTERN) Consensus pattern:
[DN]-[GN]-x(2)-[LIVMFA](3)-G-G-F-x(3)-G-x-P
and
Prosites entries PS01273 (http://prosite.expasy.org/cgi-bin/prosite/prosite-search-ac?PDOC00980)
COA_TRANSF_2, PS01274; Coenzyme A transferases signature 2 (PATTERN) Consensus pattern:
[LF]-[HQ]-S-E-N-G-[LIVF](2)-[GA]
E (glutamic acid) is an active site residue.

The family II of CoA-transferases consists of the homodimeric α-subunits of citrate lyase (EC 2.8.3.10) and citramalate lyase (EC 2.8.3.11). These enzymes catalyse the transfer of acyl carrier protein (ACP) which contains a covalently bound CoA-derivative. It was shown that such enzymes also accept free CoA-thioester in vitro, such as acetyl-CoA, propionyl-CoA, butyryl-CoA in the case of citrate lyase (Dimroth et al., Eur. J. Biochem. 80 (1977), 479-488) and acetyl-CoA and succinyl-CoA in the case of citramalate lyase (Dimroth et al., Eur. J. Biochem. 80 (1977), 469-477).

According to Heider (loc. cit.) family Ill of CoA-transferases consists of formyl-CoA: oxalate CoA-transferase, succinyl-CoA:(R)-benzylsuccinate CoA-transferase, (E)-cinnamoyl-CoA:(R)-phenyllactate CoA-transferase and butyrobetainyl-CoA:(R)-carnitine CoA-transferase. A further member of family Ill is succinyl-CoA:L-malate CoA-transferase whose function in autrophic CO₂ fixation of Chloroflexus aurantiacus is to activate L-malate to its CoA thioester with succinyl-CoA as the CoA donor (Friedman S. et al. J. Bacteriol. 188 (2006), 2646-2655). The amino acid sequences of the CoA-tranferase of this family show only a low degree of sequence identity to those of families I and II. These CoA-transferases occur in prokaryotes and eukaryotes.

In a preferred embodiment the CoA-transferase employed in a method according to the present invention is a CoA-transferase which belongs to family I or II as described herein-above.

Preferably, the CoA-transferase employed in a method according to the present invention for the direct conversion of 3-methylcrotonyl-CoA into 3-methylcrotonic acid is selected from the group consisting of:
propionate:acetate-CoA transferase (EC 2.8.3.1);
acetate CoA-transferase (EC 2.8.3.8); and
butyrate-acetoacetate CoA-transferase (EC 2.8.3.9).

Thus, in one preferred embodiment the direct conversion of 3-methylcrotonyl-CoA into 3-methylcrotonic acid is achieved by making use of an acetate CoA-transferase (EC 2.8.3.8). Acetate CoA-transferases are enzymes which catalyze the following reaction:

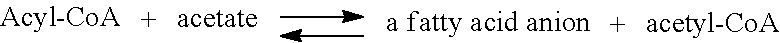

This enzyme occurs in a variety of bacteria and has, e.g., been described in Anaerostipes caccae, Eubacterium hallii, Faecalibacterium prausnitzii, Roseburia hominis, Roseburia intestinalis, Coprococcus sp. and Escherichia coli.

In another preferred embodiment the direct conversion of 3-methylcrotonyl-CoA into 3-methylcrotonic acid is achieved by making use of a butyrate-acetoacetate CoA-transferase (EC 2.8.3.9). Butyrate-acetoacetate CoA-transferase are enzymes which catalyze the following reaction:

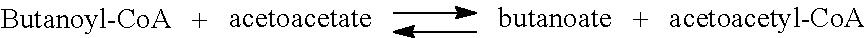

This enzyme occurs in a variety of organism, including eukaryotic and prokaryotic organisms, such as animals and bacteria. The enzyme has, e.g., been described in Bos taurus, Clostridium sp. and Escherichia coli.

In another preferred embodiment the direct conversion of 3-methylcrotonyl-CoA into 3-methylcrotonic acid is achieved by making use of a propionate:acetate-CoA transferase (EC 2.8.3.1). Propionate:acetate-CoA transferases are enzymes which catalyze the following reaction:

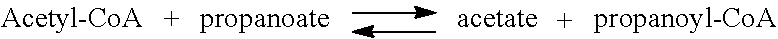

This enzyme occurs in a variety of organism including prokaryotic organisms and the enzyme has, e.g., been described in Clostridium kluyveri, Clostridium propionicum, Clostridium propionicum JCM1430, Cupriavidus necator and Emericella nidulans.

In another preferred embodiment the direct conversion of 3-methylcrotonyl-CoA into 3-methylcrotonic acid is achieved by making use of a succinyl-CoA:acetate-CoA transferase (EC 2.8.3.18). Succinyl-CoA:acetate CoA-transferases are enzymes which catalyze the following reaction:

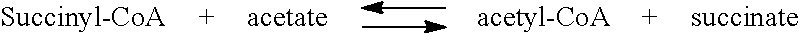

This enzyme occurs in a variety of organism, including prokaryotic organisms, and the enzyme has, e.g., been described in Acetobacter aceti, Trichomonas vaginalis, Tritrichomonas foetus, Tritrichomonas foetus ATCC 30924 and Trypanosoma brucei.

In another preferred embodiment, the direct conversion of 3-methylcrotonyl-CoA into 3-methylcrotonic acid is achieved by making use of a CoA-transferase derived from Megasphaera sp. (Uniprot accession number S7HFR5), an enzyme which belongs to the of CoA-transferases (EC 2.8.3.-) as defined herein-above.

In a preferred embodiment, the CoA-transferase employed in the method of the present invention is a CoA-transferase derived from Megasphaera sp. (Uniprot accession number S7HFR5; SEQ ID NO:84).

In a preferred embodiment of the present invention the CoA-transferase is an enzyme comprising the amino acid sequence of SEQ ID NO: 84 or a sequence which is at least n % identical to SEQ ID NO: 84 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of directly converting 3-methylcrotonyl-CoA into 3-methylcrotonic acid. As regards the determination of the sequence identity, the same applies as has been set forth above.

The enzymatic conversion of 3-methylcrotonyl-CoA into 3-methylcrotonic acid: an alternative route to the above-described step VI

In another preferred embodiment, the conversion of 3-methylcrotonyl-CoA into 3-methylcrotonic acid is achieved by an alternative route wherein 3-methylcrotonyl-CoA is first enzymatically converted into 3-methylbutyryl-CoA which is then enzymatically converted into 3-methylbutyric acid which is then ultimately converted into 3-methylcrotonic acid. This alternative conversion of 3-methylcrotonyl-CoA into 3-methylcrotonic acid via 3-methylbutyryl-CoA and 3-methylbutyric acid is schematically illustrated in FIG. 32.

Accordingly, the present invention relates to a method for producing isobutene from 3-methylcrotonyl-CoA in which 3-methylcrotonyl-CoA is first enzymatically converted into 3-methylbutyryl-CoA which is then enzymatically converted into 3-methylbutyric acid which is then converted into 3-methylcrotonic acid which is then further converted into isobutene as described herein above.

The first enzymatic conversion, i.e., the conversion of 3-methylcrotonyl-CoA into 3-methylbutyryl-CoA, is a desaturation reaction, i.e., reduction of the double bond C═C of 3-methylcrotonyl-CoA into 3-methylbutyryl-CoA. The enzymatic conversion of 3-methylcrotonyl-CoA into 3-methylbutyryl-CoA, i.e. the reduction of the double bond in 3-methylcrotonyl-CoA, can, for example, be achieved by employing an enzyme classified as EC 1.3._._. Enzymes classified as EC 1.3._._are oxidoreductases acting on the CH—CH group of a donor molecule. This subclass contains enzymes that reversibly catalyze the conversion of a carbon-carbon single bond to a carbon-carbon double bond between two carbon atoms. Sub-classes of EC 1.3 are classified depending on the acceptor. In one preferred embodiment the enzyme is an enzyme which is classified as EC 1.3._._ and which uses NADH or NADPH as co-factor.

In one particularly preferred embodiment the enzyme is an enzyme which uses NADPH as a co-factor. In a preferred embodiment the enzyme is selected from the group consisting of:
acyl-CoA dehydrogenase (NADP+) (EC 1.3.1.8);
enoyl-[acyl-carrier-protein]reductase (NADPH, Si-specific) (EC 1.3.1.10);
cis-2-enoyl-CoA reductase (NADPH) (EC 1.3.1.37);
trans-2-enoyl-CoA reductase (NADPH) (EC 1.3.1.38);
enoyl-[acyl-carrier-protein]reductase (NADPH, Re-specific) (EC 1.3.1.39); and
crotonyl-CoA reductase (EC 1.3.1.86).

Thus, in one preferred embodiment the conversion of 3-methylcrotonyl-CoA into 3-methylbutyryl-CoA is achieved by making use of an acyl-CoA dehydrogenase (NADP+) (EC 1.3.1.8). Acyl-CoA dehydrogenases are enzymes which catalyze the following reaction:

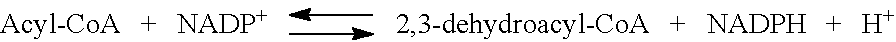

This enzyme occurs in a variety of organism, including eukaryotic and prokaryotic organisms, such as plants, animals, fungi and bacteria. The enzyme has, e.g., been described in Bos, taurus, Rattus novegicus, Mus musculus, Columba sp., Arabidopsis thaliana, Nicotiana benthamiana, Allium ampeloprasum, Euglena gracilis, Candida albicans, Streptococcus collinus, Rhodobacter sphaeroides and Mycobacterium smegmatis.

In a further preferred embodiment the conversion of 3-methylcrotonyl-CoA into 3-methylbutyryl-CoA is achieved by making use of an enoyl-[acyl-carrier-protein]reductase (NADPH, Si-specific) (EC 1.3.1.10). Enoyl-[acyl-carrier-protein]reductases (NADPH, Si-specific) are enzymes which catalyze the following reaction:

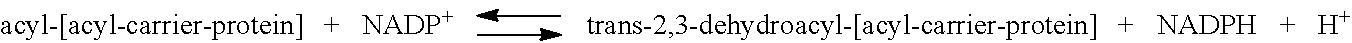

This enzyme occurs in a variety of organism, including eukaryotic and prokaryotic organisms, such as plants, fungi and bacteria. The enzyme has, e.g., been described in Carthamus tinctorius, Candida tropicalis, Saccharomyces cerevisiae, Streptococcus collinus, Streptococcus pneumoniae, Staphylococcus aureus, Bacillus subtilis, Bacillus cereus, Porphyromonas gingivalis, Escherichia coli and Salmonella enterica.

In a further preferred embodiment the conversion of 3-methylcrotonyl-CoA into 3-methylbutyryl-CoA is achieved by making use of a cis-2-enoyl-CoA reductase (NADPH) (EC 1.3.1.37). Cis-2-enoyl-CoA reductases (NADPH) are enzymes which catalyze the following reaction:

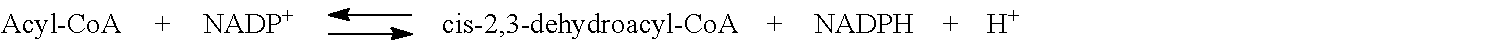

This enzyme has been described to occur in Escherichia coli.

In a further preferred embodiment the conversion of 3-methylcrotonyl-CoA into 3-methylbutyryl-CoA is achieved by making use of a trans-2-enoyl-CoA reductase (NADPH) (EC 1.3.1.38). Trans-2-enoyl-CoA reductases (NADPH) are enzymes which catalyze the following reaction:

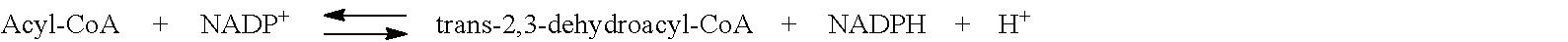

This enzyme occurs in a variety of organism, including eukaryotic and prokaryotic organisms, such as plants, animals and bacteria. The enzyme has, e.g., been described in Homo sapien, Rattus norvegicus, Mus musculus, Cavia porcellus, Caenorhabditis elegans, Phalaenopsis amabilis, Gossypium hirsutum, Mycobacterium tuberculosis, Streptococcus collinu and Escherichia coli.

In a further preferred embodiment the conversion of 3-methylcrotonyl-CoA into 3-methylbutyryl-CoA is achieved by making use of an enoyl-[acyl-carrier-protein]reductase (NADPH, Re-specific) (EC 1.3.1.39). Enoyl-[acyl-carrier-protein]reductases (NADPH, Re-specific) are enzymes which catalyze the following reaction:

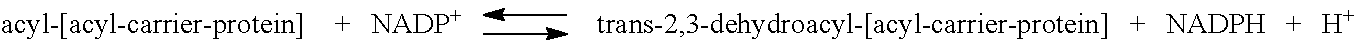

This enzyme occurs in a variety of organism, including eukaryotic and prokaryotic organisms, such as animals and bacteria. The enzyme has, e.g., been described in Gallus gallus, Pigeon, Rattus norvegicus, Cavia porcellus, Staphylococcus aureus, Bacillus subtilis and Porphyromonas gingivalis.

In a further preferred embodiment the conversion of 3-methylcrotonyl-CoA into 3-methylbutyryl-CoA is achieved by making use of a crotonyl-CoA reductase (EC 1.3.1.86). Crotonyl-CoA reductases are enzymes which catalyze the following reaction:

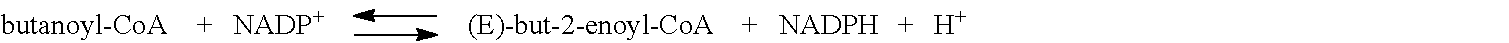

This enzyme occurs in a variety of organism, including eukaryotic and prokaryotic organisms, such as animals, fungi and bacteria. The enzyme has, e.g., been described in Bos taurus, Salinospora tropica, Clostridium difficile, Streptomyces collinus, Streptomyces cinnamonensis and Streptomyces hygroscopicus.

The second enzymatic conversion, i.e., the conversion of 3-methylbutyryl-CoA into 3-methylbutyric acid, can be achieved by different enzymatic conversions. One possibility is the direct conversion via a hydrolysis reaction. Another possibility is the direct conversion via a reaction catalyzed by a CoA-transferase and a third possibility is a two-step conversion via 3-methylbutyryl phosphate.

Thus, according to the present invention, the enzymatic conversion of 3-methylbutyryl-CoA into 3-methylbutyric acid is achieved by
(a) a single enzymatic reaction in which 3-methylbutyryl-CoA is directly converted into 3-methylbutyric acid, preferably by making use of a CoA transferase (EC 2.8.3.-), preferably a propionate:acetate-CoA transferase (EC 2.8.3.1), an acetate CoA-transferase (EC 2.8.3.8) or a succinyl-CoA:acetate CoA-transferase (EC 2.8.3.18);
(b) a single enzymatic reaction in which 3-methylbutyryl-CoA is directly converted into 3-methylbutyric acid, preferably by making use of a thioester hydrolase (EC 3.1.2.-), preferably acetyl-CoA hydrolase (EC 3.1.2.1), an ADP-dependent short-chain-acyl-CoA hydrolase (EC 3.1.2.18) or an acyl-CoA hydrolase (EC 3.1.2.20); or
(c) two enzymatic steps comprising
   (i) first enzymatically converting 3-methylbutyryl-CoA into 3-methylbutyryl phosphate; and
   (ii) then enzymatically converting the thus obtained 3-methylbutyryl phosphate into said 3-methylbutyric acid.

As regards the preferred embodiments for the CoA transferase (EC 2.8.3.-), the propionate:acetate-CoA transferase (EC 2.8.3.1), the acetate CoA-transferase (EC 2.8.3.8) or a succinyl-CoA:acetate CoA-transferase (EC 2.8.3.18), the thioester hydrolase (EC 3.1.2.-), the acetyl-CoA hydrolase (EC 3.1.2.1), the ADP-dependent short-chain-acyl-CoA hydrolase (EC 3.1.2.18), the acyl-CoA hydrolase (EC 3.1.2.20), the enzyme capable of converting 3-methylbutyryl-CoA into 3-methylbutyryl phosphate and the enzyme capable of converting 3-methylbutyryl phosphate into said 3-methylbutyric acid, the same applies as has been set forth above in connection with the enzymatic conversion of step VIa, step VIb and step VIc according to the invention.

The third enzymatic conversion, i.e., the conversion of 3-methylbutyric acid into 3-methylcrotonic acid can, e.g., be achieved by a 2-enoate reductase (EC 1.3.1.31). 2-enoate reductases are enzymes which naturally catalyze the following reaction:

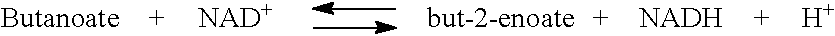

This enzyme occurs in a variety of organism, including eukaryotic and prokaryotic organisms, such as animals, fungi and bacteria. The enzyme has, e.g., been described in Cichorium intybus, Marchantia polymorpha, Solanum lycopersicum, Absidia glauca, Kluyveromyces lactis, Penicillium citrinum; Rhodosporidium, Saccharomyces cerevisiae, Clostridium kluyveri, Clostridium bifermentans, Clostridium botulinum, Clostridium difficile, Clostridium ghonii, Clostridium mangenotii, Clostridium oceanicum, Clostridium sordellii, Clostridium sporogenes, Clostridium sticklandii, Clostridium tyrobutyricum, Achromobacter sp., Burkholderia sp., Gluconobacter oxydans, Lactobacillus casei, Pseudomonas putida, Shewanella sp., Yersinia bercovieri, Bacillus subtilis, Moorella thermoacetica and Peptostreptococcus anaerobius. The enoate reductase of Clostridiae has been described, e.g., in Tischler et al. (Eur. J. Bioche. 97 (1979), 103-112).

The enzymatic conversion of 3-methylglutaconyl-CoA into 3-methylcrotonyl-CoA: step VII as shown in FIG. 1

The 3-methylcrotonyl-CoA which is converted according to the method of the present invention into 3-methylcrotonic acid according to any of the above described methods (and further converted according to the method of the present invention into isobutene according to any of the above described methods) may itself be provided by an enzymatic reaction, namely the enzymatic conversion of 3-methylglutaconyl-CoA into 3-methylcrotonyl-CoA. The conversion of 3-methylglutaconyl-CoA into 3-methylcrotonyl-CoA is schematically illustrated in FIG. 12.

Accordingly, the present invention relates to a method for producing isobutene from 3-methylglutaconyl-CoA in which 3-methylglutaconyl-CoA is first converted into 3-methylcrotonyl-CoA which is then further converted into 3-methylcrotonic acid which is then further converted into isobutene as described herein above.

The conversion of 3-methylglutaconyl-CoA into 3-methylcrotonyl-CoA may be catalyzed by different enzymes. According to the present invention, the conversion of 3-methylglutaconyl-CoA into said 3-methylcrotonyl-CoA preferably makes use of (i) a methylcrotonyl-CoA carboxylase (EC 6.4.1.4); or (ii) a geranoyl-CoA carboxylase (EC 6.4.1.5) (as shown in step VII of FIG. 1).

Methylcrotonyl-CoA carboxylases (EC 6.4.1.4) and geranoyl-CoA carboxylases (EC 6.4.1.5) as well as preferred enzymes of these enzyme classes have already been described above. Accordingly, as regards these enzymes, the same applies to the conversion of 3-methylglutaconyl-CoA into 3-methylcrotonyl-CoA as has been set forth above.

In another preferred embodiment the conversion of 3-methylglutaconyl-CoA via decarboxylation into 3-methylcrotonyl-CoA is catalyzed by a 3-methylglutaconyl-CoA decarboxylase, e.g. a 3-methylglutaconyl-CoA decarboxylase of Myxococcus xanthus encoded by the IiuB gene. This gene codes for an enzyme having the two subunits AibA and AibB (Li et al., Angew. Chem. Int. Ed. 52 (2013), 1304-1308).

This enzyme has already described above as a methylcrotonyl-CoA carboxylase derived from Myxcoxoccus xanthus in the context of conversion of 3-methylcrotonic acid into isobutene.

The same enzyme derived from Myxococcus xanthus encoded by the IiuB gene having the two subunits AibA and AibB (Li et al., Angew. Chem. Int. Ed. 52 (2013), 1304-1308) has been described above with reference to SEQ ID NOs: 100 and 101 and can also be used for the conversion of 3-methylglutaconyl-CoA via decarboxylation into 3-methylcrotonyl-CoA.

In a preferred embodiment of the present invention the 3-methylglutaconyl-CoA decarboxylase is an enzyme comprising the amino acid sequence of SEQ ID NO: 100 or a sequence which is at least n % identical to SEQ ID NO: 100 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylglutaconyl-CoA into 3-methylcrotonyl-CoA. As regards the determination of the sequence identity, the same applies as has been set forth above. In another preferred embodiment of the present invention the 3-methylglutaconyl-CoA decarboxylase is an enzyme comprising the amino acid sequence of SEQ ID NO: 101 or a sequence which is at least n % identical to SEQ ID NO: 101 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylglutaconyl-CoA into 3-methylcrotonyl-CoA. As regards the determination of the sequence identity, the same applies as has been set forth above.

In another preferred embodiment of the present invention the 3-methylglutaconyl-CoA decarboxylase is a heterodimeric enzyme comprising a combination of the amino acid sequence of SEQ ID NO: 100 and 101 or a sequence which is at least n % identical to SEQ ID NO: 100 and 101 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylglutaconyl-CoA into 3-methylcrotonyl-CoA. As regards the determination of the sequence identity, the same applies as has been set forth above.

The enzymatic conversion of 3-hydroxy-3-methylglutaryl-CoA into 3-methylglutaconyl-CoA: step VIII as shown in FIG. 1

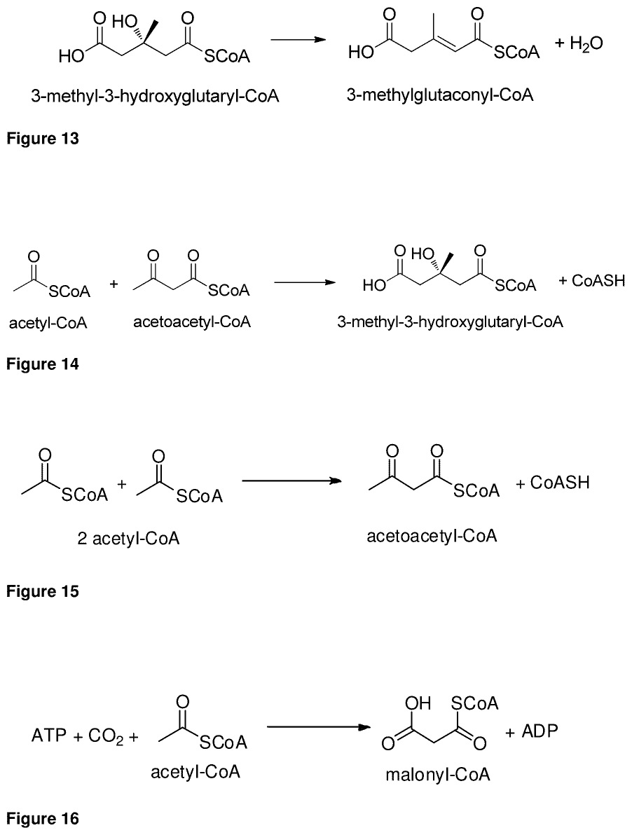

The 3-methylglutaconyl-CoA which is converted into 3-methylcrotonyl-CoA may itself be provided by an enzymatic reaction, namely the enzymatic conversion of 3-hydroxy-3-methylglutaryl-CoA into 3-methylglutaconyl-CoA; see FIG. 13.

Accordingly, the present invention also relates to a method for producing isobutene from 3-hydroxy-3-methylglutaryl-CoA in which 3-hydroxy-3-methylglutaryl-CoA is first converted into 3-methylglutaconyl-CoA which is then converted into 3-methylcrotonyl-CoA which is then further converted into 3-methylcrotonic acid which is then further converted into isobutene as described herein above.

According to the present invention, the enzymatic conversion of 3-hydroxy-3-methylglutaryl-CoA into 3-methylglutaconyl-CoA is an enzymatic dehydration reaction which occurs naturally, and which is catalyzed, e.g., by enzymes classified as 3-methylglutaconyl-coenzyme A hydratase (EC 4.2.1.18). Accordingly, the enzymatic conversion of 3-hydroxy-3-methylglutaryl-CoA into 3-methylglutaconyl-CoA preferably makes use of a 3-methylglutaconyl-coenzyme A hydratase (EC 4.2.1.18) (as shown in step VIII of FIG. 1).

3-methylglutaconyl-coenzyme A hydratases are enzymes which catalyze the following reaction:

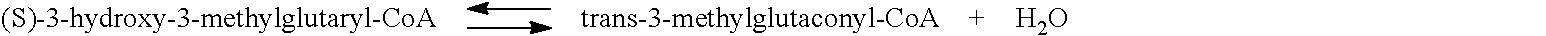

This enzyme occurs in a variety of organisms, including eukaryotic and prokaryotic organisms, such as plants, animals and bacteria. The enzyme has, e.g., been described in Catharantus roseus, Homo sapiens, Bos taurus, Ovis aries, Acinetobacter sp., Myxococcus sp. and Pseudomonas putida. In a preferred embodiment the 3-methylglutaconyl-coenzyme A hydratase is an enzyme from Myxococcus sp., and even more preferably an enzyme which has an amino acid sequence as shown in SEQ ID NO: 35 or shows an amino acid sequence which is at least x % homologous to SEQ ID NO: 35 and has the activity of a 3-methylglutaconyl-coenzyme A hydratase with x being an integer between 30 and 100, preferably 35, 40, 45, 50, 55, 60, 65, 70, 75, 80, 85, 90, 91, 92, 93, 94, 95, 96, 97, 98 or 99 wherein such an enzyme is capable of converting 3-hydroxy-3-methylglutaryl-CoA into 3-methylglutaconyl-CoA as set forth herein above. As regards the determination of the degree of identity, the same applies as has been set forth herein above.

The conversion of 3-hydroxy-3-methylglutaryl-CoA into 3-methylglutaconyl-CoA can also be achieved by making use of a 3-hydroxy-3-methylglutaryl-coenzyme A dehydratase activity which has been identified, e.g., in Myxococcus xanthus and which is encoded by the IiuC gene (Li et al., Angew. Chem. Int. Ed. 52 (2013), 1304-1308). The 3-hydroxy-3-methylglutaryl-coenzyme A dehydratase derived from Myxococcus xanthus has the Uniprot Accession number Q1 D5Y4.

Thus, in a preferred embodiment, the 3-hydroxy-3-methylglutaryl-coenzyme A dehydratase employed in the method of the present invention is an enzyme derived from Myxococcus xanthus (Uniprot Accession number Q1 D5Y4; SEQ ID NO:98).

In a preferred embodiment of the present invention the 3-hydroxy-3-methylglutaryl-coenzyme A dehydratase is an enzyme comprising an amino acid sequence of SEQ ID NO:98 or a sequence which is at least n % identical to SEQ ID NO:98 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-hydroxy-3-methylglutaryl-CoA into 3-methylglutaconyl-CoA. As regards the determination of the sequence identity, the same applies as has been set forth above.

The enzymatic conversion of 3-hydroxy-3-methylglutaryl-CoA into 3-methylglutaconyl-CoA can also be achieved by making use of a 3-hydroxyacyl-CoA dehydratase or an enoyl-CoA hydratase. 3-hydroxyacyl-CoA dehydratases and enoyl-CoA hydratases catalyze the same reaction while the name of one of these enzymes denotes one direction of the corresponding reaction while the other name denotes the reverse reaction. As the reaction is reversible, both enzyme names can be used.

3-hydroxyacyl-CoA dehydratases and enoyl-CoA hydratases belong to enzymes classified as EC 4.2.1.-.

3-hydroxyacyl-CoA dehydratases and enoyl-CoA hydratases have, e.g., been identified in Pseudomonas sp., Acinetobacter baumanii (Uniprot accession number A0A0D5YDD4), Pseudomonas aeruginosa (Uniprot accession number Q9HZV7), Marinobacter santoriniensis (Uniprot accession number M7CV63), Pseudomonas knackmussii, Pseudomonas pseudoalcaligenes (Uniprot accession number L8MQT6), Pseudomonas flexibilis and Alcanivorax dieselolei as well as in Ustilago maydis (Uniprot accession number Q4PEN0), Bacillus sp. GeD10 (Uniprot accession number N1LWG2) and in Labilithrix luteola (Uniprot accession number A0A0K1 PN19).

In a preferred embodiment, the 3-hydroxyacyl-CoA dehydratase/enoyl-CoA hydratase employed in the method of the present invention for the conversion of 3-hydroxy-3-methylglutaryl-CoA into 3-methylglutaconyl-CoA is an enzyme derived from Pseudomonas sp. (SEQ ID NO:85), Acinetobacter baumanii (Uniprot accession number A0A0D5YDD4; SEQ ID NO:86), Pseudomonas aeruginosa (Uniprot accession number Q9HZV7; SEQ ID NO:87), Marinobacter santoriniensis (Uniprot accession number Q9HZV7; SEQ ID NO:88), Pseudomonas knackmussii (SEQ ID NO:89), Pseudomonas pseudoalcaligenes (Uniprot accession number L8MQT6; SEQ ID NO:90), Pseudomonas flexibilis (SEQ ID NO:91), Alcanivorax dieselolei (SEQ ID NO:92), Ustilago maydis (Uniprot accession number Q4PEN0; SEQ ID NO:95), Bacillus sp. GeD10 (Uniprot accession number N1LWG2; SEQ ID NO:96) or Labilithrix luteola (Uniprot accession number A0A0K1 PN19; SEQ ID NO:97).

In a preferred embodiment of the present invention the 3-hydroxyacyl-CoA dehydratase/enoyl-CoA hydratase is an enzyme comprising an amino acid sequence selected from the group consisting of SEQ ID NOs: 85 to 92 and SEQ ID NOs: 95 to 97 or a sequence which is at least n % identical to any of SEQ ID NOs: 85 to 92 and SEQ ID NOs: 95 to 97 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-hydroxy-3-methylglutaryl-CoA into 3-methylglutaconyl-CoA. As regards the determination of the sequence identity, the same applies as has been set forth above.

The enzymatic conversion of acetoacetyl-CoA into 3-hydroxy-3-methylglutaryl-CoA: step IX as shown in FIG. 1

The 3-hydroxy-3-methylglutaryl-CoA which is converted into 3-methylglutaconyl-CoA may itself be provided by an enzymatic reaction, namely the enzymatic condensation of acetoacetyl-CoA and acetyl-CoA into 3-hydroxy-3-methylglutaryl-CoA; see FIG. 14.

Accordingly, the present invention also relates to a method for producing isobutene from acetoacetyl-CoA and acetyl-CoA in which acetoacetyl-CoA and acetyl-CoA are first condensed into 3-hydroxy-3-methylglutaryl-CoA which is then converted into 3-methylglutaconyl-CoA which is then converted into 3-methylcrotonyl-CoA which is then further converted into 3-methylcrotonic acid which is then further converted into isobutene as described herein above.

According to the present invention, the enzymatic condensation of acetoacetyl-CoA and acetyl-CoA into 3-hydroxy-3-methylglutaryl-CoA makes preferably use of a 3-hydroxy-3-methylglutaryl-CoA synthase (see step IX of FIG. 1).

The condensation of acetyl-CoA and acetoacetyl-CoA is a reaction which is naturally catalyzed by the enzyme 3-hydroxy-3-methylglutaryl-CoA synthase (also referred to as HMG-CoA synthase). Thus, preferably, the condensation of acetyl-CoA and acetoacetyl-CoA into 3-hydroxy-3-methylglutaryl-CoA makes use of a 3-hydroxy-3-methylglutaryl-CoA synthase (also referred to as HMG-CoA synthase). HMG-CoA synthases are classified in EC 2.3.3.10 (formerly, HMG-CoA synthase has been classified as EC 4.1.3.5 but has been transferred to EC 2.3.3.10). The term “HMG-CoA synthase” refers to any enzyme which is able to catalyze the reaction where acetyl-CoA condenses with acetoacetyl-CoA to form 3-hydroxy-3-methylglutaryl-CoA (HMG-CoA) (see FIG. 14). HMG-CoA synthase is part of the mevalonate pathway. Two pathways have been identified for the synthesis of isopentenyl pyrophosphate (IPP), i.e. the mevalonate pathway and the glyceraldehyde 3-phosphate-pyruvate pathway. HMG-CoA synthase catalyzes the biological Claisen condensation of acetyl-CoA with acetoacetyl-CoA and is a member of a superfamily of acyl-condensing enzymes that includes beta-ketothiolases, fatty acid synthases (beta-ketoacyl carrier protein synthase) and polyketide synthases.

HMG-CoA synthase has been described for various organisms. Also amino acid and nucleic acid sequences encoding HMG-CoA synthases from numerous sources are available. Generally, the sequences only share a low degree of overall sequence identity. For example, the enzymes from Staphylococcus or Streptococcus show only about 20% identity to those of human and avian HMG-CoA synthase. In some sources it is reported that the bacterial HMG-CoA synthases and their animal counterparts exhibit only about 10% overall sequence identity (Sutherlin et al., J. Bacteriol. 184 (2002), 4065-4070). However, the amino acid residues involved in the acetylation and condensation reactions are conserved among bacterial and eukaryotic HMG-CoA synthases (Campobasso et al., J. Biol. Chem. 279 (2004), 44883-44888). The three-dimensional structure of three HMG-CoA synthase enzymes has been determined and the amino acids crucial for the enzymatic reaction are in principle well characterized (Campobasso et al., loc. cit.; Chun et al., J. Biol. Chem. 275 (2000), 17946-17953; Nagegowda et al., Biochem. J. 383 (2004), 517-527; Hegardt, Biochem. J. 338 (1999), 569-582). In eukaryotes, there exist two forms of the HMG-CoA synthase, i.e. a cytosolic and a mitochondrial form. The cytosolic form plays a key role in the production of cholesterol and other isoprenoids and the mitochondrial form is involved in the production of ketone bodies.

In principle any HMG-CoA synthase enzyme can be used in the context of the present invention, in particular from prokaryotic or eukaryotic organisms.

Prokaryotic HMG-CoA synthases are described, e.g., from Staphylococcus aureus (Campobasso et al., loc. cit.; Uniprot accession number Q9FD87), Staphylococcus epidermidis (Uniprot accession number Q9FD76), Staphylococcus haemolyticus (Uniprot accession number Q9FD82), Enterococcus faecalis (Sutherlin et al., loc. cit.; UniProt accession number Q9FD71; SEQ ID NO:99), Enterococcus faecium (Uniprot accession number Q9FD66), Streptococcus pneumonia (Uniprot accession number Q9FD56), Streptococcus pyogenes (Uniprot accession number Q9FD61) and Methanobacterium thermoautotrophicum (accession number AE000857), Borrelia burgdorferi (NCBI accession number BB0683). Further HMG-CoA synthases are, e.g., described in WO 2011/032934. A preferred HMG-CoA synthase is the enzyme from Schizosaccharomyces pombe (Uniprot P54874). In a particularly preferred embodiment, the HMG-CoA synthase employed in the method of the invention has an amino acid sequence as shown in SEQ ID NO: 36 or SEQ ID NO:99 or shows an amino acid sequence which is at least x % homologous to SEQ ID NO: 36 or SEQ ID NO:99 and has the activity of a HMG-CoA synthase with x being an integer between 30 and 100, preferably 35, 40, 45, 50, 55, 60, 65, 70, 75, 80, 85, 90, 91, 92, 93, 94, 95, 96, 97, 98 or 99 wherein such an enzyme is capable of catalyzing the condensation of acetyl-CoA and acetoacetyl-CoA into 3-hydroxy-3-methylglutaryl-CoA. As regards the determination of the degree of identity, the same applies as has been set forth herein above.

The enzymatic conversion of acetyl-CoA into acetoacetyl-CoA: steps XIII, XIV and XV as shown in FIG. 1

The acetoacetyl-CoA which is either converted into 3-hydroxy-3-methylglutaryl-CoA or which is converted into acetoacetate may itself be provided by an enzymatic reaction, namely the enzymatic conversion of acetyl-CoA into acetoacetyl-CoA.

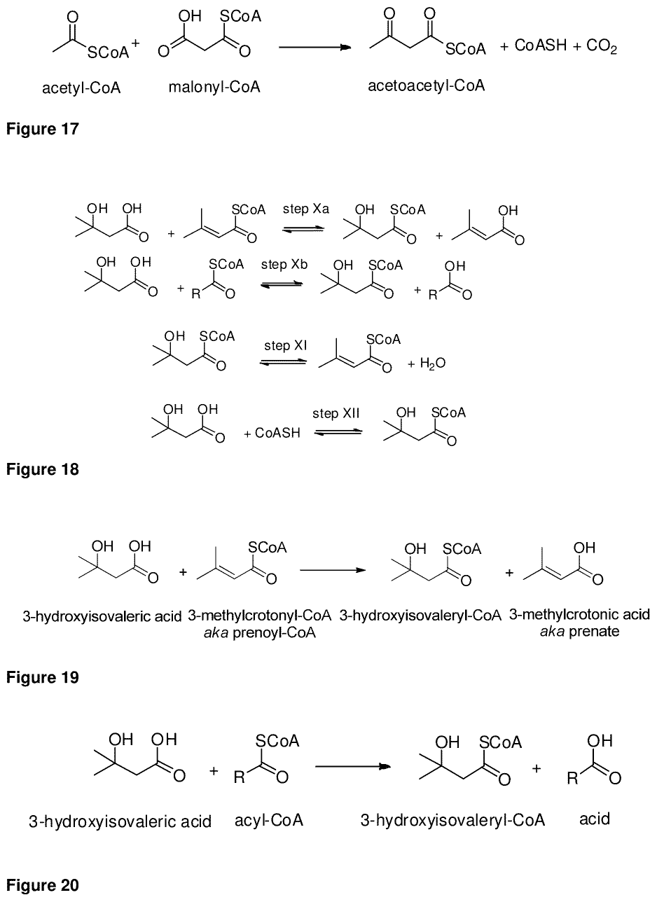

According to the present invention, the conversion of acetyl-CoA into said acetoacetyl-CoA can be achieved by different routes. One possibility is to first convert acetyl-CoA into malonyl-CoA (step XIV as shown in FIG. 1) and then to further condense said malonyl-CoA and acetyl-CoA into acetoacetyl-CoA (step XV as shown in FIG. 1). Another possibility is to directly condense in a single enzymatic reaction two molecules of acetyl-CoA into acetoacetyl-CoA (step XIII as shown in FIG. 1). These reactions are schematically shown in FIG. 15 (step XIII), FIG. 16 (step XIV) and FIG. 17 (step XV), respectively.

Thus, the present invention also relates to a method for producing isobutene from acetyl-CoA in which acetyl-CoA is first converted into acetoacetyl-CoA by any of the above-mentioned routes which is then condensed with acetyl-CoA into 3-hydroxy-3-methylglutaryl-CoA which is then converted into 3-methylglutaconyl-CoA which is then converted into 3-methylcrotonyl-CoA which is then further converted into 3-methylcrotonic acid which is then further converted into isobutene as described herein above.

Moreover, the present invention also relates to a method for producing isobutene from acetyl-CoA in which acetyl-CoA is first converted into acetoacetyl-CoA by any of the above-mentioned routes by any of the above-mentioned routes which is then converted into acetoacetate which is then converted into acetone which is then condensed with acetyl-CoA into 3-hydroxyisovalerate (HIV) which is then converted into 3-methylcrotonic acid as described herein above. Further, said 3-methylcrotonic acid is then further converted into isobutene as described herein above.

According to the present invention, the enzymatic conversion of acetyl-CoA into malonyl-CoA preferably makes use of an acetyl-CoA carboxylase (EC 6.4.1.2) (step XIV as shown in FIG. 1). This naturally occurring reaction fixes CO₂ on acetyl-CoA utilizing ATP resulting in malonyl-CoA. Enzymes classified as an acetyl-CoA carboxylases (EC 6.4.1.2) catalyze the following reaction:

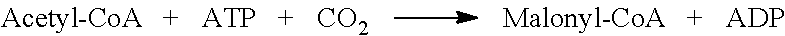

Moreover, according to the present invention, the enzymatic condensation of malonyl-CoA and acetyl-CoA into said acetoacetyl-CoA preferably makes use of an acetoacetyl-CoA synthase (EC 2.3.1.194) (step XV as shown in FIG. 1). This is a natural occurring reaction and condenses malonyl-CoA and acetyl-CoA in a decarboxylation reaction. Enzymes classified as acetoacetyl-CoA synthases (EC 2.3.1.194) catalyze the enzymatic conversion of acetyl-CoA and malonyl-CoA into acetoacetyl-CoA according to the following reaction.

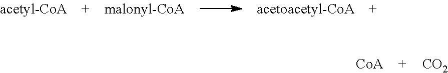

This reaction is catalyzed by an enzyme called acetoacetyl-CoA synthase (EC 2.3.1.194). The gene encoding this enzyme was identified in the mevalonate pathway gene cluster for terpenoid production in a soil-isolated Gram-positive Streptomyces sp. Strain CL190 (Okamura et al., PNAS USA 107 (2010), 11265-11270, 2010). Moreover a biosynthetic pathway using this enzyme for acetoacetyl-CoA production was recently developed in E. coli (Matsumoto K et al., Biosci. Biotechnol. Biochem, 75 (2011), 364-366).

Alternatively, the enzymatic conversion of acetyl-CoA into said acetoacetyl-CoA consists of a single enzymatic reaction in which acetyl-CoA is directly converted into acetoacetyl-CoA by the enzymatic condensation of two molecules of acetyl-CoA into acetoacetyl-CoA. Preferably, the enzymatic conversion of acetyl-CoA into acetoacetyl-CoA is achieved by making use of an acetyl-CoA acetyltransferase (EC 2.3.1.9).

Thus, acetoacetyl-CoA can be produced from acetyl-CoA as, e.g., described in WO 2013/057194. Therefore, according to the present invention, acetyl-CoA can, for example, be converted into acetoacetyl-CoA by the following reaction:

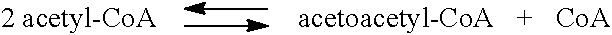

This reaction is a naturally occurring reaction and is catalyzed by enzymes called acetyl-CoA C-acetyltransferases which are classified as EC 2.3.1.9. Enzymes belonging to this class and catalyzing the above shown conversion of two molecules of acetyl-CoA into acetoacetyl-CoA and CoA occur in organisms of all kingdoms, i.e. plants, animals, fungi, bacteria etc. and have extensively been described in the literature. Nucleotide and/or amino acid sequences for such enzymes have been determined for a variety of organisms, like Homo sapiens, Arabidopsis thaliana, E. coli, Bacillus subtilis, Clostridium acetobutylicum and Candida, to name just some examples. In principle, any acetyl-CoA C-acetyltransferase (EC 2.3.1.9) can be used in the context of the present invention. In one preferred embodiment the enzyme is an acetyl-CoA acetyltransferase from Clostridium acetobutylicum (Uniprot P45359). In a particularly preferred embodiment, the acetyl-CoA acetyltransferase employed in the method of the invention has an amino acid sequence as shown in SEQ ID NO: 37 or shows an amino acid sequence which is at least x % homologous to SEQ ID NO: 37 and has the activity of an acetyl-CoA acetyltransferase with x being an integer between 30 and 100, preferably 35, 40, 45, 50, 55, 60, 65, 70, 75, 80, 85, 90, 91, 92, 93, 94, 95, 96, 97, 98 or 99 wherein such an enzyme is capable of converting acetyl-CoA into acetoacetyl-CoA as set forth herein above.

As regards the determination of the degree of identity, the same applies as has been set forth herein above.

The enzymatic recycling of metabolites occurring in the pathway of the present invention: steps Xa, Xb, XI and XII as shown in FIG. 1

The above-described method of the present invention for producing isobutene from acetyl-CoA may be supplemented by one or more of the following reactions as shown in step Xa, step Xb, step XI and step XII of FIG. 18.

These steps relate to alternative bioconversions which may occur concomitantly to any of the above-described methods for producing isobutene.

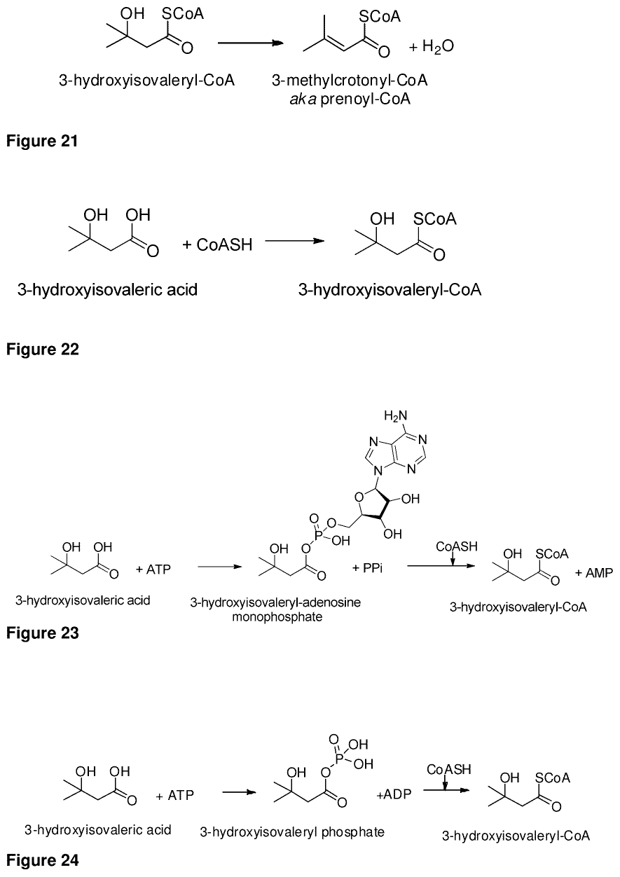

Thus, the present invention relates to any of the above-described methods for producing isobutene from 3-methylcrotonic acid (or from any of the above-described intermediates in the described pathways from acetyl-CoA into isobutene) wherein additionally
a) 3-hydroxyisovalerate (HIV) is enzymatically converted into 3-methylcrotonic acid with a concomitant transfer of CoA from 3-methylcrotonyl-CoA on 3-hydroxyisovalerate (HIV) to result in 3-hydroxyisovaleryl-CoA (step Xa as schematically shown in FIG. 19); and/or
b) 3-hydroxyisovalerate (HIV) is enzymatically converted into 3-hydroxyisovaleryl-CoA (step Xb as schematically shown in FIG. 20); and/or
c) 3-hydroxyisovaleryl-CoA is enzymatically converted into 3-methylcrotonyl-CoA (step XI as schematically shown in FIG. 21); and/or
d) 3-hydroxyisovalerate (HIV) is enzymatically converted into 3-hydroxyisovaleryl-CoA (step XII as schematically shown in FIG. 22).

These reactions which which will be described in more detail in the following, may occur concomitantly to any of the above-described methods for producing isobutene are beneficial for several reasons. First, it is known that the hydration of an enoyl-CoA (such as, e.g., 3-methylcrotonyl-CoA) is a favoured reaction in vivo in an aqueous medium. Accordingly, the above reactions represent possibilities which allow to drive the metabolic flux toward the precursor of isobutene, i.e., 3-methylcrotonic acid, even in case the pathway “leaks” into the direction of 3-hydroxyisovalerate (HIV) and/or 3-hydroxyisvaleryl-CoA. Second, the above conversions beneficially involve the conservation of energy into a thioester CoA bond via a transfer of a thioester group.

The enzymatic conversion of 3-hydroxyisovalerate (HIV) into 3-methylcrotonic acid with a concomitant transfer of CoA from 3-methylcrotonyl-CoA on 3-hydroxyisovalerate (HIV) to result in 3-hydroxyisovaleryl-CoA as shown in step Xa of FIG. 18

Thus, in a first aspect, the 3-methylcrotonic acid which is converted into isobutene may be provided by an enzymatic reaction wherein 3-hydroxyisovalerate (HIV) is enzymatically converted into 3-methylcrotonic acid with a concomitant transfer of CoA from 3-methylcrotonyl-CoA to 3-hydroxyisovalerate (HIV) to result in 3-hydroxyisovaleryl-CoA (step Xa as shown in FIG. 18). This reaction is schematically illustrated in FIG. 19.

Thus, the present invention also relates to a method for producing isobutene from 3-hydroxyisovalerate (HIV) wherein 3-hydroxyisovalerate (HIV) is enzymatically converted into 3-methylcrotonic acid with a concomitant transfer of CoA from 3-methylcrotonyl-CoA on 3-hydroxyisovalerate (HIV) to result in 3-hydroxyisovaleryl-CoA. Further, the thus produced 3-methylcrotonic acid is then enzymatically converted into isobutene as described herein above.

Moreover, the present invention also relates to a method for producing 3-methylcrotonic acid and 3-hydroxyisovaleryl-CoA from 3-hydroxyisovalerate (HIV) and from 3-methylcrotonyl-CoA wherein 3-hydroxyisovalerate (HIV) is enzymatically converted into 3-methylcrotonic acid with a concomitant transfer of CoA from 3-methylcrotonyl-CoA on 3-hydroxyisovalerate (HIV) to result in 3-hydroxyisovaleryl-CoA.

According to the present invention, the conversion of 3-hydroxyisovalerate (HIV) and 3-methylcrotonyl-CoA into 3-methylcrotonic acid and 3-hydroxyisovaleryl-CoA wherein 3-hydroxyisovalerate (HIV) is enzymatically converted into 3-methylcrotonic acid with a concomitant transfer of CoA from 3-methylcrotonyl-CoA on 3-hydroxyisovalerate (HIV) to result in 3-hydroxyisovaleryl-CoA preferably makes use of an enzyme which is classified as a CoA-transferase (EC 2.8.3.-) capable of transferring the CoA group of 3-methylcrotonyl-CoA to a carboxylic acid, i.e., 3-hydroxyisovalerate (HIV).

CoA-transferases (EC 2.8.3.-) as well as preferred enzymes of this enzyme class have already been described above. Accordingly, as regards these enzymes, the same applies to the conversion of 3-hydroxyisovalerate (HIV) and 3-methylcrotonyl-CoA into 3-methylcrotonic acid and 3-hydroxyisovaleryl-CoA as has been set forth above.

Preferably, the CoA-transferase employed in a method according to the present invention in the enzymatic conversion of 3-hydroxyisovalerate (HIV) into 3-methylcrotonic acid with a concomitant transfer of CoA from 3-methylcrotonyl-CoA on 3-hydroxyisovalerate (HIV) to result in 3-hydroxyisovaleryl-CoA is a CoA-transferase selected from the group consisting of:
propionate:acetate-CoA transferase (EC 2.8.3.1);
acetate CoA-transferase (EC 2.8.3.8); and
butyrate-acetoacetate CoA-transferase (EC 2.8.3.9).

Propionate:acetate-CoA transferases (EC 2.8.3.1), acetate CoA-transferases (EC 2.8.3.8) and butyrate-acetoacetate CoA-transferases (EC 2.8.3.9) as well as preferred enzymes of these enzyme classes have already been described above. Accordingly, as regards these enzymes, the same applies to the conversion of 3-hydroxyisovalerate (HIV) and 3-methylcrotonyl-CoA into 3-methylcrotonic acid and 3-hydroxyisovaleryl-CoA as has been set forth above.

The enzymatic conversion of 3-hydroxyisovalerate (HIV) into 3-hydroxyisovaleryl-CoA as shown in step Xb of FIG. 18

In addition or in the alternative to the above-described methods (step Xa), the 3-hydroxyisovaleryl-CoA may also be provided by an enzymatic conversion of 3-hydroxyisovalerate into said 3-hydroxyisovaleryl-CoA (step Xb as shown in FIG. 18). In this reaction, 3-hydroxyisovalerate reacts with an acyl-CoA to result in 3-hydroxyisovaleryl-CoA and an acid. This reaction is schematically illustrated in FIG. 19.

Preferably, said acyl-CoA is acetyl-CoA.

Thus, the present invention also relates to a method for producing 3-hydroxyisovaleryl-CoA from 3-hydroxyisovalerate (HIV) wherein 3-hydroxyisovalerate reacts with an acyl-CoA, preferably with acetyl-CoA, to result in 3-hydroxyisovaleryl-CoA and a respective acid.

Preferably, this conversion is achieved by making use of an enzyme which is classified as a CoA-transferase (EC 2.8.3.-). As regards the preferred embodiments of said CoA-transferase (EC 2.8.3.-) in the context of step Xb, the same applies, mutatis mutandis, as has been set forth above with respect to the CoA-transferases (EC 2.8.3.-) in the enzymatic conversion of 3-hydroxyisovalerate (HIV) into 3-methylcrotonic acid with a concomitant transfer of CoA from 3-methylcrotonyl-CoA on 3-hydroxyisovalerate (HIV) to result in 3-hydroxyisovaleryl-CoA (step Xa as shown in FIG. 18).

The enzymatic conversion of 3-hydroxyisovaleryl-CoA into 3-methylcrotonyl-CoA as shown in step XI of FIG. 18

In addition or in the alternative to the above-described methods (step VII), the 3-methylcrotonyl-CoA may be provided by an enzymatic reaction wherein 3-hydroxyisovaleryl-CoA is enzymatically converted into 3-methylcrotonyl-CoA (step XI as shown in FIG. 18). This reversible reaction is a dehydration reaction wherein 3-hydroxyisovaleryl-CoA is dehydrated into 3-methylcrotonyl-CoA and is schematically illustrated in FIG. 21.

Thus, the present invention also relates to a method for producing isobutene from 3-hydroxyisovaleryl-CoA wherein 3-hydroxyisovaleryl-CoA is first enzymatically converted into 3-methylcrotonyl-CoA wherein 3-methylcrotonyl-CoA is further enzymatically converted into 3-methylcrotonic acid according to any of the above-described methods. Further, the thus produced 3-methylcrotonic acid is then enzymatically converted into isobutene as described herein above.

According to the present invention, the enzymatic conversion of 3-hydroxyisovaleryl-CoA into 3-methylcrotonyl-CoA preferably makes use of
(i) an enoyl-CoA hydratase (EC 4.2.1.17);
(ii) a long-chain-enoyl-CoA hydratase (EC 4.2.1.74);
(iii) a 3-hydroxypropionyl-CoA dehydratase (EC 4.2.1.116);
(iv) a 3-hydroxybutyryl-CoA dehydratase (EC 4.2.1.55);
(v) a 3-hydroxyoctanoyl-[acyl-carrier-protein]dehydratase (EC 4.2.1.59);
(vi) a crotonyl-[acyl-carrier-protein]hydratase (EC 4.2.1.58);
(vii) a 3-hydroxydecanoyl-[acyl-carrier-protein]dehydratase (EC 4.2.1.60);
(viii) a 3-hydroxypalmitoyl-[acyl-carrier-protein]dehydratase (EC 4.2.1.61); or
(ix) a 3-methylglutaconyl-CoA hydratase (EC 4.2.1.18).

In a preferred embodiment of the method according to the invention the conversion of 3-hydroxyisovaleryl-CoA into 3-methylcrotonyl-CoA is achieved by the use of an enoyl-CoA hydratase (EC 4.2.1.17). Enoyl-CoA hydratases (EC 4.2.1.17) as well as preferred enzymes of this enzyme class have already been described above. Accordingly, as regards these enzymes, the same applies to the conversion of 3-hydroxyisovaleryl-CoA into 3-methylcrotonyl-CoA as has been set forth above.

In another preferred embodiment of the method according to the invention the conversion of 3-hydroxyisovaleryl-CoA into 3-methylcrotonyl-CoA is achieved by the use of a long-chain-enoyl-CoA hydratase (EC 4.2.1.74). Long-chain-enoyl-CoA hydratases (EC 4.2.1.74) catalyze the following reaction:

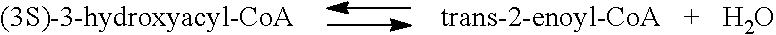

This enzyme belongs to the family of lyases, specifically the hydro-lyases, which cleave carbon-oxygen bonds. The systematic name of this enzyme class is long-chain-(3S)-3-hydroxyacyl-CoA hydro-lyase. This enzyme is also called long-chain enoyl coenzyme A hydratase and it participates in fatty acid elongation in mitochondria and fatty acid metabolism. This enzyme occurs in a number of organisms, e.g., in Rattus norvegicus (Wu et al., Org. Lett. 10 (2008), 2235-2238), Sus scrofa and Cavia porcellus (Fong and Schulz, J. Biol. Chem. 252 (1977), 542-547; Schulz, Biol. Chem. 249 (1974), 2704-2709) and in principle any long-chain-enoyl-CoA hydratase which can catalyze the conversion of 3-hydroxyisovaleryl-CoA into 3-methylcrotonyl-CoA can be employed in the method of the invention.

The enzymatic conversion of 3-hydroxyisovalerate (HIV) into 3-hydroxyisovaleryl-CoA as shown in step XII of FIG. 18

In addition or in the alternative to the above-described methods (step Xa or step Xb), the 3-hydroxyisovaleryl-CoA may also be provided by an enzymatic conversion of 3-hydroxyisovalerate (HIV) into said 3-hydroxyisovaleryl-CoA (step XII as shown in FIG. 18). This general reaction wherein coenzyme A (CoASH) is fixed is schematically illustrated in FIG. 22.

Thus, the present invention also relates to a method for producing isobutene from 3-hydroxyisovalerate (HIV) in which 3-hydroxyisovalerate (HIV) is first converted into 3-hydroxyisovaleryl-CoA wherein 3-hydroxyisovaleryl-CoA is then enzymatically converted into 3-methylcrotonyl-CoA wherein 3-methylcrotonyl-CoA is further enzymatically converted into 3-methylcrotonic acid according to any of the above-described methods. Further, the thus produced 3-methylcrotonic acid is then enzymatically converted into isobutene as described herein above.

Moreover, the present invention also relates to a method for producing 3-hydroxyisovaleryl-CoA from 3-hydroxyisovalerate (HIV).

According to the present invention, the enzymatic conversion of 3-hydroxyisovalerate (HIV) into 3-hydroxyisovaleryl-CoA preferably makes use of an enzyme belonging to the family of ligases forming a carbon-sulfur bond (EC 6.2.1.-). The general reaction of the enzymatic conversion of 3-hydroxyisovalerate (HIV) into 3-hydroxyisovaleryl-CoA wherein coenzyme A (CoASH) is fixed can be catalyzed by an enzyme belonging to the family of ligases forming a carbon-sulfur bond (EC 6.2.1.-) via two alternative mechanisms. In a first alternative reaction, an acyl-AMP is generated as an intermediate before coenzyme A is fixed as schematically illustrated in FIG. 23. In a second alternative reaction, an acyl phosphate is generated as an intermediate before coenzyme A is fixed as schematically illustrated in FIG. 24.

Enzymes which belong to the family of ligases forming a carbon-sulfur bond (EC 6.2.1.-) which are capable of enzymatically converting 3-hydroxyisovalerate (HIV) into 3-hydroxyisovaleryl-CoA wherein an acyl-AMP intermediate (i.e., the acyl adenylate intermediate 3-hydroxyisovaleryl-adenosine monophosphate) is generated before coenzyme A is fixed coenzyme A (CoASH) share common structural motifs which are referenced in the InterPro (InterPro44.0; Release Sep. 25, 2013) as InterPro IPR020845, AMP-binding, conserved site (http://www.ebi.ac.uk/interpro/entry/IPR020845) and IPR000873 (http://www.ebi.ac.uk/interpro/entry/IPR000873). The accession number for these enzymes in the Pfam database is PF00501.

As regards the first alternative reaction (wherein an acyl-AMP is generated as an intermediate before coenzyme A is fixed as schematically illustrated in FIG. 23), examples of enzymes which belong to the above family of ligases forming a carbon-sulfur bond (EC 6.2.1.-) which are capable of enzymatically converting 3-hydroxyisovalerate (HIV) into 3-hydroxyisovaleryl-CoA wherein an acyl-AMP intermediate (i.e., the acyl adenylate intermediate 3-hydroxyisovaleryl-adenosine monophosphate) is generated before coenzyme A is fixed coenzyme A (CoASH) and which may be used in the method for producing 3-hydroxyisovaleryl-CoA from 3-hydroxyisovalerate (HIV) are summarized in the following Table A:

TABLE A
CoA ligases (EC 6.2.1.-) capable of enzymatically converting 3-
hydroxyisovalerate (HIV) into 3-hydroxyisovaleryl-CoA involving
an acyl-adenylate as an intermediate

| Enzyme name | EC number |
|---|---|
| Acetate-CoA ligase | 6.2.1.1 |
| Butyrate-CoA ligase | 6.2.1.2 |
| Long chain fatty-acid-CoA ligase | 6.2.1.3 |
| 4-coumarate-CoA ligase | 6.2.1.12 |
| Arachidonate-CoA ligase | 6.2.1.15 |
| Propionate-CoA ligase | 6.2.1.17 |
| Phytanate-CoA ligase | 6.2.1.24 |
| o-succinylbenzoate-CoA ligase | 6.2.1.26 |
| 3-alpha,7-alpha-dihydroxy-5- | 6.2.1.28 |
| beta-cholestanate-CoA ligase | |
| 2-furoate-CoA ligase | 6.2.1.31 |
| 4-chlorobenzoate-CoA ligase | 6.2.1.33 |
| 3-hydroxybenzoate-CoA ligase | 6.2.1.37 |
| 4-hydroxybutyrate-CoA ligase | 6.2.1.40 |
| 3-oxocholest-4-en-26-oate--CoA ligase | 6.2.1.42 |
| 3-(methylthio)propionyl-CoA ligase | 6.2.1.44 |
| Cholate-CoA ligase | 6.2.1.7 |
| Oxalate-CoA ligase | 6.2.1.8 |
| Biotin-CoA ligase | 6.2.1.11 |
| 6-carboxyhexanoate-CoA ligase | 6.2.1.14 |
| Acetoacetate-CoA ligase | 6.2.1.16 |
| Dicarboxylate-CoA ligase | 6.2.1.23 |
| Benzoate-CoA ligase | 6.2.1.25 |
| 4-hydroxybenzoate-CoA ligase | 6.2.1.27 |
| Phenylacetate-CoA ligase | 6.2.1.30 |
| Anthranilate-CoA ligase | 6.2.1.32 |
| 3-hydroxypropionyl-CoA synthase | 6.2.1.36 |
| (2,2,3-trimethy1-5-oxocyclopent-3- | 6.2.1.38 |
| enyl)acetyl-CoA synthase | |
| 3-((3aS,4S,7aS)-7a-methy1-1,5-dioxo- | 6.2.1.41 |
| octahydro-1H-inden-4-yl)propanoate- | |
| CoA ligase | |
| 2-hydroxy-7-methoxy-5-methyl- | 6.2.1.43 |
| 1-naphthoate-CoA ligase | |
| Malonate-CoA ligase | 6.2.1.n3 |

In a preferred embodiment, the enzymatic conversion of 3-hydroxyisovalerate (HIV) into 3-hydroxyisovaleryl-CoA via an acyl adenylate intermediate can, e.g., be achieved by the use of a butanoate:CoA ligase (AMP forming) (EC 6.2.1.2). Butanoate:CoA ligases are enzymes which catalyze the following reaction:

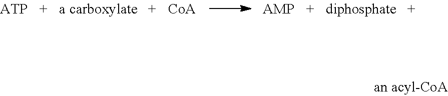

These enzymes participate in butanoate metabolism. The occurrence of these enzymes has been described for a large number of organisms, including prokaryotes and eukaryotes, in particular, bacteria, algae, fungi, plants and animals, e.g. for Methanobacterium formicum, Streptomyces coelicolor, Mycobacterium avium, Penicillium chrysogenum, Paecilomyces variotii, Pseudomonas aeruginosa, Dictyostelium discoideum, Cavia porcellus, Ovis aries, Sus scrofa, Bos taurus, Mus musculus, Rattus norvegicus, and Homo sapiens.

In a preferred embodiment, the enzymatic conversion of 3-hydroxyisovalerate (HIV) into 3-hydroxyisovaleryl-CoA via an acyl adenylate intermediate is achieved by making use of a butanoate:CoA ligase (AMP forming) (EC 6.2.1.2) derived from Methanobacterium formicum. The amino acid sequence of said protein is shown in SEQ ID NO: 77.

In a preferred embodiment of the present invention the butanoate:CoA ligase (AMP forming) is an enzyme comprising the amino acid sequence of SEQ ID NO: 77 or a sequence which is at least n % identical to SEQ ID NO: 77 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-hydroxyisovalerate (HIV) into 3-hydroxyisovaleryl-CoA. As regards the determination of the sequence identity, the same applies as has been set forth above.

As regards the second alternative reaction (wherein an acyl phosphate is generated as an intermediate before coenzyme A is fixed as schematically illustrated in FIG. 24), examples of enzymes which belong to the above family of ligases forming a carbon-sulfur bond (EC 6.2.1.-) which are capable of enzymatically converting 3-hydroxyisovalerate (HIV) into 3-hydroxyisovaleryl-CoA wherein an acyl phosphate intermediate (i.e., the acyl phosphate intermediate 3-hydroxyisovaleryl phosphate) is generated before coenzyme A is fixed coenzyme A (CoASH) and which may be used in the method for producing 3-hydroxyisovaleryl-CoA from 3-hydroxyisovalerate (HIV) are summarized in the following Table B.

TABLE B
CoA ligases (EC 6.2.1.-) capable of enzymatically converting 3-
hydroxyisovalerate (HIV) into 3-hydroxyisovaleryl-CoA involving
an acyl phosphate as an intermediate

| Enzyme name | EC number |
|---|---|
| Succinate-CoA ligase (GDP-forming) | 6.2.1.4 |
| Glutarate-CoA ligase | 6.2.1.6 |
| Acid-CoA ligase (GDP-forming) | 6.2.1 .10 |
| Citrate-CoA ligase | 6.2.1 .18 |
| Succinate-CoA ligase (ADP-forming) | 6.2.1.5 |
| Malate-CoA ligase | 6.2.1.9 |
| Acetate-CoA ligase (ADP-forming) | 6.2.1.13 |

The alternative route for the enzymatic conversion from acetyl-CoA into isobutene via 3-methyl-3-butenoyl-CoA and 3-methyl-3-butenoic acid

In an alternative to the above, the present invention also relates to a method for the production of isobutene via an alternative route as also shown in FIG. 1 wherein isobutene is produced by the enzymatic conversion of 3-methyl-3-butenoic acid into isobutene. Thus, the present invention provides a method for the production of isobutene comprising the enzymatic conversion of 3-methyl-3-butenoic acid into isobutene. Preferably, the enzymatic conversion of 3-methyl-3-butenoic acid into isobutene is achieved by making use of an 3-methyl-3-butenoic acid decarboxylase.

In accordance with this alternative route, the present invention not only relates to a method for the production of isobutene from 3-methyl-3-butenoic acid. Rather, as will be outlined in more detail further below, this conversion is preferably embedded in a pathway for the production of isobutene starting from acetyl-CoA which is a central component and an important key molecule in metabolism used in many biochemical reactions.

Therefore, the present invention also relates to a pathway starting from acetyl-CoA wherein two acetyl-CoA molecules are enzymatically condensed into acetoacetyl-CoA. Alternatively, acetyl-CoA is enzymatically converted into malonyl-CoA which may then be converted into said acetoacetyl-CoA by the enzymatic condensation of malonyl-CoA and acetyl-CoA into said acetoacetyl-CoA.

Further, the thus produced acetoacetyl-CoA can enzymatically be converted into 3-methyl-3-butenoic acid (which is then ultimately converted into isobutene) via the following briefly summarized pathway.

In this pathway, the thus produced acetoacetyl-CoA can further enzymatically be converted into 3-hydroxy-3-methylglutaryl-CoA. Moreover, the thus produced 3-hydroxy-3-methylglutaryl-CoA can further enzymatically be converted into 3-methylglutaconyl-CoA. Further, the thus produced 3-methylglutaconyl-CoA can enzymatically be converted into 3-methyl-3-butenoyl-CoA. Further, the thus produced 3-methyl-3-butenoyl-CoA can further be converted in a subsequent enzymatic reaction into 3-methyl-3-butenoic acid (which can then ultimately be converted into isobutene as described above and further below).

The enzymatic conversion of 3-methyl-3-butenoic acid into isobutene: step XVI as shown in FIG. 1

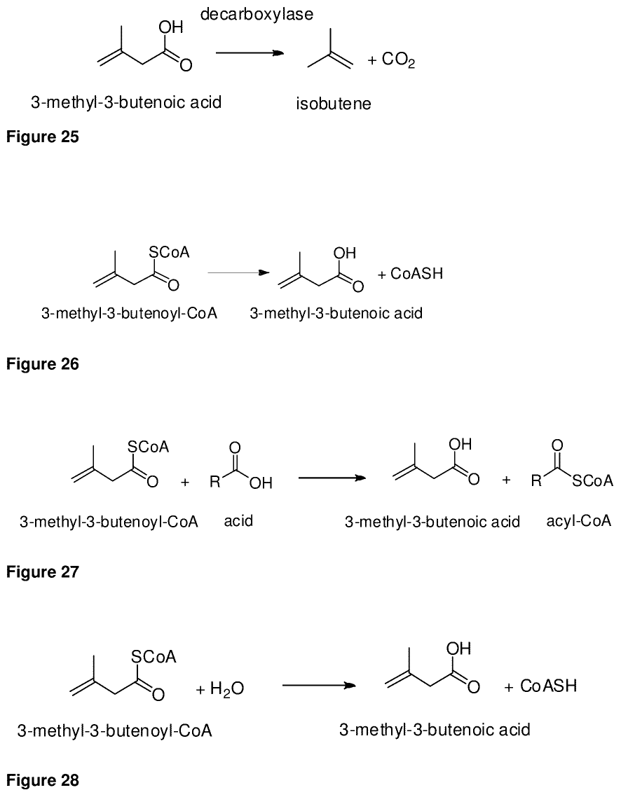

According to the present invention, the enzymatic conversion of 3-methyl-3-butenoic acid into isobutene can be achieved by a decarboxylation. “Decarboxylation” is generally a chemical reaction that removes a carboxyl group and releases carbon dioxide (CO₂); see FIG. 25.

The enzymatic conversion of 3-methyl-3-butenoic acid into isobutene can preferably be achieved by making use of an 3-methyl-3-butenoic acid decarboxylase. In accordance with the present invention, an 3-methyl-3-butenoic acid decarboxylase is an enzyme which is capable of converting 3-methyl-3-butenoic acid into isobutene. In preferred embodiments, the 3-methyl-3-butenoic acid decarboxylase is selected from the group consisting of:
(i) an FMN-dependent decarboxylase associated with an FMN prenyl transferase; or
(ii) an aconitate decarboxylase (EC 4.1.1.6); or
(iii) a methylcrotonyl-CoA carboxylase (EC 6.4.1.4); or
(iv) a geranoyl-CoA carboxylase (EC 6.4.1.5); or
(v) a protocatechuate (PCA) decarboxylase (EC 4.1.1.63).

In other preferred embodiments, the 3-methyl-3-butenoic acid decarboxylase is selected from the group consisting of: 6-methylsalicylate decarboxylase (EC 4.1.1.52), 2-oxo-3-hexenedioate decarboxylase (EC 4.1.1.77) and 5-oxopent-3-ene-1,2,5-tricarboxylate decarboxylase (EC 4.1.1.68).

As regards the afore-mentioned embodiment, for the FMN-dependent decarboxylase associated with an FMN prenyl transferase, the aconitate decarboxylase (EC 4.1.1.6), the methylcrotonyl-CoA carboxylase (EC 6.4.1.4), the geranoyl-CoA carboxylase (EC 6.4.1.5), the protocatechuate (PCA) decarboxylase (EC 4.1.1.63), the 6-methylsalicylate decarboxylase (EC 4.1.1.52), the 2-oxo-3-hexenedioate decarboxylase (EC 4.1.1.77) and the 5-oxopent-3-ene-1,2,5-tricarboxylate decarboxylase (EC 4.1.1.68), the same applies as has been set forth above in connection with other methods of the present invention.

The enzymatic conversion of 3-methyl-3-butenoyl-CoA into 3-methyl-3-butenoic acid: steps XVIIa, XVIIb or XVIIc as shown in FIG. 1

The 3-methyl-3-butenoic acid may itself be provided by an enzymatic reaction, namely the enzymatic conversion of 3-methyl-3-butenoyl-CoA into 3-methyl-3-butenoic acid; see FIG. 26.

Accordingly, the present invention relates to a method for producing isobutene from 3-methyl-3-butenoyl-CoA in which 3-methyl-3-butenoyl-CoA is first converted into 3-methyl-3-butenoic acid which is then further converted into isobutene as described herein above.

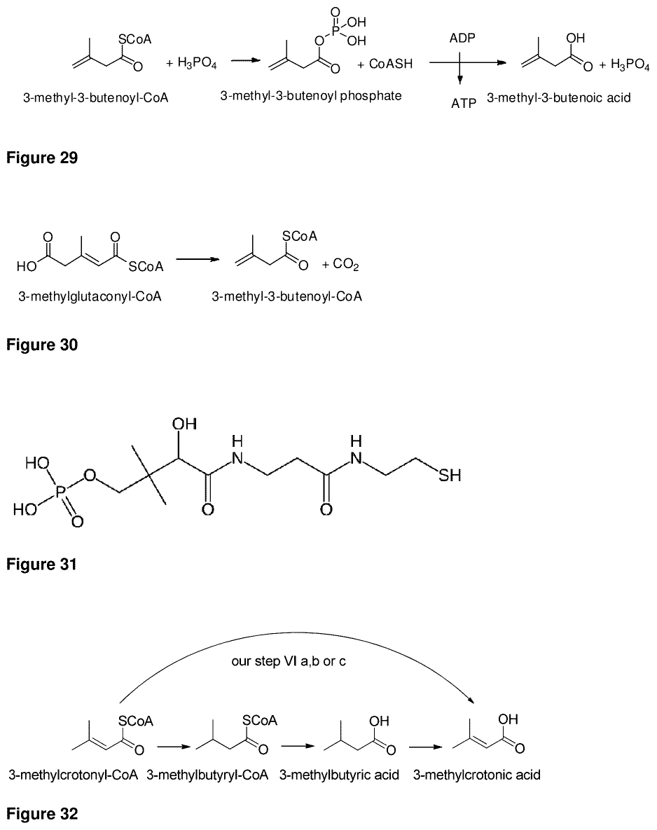

According to the present invention, the conversion of 3-methyl-3-butenoyl-CoA into 3-methyl-3-butenoic acid can, e.g., be achieved by three different alternative enzymatic routes, i.e., by:
(a) a single enzymatic reaction (see FIG. 27) in which 3-methyl-3-butenoyl-CoA is directly converted into 3-methyl-3-butenoic acid, preferably by making use of a CoA transferase (EC 2.8.3.-), preferably a propionate:acetate-CoA transferase (EC 2.8.3.1), an acetate CoA-transferase (EC 2.8.3.8) or a succinyl-CoA:acetate CoA-transferase (EC 2.8.3.18);
(b) a single enzymatic reaction(see FIG. 28) in which 3-methyl-3-butenoyl-CoA is directly converted into 3-methyl-3-butenoic acid, preferably by making use of a thioester hydrolase (EC 3.1.2.-), preferably acetyl-CoA hydrolase (EC 3.1.2.1), an ADP-dependent short-chain-acyl-CoA hydrolase (EC 3.1.2.18) or an acyl-CoA hydrolase (EC 3.1.2.20); or
(c) two enzymatic steps (see FIG. 29) comprising
   (i) first enzymatically converting 3-methyl-3-butenoyl-CoA into 3-methyl-3-butenoyl phosphate, preferably by making use of a phosphate butyryltransferase (EC 2.3.1.19) or a phosphate acetyltransferase (EC 2.3.1.8); and
   (ii) then enzymatically converting the thus obtained 3-methyl-3-butenoyl phosphate into said 3-methyl-3-butenoic acid, preferably by making use of a phosphotransferase with a carboxy group as acceptor (EC 2.7.2.-), preferably a propionate kinase (EC 2.7.2.15), an acetate kinase (EC 2.7.2.1), a butyrate kinase (EC 2.7.2.7) or a branched-chain-fatty-acid kinase (EC 2.7.2.14).

As regards the aforementioned embodiments, for the CoA transferase (EC 2.8.3.-), the propionate:acetate-CoA transferase (EC 2.8.3.1), the acetate CoA-transferase (EC 2.8.3.8), the succinyl-CoA:acetate CoA-transferase (EC 2.8.3.18), the thioester hydrolase (EC 3.1.2.-), the acetyl-CoA hydrolase (EC 3.1.2.1), the ADP-dependent short-chain-acyl-CoA hydrolase (EC 3.1.2.18), the an acyl-CoA hydrolase (EC 3.1.2.20) the phosphate butyryltransferase (EC 2.3.1.19), the phosphate acetyltransferase (EC 2.3.1.8), the phosphotransferase with a carboxy group as acceptor (EC 2.7.2.-), the propionate kinase (EC 2.7.2.15), the acetate kinase (EC 2.7.2.1), the butyrate kinase (EC 2.7.2.7) and the branched-chain-fatty-acid kinase (EC 2.7.2.14), the same applies as has been set forth above in connection with the other methods of the present invention.

The enzymatic conversion of 3-methylglutaconyl-CoA into 3-methyl-3-butenoyl-CoA: step XVIII as shown in FIG. 1

The 3-methyl-3-butenoyl-CoA may itself be provided by an enzymatic reaction, namely the enzymatic conversion of 3-methylglutaconyl-CoA into 3-methyl-3-butenoyl-CoA; see FIG. 30.

Accordingly, the present invention relates to a method for producing isobutene from 3-methyl-3-butenoyl-CoA in which 3-methylglutaconyl-CoA is first converted into 3-methyl-3-butenoyl-CoA which is then further converted into 3-methyl-3-butenoic acid which is then further converted into isobutene as described herein above.

Moreover, the present invention relates to a method for producing 3-methyl-3-butenoyl-CoA by converting 3-methylglutaconyl-CoA into 3-methyl-3-butenoyl-CoA.

According to the present invention, the conversion of 3-methylglutaconyl-CoA into 3-methyl-3-butenoyl-CoA can preferably be achieved by making use of
(a) (i) a methylcrotonyl-CoA carboxylase (EC 6.4.1.4); or (ii) a geranoyl-CoA carboxylase (EC 6.4.1.5),
(b) an N-terminal domain of CurF from Lynbya majuscula multifunctional protein or a 3-methylglutaconyl-CoA decarboxylase, preferably a 3-methylglutaconyl-CoA decarboxylase of Myxococcus xanthus encoded by the IiuB gene; or
(c) an enzyme of the 4-oxalocrotonate decarboxylase family.

As regards the aforementioned embodiments, for the methylcrotonyl-CoA carboxylase (EC 6.4.1.4), the geranoyl-CoA carboxylase (EC 6.4.1.5) and the 3-methylglutaconyl-CoA decarboxylase, preferably the 3-methylglutaconyl-CoA decarboxylase of Myxococcus xanthus encoded by the IiuB gene, the same applies as has been set forth above in connection with the other methods of the present invention.

In a preferred embodiment the conversion of 3-methylglutaconyl-CoA via decarboxylation into 3-methyl-3-butenoyl-CoA is catalyzed by an N-terminal domain of CurF from Lynbya majuscula multifunctional protein. The N-terminal domain of CurF from Lynbya majuscula multifunctional protein is a domain of a polyketide synthase (PKS)/non ribosomale peptide synthase (NRPS) of the CurF multifunctional protein from Lyngbya majuscula. This N-terminal domain of CurF has been classified as a protein belonging to the crotonase superfamily by studying the crystal structure and it naturally catalyzes the decarboxylation of 3-methylglutaconyl-ACP (Acyl Carrier Protein) into 3-methyl-crotonyl-ACP. ACP is similar to CoA as both molecules have a phosphopantetheine moiety in common (as shown in FIG. 31). Moreover, both ACP and CoA can form a thioester with a biological acid (J. Biol. Chem. 289: 35957-35963 (2007) and Chemistry & Biology 11:817-833 (2004)).

In another preferred embodiment the conversion of 3-methylglutaconyl-CoA via decarboxylation into 3-methyl-3-butenoyl-CoA is catalyzed by an enzyme of the 4-oxalocrotonate decarboxylase family (EC 4.1.1.77).

4-oxalocrotonate decarboxylases (EC 4.1.1.77) catalyse the following reaction:

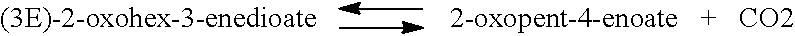

This enzyme is known from various organisms and has, e.g., been described in Bortetella sp., Cupriavidus nector, Geobacillus stearothermophilus, Pseudomonas putida and Ralstonia pickettii. Thus, in a preferred embodiment, the 4-oxalocrotonate decarboxylase used for the conversion of 3-methylglutaconyl-CoA via decarboxylation into 3-methyl-3-butenoyl-CoA is a 4-oxalocrotonate decarboxylase derived from genus Bortetella, Cupriavidus, Geobacillus, Pseudomonas pr Ralstonia, more preferably from the species Bortetella sp., Cupriavidus nector, Geobacillus stearothermophilus, Pseudomonas putida or Ralstonia pickettii. In an even more preferred embodiment, the 4-oxalocrotonate decarboxylase used for the conversion of 3-methylglutaconyl-CoA via decarboxylation into 3-methyl-3-butenoyl-CoA is the 4-oxalocrotonate decarboxylase of Geobacillus stearothermophilus (UniProt Accession number B0VXM8).

In a preferred embodiment, the 4-oxalocrotonate decarboxylase employed in the method of the present invention in the conversion of 3-methylglutaconyl-CoA via decarboxylation into 3-methyl-3-butenoyl-CoA is derived from Geobacillus stearothermophilus and has an amino acid sequence as shown SEQ ID NO:69.

In a preferred embodiment of the present invention the 4-oxalocrotonate decarboxylase is an enzyme comprising the amino acid sequence of SEQ ID NO: 69 or a sequence which is at least n % identical to SEQ ID NO: 69 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylglutaconyl-CoA via decarboxylation into 3-methyl-3-butenoyl-CoA. As regards the determination of the sequence identity, the same applies as has been set forth above.

The enzymatic conversion of 3-hydroxy-3-methylglutaryl-CoA into 3-methylglutaconyl-CoA: step VIII as shown in FIG. 1

The 3-methylglutaconyl-CoA which can be converted into 3-methyl-3-butenoyl-CoA according to any of the above described methods may itself be provided by an enzymatic reaction, namely the enzymatic conversion of 3-hydroxy-3-methylglutaryl-CoA into 3-methylglutaconyl-CoA.

Accordingly, the present invention also relates to a method for producing isobutene from 3-hydroxy-3-methylglutaryl-CoA in which 3-hydroxy-3-methylglutaryl-CoA is first converted into 3-methylglutaconyl-CoA which is then converted into 3-methyl-3-butenoyl-CoA which is then further converted into 3-methyl-3-butenoic acid which is then further converted into isobutene as described herein above.

According to the present invention, the enzymatic conversion of 3-hydroxy-3-methylglutaryl-CoA into 3-methylglutaconyl-CoA is an enzymatic dehydration reaction which occurs naturally, and which is catalyzed, e.g., by enzymes classified as 3-methylglutaconyl-coenzyme A hydratase (EC 4.2.1.18). Accordingly, the enzymatic conversion of 3-hydroxy-3-methylglutaryl-CoA into 3-methylglutaconyl-CoA preferably makes use of a 3-methylglutaconyl-coenzyme A hydratase (EC 4.2.1.18).

As regards the afore-mentioned embodiment, for the enzymes classified as 3-methylglutaconyl-coenzyme A hydratase (EC 4.2.1.18), the same applies as has been set forth above in connection with the other methods of the present invention.

The enzymatic conversion of acetoacetyl-CoA into 3-hydroxy-3-methylglutaryl-CoA: step IX as shown in FIG. 1

The 3-hydroxy-3-methylglutaryl-CoA may itself be provided by an enzymatic reaction, namely the enzymatic condensation of acetoacetyl-CoA and acetyl-CoA into 3-hydroxy-3-methylglutaryl-CoA which has already been described in detail above.

Accordingly, the present invention also relates to a method for producing isobutene from acetoacetyl-CoA and acetyl-CoA in which acetoacetyl-CoA and acetyl-CoA are first condensed into 3-hydroxy-3-methylglutaryl-CoA which is then converted into 3-methylglutaconyl-CoA which is then converted into 3-methyl-3-butenoyl-CoA which is then further converted into 3-methyl-3-butenoic acid which is then further converted into isobutene as described herein above.

The enzymatic conversion of acetyl-CoA into acetoacetyl-CoA: step XIII, step XIV and step XV as shown in FIG. 1

The acetoacetyl-CoA may itself be provided by an enzymatic reaction, namely the enzymatic conversion of acetyl-CoA into acetoacetyl-CoA via several different routes which have already been described in detail above.

Thus, the present invention also relates to a method for producing isobutene from acetyl-CoA in which acetyl-CoA is first converted into acetoacetyl-CoA by any of the above-mentioned routes which is then condensed with acetyl-CoA into 3-hydroxy-3-methylglutaryl-CoA which is then converted into 3-methylglutaconyl-CoA which is then converted into 3-methyl-3-butenoyl-CoA which is then further converted into 3-methyl-3-butenoic acid which is then further converted into isobutene as described herein above.

Summarizing the alternative route for the enzymatic conversion from acetyl-CoA into isobutene via 3-methyl-3-butenoyl-CoA and 3-methyl-3-butenoic acid as outlined above, the present invention also relates to the following embodiments as characterized by the following items 1 to 26:

1. A method for the production of isobutene comprising the enzymatic conversion of 3-methyl-3-butenoic acid into isobutene.

2. The method of item 1, wherein the enzymatic conversion of 3-methyl-3-butenoic acid into isobutene is achieved by making use of an 3-methyl-3-butenoic acid decarboxylase.

3. The method of item 2, wherein the 3-methyl-3-butenoic acid decarboxylase is:
(i) an FMN-dependent decarboxylase associated with an FMN prenyl transferase; or
(ii) an aconitate decarboxylase (EC 4.1.1.6); or
(iii) a methylcrotonyl-CoA carboxylase (EC 6.4.1.4); or
(iv) a geranoyl-CoA carboxylase (EC 6.4.1.5); or
(v) a protocatechuate (PCA) decarboxylase (EC 4.1.1.63).

4. The method of item 1 or 2, further comprising providing the 3-methyl-3-butenoic acid by the enzymatic conversion of 3-methyl-3-butenoyl-CoA into 3-methyl-3-butenoic acid.

5. The method of item 4, wherein the enzymatic conversion of 3-methyl-3-butenoyl-CoA into 3-methyl-3-butenoic acid is achieved by
(a) a single enzymatic reaction in which 3-methyl-3-butenoyl-CoA is directly converted into 3-methyl-3-butenoic acid by making use of a CoA transferase (EC 2.8.3.-), preferably a propionate:acetate-CoA transferase (EC 2.8.3.1), an acetate CoA-transferase (EC 2.8.3.8) or a succinyl-CoA:acetate CoA-transferase (EC 2.8.3.18);
(b) a single enzymatic reaction in which 3-methyl-3-butenoyl-CoA is directly converted into 3-methyl-3-butenoic acid by making use of a thioester hydrolase (EC 3.1.2.-), preferably acetyl-CoA hydrolase (EC 3.1.2.1), an ADP-dependent short-chain-acyl-CoA hydrolase (EC 3.1.2.18) or an acyl-CoA hydrolase (EC 3.1.2.20);
(c) two enzymatic steps comprising
   (i) first enzymatically converting 3-methyl-3-butenoyl-CoA into 3-methyl-3-butenoyl phosphate; and
   (ii) then enzymatically converting the thus obtained 3-methyl-3-butenoyl phosphate into said 3-methyl-3-butenoic acid.

6. The method of item 5(c), wherein the enzymatic conversion of said 3-methyl-3-butenoyl-CoA into 3-methyl-3-butenoyl phosphate is achieved by making use of a phosphate butyryltransferase (EC 2.3.1.19) or a phosphate acetyltransferase (EC 2.3.1.8) and the enzymatic conversion of said 3-methyl-3-butenoyl phosphate into said 3-methyl-3-butenoic acid is achieved by making use of a phosphotransferase with a carboxy group as acceptor (EC 2.7.2.-), preferably a propionate kinase (EC 2.7.2.15), an acetate kinase (EC 2.7.2.1), a butyrate kinase (EC 2.7.2.7) or a branched-chain-fatty-acid kinase (EC 2.7.2.14).

7. The method of any one of items 1 to 4, further comprising providing the 3-methyl-3-butenoyl-CoA by the enzymatic conversion of 3-methylglutaconyl-CoA into 3-methyl-3-butenoyl-CoA.

8. The method of item 7, wherein the enzymatic conversion of 3-methylglutaconyl-CoA into 3-methyl-3-butenoyl-CoA is achieved by making use of
(a) (i) a methylcrotonyl-CoA carboxylase (EC 6.4.1.4); or (ii) a geranoyl-CoA carboxylase (EC 6.4.1.5),
(b) an N-terminal domain of CurF from Lynbya majuscula multifunctional protein or a 3-methylglutaconyl-CoA decarboxylase, preferably a 3-methylglutaconyl-CoA decarboxylase of Myxococcus xanthus encoded by the IiuB gene; or
(c) an enzyme of the 4-oxalocrotonate decarboxylase family.

9. The method of any one of items 1 to 8, further comprising providing the 3-methylglutaconyl-CoA by the enzymatic conversion of 3-hydroxy-3-methylglutaryl-CoA into 3-methylglutaconyl-CoA.

10. The method of item 9, wherein the enzymatic conversion of 3-hydroxy-3-methylglutaryl-CoA into 3-methylglutaconyl-CoA is achieved by making use of a 3-methylglutaconyl-coenzyme A hydratase (EC 4.2.1.18), a 3-hydroxyacyl-CoA dehydratase (EC 4.2.1.-) or an enoyl-CoA hydratase (EC 4.2.1.-).

11. The method of any one of items 1 to 10, further comprising providing the 3-hydroxy-3-methylglutaryl-CoA by the enzymatic condensation of acetoacetyl-CoA and acetyl-CoA into 3-hydroxy-3-methylglutaryl-CoA.

12. The method of item 11, wherein the enzymatic condensation of acetoacetyl-CoA and acetyl-CoA into 3-hydroxy-3-methylglutaryl-CoA is achieved by making use of a 3-hydroxy-3-methylglutaryl-CoA synthase.

13. The method of any one of items 1 to 12, further comprising providing the acetoacetyl-CoA by the enzymatic conversion of acetyl-CoA into acetoacetyl-CoA comprising:
(a) two enzymatic steps comprising
   (i) first the enzymatic conversion of acetyl-CoA into malonyl-CoA; and
   (ii) then enzymatically condensing the thus obtained malonyl-CoA and acetyl-CoA into said acetoacetyl-CoA; or
(b) a single enzymatic reaction in which two molecules of acetyl-CoA are directly condensed into acetoacetyl-CoA.

14. The method of item 13(a)(i), wherein the enzymatic conversion of acetyl-CoA into malonyl-CoA is achieved by making use of an acetyl-CoA carboxylase (EC 6.4.1.2).

15. The method of item 13(a)(ii), wherein the enzymatic condensation of malonyl-CoA and acetyl-CoA into said acetoacetyl-CoA is achieved by making use of an acetoacetyl-CoA synthase (EC 2.3.1.194).

16. The method of item 13(b), wherein the direct enzymatic condensation of two molecules of acetyl-CoA into acetoacetyl-CoA is achieved by making use of an acetyl-CoA C-acetyltransferase (EC 2.3.1.9).

17. A recombinant organism or microorganism which expresses
(i) an enzyme as defined in any one of items 1 to 3; and
(ii) an enzyme as defined in any one of items 4 to 6.

18. The recombinant organism or microorganism of item 17, further expressing an enzyme as defined in item 7 or 8.

19. The recombinant organism or microorganism of item 18, further expressing an enzyme as defined in item 9 or 10.

20. The recombinant organism or microorganism of item 19, further expressing an enzyme as defined in item 11 or 12.

21. The recombinant organism or microorganism of item 20, further expressing an enzyme as defined in claim 13.

22. The recombinant organism or microorganism of item 21, further expressing an enzyme as defined in any one of claims 14 to 16.

23. Use of a recombinant organism or microorganism as defined in any one of items 17 to 22 for the production of isobutene.

24. The use of a recombinant organism or microorganism of item 23, wherein said recombinant organism or microorganism expresses an enzyme catalyzing the enzymatic conversion of 3-methyl-3-butenoic acid into isobutene.

25. Use of an enzyme catalyzing the enzymatic conversion of 3-methyl-3-butenoic acid into isobutene for the production of isobutene from 3-methyl-3-butenoic acid.

26. A composition comprising 3-methyl-3-butenoic acid and a recombinant organism or microorganism as defined in any one of items 17 to 22; or 3-methyl-3-butenoic acid and an enzyme as defined in any one of items 1 to 16.

A method according to the present invention may be carried out in vitro or in vivo. An in vitro reaction is understood to be a reaction in which no cells are employed, i.e. an acellular reaction. Thus, in vitro preferably means in a cell-free system. The term “in vitro” in one embodiment means in the presence of isolated enzymes (or enzyme systems optionally comprising possibly required cofactors). In one embodiment, the enzymes employed in the method are used in purified form.

For carrying out the method in vitro the substrates for the reaction and the enzymes are incubated under conditions (buffer, temperature, cosubstrates, cofactors etc.) allowing the enzymes to be active and the enzymatic conversion to occur. The reaction is allowed to proceed for a time sufficient to produce the respective product. The production of the respective products can be measured by methods known in the art, such as gas chromatography possibly linked to mass spectrometry detection.

The enzymes may be in any suitable form allowing the enzymatic reaction to take place. They may be purified or partially purified or in the form of crude cellular extracts or partially purified extracts. It is also possible that the enzymes are immobilized on a suitable carrier.

In another embodiment the method according to the invention is carried out in culture, in the presence of an organism, preferably a microorganism, producing the enzymes described above for the conversions of the methods according to the present invention as described herein above. A method which employs a microorganism for carrying out a method according to the invention is referred to as an “in vivo” method. It is possible to use a microorganism which naturally produces the enzymes described above for the conversions of the methods according to the present invention or a microorganism which had been genetically modified so that it expresses (including overexpresses) one or more of such enzymes. Thus, the microorganism can be an engineered microorganism which expresses enzymes described above for the conversions of the methods according to the present invention, i.e. which has in its genome a nucleotide sequence encoding such enzymes and which has been modified to overexpress them. The expression may occur constitutively or in an induced or regulated manner.

In another embodiment the microorganism can be a microorganism which has been genetically modified by the introduction of one or more nucleic acid molecules containing nucleotide sequences encoding one or more enzymes described above for the conversions of the methods according to the present invention. The nucleic acid molecule can be stably integrated into the genome of the microorganism or may be present in an extrachromosomal manner, e.g. on a plasmid.

Such a genetically modified microorganism can, e.g., be a microorganism that does not naturally express enzymes described above for the conversions of the methods according to the present invention and which has been genetically modified to express such enzymes or a microorganism which naturally expresses such enzymes and which has been genetically modified, e.g. transformed with a nucleic acid, e.g. a vector, encoding the respective enzyme(s), and/or insertion of a promoter in front of the endogenous nucleotide sequence encoding the enzyme in order to increase the respective activity in said microorganism.

However, the invention preferably excludes naturally occurring microorganisms as found in nature expressing an enzyme as described above at levels as they exist in nature. Instead, the microorganism of the present invention and employed in a method of the present invention is preferably a non-naturally occurring microorganism, whether it has been genetically modified to express (including overexpression) an exogenous enzyme of the invention not normally existing in its genome or whether it has been engineered to overexpress an exogenous enzyme.

Thus, the enzymes and (micro)organisms employed in connection with the present invention are preferably non-naturally occurring enzymes or (micro)organisms, i.e. they are enzymes or (micro)organisms which differ significantly from naturally occurring enzymes or microorganism and which do not occur in nature. As regards the enzymes, they are preferably variants of naturally occurring enzymes which do not as such occur in nature. Such variants include, for example, mutants, in particular prepared by molecular biological methods, which show improved properties, such as a higher enzyme activity, higher substrate specificity, higher temperature resistance and the like. As regards the (micro)organisms, they are preferably genetically modified organisms as described herein above which differ from naturally occurring organisms due to a genetic modification. Genetically modified organisms are organisms which do not naturally occur, i.e., which cannot be found in nature, and which differ substantially from naturally occurring organisms due to the introduction of a foreign nucleic acid molecule.

By overexpressing an exogenous or endogenous enzyme as described herein above, the concentration of the enzyme is substantially higher than what is found in nature, which can then unexpectedly force the reaction of the present invention which uses a non-natural for the respective enzyme. Preferably, the concentration of the overexpressed enzyme is at least 5%, 10%, 20%, 30% or 40% of the total host cell protein.

A “non-natural” substrate is understood to be a molecule that is not acted upon by the respective enzyme in nature, even though it may actually coexist in the microorganism along with the endogenous enzyme. This “non-natural” substrate is not converted by the microorganism in nature as other substrates are preferred (e.g. the “natural substrate”). Thus, the present invention contemplates utilizing a non-natural substrate with the enzymes described above in an environment not found in nature.

Thus, it is also possible in the context of the present invention that the microorganism is a microorganism which naturally does not have the respective enzyme activity but which is genetically modified so as to comprise a nucleotide sequence allowing the expression of a corresponding enzyme. Similarly, the microorganism may also be a microorganism which naturally has the respective enzyme activity but which is genetically modified so as to enhance such an activity, e.g. by the introduction of an exogenous nucleotide sequence encoding a corresponding enzyme or by the introduction of a promoter for the endogenous gene encoding the enzyme to increase endogenous production to overexpressed (non-natural) levels.

If a microorganism is used which naturally expresses a corresponding enzyme, it is possible to modify such a microorganism so that the respective activity is overexpressed in the microorganism. This can, e.g., be achieved by effecting mutations in the promoter region of the corresponding gene or introduction of a high expressing promoter so as to lead to a promoter which ensures a higher expression of the gene. Alternatively, it is also possible to mutate the gene as such so as to lead to an enzyme showing a higher activity.

By using microorganisms which express enzymes described above for the conversions of the methods according to the present invention, it is possible to carry out the methods according to the invention directly in the culture medium, without the need to separate or purify the enzymes.

In one embodiment the organism employed in a method according to the invention is a microorganism which has been genetically modified to contain a foreign nucleic acid molecule encoding at least one enzyme described above for the conversions of the methods according to the present invention. The term “foreign” or “exogenous” in this context means that the nucleic acid molecule does not naturally occur in said microorganism. This means that it does not occur in the same structure or at the same location in the microorganism. In one preferred embodiment, the foreign nucleic acid molecule is a recombinant molecule comprising a promoter and a coding sequence encoding the respective enzyme in which the promoter driving expression of the coding sequence is heterologous with respect to the coding sequence. “Heterologous” in this context means that the promoter is not the promoter naturally driving the expression of said coding sequence but is a promoter naturally driving expression of a different coding sequence, i.e., it is derived from another gene, or is a synthetic promoter or a chimeric promoter. Preferably, the promoter is a promoter heterologous to the microorganism, i.e. a promoter which does naturally not occur in the respective microorganism. Even more preferably, the promoter is an inducible promoter. Promoters for driving expression in different types of organisms, in particular in microorganisms, are well known to the person skilled in the art.

In a further embodiment the nucleic acid molecule is foreign to the microorganism in that the encoded enzyme is not endogenous to the microorganism, i.e. is naturally not expressed by the microorganism when it is not genetically modified. In other words, the encoded enzyme is heterologous with respect to the microorganism. The foreign nucleic acid molecule may be present in the microorganism in extrachromosomal form, e.g. as a plasmid, or stably integrated in the chromosome. A stable integration is preferred. Thus, the genetic modification can consist, e.g. in integrating the corresponding gene(s) encoding the enzyme(s) into the chromosome, or in expressing the enzyme(s) from a plasmid containing a promoter upstream of the enzyme-coding sequence, the promoter and coding sequence preferably originating from different organisms, or any other method known to one of skill in the art.

The term “microorganism” in the context of the present invention refers to bacteria, as well as to fungi, such as yeasts, and also to algae and archaea. In one preferred embodiment, the microorganism is a bacterium. In principle any bacterium can be used. Preferred bacteria to be employed in the process according to the invention are bacteria of the genus Bacillus, Clostridium, Corynebacterium, Pseudomonas, Zymomonas or Escherichia. In a particularly preferred embodiment the bacterium belongs to the genus Escherichia and even more preferred to the species Escherichia coli. In another preferred embodiment the bacterium belongs to the species Pseudomonas putida or to the species Zymomonas mobilis or to the species Corynebacterium glutamicum or to the species Bacillus subtilis.

It is also possible to employ an extremophilic bacterium such as Thermus thermophilus, or anaerobic bacteria from the family Clostridiae.

In another preferred embodiment the microorganism is a fungus, more preferably a fungus of the genus Saccharomyces, Schizosaccharomyces, Aspergillus, Trichoderma, Kluyveromyces or Pichia and even more preferably of the species Saccharomyces cerevisiae, Schizosaccharomyces pombe, Aspergillus niger, Trichoderma reesei, Kluyveromyces marxianus, Kluyveromyces lactis, Pichia pastoris, Pichia torula or Pichia utilis.

In another embodiment, the method according to the invention makes use of a photosynthetic microorganism expressing at least one enzyme for the conversion according to the invention as described above. Preferably, the microorganism is a photosynthetic bacterium, or a microalgae. In a further embodiment the microorganism is an algae, more preferably an algae belonging to the diatomeae.

It is also conceivable to use in the method according to the invention a combination of microorganisms wherein different microorganisms express different enzymes as described above. The genetic modification of microorganisms to express an enzyme of interest will also be further described in detail below.

In a preferred embodiment, the method of the present invention makes use of an organism, preferably a microorganism, which is genetically modified in order to avoid the leakage of acetyl-CoA, thereby increasing the intracellular concentration of acetyl-CoA. Genetic modifications leading to an increase in the intracellular concentration of acetyl-CoA are known in the art. Without being bound to theory, such an organism, preferably a microorganism, may preferably be genetically modified by deleting or inactivating the following genes:

ΔackA (acetate kinase), Δldh (lactate dehydrogenase), ΔadhE (alcohol dehydrogenase), ΔfrdB and/or ΔfrdC (fumarate reductase and fumarate dehydrogenase).

Alternatively, or in addition to any of the above deletions, the organism or microorganism may genetically be modified by overexpressing the gene panK/coaA encoding Pantothenate kinase, thereby increasing the CoA/acetyl-CoA intracellular pool.

These modifications which avoid the leakage of acetyl-CoA are known in the art and corresponding modified organisms have been used in methods for the bioconversion of exogenous isoamyl alcohol into isoamyl acetate by an E. coli strain expressing ATF2 (Metab. Eng. 6 (2004), 294-309).

In another embodiment, the method of the invention comprises the step of providing the organism, preferably the microorganism carrying the respective enzyme activity or activities in the form of a (cell) culture, preferably in the form of a liquid cell culture, a subsequent step of cultivating the organism, preferably the microorganism in a fermenter (often also referred to a bioreactor) under suitable conditions allowing the expression of the respective enzyme and further comprising the step of effecting an enzymatic conversion of a method of the invention as described herein above. Suitable fermenter or bioreactor devices and fermentation conditions are known to the person skilled in the art. A bioreactor or a fermenter refers to any manufactured or engineered device or system known in the art that supports a biologically active environment. Thus, a bioreactor or a fermenter may be a vessel in which a chemical/biochemical like the method of the present invention is carried out which involves organisms, preferably microorganisms and/or biochemically active substances, i.e., the enzyme(s) described above derived from such organisms or organisms harbouring the above described enzyme(s). In a bioreactor or a fermenter, this process can either be aerobic or anaerobic. These bioreactors are commonly cylindrical, and may range in size from litres to cubic metres, and are often made of stainless steel. In this respect, without being bound by theory, the fermenter or bioreactor may be designed in a way that it is suitable to cultivate the organisms, preferably microorganisms, in, e.g., a batch-culture, feed-batch-culture, perfusion culture or chemostate-culture, all of which are generally known in the art.

The culture medium can be any culture medium suitable for cultivating the respective organism or microorganism.

In a preferred embodiment the method according to the present invention also comprises the step of recovering the isobutene produced by the method. For example, if the method according to the present invention is carried out in vivo by fermenting a corresponding microorganism expressing the necessary enzymes, the isobutene can be recovered from the fermentation off-gas by methods known to the person skilled in the art.

In a preferred embodiment, the present invention relates to a method as described herein above in which a microorganism as described herein above is employed, wherein the microorganism is capable of enzymatically converting 3-methylcrotonic acid into isobutene, wherein said method comprises culturing the microorganism in a culture medium.

The enzymes used in the method according to the invention can be naturally occurring enzymes or enzymes which are derived from a naturally occurring enzymes, e.g. by the introduction of mutations or other alterations which, e.g., alter or improve the enzymatic activity, the stability, etc.

Methods for modifying and/or improving the desired enzymatic activities of proteins are well-known to the person skilled in the art and include, e.g., random mutagenesis or site-directed mutagenesis and subsequent selection of enzymes having the desired properties or approaches of the so-called “directed evolution”.

For example, for genetic modification in prokaryotic cells, a nucleic acid molecule encoding a corresponding enzyme can be introduced into plasmids which permit mutagenesis or sequence modification by recombination of DNA sequences. Standard methods (see Sambrook and Russell (2001), Molecular Cloning: A Laboratory Manual, CSH Press, Cold Spring Harbor, NY, USA) allow base exchanges to be performed or natural or synthetic sequences to be added. DNA fragments can be ligated by using adapters and linkers complementary to the fragments. Moreover, engineering measures which provide suitable restriction sites or remove surplus DNA or restriction sites can be used. In those cases, in which insertions, deletions or substitutions are possible, in vitro mutagenesis, “primer repair”, restriction or ligation can be used. In general, a sequence analysis, restriction analysis and other methods of biochemistry and molecular biology are carried out as analysis methods. The resulting enzyme variants are then tested for the desired activity, e.g., enzymatic activity, with an assay as described above and in particular for their increased enzyme activity.

As described above, the microorganism employed in a method of the invention or contained in the composition of the invention may be a microorganism which has been genetically modified by the introduction of a nucleic acid molecule encoding a corresponding enzyme. Thus, in a preferred embodiment, the microorganism is a recombinant microorganism which has been genetically modified to have an increased activity of at least one enzyme described above for the conversions of the method according to the present invention. This can be achieved e.g. by transforming the microorganism with a nucleic acid encoding a corresponding enzyme. A detailed description of genetic modification of microorganisms will be given further below. Preferably, the nucleic acid molecule introduced into the microorganism is a nucleic acid molecule which is heterologous with respect to the microorganism, i.e. it does not naturally occur in said microorganism.

In the context of the present invention, an “increased activity” means that the expression and/or the activity of an enzyme in the genetically modified microorganism is at least 10%, preferably at least 20%, more preferably at least 30% or 50%, even more preferably at least 70% or 80% and particularly preferred at least 90% or 100% higher than in the corresponding non-modified microorganism. In even more preferred embodiments the increase in expression and/or activity may be at least 150%, at least 200% or at least 500%. In particularly preferred embodiments the expression is at least 10-fold, more preferably at least 100-fold and even more preferred at least 1000-fold higher than in the corresponding non-modified microorganism.

The term “increased” expression/activity also covers the situation in which the corresponding non-modified microorganism does not express a corresponding enzyme so that the corresponding expression/activity in the non-modified microorganism is zero. Preferably, the concentration of the overexpressed enzyme is at least 5%, 10%, 20%, 30%, or 40% of the total host cell protein.

Methods for measuring the level of expression of a given protein in a cell are well known to the person skilled in the art. In one embodiment, the measurement of the level of expression is done by measuring the amount of the corresponding protein. Corresponding methods are well known to the person skilled in the art and include Western Blot, ELISA etc. In another embodiment the measurement of the level of expression is done by measuring the amount of the corresponding RNA. Corresponding methods are well known to the person skilled in the art and include, e.g., Northern Blot.

In the context of the present invention the term “recombinant” means that the microorganism is genetically modified so as to contain a nucleic acid molecule encoding an enzyme as defined above as compared to a wild-type or non-modified microorganism. A nucleic acid molecule encoding an enzyme as defined above can be used alone or as part of a vector.

The nucleic acid molecules can further comprise expression control sequences operably linked to the polynucleotide comprised in the nucleic acid molecule. The term “operatively linked” or “operably linked”, as used throughout the present description, refers to a linkage between one or more expression control sequences and the coding region in the polynucleotide to be expressed in such a way that expression is achieved under conditions compatible with the expression control sequence.

Expression comprises transcription of the heterologous DNA sequence, preferably into a translatable mRNA. Regulatory elements ensuring expression in fungi as well as in bacteria, are well known to those skilled in the art. They encompass promoters, enhancers, termination signals, targeting signals and the like. Examples are given further below in connection with explanations concerning vectors.

Promoters for use in connection with the nucleic acid molecule may be homologous or heterologous with regard to its origin and/or with regard to the gene to be expressed. Suitable promoters are for instance promoters which lend themselves to constitutive expression. However, promoters which are only activated at a point in time determined by external influences can also be used. Artificial and/or chemically inducible promoters may be used in this context.

The vectors can further comprise expression control sequences operably linked to said polynucleotides contained in the vectors. These expression control sequences may be suited to ensure transcription and synthesis of a translatable RNA in bacteria or fungi.

In addition, it is possible to insert different mutations into the polynucleotides by methods usual in molecular biology (see for instance Sambrook and Russell (2001), Molecular Cloning: A Laboratory Manual, CSH Press, Cold Spring Harbor, NY, USA), leading to the synthesis of polypeptides possibly having modified biological properties. The introduction of point mutations is conceivable at positions at which a modification of the amino acid sequence for instance influences the biological activity or the regulation of the polypeptide.

Moreover, mutants possessing a modified substrate or product specificity can be prepared. Preferably, such mutants show an increased activity. Alternatively, mutants can be prepared the catalytic activity of which is abolished without losing substrate binding activity.

Furthermore, the introduction of mutations into the polynucleotides encoding an enzyme as defined above allows the gene expression rate and/or the activity of the enzymes encoded by said polynucleotides to be reduced or increased.

For genetically modifying bacteria or fungi, the polynucleotides encoding an enzyme as defined above or parts of these molecules can be introduced into plasmids which permit mutagenesis or sequence modification by recombination of DNA sequences. Standard methods (see Sambrook and Russell (2001), Molecular Cloning: A Laboratory Manual, CSH Press, Cold Spring Harbor, NY, USA) allow base exchanges to be performed or natural or synthetic sequences to be added. DNA fragments can be connected to each other by applying adapters and linkers to the fragments. Moreover, engineering measures which provide suitable restriction sites or remove surplus DNA or restriction sites can be used. In those cases, in which insertions, deletions or substitutions are possible, in vitro mutagenesis, “primer repair”, restriction or ligation can be used. In general, a sequence analysis, restriction analysis and other methods of biochemistry and molecular biology are carried out as analysis methods.

Thus, in accordance with the present invention a recombinant microorganism can be produced by genetically modifying fungi or bacteria comprising introducing the above-described polynucleotides, nucleic acid molecules or vectors into a fungus or bacterium.

The polynucleotide encoding the respective enzyme is expressed so as to lead to the production of a polypeptide having any of the activities described above. An overview of different expression systems is for instance contained in Methods in Enzymology 153 (1987), 385-516, in Bitter et al. (Methods in Enzymology 153 (1987), 516-544) and in Sawers et al. (Applied Microbiology and Biotechnology 46 (1996), 1-9), Billman-Jacobe (Current Opinion in Biotechnology 7 (1996), 500-4), Hockney (Trends in Biotechnology 12 (1994), 456-463), Griffiths et al., (Methods in Molecular Biology 75 (1997), 427-440). An overview of yeast expression systems is for instance given by Hensing et al. (Antonie van Leuwenhoek 67 (1995), 261-279), Bussineau et al. (Developments in Biological Standardization 83 (1994), 13-19), Gellissen et al. (Antonie van Leuwenhoek 62 (1992), 79-93, Fleer (Current Opinion in Biotechnology 3 (1992), 486-496), Vedvick (Current Opinion in Biotechnology 2 (1991), 742-745) and Buckholz (Bio/Technology 9 (1991), 1067-1072).

Expression vectors have been widely described in the literature. As a rule, they contain not only a selection marker gene and a replication-origin ensuring replication in the host selected, but also a bacterial or viral promoter, and in most cases a termination signal for transcription. Between the promoter and the termination signal there is in general at least one restriction site or a polylinker which enables the insertion of a coding DNA sequence. The DNA sequence naturally controlling the transcription of the corresponding gene can be used as the promoter sequence, if it is active in the selected host organism. However, this sequence can also be exchanged for other promoter sequences. It is possible to use promoters ensuring constitutive expression of the gene and inducible promoters which permit a deliberate control of the expression of the gene. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of a downstream sequence are for instance the T7 promoter (Studier et al., Methods in Enzymology 185 (1990), 60-89), lacUV5, trp, trp-lacUV5 (DeBoer et al., in Rodriguez and Chamberlin (Eds), Promoters, Structure and Function; Praeger, New York, (1982), 462-481; DeBoer et al., Proc. Natl. Acad. Sci. USA (1983), 21-25), 11, rac (Boros et al., Gene 42 (1986), 97-100). Inducible promoters are preferably used for the synthesis of polypeptides. These promoters often lead to higher polypeptide yields than do constitutive promoters. In order to obtain an optimum amount of polypeptide, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (=isopropyl-β-D-thiogalactopyranoside) (deBoer et al., Proc. Natl. Acad. Sci. USA 80 (1983), 21-25). Termination signals for transcription are also described in the literature.

The transformation of the host cell with a polynucleotide or vector as described above can be carried out by standard methods, as for instance described in Sambrook and Russell (2001), Molecular Cloning: A Laboratory Manual, CSH Press, Cold Spring Harbor, NY, USA; Methods in Yeast Genetics, A Laboratory Course Manual, Cold Spring Harbor Laboratory Press, 1990. The host cell is cultured in nutrient media meeting the requirements of the particular host cell used, in particular in respect of the pH value, temperature, salt concentration, aeration, antibiotics, vitamins, trace elements etc.

Recombinant organisms or microorganisms expressing enzymes of step I and step II, and optionally further expressing enzymes of step III, step IV and step V as well as optionally further expressing enzymes of steps XIII, XIV and XV

The present invention also relates to a recombinant organism or microorganism which expresses (i) an enzyme capable of enzymatically converting 3-methylcrotonic acid into isobutene (step I as shown in FIG. 1); and (ii) an enzyme capable of enzymatically converting 3-hydroxyisovalerate (HIV) into 3-methylcrotonic acid (step II as shown in FIG. 1).

In a preferred embodiment, the enzyme capable of converting 3-methylcrotonic acid into isobutene is a 3-methylcrotonic acid decarboxylase as defined herein above.

More preferably, the 3-methylcrotonic acid decarboxylase is
(i) an FMN-dependent decarboxylase associated with an FMN prenyl transferase; or
(ii) an aconitate decarboxylase (EC 4.1.1.6); or
(iii) a methylcrotonyl-CoA carboxylase (EC 6.4.1.4); or
(iv) a geranoyl-CoA carboxylase (EC 6.4.1.5); or
(v) a protocatechuate (PCA) decarboxylase (EC 4.1.1.63)
   as defined herein above.

In another preferred embodiment, this recombinant organism or microorganism is a recombinant organism or microorganism, wherein the 3-methylcrotonic acid decarboxylase is selected from the group consisting of: 6-methylsalicylate decarboxylase (EC 4.1.1.52), 2-oxo-3-hexenedioate decarboxylase (EC 4.1.1.77) and 5-oxopent-3-ene-1,2,5-tricarboxylate decarboxylase (EC 4.1.1.68).

As regards the 3-methylcrotonic acid decarboxylase, the FMN-dependent decarboxylase, the associated FMN prenyl transferase, the aconitate decarboxylase (EC 4.1.1.6), the methylcrotonyl-CoA carboxylase (EC 6.4.1.4), and the geranoyl-CoA carboxylase (EC 6.4.1.5) as well as preferred embodiments of said 3-methylcrotonic acid decarboxylase, said protocatechuate (PCA) decarboxylase (EC 4.1.1.63), said FMN-dependent decarboxylase, said associated FMN prenyl transferase, said aconitate decarboxylase (EC 4.1.1.6), said methylcrotonyl-CoA carboxylase (EC 6.4.1.4) and said geranoyl-CoA carboxylase (EC 6.4.1.5), as well as said 6-methylsalicylate decarboxylase (EC 4.1.1.52), 2-oxo-3-hexenedioate decarboxylase (EC 4.1.1.77) and 5-oxopent-3-ene-1,2,5-tricarboxylate decarboxylase (EC 4.1.1.68), the same applies to the recombinant organism or microorganism as has been set forth above for the methods according to the present invention.

In a preferred embodiment, the recombinant organism or microorganism which expresses (i) an enzyme capable of enzymatically converting 3-methylcrotonic acid into isobutene (step I as shown in FIG. 1); and (ii) an enzyme capable of enzymatically converting 3-hydroxyisovalerate (HIV) into 3-methylcrotonic acid (step II as shown in FIG. 1) is a recombinant organism or microorganism, wherein the enzyme capable of enzymatically converting 3-hydroxyisovalerate (HIV) into 3-methylcrotonic acid is a hydro-lyase (EC 4.2.-.-) as defined herein above, preferably an aconitase (EC 4.2.1.3), a fumarase (EC 4.2.1.2) or an enoyl-CoA hydratase/dehydratease (EC 4.2.1.17) as defined herein above.

As regards the hydro-lyase (EC 4.2.-.-), the aconitase (EC 4.2.1.3), the fumarase (EC 4.2.1.2) and the enoyl-CoA hydratase/dehydratease (EC 4.2.1.17) as well as the preferred embodiments of said hydro-lyase (EC 4.2.-.-), said aconitase (EC 4.2.1.3), said fumarase (EC 4.2.1.2) and said enoyl-CoA hydratase/dehydratease (EC 4.2.1.17) the same applies to the recombinant organism or microorganism as has been set forth above for the methods according to the present invention.

In a further aspect, the above recombinant organism or microorganism is an organism or microorganism which further expresses an enzyme capable of enzymatically condensing acetone and acetyl-CoA into 3-hydroxyisovalerate (HIV) (step III as shown in FIG. 1). In a preferred embodiment, the enzyme capable of enzymatically condensing acetone and acetyl-CoA into 3-hydroxyisovalerate (HIV) is a HMG CoA synthase (EC 2.3.3.10) or a PksG protein or an enzyme with the activity of a C—C bond cleavage/condensation lyase, such as a HMG CoA lyase (EC 4.1.3.4) as defined herein above.

As regards the HMG CoA synthase (EC 2.3.3.10), the PksG protein, the enzyme with the activity of a C—C bond cleavage/condensation lyase and the HMG CoA lyase (EC 4.1.3.4) as well as the preferred embodiments of said enzymes the same applies to the recombinant organism or microorganism as has been set forth above for the methods according to the present invention.

In a further aspect, the above recombinant organism or microorganism is an organism or microorganism which further expresses an enzyme capable of enzymatically converting acetoacetate into acetone (step IV as shown in FIG. 1), preferably an acetoacetate decarboxylase (EC 4.1.1.4) as described herein above.

As regards said enzyme capable of enzymatically converting acetoacetate into acetone and said acetoacetate decarboxylase (EC 4.1.1.4) as well as preferred embodiments of said enzymes, the same applies to the recombinant organism or microorganism as has been set forth above for the methods according to the present invention.

In a further aspect, the above recombinant organism or microorganism is an organism or microorganism which further expresses an enzyme capable of converting acetoacetyl-CoA into acetoacetate (step Va or Vb as shown in FIG. 1), preferably
(i) an acetoacetyl-CoA hydrolase (EC 3.1.2.11); or
(ii) an enzyme which is capable of transferring the CoA group of acetoacetyl-CoA on acetate
   as described herein above.

In a preferred embodiment, the enzyme capable of transferring the CoA group of acetoacetyl-CoA on acetate is a CoA transferase (EC 2.8.3.-), preferably an acetate CoA transferase (EC 2.8.3.8) as described herein above.

As regards said enzyme which is capable of converting acetoacetyl-CoA into acetoacetate, said acetoacetyl-CoA hydrolase (EC 3.1.2.11), said enzyme which is capable of transferring the CoA group of acetoacetyl-CoA, the CoA transferase (EC 2.8.3.-) and said acetate CoA transferase (EC 2.8.3.8) as well as the preferred embodiments of said enzymes, the same applies to the recombinant organism or microorganism as has been set forth above for the methods according to the present invention.

In a further aspect, the above recombinant organism or microorganism is an organism or microorganism which further expresses an enzyme capable of enzymatically converting acetyl-CoA into acetoacetyl-CoA comprising
(a) (i) an enzyme capable of converting acetyl-CoA into malonyl-CoA (step XIV as shown in FIG. 1); and
   (ii) an enzyme capable of condensing malonyl-CoA and acetyl-CoA into acetoacetyl-CoA (step XV as shown in FIG. 1); or
(b) an enzyme capable of directly condensing two molecules of acetyl-CoA into acetoacetyl-CoA (step XIII as shown in FIG. 1).

In a preferred embodiment, the enzyme capable of converting acetyl-CoA into malonyl-CoA is an acetyl-CoA carboxylase (EC 6.4.1.2) as described herein above.

In another preferred embodiment, the enzyme capable of condensing malonyl-CoA and acetyl-CoA into acetoacetyl-CoA is an acetoacetyl-CoA synthetase (EC 2.3.1.194) as described herein above.

In a preferred embodiment, the enzyme capable of directly condensing two molecules of acetyl-CoA into acetoacetyl-CoA is an acetyl-CoA C-acetyltransferase (EC 2.3.1.9) as described herein above.

As regards the enzyme which is capable of converting acetyl-CoA into malonyl-CoA, the enzyme capable of condensing malonyl-CoA and acetyl-CoA into acetoacetyl-CoA, the acetyl-CoA carboxylase (EC 6.4.1.2), the acetoacetyl-CoA synthetase (EC 2.3.1.194), the enzyme capable of directly condensing two molecules of acetyl-CoA into acetoacetyl-CoA and the acetyl-CoA C-acetyltransferase (EC 2.3.1.9) as well as the preferred embodiments of said enzymes, the same applies to the recombinant organism or microorganism as has been set forth above for the methods according to the present invention.

Recombinant organisms or microorganisms expressing enzymes of step I and step VI, and optionally further expressing enzymes of step VII, step VIII and step IX as well as optionally further expressing enzymes of steps XIII, XIV and XV

The present invention also relates to a recombinant organism or microorganism which expresses (i) an enzyme capable of enzymatically converting 3-methylcrotonic acid into isobutene (step I as shown in FIG. 1); and (ii) an enzyme capable of enzymatically converting 3-methylcrotonyl-CoA into 3-methylcrotonic acid (step VIa, VIb or VIc as shown in FIG. 1).

In a preferred embodiment, the enzyme capable of converting 3-methylcrotonic acid into isobutene is a 3-methylcrotonic acid decarboxylase, preferably
(i) an FMN-dependent decarboxylase associated with an FMN prenyl transferase; or
(ii) an aconitate decarboxylase (EC 4.1.1.6); or
(iii) a methylcrotonyl-CoA carboxylase (EC 6.4.1.4); or
(iv) a geranoyl-CoA carboxylase (EC 6.4.1.5); or
(v) a protocatechuate (PCA) decarboxylase (EC 4.1.1.63)
   as defined herein above.

As regards the 3-methylcrotonic acid decarboxylase, the FMN-dependent decarboxylase, the associated FMN prenyl transferase, the aconitate decarboxylase (EC 4.1.1.6), the methylcrotonyl-CoA carboxylase (EC 6.4.1.4), the (v) protocatechuate (PCA) decarboxylase (EC 4.1.1.63) and the geranoyl-CoA carboxylase (EC 6.4.1.5) as well as preferred embodiments of said enzymes, the same applies to the recombinant organism or microorganism as has been set forth above for the methods according to the present invention.

In a preferred embodiment, the enzyme capable of enzymatically converting 3-methylcrotonyl-CoA into 3-methylcrotonic acid is
(a) an enzyme capable of directly converting 3-methylcrotonyl-CoA into 3-methylcrotonic acid wherein said enzyme capable of directly converting 3-methylcrotonyl-CoA into 3-methylcrotonic acid is a CoA transferase (EC 2.8.3.-), preferably a propionate:acetate-CoA transferase (EC 2.8.3.1), an acetate CoA-transferase (EC 2.8.3.8) or a succinyl-CoA:acetate CoA-transferase (EC 2.8.3.18) (step VIa as shown in FIG. 1) as described herein above; or
(b) an enzyme capable of directly converting 3-methylcrotonyl-CoA into 3-methylcrotonic acid wherein said enzyme capable of directly converting 3-methylcrotonyl-CoA into 3-methylcrotonic acid is a thioester hydrolase (EC 3.1.2.-), preferably acetyl-CoA hydrolase (EC 3.1.2.1), an ADP-dependent short-chain-acyl-CoA hydrolase (EC 3.1.2.18) or an acyl-CoA hydrolase (EC 3.1.2.20) (step VIb as shown in FIG. 1) as described herein above.

In another preferred embodiment, the recombinant organism or microorganism is a recombinant organism or microorganism which expresses the following two enzymes, namely
(c) (i) an enzyme capable of enzymatically converting 3-methylcrotonyl-CoA into 3-methylcrotonyl phosphate as described herein above; and
   (ii) an enzyme capable of converting 3-methylcrotonyl phosphate into 3-methylcrotonic acid (step VIc as shown in FIG. 1) as described herein above.

In a preferred embodiment, the enzyme capable of converting 3-methylcrotonyl-CoA into 3-methylcrotonyl phosphate is a phosphate butyryltransferase (EC 2.3.1.19) or a phosphate acetyltransferase (EC 2.3.1.8) and the enzyme capable of converting 3-methylcrotonyl phosphate into 3-methylcrotonic acid is a phosphotransferase with a carboxy group as acceptor (EC 2.7.2.-), preferably a propionate kinase (EC 2.7.2.15), an acetate kinase (EC 2.7.2.1), a butyrate kinase (EC 2.7.2.7) or a branched-chain-fatty-acid kinase (EC 2.7.2.14) as described herein above.

As regards the above-mentioned enzymes, the same applies to the recombinant organism or microorganism as has been set forth above for the methods according to the present invention.

In a further aspect, the above recombinant organism or microorganism is an organism or microorganism which further expresses an enzyme capable of enzymatically converting 3-methylglutaconyl-CoA into 3-methylcrotonyl-CoA (step VII as shown in FIG. 1), preferably (i) a methylcrotonyl-CoA carboxylase (EC 6.4.1.4); or (ii) a geranoyl-CoA carboxylase (EC 6.4.1.5) as described herein above.

As regards said enzymes as well as preferred embodiments of said enzymes, the same applies to the recombinant organism or microorganism as has been set forth above for the methods according to the present invention.

In a further aspect, the above recombinant organism or microorganism is an organism or microorganism which further expresses an enzyme capable of enzymatically converting 3-hydroxy-3-methylglutaryl-CoA into 3-methylglutaconyl-CoA (step VIII as shown in FIG. 1), preferably a 3-methylglutaconyl-coenzyme A hydratase (EC 4.2.1.18), a 3-hydroxyacyl-CoA dehydratase (EC 4.2.1.-) or an enoyl-CoA hydratase (EC 4.2.1.-).

As regards said enzyme as well as preferred embodiments of said enzymes, the same applies to the recombinant organism or microorganism as has been set forth above for the methods according to the present invention.

In a further aspect, the above recombinant organism or microorganism is an organism or microorganism which further expresses an enzyme capable of enzymatically condensing acetoacetyl-CoA and acetyl-CoA into 3-hydroxy-3-methylglutaryl-CoA (step IX as shown in FIG. 1), preferably a 3-hydroxy-3-methylglutaryl-CoA synthase.

As regards said enzyme as well as preferred embodiments of said enzyme, the same applies to the recombinant organism or microorganism as has been set forth above for the methods according to the present invention.

In a further aspect, the above recombinant organism or microorganism which expresses (i) an enzyme capable of enzymatically converting 3-methylcrotonic acid into isobutene (step I as shown in FIG. 1); and (ii) an enzyme capable of enzymatically converting 3-methylcrotonyl-CoA into 3-methylcrotonic acid (step VIa, VIb or VIc as shown in FIG. 1) (and optionally further expressing an enzyme capable of enzymatically converting 3-methylglutaconyl-CoA into 3-methylcrotonyl-CoA and optionally further expressing an enzyme capable of enzymatically converting 3-hydroxy3-methylglutaryl-CoA into 3-methylgutaconyl-CoA and optionally further expressing an enzyme capable of enzymatically condensing acetoacetyl-CoA and acetyl-CoA into 3-hydroxy-3-methylglutaryl-CoA) is preferably an organism or microorganism which further expresses an enzyme capable of enzymatically converting acetyl-CoA into acetoacetyl-CoA comprising an enzyme capable of directly condensing two molecules of acetyl-CoA into acetoacetyl-CoA (step XIII as shown in FIG. 1).

In another preferred embodiment, the recombinant organism or microorganism is a recombinant organism or microorganism which expresses the following two enzymes, namely
(i) an enzyme capable of converting acetyl-CoA into malonyl-CoA (step XIV as shown in FIG. 1); and
(ii) an enzyme capable of condensing malonyl-CoA and acetyl-CoA into acetoacetyl-CoA (step XV as shown in FIG. 1).

In a preferred embodiment, the enzyme capable of converting acetyl-CoA into malonyl-CoA is an acetyl-CoA carboxylase (EC 6.4.1.2) as described herein above.

In another preferred embodiment, the enzyme capable of condensing malonyl-CoA and acetyl-CoA into acetoacetyl-CoA is an acetoacetyl-CoA synthetase (EC 2.3.1.194) as described herein above.

In a preferred embodiment, the enzyme capable of directly condensing two molecules of acetyl-CoA into acetoacetyl-CoA is an acetyl-CoA C-acetyltransferase (EC 2.3.1.9) as described herein above.

As regards the above-mentioned enzymes as well as the preferred embodiments of said enzymes, the same applies to the recombinant organism or microorganism as has been set forth above for the methods according to the present invention.

Recombinant organisms or microorganisms of the alternative route for the enzymatic conversion from acetyl-CoA into isobutene via 3-methyl-3-butenoyl-CoA and 3-methyl-3-butenoic acid: recombinant organisms or microorganisms expressing enzymes of step XVI and step XVII, and optionally further expressing enzymes of step XVIII, step VIII and step IX as well as optionally further expressing enzymes of steps XIII, XIV and XV

As mentioned above, in an alternative to the above first route for the production of isobutene via 3-methylcrotonic acid, the present invention also relates to a method for the production of isobutene via an alternative route wherein isobutene is produced by the enzymatic conversion of 3-methyl-3-butenoic acid into isobutene. In the following, the recombinant organisms or microorganisms of this alternative route for the enzymatic conversion from acetyl-CoA into isobutene via 3-methyl-3-butenoyl-CoA and 3-methyl-3-butenoic acid are described.

The present invention also relates to a recombinant organism or microorganism which expresses (i) an enzyme capable of enzymatically converting 3-methyl-3-butenoic acid into isobutene (step XVI as shown in FIG. 1) and (ii) an enzyme capable of enzymatically converting 3-methyl-3-butenoyl-CoA into 3-methyl-3-butenoic acid (step XVII as shown in FIG. 1).

In a preferred embodiment, the enzyme capable of enzymatically converting 3-methyl-3-butenoic acid into isobutene is an 3-methyl-3-butenoic acid decarboxylase as described herein above, more preferably
(i) an FMN-dependent decarboxylase associated with an FMN prenyl transferase; or
(ii) an aconitate decarboxylase (EC 4.1.1.6); or
(iii) a methylcrotonyl-CoA carboxylase (EC 6.4.1.4); or
(iv) a geranoyl-CoA carboxylase (EC 6.4.1.5); or
(v) a protocatechuate (PCA) decarboxylase (EC 4.1.1.63)
   as described herein above.

In another preferred embodiment, the 3-methyl-3-butenoic acid decarboxylase is selected from the group consisting of 6-methylsalicylate decarboxylase (EC 4.1.1.52), 2-oxo-3-hexenedioate decarboxylase (EC 4.1.1.77) and 5-oxopent-3-ene-1,2,5-tricarboxylate decarboxylase (EC 4.1.1.68) as described herein above.

As regards the above-mentioned enzymes as well as preferred embodiments of said enzymes, the same applies to the recombinant organism or microorganism as has been set forth above for the methods according to the present invention.

In a preferred embodiment, the enzyme capable of enzymatically converting 3-methyl-3-butenoyl-CoA into 3-methyl-3-butenoic acid is
(a) an enzyme capable of directedly converting 3-methyl-3-butenoyl-CoA into 3-methyl-3-butenoic acid, wherein said enzyme capable of directedly converting 3-methyl-3-butenoyl-CoA into 3-methyl-3-butenoic acid is a CoA transferase (EC 2.8.3.-), preferably a propionate:acetate-CoA transferase (EC 2.8.3.1), an acetate CoA-transferase (EC 2.8.3.8) or a succinyl-CoA:acetate CoA-transferase (EC 2.8.3.18) (step XVIIa as shown in FIG. 1) as described herein above.

In another preferred embodiment, the recombinant organism or microorganism is a recombinant organism or microorganism which expresses the following two enzymes, namely
(b) an enzyme capable of directedly converting 3-methyl-3-butenoyl-CoA into 3-methyl-3-butenoic acid, wherein said enzyme capable of directedly converting 3-methyl-3-butenoyl-CoA into 3-methyl-3-butenoic acid is a thioester hydrolase (EC 3.1.2.-), preferably acetyl-CoA hydrolase (EC 3.1.2.1), an ADP-dependent short-chain-acyl-CoA hydrolase (EC 3.1.2.18) or an acyl-CoA hydrolase (EC 3.1.2.20) (step XVIIb as shown in FIG. 1) as described herein above; or
(c) (i) an enzyme capable of enzymatically converting 3-methyl-3-butenoyl-CoA into 3-methyl-3-butenoyl phosphate; and
   (ii) an enzyme capable of enzymatically converting 3-methyl-3-butenoyl phosphate into said 3-methyl-3-butenoic acid(step XVIIc as shown in FIG. 1) as described herein above.

In a preferred embodiment, the enzyme capable of enzymatically converting said 3-methyl-3-butenoyl-CoA into 3-methyl-3-butenoyl phosphate is a phosphate butyryltransferase (EC 2.3.1.19) or a phosphate acetyltransferase (EC 2.3.1.8) and the enzyme capable of enzymatically converting 3-methyl-3-butenoyl phosphate into 3-methyl-3-butenoic acid is a phosphotransferase with a carboxy group as acceptor (EC 2.7.2.-), preferably a propionate kinase (EC 2.7.2.15), an acetate kinase (EC 2.7.2.1), a butyrate kinase (EC 2.7.2.7) or a branched-chain-fatty-acid kinase (EC 2.7.2.14) as described herein above.

As regards the above-mentioned enzymes as well as preferred embodiments of said enzymes, the same applies to the recombinant organism or microorganism as has been set forth above for the methods according to the present invention.

In a further aspect, the above recombinant organism or microorganism is an organism or microorganism which further expresses an enzyme capable of enzymatically converting 3-methylglutaconyl-CoA into 3-methyl-3-butenoyl-CoA (step XVIII as shown in FIG. 1), preferably
(a) (i) a methylcrotonyl-CoA carboxylase (EC 6.4.1.4); or (ii) a geranoyl-CoA carboxylase (EC 6.4.1.5), or
(b) an N-terminal domain of CurF from Lynbya majuscula multifunctional protein or a 3-methylglutaconyl-CoA decarboxylase, preferably a 3-methylglutaconyl-CoA decarboxylase of Myxococcus xanthus encoded by the IiuB gene; or
(c) an enzyme of the 4-oxalocrotonate decarboxylase family,
   as described herein above.

As regards the above-mentioned enzymes as well as preferred embodiments of said enzymes, the same applies to the recombinant organism or microorganism as has been set forth above for the methods according to the present invention.

In a further aspect, the above recombinant organism or microorganism is an organism or microorganism which further expresses an enzyme capable of enzymatically converting 3-hydroxy-3-methylglutaryl-CoA into 3-methylglutaconyl-CoA (step VIII as shown in FIG. 1),preferably a 3-methylglutaconyl-coenzyme A hydratase (EC 4.2.1.18), a 3-hydroxyacyl-CoA dehydratase (EC 4.2.1.-) or an enoyl-CoA hydratase (EC 4.2.1.-).

As regards the above-mentioned enzyme as well as preferred embodiments of said enzyme, the same applies to the recombinant organism or microorganism as has been set forth above for the methods according to the present invention.

In a further aspect, the above recombinant organism or microorganism is an organism or microorganism which further expresses an enzyme capable of enzymatically condensing acetoacetyl-CoA and acetyl-CoA into 3-hydroxy-3-methylglutaryl-CoA (step IX as shown in FIG. 1).

In a preferred embodiment, the enzyme capable of enzymatically condensing acetoacetyl-CoA and acetyl-CoA into 3-hydroxy-3-methylglutaryl-CoA is a 3-hydroxy-3-methylglutaryl-CoA synthase.

As regards the afore-mentioned enzyme as well as preferred embodiments of said enzyme, the same applies to the recombinant organism or microorganism as has been set forth above for the methods according to the present invention.

In a further aspect, the above recombinant organism or microorganism is an organism or microorganism which further expresses an enzyme or several enzymes capable of enzymatically converting acetyl-CoA into acetoacetyl-CoA.

In one preferred embodiment, the recombinant organism or microorganism expresses a combination of enzymes, namely
(i) an enzyme capable of converting acetyl-CoA into malonyl-CoA (step XIV as shown in FIG. 1); and
(ii) an enzyme capable of condensing malonyl-CoA and acetyl-CoA into acetoacetyl-CoA (step XV as shown in FIG. 1).

In an alternative embodiment, the recombinant organism or microorganism expresses an enzyme capable of directly condensing two molecules of acetyl-CoA into acetoacetyl-CoA (step XIII as shown in FIG. 1).

As regards the first above-mentioned embodiment, the enzyme capable of converting acetyl-CoA into malonyl-CoA is preferably an acetyl-CoA carboxylase (EC 6.4.1.2) as described herein above.

Moreover, the enzyme capable of condensing malonyl-CoA and acetyl-CoA into acetoacetyl-CoA is an acetoacetyl-CoA synthetase (EC 2.3.1.194) as described herein above.

As regards the second above-mentioned embodiment, the enzyme capable of directly condensing two molecules of acetyl-CoA into acetoacetyl-CoA is preferably an acetyl-CoA C-acetyltransferase (EC 2.3.1.9) as described herein above.

As regards the above-mentioned enzymes as well as the preferred embodiments of said enzymes, the same applies to the recombinant organism or microorganism as has been set forth above for the methods according to the present invention.

Recombinant organisms or microorganisms expressing enzymes of the additional/supplemental Pathways of steps Xa, Xb, XI and XII

As mentioned above, the above-described methods of the present invention for producing isobutene from acetyl-CoA may be supplemented by one or more of the reactions as shown in step Xa, step Xb, step XI and step XII of FIG. 18 and as described in detail herein above.

Thus, in a further aspect, the present invention relates to any of the above-described recombinant organism or microorganism wherein the organism or microorganism which additionally further expresses
a) an enzyme capable of enzymatically converting 3-hydroxyisovalerate (HIV) into 3-methylcrotonic acid with a concomitant transfer of CoA from 3-methylcrotonyl-CoA on 3-hydroxyisovalerate (HIV) to result in 3-hydroxyisovaleryl-CoA (step Xa as schematically shown in FIG. 19); and/or
b) an enzyme capable of enzymatically converting 3-hydroxyisovalerate (HIV) into 3-hydroxyisovaleryl-CoA (step Xb as schematically shown in FIG. 20); and/or
c) an enzyme capable of enzymatically converting 3-hydroxyisovaleryl-CoA into 3-methylcrotonyl-CoA (step XI as schematically shown in FIG. 21); and/or
d) an enzyme capable of enzymatically converting 3-hydroxyisovalerate (HIV) into 3-hydroxyisovaleryl-CoA (step XII as schematically shown in FIG. 22)
   as described herein above.

As regards the above-mentioned enzymes as well as preferred embodiments of said enzymes, the same applies to the recombinant organism or microorganism as has been set forth above for the methods according to the present invention.

The above microorganism is preferably a bacterium, a yeast or a fungus. In another preferred embodiment, the organism is a plant or a non-human animal. As regards other preferred embodiments of the bacterium, recombinant organism or microorganism, the same applies as has been set forth above in connection with the methods according to the present invention.

The present invention also relates to the use of any of the above-described recombinant organisms or microorganisms for the production of isobutene. Thus, the present invention furthermore relates to the use of a recombinant organism or microorganism for the production of isobutene, wherein said recombinant organism or microorganism expresses (i) an enzyme capable of enzymatically converting 3-methylcrotonic acid into isobutene (step I as shown in FIG. 1); and (ii) an enzyme capable of enzymatically converting 3-hydroxyisovalerate (HIV) into 3-methylcrotonic acid (step II as shown in FIG. 1).

In another preferred embodiment, the present invention relates to the use of a recombinant organism or microorganism for the production of isobutene, wherein said recombinant organism or microorganism expresses (i) an enzyme capable of enzymatically converting 3-methylcrotonic acid into isobutene (step I as shown in FIG. 1); and (ii) an enzyme capable of enzymatically converting 3-hydroxyisovalerate (HIV) into 3-methylcrotonic acid (step II as shown in FIG. 1) which further expresses an enzyme capable of enzymatically condensing acetone and acetyl-CoA into 3-hydroxyisovalerate (HIV) (step III as shown in FIG. 1).

In another preferred embodiment, the present invention relates to the use of a recombinant organism or microorganism for the production of isobutene, wherein said recombinant organism or microorganism expresses (i) an enzyme capable of enzymatically converting 3-methylcrotonic acid into isobutene (step I as shown in FIG. 1); and (ii) an enzyme capable of enzymatically converting 3-hydroxyisovalerate (HIV) into 3-methylcrotonic acid (step II as shown in FIG. 1), which further expresses an enzyme capable of enzymatically condensing acetone and acetyl-CoA into 3-hydroxyisovalerate (HIV) (step III as shown in FIG. 1) and which further expresses an enzyme capable of enzymatically converting acetoacetate into acetone (step IV as shown in FIG. 1).

In another preferred embodiment, the present invention relates to the use of a recombinant organism or microorganism for the production of isobutene, wherein said recombinant organism or microorganism expresses (i) an enzyme capable of enzymatically converting 3-methylcrotonic acid into isobutene (step I as shown in FIG. 1); and (ii) an enzyme capable of enzymatically converting 3-hydroxyisovalerate (HIV) into 3-methylcrotonic acid (step II as shown in FIG. 1), which further expresses an enzyme capable of enzymatically condensing acetone and acetyl-CoA into 3-hydroxyisovalerate (HIV) (step II as shown in FIG. 1), which further expresses an enzyme capable of enzymatically converting acetoacetate into acetone (step IV as shown in FIG. 1) and which further expresses an enzyme capable of converting acetoacetyl-CoA into acetoacetate (step Va or Vb as shown in FIG. 1).

In another preferred embodiment, the present invention relates to the use of a recombinant organism or microorganism for the production of isobutene, wherein said recombinant organism or microorganism expresses (i) an enzyme capable of enzymatically converting 3-methylcrotonic acid into isobutene (step I as shown in FIG. 1); and (ii) an enzyme capable of enzymatically converting 3-hydroxyisovalerate (HIV) into 3-methylcrotonic acid (step II as shown in FIG. 1), which further expresses an enzyme capable of enzymatically condensing acetone and acetyl-CoA into 3-hydroxyisovalerate (HIV) (step II as shown in FIG. 1), which further expresses an enzyme capable of enzymatically converting acetoacetate into acetone (step IV as shown in FIG. 1), which further expresses an enzyme capable of converting acetoacetyl-CoA into acetoacetate (step Va or Vb as shown in FIG. 1) and which further expresses an enzyme capable of enzymatically converting acetyl-CoA into acetoacetyl-CoA comprising (a) (i) an enzyme capable of converting acetyl-CoA into malonyl-CoA (step XIV as shown in FIG. 1); and (ii) an enzyme capable of condensing malonyl-CoA and acetyl-CoA into acetoacetyl-CoA (step XV as shown in FIG. 1); or (b) an enzyme capable of directly condensing two molecules of acetyl-CoA into acetoacetyl-CoA (step XIII as shown in FIG. 1).

In a more preferred embodiment, the present invention relates to any of the above uses of a recombinant organisms or microorganisms for the production of isobutene wherein said recombinant organism or microorganism expresses an enzyme catalyzing the enzymatic conversion of 3-methylcrotonic acid into isobutene.

As regards the above-mentioned enzymes as well as preferred embodiments of said enzymes, the same applies to the use of the recombinant organism or microorganism for the production of isobutene as has been set forth above for the methods and recombinant organisms or microorganisms according to the present invention.

The present invention furthermore relates to the use of a recombinant organism or microorganism for the production of isobutene, wherein said recombinant organism or microorganism expresses (i) an enzyme capable of enzymatically converting 3-methylcrotonic acid into isobutene (step I as shown in FIG. 1); and (ii) an enzyme capable of enzymatically converting 3-methylcrotonyl-CoA into 3-methylcrotonic acid (step VIa, VIb or VIc as shown in FIG. 1).

In another preferred embodiment, the present invention relates to the use of a recombinant organism or microorganism for the production of isobutene, wherein said recombinant organism or microorganism expresses (i) an enzyme capable of enzymatically converting 3-methylcrotonic acid into isobutene (step I as shown in FIG. 1); and (ii) an enzyme capable of enzymatically converting 3-methylcrotonyl-CoA into 3-methylcrotonic acid (step VIa, VIb or VIc as shown in FIG. 1) and which further expresses an enzyme capable of enzymatically converting 3-methylglutaconyl-CoA into 3-methylcrotonyl-CoA (step VII as shown in FIG. 1).

In another preferred embodiment, the present invention relates to the use of a recombinant organism or microorganism for the production of isobutene, wherein said recombinant organism or microorganism expresses (i) an enzyme capable of enzymatically converting 3-methylcrotonic acid into isobutene (step I as shown in FIG. 1); and (ii) an enzyme capable of enzymatically converting 3-methylcrotonyl-CoA into 3-methylcrotonic acid (step VIa, VIb or VIc as shown in FIG. 1), which further expresses an enzyme capable of enzymatically converting 3-methylglutaconyl-CoA into 3-methylcrotonyl-CoA (step VII as shown in FIG. 1) and which further expresses an enzyme capable of enzymatically converting 3-hydroxy-3-methylglutaryl-CoA into 3-methylglutaconyl-CoA (step VIII as shown in FIG. 1).

In another preferred embodiment, the present invention relates to the use of a recombinant organism or microorganism for the production of isobutene, wherein said recombinant organism or microorganism expresses (i) an enzyme capable of enzymatically converting 3-methylcrotonic acid into isobutene (step I as shown in FIG. 1); and (ii) an enzyme capable of enzymatically converting 3-methylcrotonyl-CoA into 3-methylcrotonic acid (step VIa, VIb or VIc as shown in FIG. 1), which further expresses an enzyme capable of enzymatically converting 3-methylglutaconyl-CoA into 3-methylcrotonyl-CoA (step VII as shown in FIG. 1), which further expresses an enzyme capable of enzymatically converting 3-hydroxy-3-methylglutaryl-CoA into 3-methylglutaconyl-CoA (step VIII as shown in FIG. 1) and which further expresses an enzyme capable of enzymatically condensing acetoacetyl-CoA and acetyl-CoA into 3-hydroxy-3-methylglutaryl-CoA (step IX as shown in FIG. 1).

In another preferred embodiment, the present invention relates to the use of a recombinant organism or microorganism for the production of isobutene, wherein said recombinant organism or microorganism expresses (i) an enzyme capable of enzymatically converting 3-methylcrotonic acid into isobutene (step I as shown in FIG. 1); and (ii) an enzyme capable of enzymatically converting 3-methylcrotonyl-CoA into 3-methylcrotonic acid (step VIa, VIb or VIc as shown in FIG. 1), which further expresses an enzyme capable of enzymatically converting 3-methylglutaconyl-CoA into 3-methylcrotonyl-CoA (step VII as shown in FIG. 1), which further expresses an enzyme capable of enzymatically converting 3-hydroxy-3-methylglutaryl-CoA into 3-methylglutaconyl-CoA (step VIII as shown in FIG. 1), which further expresses an enzyme capable of enzymatically condensing acetoacetyl-CoA and acetyl-CoA into 3-hydroxy-3-methylglutaryl-CoA (step IX as shown in FIG. 1) and which further expresses an enzyme capable of enzymatically converting acetyl-CoA into acetoacetyl-CoA comprising (a) (i) an enzyme capable of converting acetyl-CoA into malonyl-CoA (step XIV as shown in FIG. 1); and (ii) an enzyme capable of condensing malonyl-CoA and acetyl-CoA into acetoacetyl-CoA (step XV as shown in FIG. 1); or (b) an enzyme capable of directly condensing two molecules of acetyl-CoA into acetoacetyl-CoA (step XIII as shown in FIG. 1).

In a more preferred embodiment, the present invention relates to any of the above uses of a recombinant organisms or microorganisms for the production of isobutene wherein said recombinant organism or microorganism expresses an enzyme catalyzing the enzymatic conversion of 3-methylcrotonic acid into isobutene.

As regards the above-mentioned enzymes as well as preferred embodiments of said enzymes, the same applies to the use of the recombinant organism or microorganism for the production of isobutene as has been set forth above for the methods and recombinant organisms or microorganisms according to the present invention.

The present invention furthermore relates to the use of a recombinant organism or microorganism for the production of isobutene, wherein said recombinant organism or microorganism expresses (i) an enzyme capable of enzymatically converting 3-methyl-3-butenoic acid into isobutene (step XVI as shown in FIG. 1) and (ii) an enzyme capable of enzymatically converting 3-methyl-3-butenoyl-CoA into 3-methyl-3-butenoic acid (step XVII as shown in FIG. 1).

In another preferred embodiment, the present invention relates to the use of a recombinant organism or microorganism for the production of isobutene, wherein said recombinant organism or microorganism expresses (i) an enzyme capable of enzymatically converting 3-methyl-3-butenoic acid into isobutene (step XVI as shown in FIG. 1) and (ii) an enzyme capable of enzymatically converting 3-methyl-3-butenoyl-CoA into 3-methyl-3-butenoic acid (step XVII as shown in FIG. 1) and which further expresses an enzyme capable of enzymatically converting 3-methylglutaconyl-CoA into 3-methyl-3-butenoyl-CoA (step XVIII as shown in FIG. 1).

In another preferred embodiment, the present invention relates to the use of a recombinant organism or microorganism for the production of isobutene, wherein said recombinant organism or microorganism expresses (i) an enzyme capable of enzymatically converting 3-methyl-3-butenoic acid into isobutene (step XVI as shown in FIG. 1) and (ii) an enzyme capable of enzymatically converting 3-methyl-3-butenoyl-CoA into 3-methyl-3-butenoic acid (step XVII as shown in FIG. 1), which further expresses an enzyme capable of enzymatically converting 3-methylglutaconyl-CoA into 3-methyl-3-butenoyl-CoA (step XVIII as shown in FIG. 1) and which further expresses an enzyme capable of enzymatically converting 3-hydroxy-3-methylglutaryl-CoA into 3-methylglutaconyl-CoA (step VIII as shown in FIG. 1).

In another preferred embodiment, the present invention relates to the use of a recombinant organism or microorganism for the production of isobutene, wherein said recombinant organism or microorganism expresses (i) an enzyme capable of enzymatically converting 3-methyl-3-butenoic acid into isobutene (step XVI as shown in FIG. 1) and (ii) an enzyme capable of enzymatically converting 3-methyl-3-butenoyl-CoA into 3-methyl-3-butenoic acid (step XVII as shown in FIG. 1), which further expresses an enzyme capable of enzymatically converting 3-methylglutaconyl-CoA into 3-methyl-3-butenoyl-CoA (step XVIII as shown in FIG. 1), which further expresses an enzyme capable of enzymatically converting 3-hydroxy-3-methylglutaryl-CoA into 3-methylglutaconyl-CoA (step VIII as shown in FIG. 1) and which further expresses an enzyme capable of enzymatically condensing acetoacetyl-CoA and acetyl-CoA into 3-hydroxy-3-methylglutaryl-CoA (step IX as shown in FIG. 1).

In another preferred embodiment, the present invention relates to the use of a recombinant organism or microorganism for the production of isobutene, wherein said recombinant organism or microorganism expresses (i) an enzyme capable of enzymatically converting 3-methyl-3-butenoic acid into isobutene (step XVI as shown in FIG. 1) and (ii) an enzyme capable of enzymatically converting 3-methyl-3-butenoyl-CoA into 3-methyl-3-butenoic acid (step XVII as shown in FIG. 1), which further expresses an enzyme capable of enzymatically converting 3-methylglutaconyl-CoA into 3-methyl-3-butenoyl-CoA (step XVIII as shown in FIG. 1), which further expresses an enzyme capable of enzymatically converting 3-hydroxy-3-methylglutaryl-CoA into 3-methylglutaconyl-CoA (step VIII as shown in FIG. 1), which further expresses an enzyme capable of enzymatically condensing acetoacetyl-CoA and acetyl-CoA into 3-hydroxy-3-methylglutaryl-CoA (step IX as shown in FIG. 1) and which further expresses an enzyme capable of enzymatically converting acetyl-CoA into acetoacetyl-CoA comprising (a) (i) an enzyme capable of converting acetyl-CoA into malonyl-CoA (step XIV as shown in FIG. 1); and (ii) an enzyme capable of condensing malonyl-CoA and acetyl-CoA into acetoacetyl-CoA (step XV as shown in FIG. 1); or (b) an enzyme capable of directly condensing two molecules of acetyl-CoA into acetoacetyl-CoA (step XIII as shown in FIG. 1).

In a more preferred embodiment, the present invention relates to any of the above uses of a recombinant organisms or microorganisms for the production of isobutene wherein said recombinant organism or microorganism expresses an enzyme catalyzing the enzymatic conversion of 3-methyl-3-butenoic acid into isobutene.

As regards the above-mentioned enzymes as well as preferred embodiments of said enzymes, the same applies to the use of the recombinant organism or microorganism for the production of isobutene as has been set forth above for the methods and recombinant organisms or microorganisms according to the present invention.

In a further aspect, the present invention relates to the use of any of the above-described recombinant organism or microorganism for the production of isobutene, wherein the organism or microorganism is an organism or microorganism which additionally further expresses
a) an enzyme capable of enzymatically converting 3-hydroxyisovalerate (HIV) into 3-methylcrotonic acid with a concomitant transfer of CoA from 3-methylcrotonyl-CoA on 3-hydroxyisovalerate (HIV) to result in 3-hydroxyisovaleryl-CoA (step Xa as schematically shown in FIG. 19); and/or
b) an enzyme capable of enzymatically converting 3-hydroxyisovalerate (HIV) into 3-hydroxyisovaleryl-CoA (step Xb as schematically shown in FIG. 20); and/or
c) an enzyme capable of enzymatically converting 3-hydroxyisovaleryl-CoA into 3-methylcrotonyl-CoA (step XI as schematically shown in FIG. 21); and/or
d) an enzyme capable of enzymatically converting 3-hydroxyisovalerate (HIV)
   into 3-hydroxyisovaleryl-CoA (step XII as schematically shown in FIG. 22) as described herein above.

As regards the above-mentioned enzymes as well as preferred embodiments of said enzymes, the same applies to the recombinant organism or microorganism as has been set forth above for the methods according to the present invention.

The present invention furthermore relates to the use of an enzyme catalyzing the enzymatic conversion of 3-methylcrotonic acid into isobutene for the production of isobutene from 3-methylcrotonic acid.

The present invention furthermore relates to the use of (i) an enzyme capable of enzymatically converting 3-methylcrotonic acid into isobutene (step I as shown in FIG. 1); and (ii) an enzyme capable of enzymatically converting 3-hydroxyisovalerate (HIV) into 3-methylcrotonic acid (step II as shown in FIG. 1) for the production of isobutene.

In another preferred embodiment, the present invention relates to the use (i) an enzyme capable of enzymatically converting 3-methylcrotonic acid into isobutene (step I as shown in FIG. 1); and (ii) an enzyme capable of enzymatically converting 3-hydroxyisovalerate (HIV) into 3-methylcrotonic acid (step II as shown in FIG. 1) and an enzyme capable of enzymatically condensing acetone and acetyl-CoA into 3-hydroxyisovalerate (HIV) (step II as shown in FIG. 1) for the production of isobutene.

In another preferred embodiment, the present invention relates to the use of (i) an enzyme capable of enzymatically converting 3-methylcrotonic acid into isobutene (step I as shown in FIG. 1); and (ii) an enzyme capable of enzymatically converting 3-hydroxyisovalerate (HIV) into 3-methylcrotonic acid (step II as shown in FIG. 1), an enzyme capable of enzymatically condensing acetone and acetyl-CoA into 3-hydroxyisovalerate (HIV) (step II as shown in FIG. 1) and an enzyme capable of enzymatically converting acetoacetate into acetone (step IV as shown in FIG. 1) for the production of isobutene.

In another preferred embodiment, the present invention relates to the use of (i) an enzyme capable of enzymatically converting 3-methylcrotonic acid into isobutene (step I as shown in FIG. 1); and (ii) an enzyme capable of enzymatically converting 3-hydroxyisovalerate (HIV) into 3-methylcrotonic acid (step II as shown in FIG. 1); an enzyme capable of enzymatically condensing acetone and acetyl-CoA into 3-hydroxyisovalerate (HIV) (step II as shown in FIG. 1), an enzyme capable of enzymatically converting acetoacetate into acetone (step IV as shown in FIG. 1) and an enzyme capable of converting acetoacetyl-CoA into acetoacetate (step Va or Vb as shown in FIG. 1) for the production of isobutene.

In another preferred embodiment, the present invention relates to the use of (i) an enzyme capable of enzymatically converting 3-methylcrotonic acid into isobutene (step I as shown in FIG. 1); and (ii) an enzyme capable of enzymatically converting 3-hydroxyisovalerate (HIV) into 3-methylcrotonic acid (step II as shown in FIG. 1); an enzyme capable of enzymatically condensing acetone and acetyl-CoA into 3-hydroxyisovalerate (HIV) (step II as shown in FIG. 1), an enzyme capable of enzymatically converting acetoacetate into acetone (step IV as shown in FIG. 1), an enzyme capable of converting acetoacetyl-CoA into acetoacetate (step Va or Vb as shown in FIG. 1) and an enzyme capable of enzymatically converting acetyl-CoA into acetoacetyl-CoA comprising (a) (i) an enzyme capable of converting acetyl-CoA into malonyl-CoA (step XIV as shown in FIG. 1); and (ii) an enzyme capable of condensing malonyl-CoA and acetyl-CoA into acetoacetyl-CoA (step XV as shown in FIG. 1); or (b) an enzyme capable of directly condensing two molecules of acetyl-CoA into acetoacetyl-CoA (step XIII as shown in FIG. 1) for the production of isobutene.

As regards the above-mentioned enzymes as well as preferred embodiments of said enzymes, the same applies to the use of the recombinant organism or microorganism for the production of isobutene as has been set forth above for the methods and recombinant organisms or microorganisms according to the present invention.

The present invention furthermore relates to the use of (i) an enzyme capable of enzymatically converting 3-methylcrotonic acid into isobutene (step I as shown in FIG. 1); and (ii) an enzyme capable of enzymatically converting 3-methylcrotonyl-CoA into 3-methylcrotonic acid (step VIa, VIb or VIc as shown in FIG. 1) for the production of isobutene.

In another preferred embodiment, the present invention relates to the use of (i) an enzyme capable of enzymatically converting 3-methylcrotonic acid into isobutene (step I as shown in FIG. 1); and (ii) an enzyme capable of enzymatically converting 3-methylcrotonyl-CoA into 3-methylcrotonic acid (step VIa, VIb or VIc as shown in FIG. 1) and an enzyme capable of enzymatically converting 3-methylglutaconyl-CoA into 3-methylcrotonyl-CoA (step VII as shown in FIG. 1) for the production of isobutene.

In another preferred embodiment, the present invention relates to the use of (i) an enzyme capable of enzymatically converting 3-methylcrotonic acid into isobutene (step I as shown in FIG. 1); and (ii) an enzyme capable of enzymatically converting 3-methylcrotonyl-CoA into 3-methylcrotonic acid (step VIa, VIb or VIc as shown in FIG. 1); an enzyme capable of enzymatically converting 3-methylglutaconyl-CoA into 3-methylcrotonyl-CoA (step VII as shown in FIG. 1) and an enzyme capable of enzymatically converting 3-hydroxy-3-methylglutaryl-CoA into 3-methylglutaconyl-CoA (step VIII as shown in FIG. 1) for the production of isobutene.

In another preferred embodiment, the present invention relates to the use of (i) an enzyme capable of enzymatically converting 3-methylcrotonic acid into isobutene (step I as shown in FIG. 1); and (ii) an enzyme capable of enzymatically converting 3-methylcrotonyl-CoA into 3-methylcrotonic acid (step VIa, VIb or VIc as shown in FIG. 1); an enzyme capable of enzymatically converting 3-methylglutaconyl-CoA into 3-methylcrotonyl-CoA (step VII as shown in FIG. 1); an enzyme capable of enzymatically converting 3-hydroxy-3-methylglutaryl-CoA into 3-methylglutaconyl-CoA (step VIII as shown in FIG. 1) and an enzyme capable of enzymatically condensing acetoacetyl-CoA and acetyl-CoA into 3-hydroxy-3-methylglutaryl-CoA (step IX as shown in FIG. 1) for the production of isobutene.

In another preferred embodiment, the present invention relates to the use of (i) an enzyme capable of enzymatically converting 3-methylcrotonic acid into isobutene (step I as shown in FIG. 1); and (ii) an enzyme capable of enzymatically converting 3-methylcrotonyl-CoA into 3-methylcrotonic acid (step VIa, VIb or VIc as shown in FIG. 1); an enzyme capable of enzymatically converting 3-methylglutaconyl-CoA into 3-methylcrotonyl-CoA (step VII as shown in FIG. 1); an enzyme capable of enzymatically converting 3-hydroxy-3-methylglutaryl-CoA into 3-methylglutaconyl-CoA (step VIII as shown in FIG. 1); an enzyme capable of enzymatically condensing acetoacetyl-CoA and acetyl-CoA into 3-hydroxy-3-methylglutaryl-CoA (step IX as shown in FIG. 1) and an enzyme capable of enzymatically converting acetyl-CoA into acetoacetyl-CoA comprising (a) (i) an enzyme capable of converting acetyl-CoA into malonyl-CoA (step XIV as shown in FIG. 1); and (ii) an enzyme capable of condensing malonyl-CoA and acetyl-CoA into acetoacetyl-CoA (step XV as shown in FIG. 1); or (b) an enzyme capable of directly condensing two molecules of acetyl-CoA into acetoacetyl-CoA (step XIII as shown in FIG. 1) for the production of isobutene.

As regards the above-mentioned enzymes as well as preferred embodiments of said enzymes, the same applies to the use of the recombinant organism or microorganism for the production of isobutene as has been set forth above for the methods and recombinant organisms or microorganisms according to the present invention.

The present invention furthermore relates to the use of an enzyme catalyzing the enzymatic conversion of 3-methyl-3-butenoic acid into isobutene for the production of isobutene from 3-methyl-3-butenoic acid.

The present invention furthermore relates to the use of (i) an enzyme capable of enzymatically converting 3-methyl-3-butenoic acid into isobutene (step XVI as shown in FIG. 1) and (ii) an enzyme capable of enzymatically converting 3-methyl-3-butenoyl-CoA into 3-methyl-3-butenoic acid (step XVII as shown in FIG. 1) for the production of isobutene.

In another preferred embodiment, the present invention relates to the use of (i) an enzyme capable of enzymatically converting 3-methyl-3-butenoic acid into isobutene (step XVI as shown in FIG. 1) and (ii) an enzyme capable of enzymatically converting 3-methyl-3-butenoyl-CoA into 3-methyl-3-butenoic acid (step XVII as shown in FIG. 1) an enzyme capable of enzymatically converting 3-methylglutaconyl-CoA into 3-methyl-3-butenoyl-CoA (step XVIII as shown in FIG. 1) for the production of isobutene.

In another preferred embodiment, the present invention relates to the use of (i) an enzyme capable of enzymatically converting 3-methyl-3-butenoic acid into isobutene (step XVI as shown in FIG. 1) and (ii) an enzyme capable of enzymatically converting 3-methyl-3-butenoyl-CoA into 3-methyl-3-butenoic acid (step XVII as shown in FIG. 1), an enzyme capable of enzymatically converting 3-methylglutaconyl-CoA into 3-methyl-3-butenoyl-CoA (step XVIII as shown in FIG. 1) and an enzyme capable of enzymatically converting 3-hydroxy-3-methylglutaryl-CoA into 3-methylglutaconyl-CoA (step VIII as shown in FIG. 1) for the production of isobutene.

In another preferred embodiment, the present invention relates to the use of (i) an enzyme capable of enzymatically converting 3-methyl-3-butenoic acid into isobutene (step XVI as shown in FIG. 1) and (ii) an enzyme capable of enzymatically converting 3-methyl-3-butenoyl-CoA into 3-methyl-3-butenoic acid (step XVII as shown in FIG. 1); an enzyme capable of enzymatically converting 3-methylglutaconyl-CoA into 3-methyl-3-butenoyl-CoA (step XVIII as shown in FIG. 1); an enzyme capable of enzymatically converting 3-hydroxy-3-methylglutaryl-CoA into 3-methylglutaconyl-CoA (step VIII as shown in FIG. 1) and an enzyme capable of enzymatically condensing acetoacetyl-CoA and acetyl-CoA into 3-hydroxy-3-methylglutaryl-CoA (step IX as shown in FIG. 1) for the production of isobutene.

In another preferred embodiment, the present invention relates to the use of (i) an enzyme capable of enzymatically converting 3-methyl-3-butenoic acid into isobutene (step XVI as shown in FIG. 1) and (ii) an enzyme capable of enzymatically converting 3-methyl-3-butenoyl-CoA into 3-methyl-3-butenoic acid (step XVII as shown in FIG. 1); an enzyme capable of enzymatically converting 3-methylglutaconyl-CoA into 3-methyl-3-butenoyl-CoA (step XVIII as shown in FIG. 1); an enzyme capable of enzymatically converting 3-hydroxy-3-methylglutaryl-CoA into 3-methylglutaconyl-CoA (step VIII as shown in FIG. 1); an enzyme capable of enzymatically condensing acetoacetyl-CoA and acetyl-CoA into 3-hydroxy-3-methylglutaryl-CoA (step IX as shown in FIG. 1) and an enzyme capable of enzymatically converting acetyl-CoA into acetoacetyl-CoA comprising (a) (i) an enzyme capable of converting acetyl-CoA into malonyl-CoA (step XIV as shown in FIG. 1); and (ii) an enzyme capable of condensing malonyl-CoA and acetyl-CoA into acetoacetyl-CoA (step XV as shown in FIG. 1); or (b) an enzyme capable of directly condensing two molecules of acetyl-CoA into acetoacetyl-CoA (step XIII as shown in FIG. 1) for the production of isobutene.

As regards the above-mentioned enzymes as well as preferred embodiments of said enzymes, the same applies to the use of the recombinant organism or microorganism for the production of isobutene as has been set forth above for the methods and recombinant organisms or microorganisms according to the present invention.

In a further aspect, the present invention relates to any of the above uses of enzymes for the production of isobutene, wherein additionally
a) an enzyme capable of enzymatically converting 3-hydroxyisovalerate (HIV) into 3-methylcrotonic acid with a concomitant transfer of CoA from 3-methylcrotonyl-CoA on 3-hydroxyisovalerate (HIV) to result in 3-hydroxyisovaleryl-CoA (step Xa as schematically shown in FIG. 19); and/or
b) an enzyme capable of enzymatically converting 3-hydroxyisovalerate (HIV) into 3-hydroxyisovaleryl-CoA (step Xb as schematically shown in FIG. 20); and/or
c) an enzyme capable of enzymatically converting 3-hydroxyisovaleryl-CoA into 3-methylcrotonyl-CoA (step XI as schematically shown in FIG. 21); and/or
d) an enzyme capable of enzymatically converting 3-hydroxyisovalerate (HIV) into 3-hydroxyisovaleryl-CoA (step XII as schematically shown in FIG. 22)
   as described herein above is used.

As regards the above-mentioned enzymes as well as preferred embodiments of said enzymes, the same applies to the recombinant organism or microorganism as has been set forth above for the methods according to the present invention.

Furthermore, the present invention relates to a composition comprising 3-methylcrotonic acid and a recombinant organism or microorganism, wherein said recombinant organism or microorganism expresses (i) an enzyme capable of enzymatically converting 3-methylcrotonic acid into isobutene (step I as shown in FIG. 1); and/or (ii) an enzyme capable of enzymatically converting 3-hydroxyisovalerate (HIV) into 3-methylcrotonic acid (step II as shown in FIG. 1), and/or which further expresses an enzyme capable of enzymatically condensing acetone and acetyl-CoA into 3-hydroxyisovalerate (HIV) (step II as shown in FIG. 1), and/or which further expresses an enzyme capable of enzymatically converting acetoacetate into acetone (step IV as shown in FIG. 1), and/or which further expresses an enzyme capable of converting acetoacetyl-CoA into acetoacetate (step Va or Vb as shown in FIG. 1) and/or which further expresses an enzyme capable of enzymatically converting acetyl-CoA into acetoacetyl-CoA comprising (a) (i) an enzyme capable of converting acetyl-CoA into malonyl-CoA (step XIV as shown in FIG. 1); and (ii) an enzyme capable of condensing malonyl-CoA and acetyl-CoA into acetoacetyl-CoA (step XV as shown in FIG. 1); or (b) an enzyme capable of directly condensing two molecules of acetyl-CoA into acetoacetyl-CoA (step XIII as shown in FIG. 1).

Furthermore, the present invention relates to a composition comprising 3-methylcrotonic acid (i) an enzyme capable of enzymatically converting 3-methylcrotonic acid into isobutene (step I as shown in FIG. 1); and/or (ii) an enzyme capable of enzymatically converting 3-hydroxyisovalerate (HIV) into 3-methylcrotonic acid (step II as shown in FIG. 1); and/or an enzyme capable of enzymatically condensing acetone and acetyl-CoA into 3-hydroxyisovalerate (HIV) (step III as shown in FIG. 1), and/or an enzyme capable of enzymatically converting acetoacetate into acetone (step IV as shown in FIG. 1), and/or an enzyme capable of converting acetoacetyl-CoA into acetoacetate (step Va or Vb as shown in FIG. 1) and/or an enzyme capable of enzymatically converting acetyl-CoA into acetoacetyl-CoA comprising (a) (i) an enzyme capable of converting acetyl-CoA into malonyl-CoA (step XIV as shown in FIG. 1); and (ii) an enzyme capable of condensing malonyl-CoA and acetyl-CoA into acetoacetyl-CoA (step XV as shown in FIG. 1); or (b) an enzyme capable of directly condensing two molecules of acetyl-CoA into acetoacetyl-CoA (step XIII as shown in FIG. 1).

As regards the above-mentioned enzymes as well as preferred embodiments of said enzymes, the same applies to the use of the recombinant organism or microorganism for the production of isobutene as has been set forth above for the methods and recombinant organisms or microorganisms according to the present invention.

Furthermore, the present invention relates to a composition comprising 3-methylcrotonic acid and a recombinant organism or microorganism, wherein said recombinant organism or microorganism expresses (i) an enzyme capable of enzymatically converting 3-methylcrotonic acid into isobutene (step I as shown in FIG. 1); and/or (ii) an enzyme capable of enzymatically converting 3-methylcrotonyl-CoA into 3-methylcrotonic acid (step VIa, VIb or VIc as shown in FIG. 1), and/or which further expresses an enzyme capable of enzymatically converting 3-methylglutaconyl-CoA into 3-methylcrotonyl-CoA (step VII as shown in FIG. 1), and/or which further expresses an enzyme capable of enzymatically converting 3-hydroxy-3-methylglutaryl-CoA into 3-methylglutaconyl-CoA (step VIII as shown in FIG. 1), and/or which further expresses an enzyme capable of enzymatically condensing acetoacetyl-CoA and acetyl-CoA into 3-hydroxy-3-methylglutaryl-CoA (step IX as shown in FIG. 1) and/or which further expresses an enzyme capable of enzymatically converting acetyl-CoA into acetoacetyl-CoA comprising (a) (i) an enzyme capable of converting acetyl-CoA into malonyl-CoA (step XIV as shown in FIG. 1); and (ii) an enzyme capable of condensing malonyl-CoA and acetyl-CoA into acetoacetyl-CoA (step XV as shown in FIG. 1); or (b) an enzyme capable of directly condensing two molecules of acetyl-CoA into acetoacetyl-CoA (step XIII as shown in FIG. 1).

Furthermore, the present invention relates to a composition comprising 3-methylcrotonic acid and (i) an enzyme capable of enzymatically converting 3-methylcrotonic acid into isobutene (step I as shown in FIG. 1); and/or (ii) an enzyme capable of enzymatically converting 3-methylcrotonyl-CoA into 3-methylcrotonic acid (step VIa, VIb or VIc as shown in FIG. 1); and/or an enzyme capable of enzymatically converting 3-methylglutaconyl-CoA into 3-methylcrotonyl-CoA (step VII as shown in FIG. 1); and/or an enzyme capable of enzymatically converting 3-hydroxy-3-methylglutaryl-CoA into 3-methylglutaconyl-CoA (step VIII as shown in FIG. 1); and/or an enzyme capable of enzymatically condensing acetoacetyl-CoA and acetyl-CoA into 3-hydroxy-3-methylglutaryl-CoA (step IX as shown in FIG. 1) and/or an enzyme capable of enzymatically converting acetyl-CoA into acetoacetyl-CoA comprising (a) (i) an enzyme capable of converting acetyl-CoA into malonyl-CoA (step XIV as shown in FIG. 1); and (ii) an enzyme capable of condensing malonyl-CoA and acetyl-CoA into acetoacetyl-CoA (step XV as shown in FIG. 1); or (b) an enzyme capable of directly condensing two molecules of acetyl-CoA into acetoacetyl-CoA (step XIII as shown in FIG. 1).

As regards the above-mentioned enzymes as well as preferred embodiments of said enzymes, the same applies to the use of the recombinant organism or microorganism for the production of isobutene as has been set forth above for the methods and recombinant organisms or microorganisms according to the present invention.

Furthermore, the present invention relates to a composition comprising 3-methyl-3-butenoic acid and a recombinant organism or microorganism, wherein said recombinant organism or microorganism expresses (i) an enzyme capable of enzymatically converting 3-methyl-3-butenoic acid into isobutene (step XVI as shown in FIG. 1) and/or (ii) an enzyme capable of enzymatically converting 3-methyl-3-butenoyl-CoA into 3-methyl-3-butenoic acid (step XVII as shown in FIG. 1), and/or which further expresses an enzyme capable of enzymatically converting 3-methylglutaconyl-CoA into 3-methyl-3-butenoyl-CoA (step XVIII as shown in FIG. 1), and/or which further expresses an enzyme capable of enzymatically converting 3-hydroxy-3-methylglutaryl-CoA into 3-methylglutaconyl-CoA (step VIII as shown in FIG. 1), and/or which further expresses an enzyme capable of enzymatically condensing acetoacetyl-CoA and acetyl-CoA into 3-hydroxy-3-methylglutaryl-CoA (step IX as shown in FIG. 1) and/or which further expresses an enzyme capable of enzymatically converting acetyl-CoA into acetoacetyl-CoA comprising (a) (i) an enzyme capable of converting acetyl-CoA into malonyl-CoA (step XIV as shown in FIG. 1); and (ii) an enzyme capable of condensing malonyl-CoA and acetyl-CoA into acetoacetyl-CoA (step XV as shown in FIG. 1); or (b) an enzyme capable of directly condensing two molecules of acetyl-CoA into acetoacetyl-CoA (step XIII as shown in FIG. 1).

Furthermore, the present invention relates to a composition comprising 3-methyl-3-butenoic acid and (i) an enzyme capable of enzymatically converting 3-methyl-3-butenoic acid into isobutene (step XVI as shown in FIG. 1) and/or (ii) an enzyme capable of enzymatically converting 3-methyl-3-butenoyl-CoA into 3-methyl-3-butenoic acid (step XVII as shown in FIG. 1); and/or an enzyme capable of enzymatically converting 3-methylglutaconyl-CoA into 3-methyl-3-butenoyl-CoA (step XVIII as shown in FIG. 1); and/or an enzyme capable of enzymatically converting 3-hydroxy-3-methylglutaryl-CoA into 3-methylglutaconyl-CoA (step VIII as shown in FIG. 1); and/or an enzyme capable of enzymatically condensing acetoacetyl-CoA and acetyl-CoA into 3-hydroxy-3-methylglutaryl-CoA (step IX as shown in FIG. 1) and/or an enzyme capable of enzymatically converting acetyl-CoA into acetoacetyl-CoA comprising (a) (i) an enzyme capable of converting acetyl-CoA into malonyl-CoA (step XIV as shown in FIG. 1); and (ii) an enzyme capable of condensing malonyl-CoA and acetyl-CoA into acetoacetyl-CoA (step XV as shown in FIG. 1); or (b) an enzyme capable of directly condensing two molecules of acetyl-CoA into acetoacetyl-CoA (step XIII as shown in FIG. 1) for the production of isobutene.

As regards the above-mentioned enzymes as well as preferred embodiments of said enzymes, the same applies to the use of the recombinant organism or microorganism for the production of isobutene as has been set forth above for the methods and recombinant organisms or microorganisms according to the present invention.

In a further aspect, the present invention relates to any of the above-described compositions, wherein the organism or microorganism is an organism or microorganism which additionally further expresses
a) an enzyme capable of enzymatically converting 3-hydroxyisovalerate (HIV) into 3-methylcrotonic acid with a concomitant transfer of CoA from 3-methylcrotonyl-CoA on 3-hydroxyisovalerate (HIV) to result in 3-hydroxyisovaleryl-CoA (step Xa as schematically shown in FIG. 19); and/or
b) an enzyme capable of enzymatically converting 3-hydroxyisovalerate (HIV) into 3-hydroxyisovaleryl-CoA (step Xb as schematically shown in FIG. 20); and/or
c) an enzyme capable of enzymatically converting 3-hydroxyisovaleryl-CoA into 3-methylcrotonyl-CoA (step XI as schematically shown in FIG. 21); and/or
d) an enzyme capable of enzymatically converting 3-hydroxyisovalerate (HIV) into 3-hydroxyisovaleryl-CoA (step XII as schematically shown in FIG. 22)
   as described herein above.

In a further aspect, the present invention relates to any of the above-described compositions which further additionally comprises
a) an enzyme capable of enzymatically converting 3-hydroxyisovalerate (HIV) into 3-methylcrotonic acid with a concomitant transfer of CoA from 3-methylcrotonyl-CoA on 3-hydroxyisovalerate (HIV) to result in 3-hydroxyisovaleryl-CoA (step Xa as schematically shown in FIG. 19); and/or
b) and enzyme capable of enzymatically converting 3-hydroxyisovalerate (HIV) into 3-hydroxyisovaleryl-CoA (step Xb as schematically shown in FIG. 20); and/or
c) an enzyme capable of enzymatically converting 3-hydroxyisovaleryl-CoA into 3-methylcrotonyl-CoA (step XI as schematically shown in FIG. 21); and/or
d) an enzyme capable of enzymatically converting 3-hydroxyisovalerate (HIV) into 3-hydroxyisovaleryl-CoA (step XII as schematically shown in FIG. 22)
   as described herein above.

As regards the above-mentioned enzymes as well as preferred embodiments of said enzymes, the same applies to the recombinant organism or microorganism as has been set forth above for the methods according to the present invention.

FIG. 1: shows an artificial pathway for isobutene production from acetyl-CoA via 3-methylcrotonic acid. Moreover, enzymatic recycling of metabolites which may occur during the pathway are shown in steps Xa, Xb, XI and XII.

FIG. 2A: Schematic reaction of the enzymatic prenylation of a flavin mononucleotide (FMN) into the corresponding modified (prenylated) flavin cofactor.

FIG. 2B: Schematic reaction of the enzymatic conversion of 3-methylcrotonic acid into isobutene.

FIG. 3: Schematic reaction of the enzymatic conversion of 3-hydroxyisovalerate (HIV) into 3-methylcrotonic acid.

FIG. 4: Schematic reaction of the enzymatic condensation of acetyl-CoA and acetone into 3-hydroxyisovalerate.

FIG. 5: Schematic reaction of the enzymatic conversion of acetoacetate into acetone.

FIG. 6: Schematic reaction of the enzymatic conversion of acetoacetyl-CoA into acetoacetate by hydrolysing the CoA thioester of acetoacetyl-CoA resulting in acetoacetate.

FIG. 7: Schematic reaction of the enzymatic conversion of acetoacetyl-CoA into acetoacetate by transferring the CoA group of acetoacetyl-CoA on acetate, resulting in the formation of acetoacetate and acetyl-CoA.

FIG. 8: Schematic reaction of the enzymatic conversion of 3-methylcrotonyl-CoA into 3-methylcrotonic acid.

FIG. 9: Schematic reaction of the enzymatic conversion of 3-methylcrotonyl-CoA into 3-methylcrotonic acid via step VIa as shown in FIG. 1.

FIG. 10: Schematic reaction of the enzymatic conversion of 3-methylcrotonyl-CoA into 3-methylcrotonic acid via step VIb as shown in FIG. 1.

FIG. 11: Schematic reaction of the enzymatic conversion of 3-methylcrotonyl-CoA into 3-methylcrotonic acid via step VIc as shown in FIG. 1.

FIG. 12: Schematic reaction of the enzymatic conversion of 3-methylglutaconyl-CoA into 3-methylcrotonyl-CoA.

FIG. 13: Schematic reaction of the enzymatic conversion of 3-hydroxy-3-methylglutaryl-CoA into 3-methylglutaconyl-CoA.

FIG. 14: Schematic reaction of the enzymatic condensation of acetylCoA and acetoacetyl-CoA into 3-hydroxy-3-methylglutaryl-CoA.

FIG. 15: Schematic reaction of the enzymatic condensation of two molecules of acetyl-CoA into acetoacetyl-CoA.

FIG. 16: Schematic reaction of the enzymatic conversion of acetyl-CoA into malonyl-CoA.

FIG. 17: Schematic reaction of the enzymatic condensation of malonyl-CoA and acetyl-CoA into acetoacetyl-CoA.

FIG. 18: shows enzymatic recycling steps of metabolites (steps Xa, Xb, XI and XII as also shown in FIG. 1) which may occur during the pathway of isobutene production from acetyl-CoA via 3-methylcrotonic acid.

FIG. 19: Schematic reaction of the enzymatic conversion of 3-hydroxyisovalerate (HIV) into 3-methylcrotonic acid with a concomitant transfer of CoA from 3-methylcrotonyl-CoA on 3-hydroxyisovalerate (HIV) to result in 3-hydroxyisovaleryl-CoA.

FIG. 20: Schematic reaction of the enzymatic conversion of 3-hydroxyisovalerate (HIV) into 3-hydroxyisovaleryl-CoA.

FIG. 21: Schematic reaction of the enzymatic conversion of 3-hydroxyisovaleryl-CoA into 3-methylcrotonyl-CoA.

FIG. 22: Schematic reaction of the general enzymatic conversion of 3-hydroxyisovalerate (HIV) into 3-hydroxyisovaleryl-CoA.

FIG. 23: Schematic reaction of the enzymatic conversion of 3-hydroxyisovalerate (HIV) into 3-hydroxyisovaleryl-CoA via 3-hydroxyisovaleryl-adenosine monophosphate.

FIG. 24: Schematic reaction of the enzymatic conversion of 3-hydroxyisovalerate (HIV) into 3-hydroxyisovaleryl-CoA via 3-hydroxyisovaleryl phosphate.

FIG. 25: Schematic reaction of the enzymatic conversion of 3-methyl-3-butenoic acid into isobutene.

FIG. 26: Schematic reaction of the enzymatic conversion of 3-methyl-3-butenoyl-CoA into 3-methyl-3-butenoic acid.

FIG. 27: Schematic reaction of the enzymatic conversion of 3-methyl-3-butenoyl-CoA into 3-methyl-3-butenoic acid by making use of a CoA-transferase.

FIG. 28: Schematic reaction of the enzymatic conversion of 3-methyl-3-butenoyl-CoA into 3-methyl-3-butenoic acid by making use of a thioester hydrolase.

FIG. 29: Schematic reaction of the enzymatic conversion of 3-methyl-3-butenoyl-CoA into 3-methyl-3-butenoic acid in a two-step reaction via 3-methyl-3-butenoyl phosphate.

FIG. 30: Schematic reaction of the enzymatic conversion of 3-methylglutaconyl-CoA into 3-methyl-3-butenoyl-CoA.

FIG. 31: Structure of a phosphopantetheine moiety.

FIG. 32: Schematic illustration for the conversion of 3-methylcrotonyl-CoA into 3-methylcrotonic acid via 3-methylbutyryl-CoA and 3-methylbutyric acid.

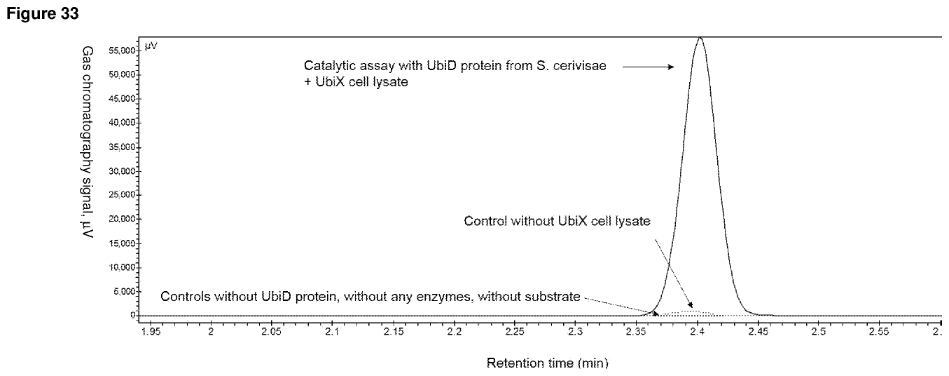

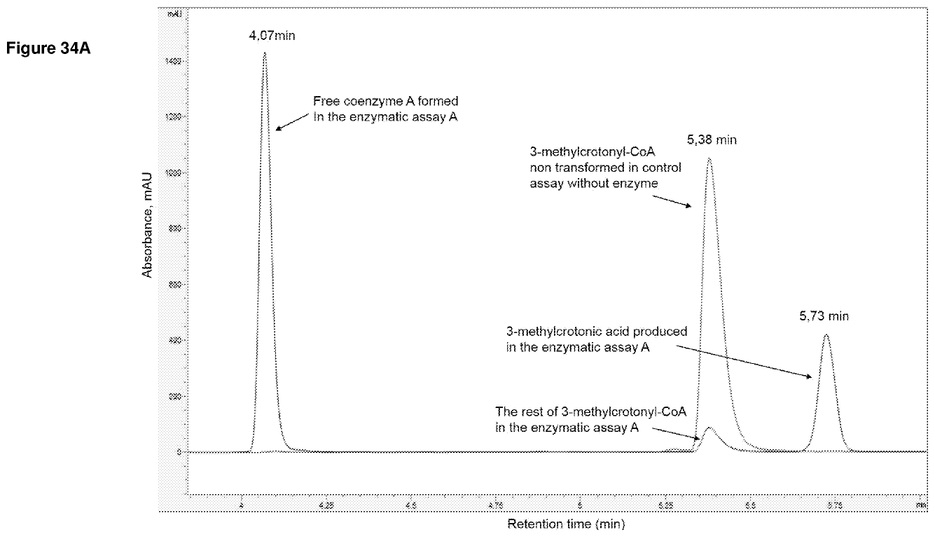

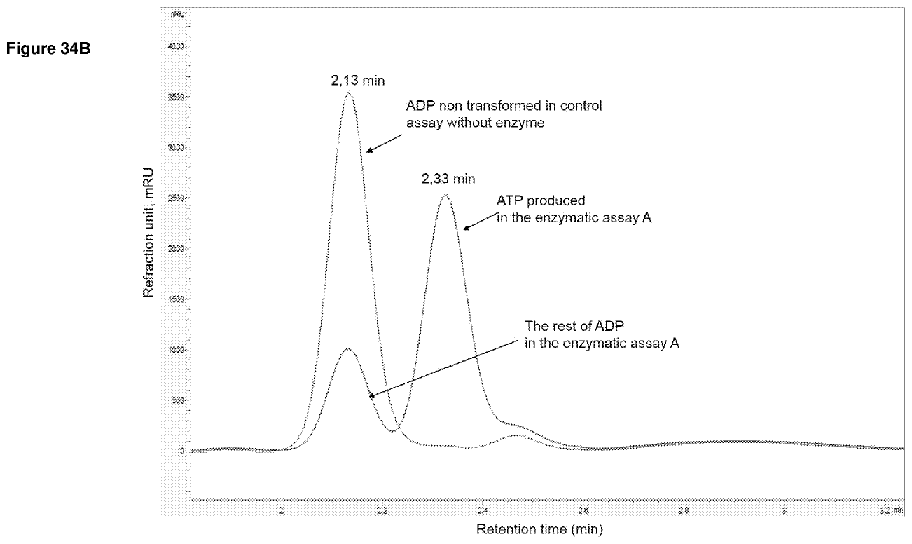

FIG. 33: shows an overlay of typical GC-chromatograms obtained for the catalytic assay of UbiD protein from Saccharomyces cerevisiae with the corresponding controls as outlined in Example 2.

FIG. 34A: shows an overlay of typical HPLC-chromatograms (analysis of 3-methylcrotonyl-CoA, 3-methylcrotonic acid and CoA-SH) obtained for the “Enzymatic assay” (assay A, Example 3) and the “Enzyme-free assay” (assay H, Example 3). The consumption of 3-methylcrotonyl-CoA with simultaneous production of CoA-SH and 3-methylcrotonic acid was observed in the enzymatic assay combining phosphate butyryltransferase with butyrate kinase.

FIG. 34B: shows an overlay of typical HPLC-chromatograms (analysis of ADP and ATP) obtained for the “Enzymatic assay” (assay A, Example 3) and the “Enzyme-free assay” (assay H, Example 3). The consumption of ADP with simultaneous production of ATP was observed in the enzymatic assay combining phosphate butyryltransferase with butyrate kinase.

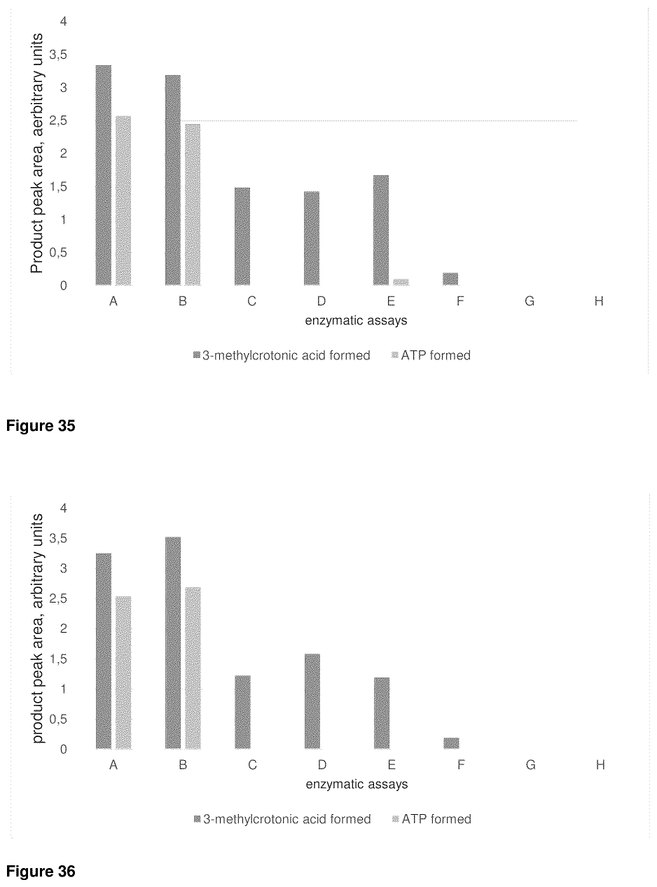

FIG. 35: shows the results of the production of 3-methylcrotonic acid and ATP in the enzymatic assays, comprising phosphate butyryltransferase from Bacillus subtilis combined with different butyrate kinases. Moreover, the production of 3-methylcrotonic acid and ATP in control assays is shown.

FIG. 36: shows the results of the production of 3-methylcrotonic acid and ATP in the enzymatic assays, comprising phosphate butyryltransferase from from Enterococcus faecalis combined with different butyrate kinases. Moreover, the production of 3-methylcrotonic acid and ATP in different control assays is shown.

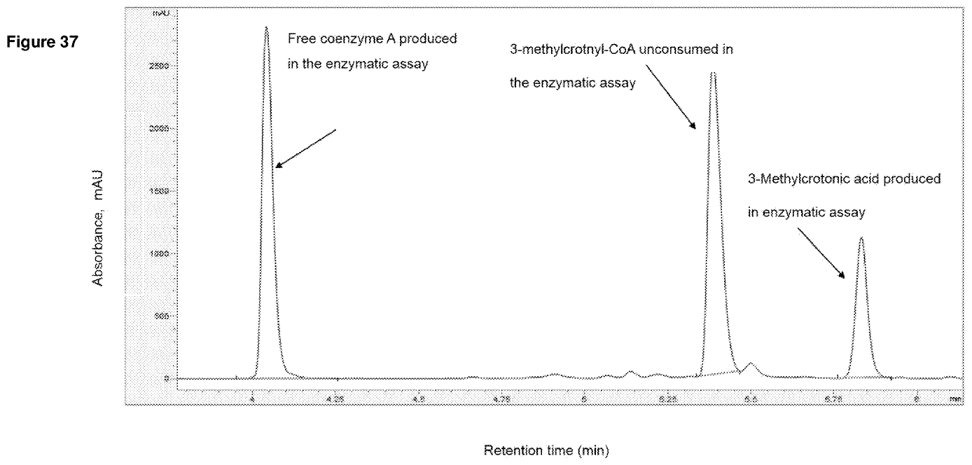

FIG. 37: shows an example of typical HPLC-chromatogram obtained for the enzymatic assay with acyl-CoA thioesterase II from Pseudomonas putida as outlined in Example 5.

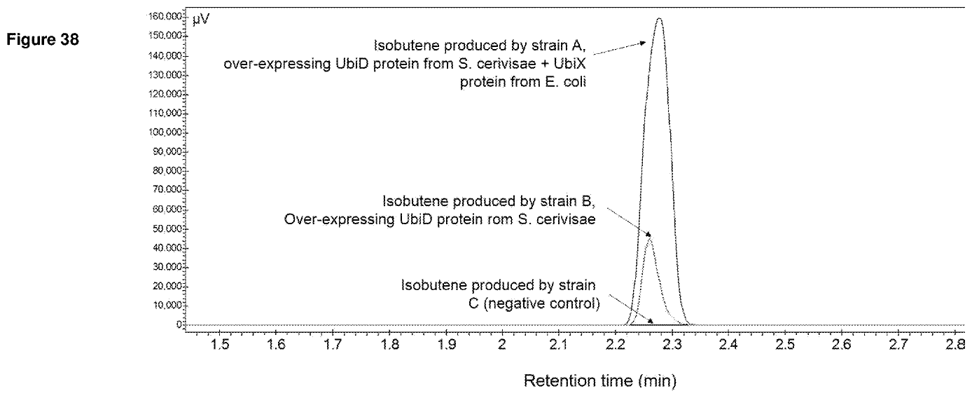

FIG. 38: shows an overlay of typical chromatograms obtained for the production of isobutene from 3-methylcrotonic in a recombinant E. coli strain overexpressing UbiD protein from Saccharomyces cerevisiae and UbiX protein from Escherichia coli (strain A) or overexpressing UbiD protein from Saccharomyces cerevisiae alone (strain B) or carrying an empty vector (negative control, strain C).

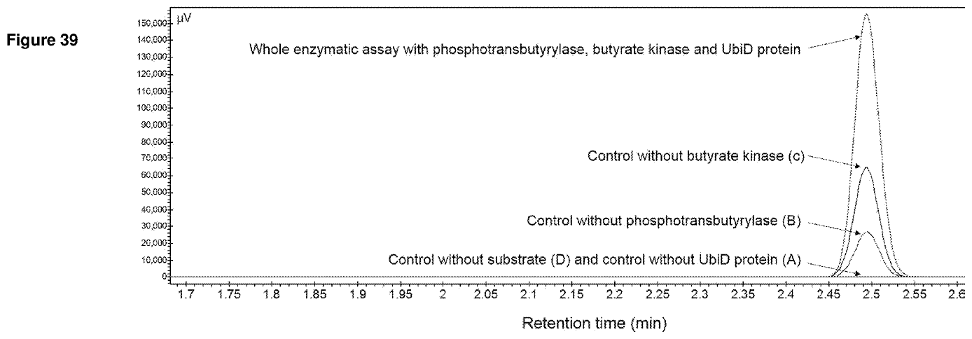

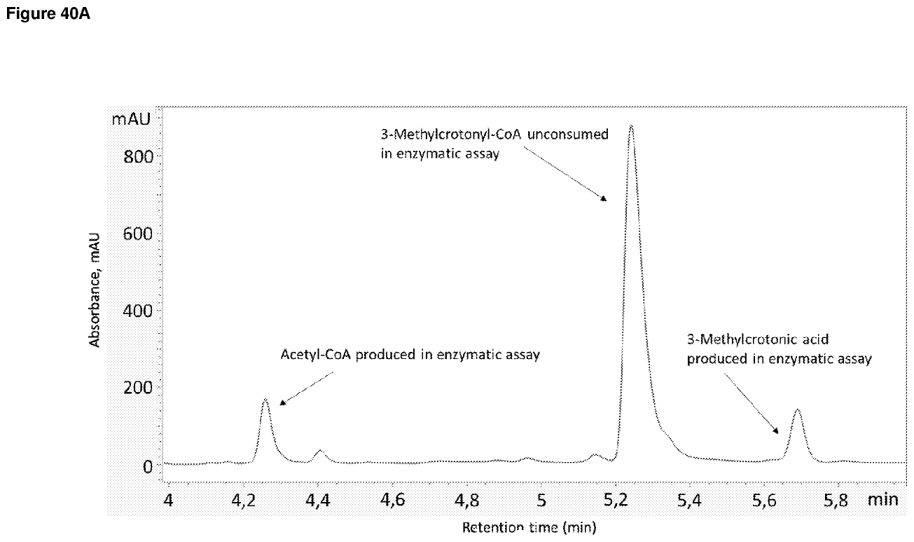

FIG. 39: shows an overlay of typical chromatograms obtained for the production of isobutene from 3-methylcrotonyl-CoA in the one pot enzymatic assay as outlined in Example 7, and the corresponding controls.

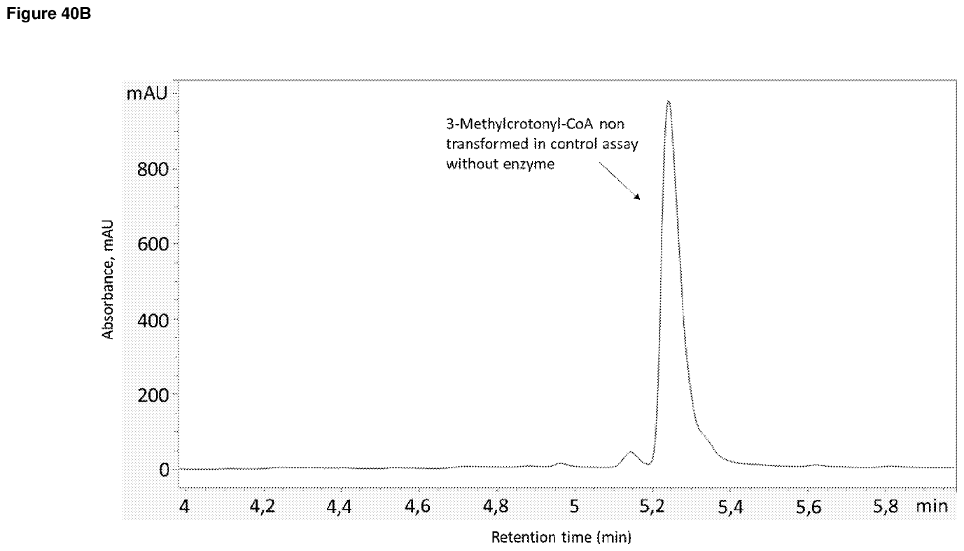

FIGS. 40A and 40B: shows chromatograms for enzymatic assays (FIG. 40A) and control assays (FIG. 40B). A significant quantity of acetyl-CoA and 3-methylcrotonic acid was produced from acetate and 3-methylcrotonyl-CoA in the presence of Co-A transferase (FIG. 40A) while no product was observed in the control assay without enzyme (FIG. 40B).

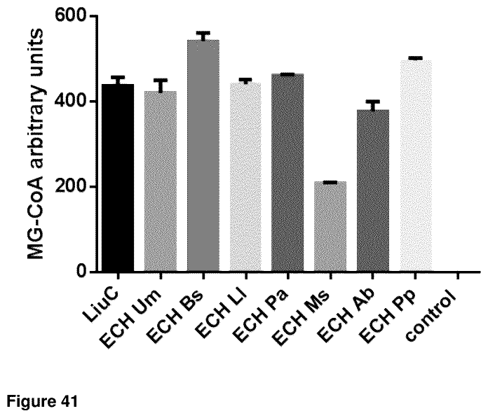

FIG. 41: shows 3-methylglutaconyl-CoA (MG-CoA) peak areas obtained from HPLC-based analysis.

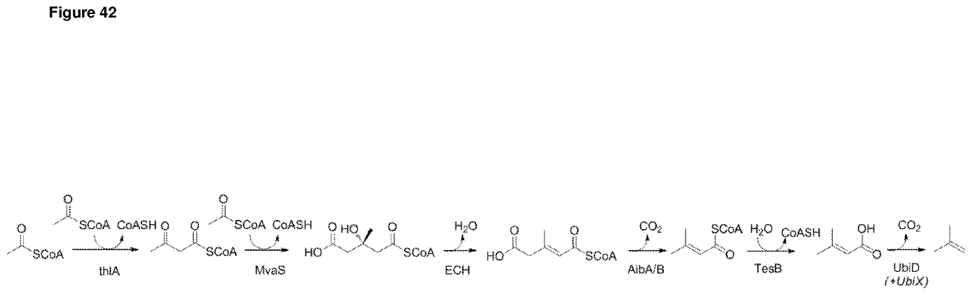

FIG. 42: Metabolic pathway for the biosynthesis of isobutene from acetyl-CoA via 3-methycrotonic acid, implemented in Escherichia coli.

In this specification, a number of documents including patent applications are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The invention will now be described by reference to the following examples which are merely illustrative and are not to be construed as a limitation of the scope of the present invention.

### EXAMPLES

### General Methods and Materials

All reagents and materials used in the experiences were obtained from Sigma-Aldrich Company (St. Louis, MO) unless otherwise specified. Materials and methods suitable for growth of bacterial cultures and protein expression are well known in the art.

### Example 1: Gene Synthesis, Cloning and Expression of Recombinant Proteins

The sequences of the studied enzymes were generated by oligonucleotide concatenation to fit the codon usage of E. coli (genes were commercially synthesized by GeneArt®). A stretch of 6 histidine codons was inserted after the methionine initiation codon to provide an affinity tag for purification. The gene thus synthesized was cloned in a pET-25b (+) expression vector (vectors were constructed by GeneArt®). Vector pCAN contained gene coding for UbiX protein (3-octaprenyl-4-hydroxybenzoate carboxy-lyase partner protein) from Escherichia coli (Uniprot Accession Number: P0AG03) was purchased from NAIST (Nara Institute of Science and Technology, Japan, ASKA collection). Provided vector contained a stretch of 6 histidine codons after the methionine initiation codon.

Competent E. coli BL21 (DE3) cells (Novagen) were transformed with these vectors according to standard heat shock procedure. The transformed cells were grown with shaking (160 rpm) using ZYM-5052 auto-induction medium (Studier FW, Prot. Exp. Pur. 41, (2005), 207-234) for 6 h at 30° C. and protein expression was continued at 18° C. overnight (approximately 16 h). For the recombinant strain over-expressing UbiX from E. coli, 500 μM of Flavin Mononucleotide (FMN) were added to the growth medium. The cells were collected by centrifugation at 4° C., 10,000 rpm for 20 min and the pellets were stored at −80° C.

### Protein Purification and Concentration

The pellets from 200 ml of cultured cells were thawed on ice and resuspended in 6 ml of 50 mM Tris-HCl buffer pH 7.5 containing 100 mM NaCl in the case of the recombinant strain overexpressing UbiX protein and in 6 ml of 50 mM Tris-HCl buffer pH 7.5, 10 mM MgCl₂, 10 mM imidazole and 5 mM DTT in the case of the recombinant strain overexpressing UbiD protein. Twenty microliters of lysonase (Novagen) were added. Cells were then incubated 10 min at room temperature, returned to ice for 20 min and the lysis was completed by sonication 3×15 seconds. The cellular lysate contained UbiX protein was reserved on ice. The bacterial extracts contained UbiD proteins were then clarified by centrifugation at 4° C., 4000 rpm for 40 min. The clarified bacterial lysates were loaded onto a PROTINO-2000 Ni-TED column (Macherey-Nagel) allowing adsorption of 6-His tagged proteins. Columns were washed and the enzymes of interest were eluted with 6 ml of 100 mM Tris-HCl buffer pH 7.5 containing 100 mM NaCl and 250 mM imidazole. Eluates were then concentrated, desalted on Amicon Ultra-4 10 kDa filter unit (Millipore) and enzymes were resuspended in 50 mM Tris-HCl buffer pH 7.5, containing 50 mM NaCl and 5 mM DTT.

The purity of proteins thus purified varied from 80% to 90% as estimated by SDS-PAGE analysis. Protein concentration was determined by direct UV 280 nm measurement on the NanoDrop 1000 spectrophotometer (Thermo Scientific) and by Bradford assay (BioRad).

### Example 2: In Vitro Decarboxylation of 3-Methylcrotonic Acid into Isobutene Catalyzed by an Association of Lysate, Containing UbiX Protein, with Purified UbiD Protein

0.5 M stock solution of 3-methylcrotonic acid was prepared in water and adjusted to pH 7.0 with 10 M solution of NaOH.

Two UbiD proteins (Table C) were purified according to the procedure described in Example 1.

Enzymatic assays were carried out in 2 ml glass vials (Interchim) under the following conditions:

50 mM Tris-HCl buffer pH 7.5

20 mM NaCl

10 mM MgCl₂

5 mM DTT

50 mM 3-methylcrotonic acid

1 mg/ml purified UbiD protein

50 μl lysate contained UbiX protein

Total volume of the assays were 300 μl.

A series of control assays were performed in parallel (Table C).

The vials were sealed and incubated for 120 min at 30° C. The assays were stopped by incubating for 2 min at 80° C. and the isobutene formed in the reaction headspace was analysed by Gas Chromatography (GC) equipped with Flame Ionization Detector (FID).

For the GC analysis, one ml of the headspace gas was separated in a Bruker GC-450 system equipped with a GS-alumina column (30 m×0.53 mm) (Agilent) using isothermal mode at 130° C. Nitrogen was used as carrier gas with a flow rate of 6 ml/min.

The enzymatic reaction product was identified by comparison with an isobutene standard. Under these GC conditions, the retention time of isobutene was 2.42 min. A significant production of isobutene from 3-methylcrotonic acid was observed in the combined assays (UbiD protein+UbiX protein). Incubation of lysate containing UbIX protein alone did not result in isobutene production. These data indicate that the two enzymes present in the assays cooperated to perform the decarboxylation of 3-methylcrotonic acid into isobutene. A typical chromatogram obtained in the assay with UbiD protein from Saccharomyces cerevisiae is shown on FIG. 33.

TABLE C

| | Isobutene |
|---|---|
| | production, arbitrary |
| Assay composition | units |
| UbiD protein from C. dubliniensis | 470 |
| (Uniprot Acession Number: | |
| B9WJ66) + lysate contained | |
| UbiX protein from E. coli + substrate | |
| UbiD protein from C. dubliniensis (Uniprot | 9.2 |
| Acession Number: B9WJ66) + substrate | |
| UbiD protein from S. cervisiae (Uniprot | 1923 |
| Acession Number : Q03034) + lysate | |
| contained UbiX protein | |
| from E. coli + substrate | |
| UbiD protein from S. cerivisae (Uniprot | 31 |
| Acession Number: Q03034) + substrate | |
| Lysate contained UbiX protein | 0 |
| from E. coli + substrate | |
| “No substrate control”: UbiD protein from | 0 |
| C. dubliniensis (Uniprot Acession | |
| Number: B9WJ66) + lysate | |
| contained UbiX protein from E. coli, | |
| without substrate | |
| “No substrate control”: UbiD protein | 0 |
| from S. cervisiae (Uniprot Acession | |
| Number : Q03034) + | |
| lysate contained UbiX protein from | |
| E. coli, without substrate | |

### Example 3: Conversion of 3-Methylcrotonyl-CoA and ADP into 3-Methylcrotonic Acid and ATP Catalysed by the Combined Action of Phosphate Butyryltransferase from Bacillus subtilis and Butyrate Kinase from Lactobacillus casei or Geobacillus sp.

The corresponding enzymes were obtained and purified according to the procedure described in Example 1.

The enzymatic assays were conducted in a total reaction volume of 0.2 ml

The standard reaction mixture contained:

50 mM potassium phosphate buffer pH 7.5

4 mM 3-methylcrotonyl-CoA

4 mM ADP

10 mM MgCl₂

10 mM NaCl

0.2 mg/ml purified phosphate butyryltransferase from Bacillus subtilis (Uniprot Accession Number: P54530)

0.2 mg/ml purified butyrate kinase from Lactobacillus casei (Uniprot Accession Number: KON529) or Geobacillus sp. (Uniprot accession number: L8A0E1).

A series of controls were performed in parallel (Assays C-H Table D).

TABLE D

| | Assay composition | | | | | | | |
|---|---|---|---|---|---|---|---|---|
| | A | B | C | D | E | F | G | H |
| 3-methylcrotonyl-CoA | + | + | + | + | + | + | + | + |
| ADP | + | + | | | + | + | + | + |
| phosphate butyryltransferase | + | + | + | + | + | | | |
| from Bacillus subtilis | | | | | | | | |
| butyrate kinase from | + | | + | | | + | | |
| Lactobacillus casei | | | | | | | | |
| butyrate kinase from | | + | | + | | | + | |
| Geobacillus sp | | | | | | | | |

Assays were incubated for 20 min with shaking at 30° C.

After an incubation period, the reactions were stopped by heating the reaction medium 4 min at 90° C. The samples were centrifuged, filtered through a 0.22 μm filter and the clarified supernatants were transferred into a clean vial for the further analysis. The consumption of ADP and 3-methylcrotonyl-CoA, and the formation of ATP, 3-methylcrotonic acid and free coenzyme A (CoA-SH) were followed by using HPLC-based methods.

### HPLC-Based Analysis of ADP and ATP

HPLC analysis was performed using 1260 Inifinity LC System (Agilent), equipped with column heating module and RI detector. 2 μl of samples were separated on Polaris C18-A column (150×4.6 mm, 5 μm particle size, column temp.30° C.) with a mobile phase flow rate of 1.5 ml/min. The separation was performed using 8.4 mM sulfuric acid in H₂O/MeOH mixed solution (99/1) (V/V). In these conditions, the retention time of ADP and ATP were 2.13 min and 2.33 min, respectively.

### HPLC Based Analysis of 3-Methylcrotonyl-CoA, 3-Methylcrotonic Acid and Free Coenzyme A (CoA-SH)

HPLC analysis was performed using 1260 Inifinity LC System (Agilent), equipped with column heating module and UV detector (260 nm). 1 μl of samples were separated on Zorbax SB-Aq column (250×4.6 mm, 5 μm particle size, column temp.30° C.), with a mobile phase flow rate of 1.5 ml/min. The separation was performed using mixed A (H₂O containing 8.4 mM sulfuric acid) and B (acetonitrile) solutions in a linear gradient (0% B at initial time 0 min→70% B at 8 min). In these conditions, the retention time of 3-methylcrotonyl-CoA, 3-methylcrotonic acid and free coenzyme A (CoA-SH) were 5.38 min, 5.73 min and 4.07 min, respectively.

Typical chromatograms obtained for the enzymatic assay A and enzyme-free assay H are shown on FIGS. 34A and 34B.

The results of HPLC analysis are summarized in FIG. 35.

The obtained data indicate that 3-methylcrotonyl-CoA was converted into 3-methylcrotonic acid with the concomitant generation of ATP from ADP in a two-step reaction, catalyzed respectively by two enzymes (assays A and B). Thus, the conversion occurred through the formation of the intermediate 3-methylcrotonyl phosphate followed by the transfer of phosphate group from this intermediate on ADP thereby releasing ATP.

A certain quantity of 3-methylcrotonic acid was produced without simultaneous generation of ATP, when phosphate butyryltransferase was used alone (assay E).

This production is due to a spontaneous hydrolysis of 3-methylcrotonyl phosphate generated by the action of phosphate butyryltransferase.

The production of 3-methylcrotonic acid was observed in the same manner for the control assays without ADP (assays C and D). This production was also due to a hydrolysis of the 3-methylcrotonyl phosphate generated by the action of phosphate butyryltransferase.

### Example 4: Conversion of 3-Methylcrotonyl-CoA and ADP into 3-Methylcrotonic Acid and ATP Catalysed by the Combined Action of the Phosphate Butyryltransferase from Enterococcus faecalis and Butyrate Kinase from Lactobacillus casei or Geobacillus sp.

The corresponding enzymes were obtained and purified according to the procedure described in Example 1.

The enzymatic assays were conducted in a total reaction volume of 0.2 ml

The standard reaction mixture contained:

50 mM potassium phosphate buffer pH 7.5

4 mM 3-methylcrotonyl-CoA

4 mM ADP

10 mM MgCl₂

10 mM NaCl

0.2 mg/ml purified phosphate butyryltransferase from Enterococcus faecalis (Uniprot Accession Number: S4BZL5)

0.2 mg/ml purified butyrate kinase from Lactobacillus casei (Uniprot Accession Number: KON529) or Geobacillus sp. (Uniprot Accession Number: L8A0E1)

A series of controls were performed in parallel (Assays C-H Table E).

TABLE E

| | Assay composition | | | | | | | |
|---|---|---|---|---|---|---|---|---|
| | A | B | C | D | E | F | G | H |
| 3-methylcrotonyl-CoA | + | + | + | + | + | + | + | + |
| ADP | + | + | | | + | + | + | + |
| phosphate | + | + | + | + | + | | | |
| butyryltransferase from | | | | | | | | |
| Enterococcus faecalis | | | | | | | | |
| butyrate kinase from | + | | + | | | + | | |
| Lactobacillus casei | | | | | | | | |
| butyrate kinase from | | + | | + | | | + | |
| Geobacillus sp | | | | | | | | |

Assays were incubated for 20 min with shaking at 30° C.

After an incubation period, the reactions were stopped by heating the reaction medium 4 min at 90° C. The samples were centrifuged, filtered through a 0.22 μm filter and the clarified supernatants were transferred into a clean vial for further analysis. The consumption of ADP and 3-methylcrotonyl-CoA, and the formation of ATP and 3-methylcrotonic acid and free coenzyme A (CoA-SH) were followed by HPLC analysis according to the methods described in Example 3.

The results of HPLC analysis are summarized in FIG. 36.

The obtained data indicate that 3-metylcrotonyl-CoA was converted into 3-methylcrotonic acid with the concomitant generation of ATP from ADP in a two-step reaction, catalyzed respectively by two enzymes (assays A and B). Thus, the conversion occurred through the formation of the intermediate 3-methylcrotonyl phosphate followed by transfer of phosphate group from this intermediate on ADP thereby releasing ATP.

A significant production of 3-methylcrotonic acid, without simultaneous generation of ATP, was observed when phosphate butyryltransferase was used alone (assay E).

This production was due to a hydrolysis of 3-methylcrotonyl phosphate generated by the action of phosphate butyryltransferase.

The production of 3-methylcrotonic acid was observed in the same manner for the control assays without ADP (assays C and D). This production was also due to a hydrolysis of the 3-methylcrotonyl phosphate generated by the action of phosphate butyryltransferase.

### Example 5: Enzyme-Catalyzed Hydrolysis of 3-Methylcrotonyl-CoA into 3-Methylcrotonic Acid and Free Coenzyme A

The gene coding for acyl-CoA thioesterase II from Pseudomonas putida was synthesized according to the procedure described in Example 1.

Vector pCAN contained gene encoding acyl-CoA thioesterase 2 (TesB) from Escherichia coli were purchased from NAIST (Nara Institute of Science and Technology, Japan, ASKA collection). Provided vector contained a stretch of 6 histidine codons after the methionine initiation codon. The corresponding enzymes were produced according to the procedure described in Example 1.

The enzymatic assays were conducted in a total reaction volume of 0.2 ml.

The standard reaction mixture contained:

50 mM HEPES pH 7.0

10 mM 3-methylcrotonyl-CoA

20 mM MgCl2

20 mM NaCl

1 mg/ml purified recombinant thioesterase.

Control assays were performed in which either no enzyme was added, or no substrate was added.

The assays were incubated for 30 min with shaking at 30° C., the reactions were stopped by the addition of 0.1 ml acetonitrile and the samples were then analysed by HPLC-based procedure.

### HPLC Based Analysis of the Consumption of 3-Methylcrotonyl-CoA and the Formation of 3-Methylcrotonic Acid and Free Coenzyme A (CoA-SH)

HPLC analysis was performed using 1260 Inifinity LC System (Agilent), equipped with column heating module and UV detector (210 nm). 5 μl of samples were separated on Zorbax SB-Aq column (250×4.6 mm, 5 μm particle size, column temp.30° C.), with a mobile phase flow rate of 1.5 ml/min. The separation was performed using mixed A (H2O containing 8.4 mM sulfuric acid) and B (acetonitrile) solutions in a linear gradient (0% B at initial time 0 min→70% B at 8 min). Commercial 3-methylcrotonyl-CoA, 3-methylcrotonic acid (Sigma-Aldrich) and CoA-SH (Sigma-Aldrich) were used as references. In these conditions, the retention time of free coenzyme A (CoA-SH), 3-methylcrotonyl-CoA and 3-methylcrotonic acid were 4.05, 5.38 and 5.83 min, respectively.

No 3-methylcrotonic acid signal was observed in control assays.

The both studied thioesterases catalyzed the hydrolysis of 3-methylcrotonyl-CoA with the formation of 3-methylcrotonic acid. An example of chromatogram obtained with acyl-CoA thioesterase II from Pseudomonas putida is shown on FIG. 37.

The production of 3-methylcrotonic acid observed in the enzymatic assays are shown in Table F.

TABLE F

| | | Uniprot Accession | 3-methylcrotonic |
|---|---|---|---|
| Gene name | Organism | Number | acid produced, mM |
| tesB | Escherichia coli | POAGG2 | 0.6 |
| tesB | Pseudomonas putida | Q88DR1 | 3.1 |

### Example 6: In Vivo Decarboxylation of 3-Methylcrotonic Acid into Isobutene Catalyzed by an Association of UbiX Protein from Escherichia coli and UbiD Protein from Saccharomyces cerevisiae

The gene coding for UbiD protein from S. cerevisiae (Uniprot Accession Number: 003034) was codon optimized for expression in E. coli and synthesized by GeneArt® (Life Technologies). This studied gene was then PCR amplified from the pMK-RQ vector (master plasmid provided by GeneArt) using forward primer with NcoI restriction site and a reverse primer, containing BamHI restriction site. The gene coding for UbiX protein from E. coli (Uniprot Accession Number: P0AG03) was amplified by PCR with a forward primer, containing NdeI restriction site and a reverse primer containing KpnI restriction site. The previously described pCAN vector (Example 1) served as template for this PCR step. These two obtained PCR products (UbiD protein and UbiX protein) were cloned into pETDuet™-1 co-expression vector (Novagen). The constructed recombinant plasmid was verified by sequencing. Competent E. coli BL21(DE3) cells (Novagen) were transformed with this vector according to standard heat shock procedure and plated out onto LB agar plates supplemented with ampicillin (0.1 mg/ml) (termed “strain A”).

BL21(DE3) strain transformed with pET-25b(+) vector, carrying only the gene of UbiD protein from S. cerevisae was also used in this study (termed “strain B”). BL21(DE3) strain transformed with an empty pET-25b(+) vector was used as a negative control in the subsequent assays (termed “strain C”).

Single transformants were used to inoculate LB medium, supplemented with ampicillin, followed by incubation at 30° C. overnight. 1 ml of this overnight culture was used to inoculate 300 ml of ZYM-5052 auto-inducing media (Studier FW (2005), local citation). The cultures were grown for 20 hours at 30° C. and 160 rpm shaking.

A volume of cultures corresponding to OD600 of 30 was removed and centrifuged. The pellet was resuspended in 30 ml of MS medium (Richaud C., Mengin-Leucreulx D., Pochet S., Johnson EJ., Cohen G N. and Marliere P, The Journal of Biological Chemistry, 268, (1993), 26827-26835), containing glucose (45 g/L) and MgSO4 (1 mM) and supplemented with 10 mM 3-methylcrotonic acid. These cultures were then incubated in 160 ml bottles, sealed with a screw cap, at 30° C. with shaking for 22 h. The pH value of the cultures was adjusted to 8.5 after 8 hours of incubation by using 30% NH₄OH.

After an incubation period, the isobutene produced in the headspace was analysed by Gas Chromatography (GC) equipped Flame Ionization Detector (FID). One ml of the headspace gas phase was separated and analysed according to the method described in Example 2.

No isobutene was formed with the control strain C carrying an empty vector. The highest production of isobutene was observed for the strain A over-expressing the both genes, UbiD protein from S. cerevisiae and UbiX protein from E. coli. A significant production of isobutene was observed for the strain B over-expressing UbiD protein alone. Thus, endogenous UbiX of E. coli can probably contribute to activate UbiD protein from S. cerevisae (FIG. 38).

### Example 7: One Pot Enzymatic Synthesis of Isobutene from 3-Methylcrotonyl-CoA Catalyzed by an Association of Phosphotransbutyrylase from Bacillus subtilis, Butyrate Kinase from Geobacillus sp. And UbiD Protein from Saccharomyces cerevisiae

A pETDuet™-1 co-expression vector, carrying the UbiD gene from Saccharomyces cerevisiae (Uniprot Accession Number Q03034) and the UbiX gene from Escherichia coli (Uniprot Accession Number P0AG03) (Example 6), was used to produce and purify UbiD protein according to the protocol described in Example 1. The phosphotransbutyrylase from Bacillus subtilis and the butyrate kinase from Geobacillus sp. were purified as described in Example 4.

The enzymatic assays were conducted in a total reaction volume of 0.3 ml.

The standard reaction mixture contained:

50 mM Tris-HCl pH 7.5

10 mM 3-methylcrotonyl-CoA

10 mM MgCl₂

10 mM NaCl

10 mM potassium phosphate buffer pH 7.5.

10 mM ADP

0.02 mg/ml purified phosphotransbutyrylase from B. subtilis

0.02 mg/ml purified butyrate kinase from Geobacillus sp.

1 mg/ml purified UbiD from S. cerevisiae

Catalysis was conducted at 30° C. during 18 h.

A series of control assays were performed in parallel in which either no UbiD protein (control A) or phosphotransbutyrylase (control B) or butyrate kinase (control C) were added or no substrate was added (control D). After the incubation period, the isobutene produced in the headspace was analysed by Gas Chromatography (GC) equipped Flame Ionization Detector (FID). One ml of the headspace gas phase was separated and analysed according to the method described in Example 2. An overlay of typical chromatogram obtained for the whole enzymatic assay, and the corresponding controls is shown on FIG. 39.

The highest production of isobutene was observed in the assay comprised phosphotransbutyrylase, butyrate kinase and UbiD protein. The control assay without phosphotransbutyrylase (control B) and control assay without butyrate kinase (control C) also showed a significant production of isobutene. These results could be explained by spontaneous hydrolysis of 3-methylcrotonyl-CoA into 3-methylcrotonic acid. Enzymatic production of isobutene from 3-methylcrotonyl-CoA can thus be achieved by three consecutive steps, through the formation of 3-methylcrotonyl phosphate and 3-methylcrotonic acid as intermediates.

### Example 8: In Vitro Screening of the UbiD Proteins for the Decarboxylation of 3-Methylcrotonic Acid into Isobutene

Several genes coding for UbiD protein were codon optimized for the expression in E. coli and synthesized by GeneArt® (Thermofisher). The corresponding enzymes were purified according to the procedure described in Example 1. The list of the studied enzymes is shown in Table G.

Enzymatic assays were carried out in 2 ml glass vials (Interchim) under the following conditions:

50 mM Tris-HCl buffer pH 7.5

20 mM NaCl

10 mM MgCl2

1 mM DTT

50 mM 3-methylcrotonic acid

1 mg/ml purified UbiD protein

100 μl lysate contained UbiX protein from E. coli

Total volume of the assays were 300 μl.

A series of control assays were performed in parallel, in which either no UbiD protein was added, or no enzymes were added (Table G).

The vials were sealed and incubated for 60 min at 30° C. The assays were stopped by incubating for 2 min at 80° C. and the isobutene formed in the reaction headspace was analysed by Gas Chromatography (GC) equipped with Flame Ionization Detector (FID), according to the procedure described in Example 2.

The results of the GC analysis are shown in Table G. No isobutene production was observed in control reactions. These results show that all the UbiD proteins, studied under the conditions of this screening assay, were able to perform the decarboxylation of 3-methylcrotonic acid into isobutene in presence of E. coli cell lysate contained UbiX protein.

TABLE G

| | | Isobutene |
|---|---|---|
| | | produced, |
| Candidate UbiD protein | Assay composition | arbitrary units |
| Saccharomyces cerevisae | UbiD protein alone | 9 |
| (Uniprot Accession | UbiD protein + Cell lysate | 945 |
| Number: Q03034) | contained UbiX protein | |
| Sphaerulina musiva (Uniprot | UbiD protein alone | 70 |
| Accession Number: M3DF95) | UbiD protein + Cell lysate | 3430 |
| | contained UbiX protein | |
| Penicillium roqueforti (Uniprot | UbiD protein alone | 34 |
| Accession Number: W6QKP7) | UbiD protein + Cell lysate | 1890 |
| | contained UbiX protein | |
| Hypocrea atroviridis (Uniprot | UbiD protein alone | 60 |
| Accession Number: G9NLP8) | UbiD protein + Cell lysate | 5200 |
| | contained UbiX protein | |
| Fusarium oxysporum sp. | UbiD protein alone | 13 |
| lycopersici (Uniprot Accession | UbiD protein + Cell lysate | 1390 |
| Number: W9LTH3) | contained UbiX protein | |
| Saccharomyces kudriavzevii | UbiD protein alone | 10 |
| (Uniprot Accession Number: | UbiD protein + Cell lysate | 920 |
| J8TRN5) | contained UbiX protein | |

| | |
|---|---|
| «No UbiD control»: Cell lysate contained UbiX protein alone | 0 |
| Control without any enzymes | 0 |

### Example 9: Conversion of 3-Methylcrotonyl-CoA and Acetate into 3-Methylcrotonic Acid and Acetyl-CoA Catalysed by Coenzyme A Transferase from Megasphaera Sp.

The enzyme was produced and purified according to the procedure described in Example 1.

The enzymatic assays were conducted in a total reaction volume of 0.2 ml

The standard reaction mixture contained:

50 mM Tris-HCl buffer pH 7.5

5 mM 3-methylcrotonyl-CoA

10 mM sodium acetate

10 mM MgCl2

10 mM NaCl

3 mg/ml purified CoA-transferase from Megasphaera sp. (Uniprot Accession Number: S7HFR5).

Control assays were performed in which either no enzyme was added, or no 3-methylcrotonyl-CoA was added. The assays were incubated for 6 h at 30° C. The assays were stopped by adding 100 μl MeCN in the medium. The samples were centrifuged, filtered through a 0.22 μm filter and the clarified supernatants were transferred into a clean vial for the HPLC-based analysis.

HPLC analysis was performed using 1260 Inifinity LC System (Agilent), equipped with a column heating module and UV detector (260 nm). 5 μl of samples were separated on Zorbax SB-Aq column (250×4.6 mm, 5 μm particle size, column temp.30° C.), with a mobile phase flow rate of 1.5 ml/min. The separation was performed using mixed A (H₂O containing 8.4 mM sulfuric acid) and B (acetonitrile) solutions in a linear gradient (0% B at initial time 0 min→70% B at 8 min). In these conditions, the retention time of 3-methylcrotonyl-CoA, 3-methylcrotonic acid and acetyl-CoA were 5.22 min, 5.70 min and 4.25 min, respectively.

Significant amounts of acetyl-CoA and 3-methylcrotonic acid were observed in the enzyme assay while none of the two compounds was not observed in control Significant amounts of acetyl-CoA and 3-methylcrotonic acid were observed in the enzyme assay while none of these two compounds was formed in control assays. Typical chromatograms for enzymatic and control assays are shown on FIG. 40.

### Example 10: Enzymatic Decarboxylation of 3-Methylcrotonic Acid into Isobutene Catalyzed in the Presence of a Lysate Containing UbiX Protein and with Purified Decarboxylase

0.5 M stock solution of 3-methylcrotonic acid was prepared in water and adjusted to pH 7.0 with 10 M solution of NaOH.

Proteins encoded by the aroY gene and one protein annotated as UbiD protein were produced according to the procedure described in Example 1.

Enzymatic assays were carried out in 2 ml glass vials (Interchim) under the following conditions:

50 mM potassium phosphate buffer pH 7.5

20 mM NaCl

10 mM MgCl2

5 mM DTT

50 mM 3-methylcrotonic acid

1 mg/ml purified AroY or UbiD protein

50 μl lysate contained UbiX protein

Total volume of the assays were 300 μl.

A series of control assays were performed in parallel (Table H).

The vials were sealed and incubated for 120 min at 30° C. The assays were stopped by incubating for 2 min at 80° C. and the isobutene formed in the reaction headspace was analysed by Gas Chromatography (GC) equipped with Flame Ionization Detector (FID).

For the GC analysis, one ml of the headspace gas was separated in a Bruker GC-450 system equipped with a GS-alumina column (30 m×0.53 mm) (Agilent) using isothermal mode at 130° C. Nitrogen was used as carrier gas with a flow rate of 6 ml/min.

The enzymatic reaction product was identified by comparison with an isobutene standard. Under these GC conditions, the retention time of isobutene was 2.42 min.

A significant production of isobutene from 3-methylcrotonic acid was observed in the combined assays (AroY or UbiD protein+UbiX protein). Incubation of lysate containing UbiX protein alone did not result in isobutene production. These data indicate that the proteins encoded by aroY gene in association with UbiX protein can catalyze the decarboxylation of 3-methylcrotonic acid into isobutene.

TABLE H

| | Isobutene production, |
|---|---|
| Assay composition | arbitrary units |
| AroY protein from K. pneumoniae | 10.5 |
| (Uniprot Acession Number: B9A9M6) + | |
| lysate contained UbiX protein from | |
| E. coli + substrate | |
| AroY protein from K. pneumoniae | 0 |
| (Uniprot Acession Number: B9A9M6) + | |
| substrate | |
| UbiD protein from E. cloacae (Uniprot | 8 |
| Acession Number: V3DX94) + lysate, | |
| contained UbiX protein from E. coli + | |
| substrate | |
| UbiD protein from E. cloacae (Uniprot | 0 |
| Acession Number: V3DX94) + substrate | |
| AroY protein from Leptolyngbya sp. | 5.5 |
| (Uniprot Acession Number: | |
| A0A0S3U6D8) +lysate, contained UbiX | |
| protein from E. coli + substrate | |
| AroY protein from Leptolyngbya sp. | 0 |
| (Uniprot Acession Number: | |
| A0A0S3U6D8) + substrate | |
| AroY protein from Phascolarctobacterium | 5.5 |
| sp. (Uniprot Acession Number: R6I1V6) + | |
| lysate, contained UbiX protein from E. | |
| coli + substrate | |
| AroY protein from Phascolarctobacterium | 0 |
| sp. (Uniprot Acession Number: R6I1V6) + | |
| substrate | |
| Lysate contained UbiX protein from E. | 0 |
| coli + substrate | |

### Example 11: Enzyme-Catalyzed Dehydration of 3-Hydroxy-3-Methylglutaryl-CoA into 3-Methylglutaconyl-CoA

The genes coding for 3-hydroxyacyl-CoA dehydratases (also termed enoyl-CoA hydratases, abbreviated in the following by ECH) (Table 1) were synthesized and the corresponding enzymes were further produced according to the procedure described in Example 1. Stock solution of 20 mM 3-hydroxy-3-methylglutaryl-CoA (HMG-CoA) was prepared in water. The enzymatic assays were conducted in total volume of 0.2 ml in the following conditions:
50 mM Tris-HCl buffer pH 7.5
100 mM NaCl
2 mM of 3-hydroxy-3-methylglutaryl-CoA (HMG-CoA)
0.1 mg/ml purified 3-hydroxyacyl-CoA dehydratase.

Enzymatic assays were started by adding the 20 μl of 20 mM substrate, were run for 10 min at 30° C. run for and stopped by adding 100 μL of acetonitrile in the reaction medium. All the enzymatic assays were performed in duplicate. The samples were then centrifuged, filtered through a 0.22 μm filter and the clarified supernatants were transferred into a clean vial for HPLC based analysis.

The analysis was performed using 1260 Inifinity LC System (Agilent), equipped with column heating module and UV detector (260 nm). 5 μl of samples were separated on Zorbax SB-Aq column (250×4.6 mm, 5 μm particle size, column temp.30° C.), with a mobile phase flow rate of 1.5 ml/min. The separation was performed using mixed A (H₂O containing 8.4 mM sulfuric acid) and B (acetonitrile) solutions in a linear gradient (0% B at initial time 0 min→70% B at 8 min). In these conditions, the retention time of HMG-CoA, 3-methylglutaconyl-CoA (MG-CoA) and free coenzyme A were respectively 4.26 min, 4.76 min and 3.96 min. FIG. 41 shows 3-methylglutaconyl-CoA (MG-CoA) peak areas obtained from the HPLC-based analysis.

TABLE I

| Enzyme's abbreviation | Source and Uniprot Accession Numbers |
|---|---|
| LiuC | 3-hydroxybutyryl-CoA dehydratase |
| | from Myxococcus xanthus |
| | (Q1D5Y4) |
| ECH Um | Putative enoyl-CoA hydratase from |
| | Ustilago maydis |
| | (Q4PEN0) |
| ECH Bs | Methylglutaconyl-CoA hydratase |
| | from Bacillus sp. |
| | GeD10 |
| | (N1LWG2) |
| ECH LI | Methylglutaconyl-CoA hydratase from |
| | Labilithrix luteola |
| | (A0A0K1PN19) |
| ECH Pa | Putative isohexenylglutaconyl-CoA |
| | hydratase from |
| | Pseudomonas aeruginosa |
| | (Q9HZV7) |
| ECH Ms | Enoyl-CoA hydratase from |
| | Marinobacter santoriniensis |
| | (M7CV63) |
| ECH Ab | Enoyl-CoA hydratase from |
| | Acinetobacter baumannii |
| | (A0A0D5YDD4) |
| ECH Pp | Isohexenylglutaconyl-CoA hydratase from |
| | Pseudomonas pseudoalcaligenes |
| | (L8MQT6) |

### Example 12: Microorganism for the Production of Isobutene from Acetyl-CoA Via 3-Methylcrotonic Acid

This example shows the direct production of isobutene by a recombinant E. coli strain which expresses exogenous genes, thereby constituting the isobutene pathway.

Like most organisms, E. coli converts glucose to acetyl-CoA. The enzymes used in this study to convert acetyl-CoA into isobutene via 3-methylcrotonic acid (FIG. 42) are summarized in Table J.

TABLE J

| | | | | Uniprot |
|---|---|---|---|---|
| | | Gene | | Accession |
| Step | Enzyme | abbreviation | NCB reference | number |
| XIII | Acetyl-CoA | thIA | WP_ | P45359 |
| | transferase from | | 010966157.1 | |
| | Clostridium | | | |
| | acetobulyticum | | | |
| | (ThIA) | | | |
| IX | Hydroxymethylglutaryl- | mvaS | WP_ | Q9FD71 |
| | CoA synthase from | | 002357756.1 | |
| | Enterococcus | | | |
| | faecalis (MvaS) | | | |
| VIII | Isohexenylglutaconyl- | ppKF707_ | WP_ | L8MQT6 |
| | CoA hydratase from | 3831 | 004422368.1 | |
| | Pseudomonas | | | |
| | pseudoalcaligenes | | | |
| | KF707 (ECH) | | | |
| VII | Glutaconate CoA- | MXAN_ | WP_ | Q1D4I3 |
| | transferase from | 4264 | 011554268.1 | |
| | Myxococcus xanthus | MXAN_ | WP_ | Q1D4I4 |
| | (AibA/B) | 4265 | 011554267.1 | |
| VI | Acyl-CoA | tesB | WP_ | P0AGG2 |
| | thioesterase 2 from | | 000075876.1 | |
| | Escherichia coli (TesB) | | | |
| I | Ferulic acid | FDC1 | XP_ | G9NLP8 |
| | decarboxylase | | 013946967.1 | |
| | from Hypocrea | | | |
| | atroviridis (UbiD) | | | |
| | Flavin prenyl | ubiX | WP_ | P0AG03 |
| | transferase from | | 000825700.1 | |
| | Escherichia coli (UbiX) | | | |

Expression of Isobutene Biosynthetic Pathway in E. coli

All the corresponding genes were codon optimized for the expression in E. coli and synthesized by GeneArt® (Life Technologies), except the gene encoding for UbiX protein which was directly amplified from the genomic DNA of E. coli MG1655. The modified version of pUC18 (New England Biolabs), containing a modified Multiple Cloning Site (pUC18 MCS) (WO 2013/007786), was used for the overexpression of the ubiX gene. This plasmid conferred ampicillin resistance to the recombinant strain. The constructed vector was named pGB 5796 and the corresponding nucleotidic sequence is indicated in Table K.

TABLE K

| Plasmid | |
|---|---|
| name | Nucleotidic sequence |
| pGB 5796 | tcgcgcgtttcggtgatgacggtgaaaacctctgacacatgcagctcccggagac |
| | ggtcacagcttgtctgtaagcggatgccgggagcagacaagcccgtcagggcgcg |
| | tcagcgggtgttggcgggtgtcggggctggcttaactatgcggcatcagagcaga |
| | ttgtactgagagtgcaccatatgcggtgtgaaataccgcacagatgcgtaaggag |
| | aaaataccgcatcaggcgccattcgccattcaggctgcgcaactgttgggaaggg |
| | cgatcggtgcgggcctcttcgctattacgccagctggcgaaagggggatgtgctg |
| | caaggcgattaagttgggtaacgccagggttttcccagtcacgacgttgtaaaac |
| | gacggccagtgccAAGCTTGCGGCCGCGGGGTTAATTAATTTCTCCTCTTTAATA |
| | AAGCAAATAAATTTTTTATGATTTGTTTAAACCTAGGCATGCCtctagaTTAtta |
| | TGCGCCCTGCCAGCGGGCAAAGAGATCTTCAGGAAGGGTTATCGCAAACTGGTCA |
| | AGAACACGATTAACCGTCTGATTTATCACATCATCAAGGGATTGCGGGCGATGAT |
| | AAAACGCCGGAACGGGAGGCATAATCACCGCACCGATTTCTGCCGCCTGAGTCAT |
| | TAAACGCAGATGGCCTAAGTGCAATGGTGTTTCACGCACGCAGAGCACCAACGGG |
| | CGACGCTCTTTCAGCACCACATCTGCCGCACGGGTCAGTAAGCCATCAGTATAGC |
| | TATGGACAATGCCGGAAAGGGTTTTGATTGAACAGGGTAAAATCACCATCCCCAG |
| | CGTCTGGAAAGAACCGGAAGAGATGCTGGCGGCAATATCGCGCGCATCGTGCGTG |
| | ACATCGGCTAATGCCTGCACTTCGCGCAGAGAAAAATCCGTTTCGAGGGATAAGG |
| | TCTGGCGCGCTGCCTGGCTCATCACCAGATGCGTTTCGATATCTGTGACATCGCG |
| | CAGAACCTGTAATAAGCGCACGCCATAAATCGCGCCGCTGGCACCGCTGATGCCT |
| | ACAATGAGTCGTTTcatAAAAAAAATGTATATCTCCTTCggtaccGAGCTCGAAC |
| | CTGCAGGAATTCgtaatcatggtcatagctgtttcctgtgtgaaattgttatccg |
| | ctcacaattccacacaacatacgagccggaagcataaagtgtaaagcctggggtg |
| | cctaatgagtgagctaactcacattaattgcgttgcgctcactgcccgctttcca |
| | gtcgggaaacctgtcgtgccagctgcattaatgaatcggccaacgcgcggggaga |
| | ggcggtttgcgtattgggcgctcttccgcttcctcgctcactgactcgctgcgct |
| | cggtcgttcggctgcggcgagcggtatcagctcactcaaaggcggtaatacggtt |
| | atccacagaatcaggggataacgcaggaaagaacatgtgagcaaaaggccagcaa |
| | aaggccaggaaccgtaaaaaggccgcgttgctggcgtttttccataggctccgcc |
| | cccctgacgagcatcacaaaaatcgacgctcaagtcagaggtggcgaaacccgac |
| | aggactataaagataccaggcgtttccccctggaagctccctcgtgcgctctcct |
| | gttccgaccctgccgcttaccggatacctgtccgcctttctcccttcgggaagcg |
| | tggcgctttctcatagctcacgctgtaggtatctcagttcggtgtaggtcgttcg |
| | ctccaagctgggctgtgtgcacgaaccccccgttcagcccgaccgctgcgcctta |
| | tccggtaactatcgtcttgagtccaacccggtaagacacgacttatcgccactgg |
| | cagcagccactggtaacaggattagcagagcgaggtatgtaggcggtgctacaga |
| | gttcttgaagtggtggcctaactacggctacactagaaggacagtatttggtatc |
| | tgcgctctgctgaagccagttaccttcggaaaaagagttggtagctcttgatccg |
| | gcaaacaaaccaccgctggtagcggtggtttttttgtttgcaagcagcagattac |
| | gcgcagaaaaaaaggatctcaagaagatcctttgatcttttctacggggtctgac |
| | gctcagtggaacgaaaactcacgttaagggattttggtcatgagattatcaaaaa |
| | ggatcttcacctagatccttttaaattaaaaatgaagttttaaatcaatctaaag |
| | tatatatgagtaaacttggtctgacagttaccaatgcttaatcagtgaggcacct |
| | atctcagcgatctgtctatttcgttcatccatagttgcctgactccccgtcgtgt |
| | agataactacgatacgggagggcttaccatctggccccagtgctgcaatgatacc |
| | gcgagacccacgctcaccggctccagatttatcagcaataaaccagccagccgga |
| | agggccgagcgcagaagtggtcctgcaactttatccgcctccatccagtctatta |
| | attgttgccgggaagctagagtaagtagttcgccagttaatagtttgcgcaacgt |
| | tgttgccattgctacaggcatcgtggtgtcacgctcgtcgtttggtatggcttca |
| | ttcagctccggttcccaacgatcaaggcgagttacatgatcccccatgttgtgca |
| | aaaaagcggttagctccttcggtcctccgatcgttgtcagaagtaagttggccgc |
| | agtgttatcactcatggttatggcagcactgcataattctcttactgtcatgcca |
| | tccgtaagatgcttttctgtgactggtgagtactcaaccaagtcattctgagaat |
| | agtgtatgcggcgaccgagttgctcttgcccggcgtcaatacgggataataccgc |
| | gccacatagcagaactttaaaagtgctcatcattggaaaacgttcttcggggcga |
| | aaactctcaaggatcttaccgctgttgagatccagttcgatgtaacccactcgtg |
| | cacccaactgatcttcagcatcttttactttcaccagcgtttctgggtgagcaaa |
| | aacaggaaggcaaaatgccgcaaaaaagggaataagggcgacacggaaatgttga |
| | atactcatactcttcctttttcaatattattgaagcatttatcagggttattgtc |
| | tcatgagcggatacatatttgaatgtatttagaaaaataaacaaataggggttcc |
| | gcgcacatttccccgaaaagtgccacctgacgtctaagaaaccattattatcatg |
| | acattaacctataaaaataggcgtatcacgaggccctttcgtc |
| | (SEQ ID NO: 93) |

An expression vector containing the origin of replication pSC was used for the expression of the genes: thlA, MvaS, ppKF707_3831, MXAN_4264/MXAN_4265, FDC1. This plasmid conferred spectinomycin resistance to the recombinant strain. The constructing vector was named pGB 5771 and the corresponding nucleotidic sequence is indicated in Table L.

TABLE L

| Plasmid | |
|---|---|
| name | Nucleotidic sequence |
| pGB 5771 | ctcactactttagtcagttccgcagtattacaaaaggatgtcgcaaacgctgttt |
| | gctcctctacaaaacagaccttaaaaccctaaaggcttaagtagcaccctcgcaa |
| | gctcgggcaaatcgctgaatattccttttgtctccgaccatcaggcacctgagtc |
| | gctgtctttttcgtgacattcagttcgctgcgctcacggctctggcagtgaatgg |
| | gggtaaatggcactacaggcgccttttatggattcatgcaaggaaactacccata |
| | atacaagaaaagcccgtcacgcttctcagggcgttttatggcgggtctgctatgt |
| | ggtgctatctgactttttgctgttcagcagttcctgccctctgattttccagtct |
| | gaccctagtcaaggccttaagtgagtcgtattacggactggccgtcgttttacaa |
| | cgtcgtgactgggaaaaccctggcgttacccaacttaatcgccttgcagcacatc |
| | cccctttcgccagctggcgtaatagcgaagaggcccgcaccgatcgcccttccca |
| | acagttgcgcagcctgaatggcgaatggcgcctgatgcggtattttctccttacg |
| | catctgtgcggtatttcacaccgCCCGGGGAACTATAgtttaaacTTTTCAATGA |
| | ATTCATTTaaGCGGCCGCatcaatTCTAGAatttaaatagtcaaaagcctccgac |
| | cggaggcttttgactgACCTATTGACAATTAAAGGCTAAAATGCTATAATTCCAC |
| | taatagaaataattttgtttaactttaggtctctatcgtaaGAAGGAGATATatg |
| | aaagaagtggtgattgccagcgcagttcgtaccgcaattggtagctatggtaaaa |
| | gcctgaaagatgttccggcagttgatctgggtgcaaccgcaattaaagaagcagt |
| | taaaaaagccggtattaaaccggaagatgtgaacgaagttattctgggtaatgtt |
| | ctgcaagcaggtctgggtcagaatccggcacgtcaggcctcgtttaaagcaggtc |
| | tgccggttgaaattccggcaatgaccattaacaaagtttgtggtagcggtctgcg |
| | taccgttagcctggcagcacagattatcaaagccggtgatgcagatgttattatt |
| | gccggtggtatggaaaatatgagccgtgcaccgtatctggcaaataatgcacgtt |
| | ggggttatcgtatgggtaatgccaaatttgtggatgagatgattaccgatggtct |
| | gtgggatgcctttaatgattatcacatgggtattaccgcagagaatattgcagaa |
| | cgttggaatattagccgtgaagaacaggatgaatttgcactggcaagccagaaaa |
| | aagcagaagaagcaattaaaagcggtcagttcaaagatgaaattgtgccggttgt |
| | tatcaaaggtcgtaaaggtgaaaccgttgttgataccgatgaacatccgcgtttt |
| | ggtagcaccattgaaggtctggcaaaactgaaaccggcattcaaaaaagatggca |
| | ccgttaccgcaggtaatgcaagcggtctgaatgattgtgcagcagttctggttat |
| | tatgagcgcagaaaaagcaaaagaactgggtgttaaaccgctggcaaaaattgtg |
| | agctatggtagtgccggtgttgatccggcaattatgggttatggtccgttttatg |
| | caaccaaagcagcaattgaaaaagcaggttggaccgttgatgaactggatctgat |
| | tgaaagcaatgaagcatttgcagcacagagcctggcagttgcaaaagacctgaaa |
| | ttcgatatgaataaagtgaatgtgaatggcggtgcaattgccctgggtcatccga |
| | ttggtgcaagcggtgcacgtattctggttaccctggttcatgcaatgcagaaacg |
| | tgatgcaaaaaaaggtctggccaccctgtgtattggtggtggtcagggcaccgca |
| | attctgctggaaaaatgctaataagcttGAAGGAGATATAATGACCATTGGTATT |
| | GATAAAATCAGCTTTTTCGTGCCTCCGTACTATATTGATATGACCGCACTGGCCG |
| | AAGCACGTAATGTTGATCCGGGTAAATTTCATATTGGTATTGGTCAGGATCAGAT |
| | GGCCGTTAATCCGATTAGCCAGGATATTGTTACCTTTGCAGCAAATGCAGCAGAA |
| | GCAATTCTGACCAAAGAAGATAAAGAGGCCATTGATATGGTTATTGTTGGCACCG |
| | AAAGCAGCATTGATGAAAGCAAAGCAGCAGCAGTTGTTCTGCATCGTCTGATGGG |
| | TATTCAGCCGTTTGCACGTAGCTTTGAAATTAAAGAAGCATGTTACGGAGCAACC |
| | GCAGGTCTGCAACTGGCAAAAAATCATGTTGCACTGCATCCGGATAAAAAAGTTC |
| | TGGTTGTTGCAGCAGATATTGCCAAATATGGTCTGAATAGCGGTGGTGAACCGAC |
| | CCAGGGTGCCGGTGCAGTTGCAATGCTGGTTGCAAGCGAACCGCGTATTCTGGCA |
| | CTGAAAGAAGATAATGTTATGCTGACCCAGGATATTTATGATTTTTGGCGTCCGA |
| | CCGGTCATCCGTATCCGATGGTTGATGGTCCGCTGAGCAATGAAACCTATATTCA |
| | GAGCTTTGCACAGGTGTGGGATGAACATAAAAAACGTACCGGTCTGGATTTCGCA |
| | GATTATGATGCACTGGCATTTCATATCCCGTATACCAAAATGGGTAAAAAAGCAC |
| | TGCTGGCCAAAATTAGCGATCAGACCGAAGCCGAACAAGAACGCATTCTGGCACG |
| | TTATGAAGAAAGCATTGTTTATAGCCGTCGTGTGGGTAATCTGTATACCGGTAGC |
| | CTGTATCTGGGTCTGATTAGCCTGCTGGAAAATGCAACCACCCTGACCGCAGGTA |
| | ATCAGATTGGTCTGTTTAGCTATGGTAGCGGTGCCGTTGCAGAATTTTTCACAGG |
| | TGAACTGGTTGCAGGTTATCAGAATCATCTGCAAAAAGAAACCCATCTGGCACTG |
| | CTGGATAATCGTACCGAACTGAGCATTGCAGAATATGAAGCAATGTTTGCAGAAA |
| | CCCTGGATACCGATATTGATCAGACCCTGGAAGATGAACTGAAATATAGCATTAG |
| | CGCCATTAATAACACCGTGCGTAGCTATCGTAACTAATAAggtaGAAGGAGATAT |
| | ACATatgagtcaggcgctaaaaaatttactgacattgttaaatctggaaaaaatt |
| | gaggaaggactctttcgcggccagagtgaagatttaggtttacgccaggtgtttg |
| | gcggccaggtcgtgggtcaggccttgtatgctgcaaaagagacGgtccctgaaga |
| | Acggctggtacattcgtttcacagctactttcttcgccctggcgatagtaagaag |
| | ccgattatttatgatgtcgaaacgctgcgtgacggtaacagcttcagcgcccgcc |
| | gggttgctgctattcaaaacggcaaaccgattttttatatgactgcctctttcca |
| | ggcaccagaagcgggtttcgaacatcaaaaaacaatgccgtccgcgccagcgcct |
| | gatggcctcccttcggaaacgcaaatcgcccaatcgctggcgcacctgctgccgc |
| | cagtgctgaaagataaattcatctgcgatcgtccgctggaagtccgtccggtgga |
| | gtttcataacccactgaaaggtcacgtcgcagaaccacatcgtcaggtgtggatT |
| | cgcgcaaatggtagcgtgccggatgacctgcgcgttcatcagtatctgctcggtt |
| | acgcttctgatcttaacttcctgccggtagctctacagccgcacggcatcggttt |
| | tctcgaaccggggattcagattgccaccattgaccattccatgtggttccatcgc |
| | ccgtttaatttgaatgaatggctgctgtatagcgtggagagcacctcggcgtcca |
| | gcgcacgtggctttgtgcgcggtgagttttatacccaagacggcgtactggttgc |
| | ctcgaccgttcaggaaggggtgatgcgtaatcacaattaataagaacGAAGGAGA |
| | TATAAtgAAAACCGCACGTTGGTGTAGCCTGGAAGAAGCAGTTGCAAGCATTCCG |
| | GATGGTGCAAGCCTGGCAACCGGTGGTTTTATGCTGGGTCGTGCACCGATGGCAC |
| | TGGTTATGGAACTGATTGCACAGGGTAAACGTGATCTGGGTCTGATTAGCCTGCC |
| | GAATCCGCTGCCAGCAGAATTTCTGGTTGCCGGTGGTTGTCTGGCTCGTCTGGAA |
| | ATTGCATTTGGTGCACTGAGTCTGCAAGGTCGTGTTCGTCCGATGCCGTGTCTGA |
| | AACGTGCAATGGAACAGGGCACCCTGGCATGGCGTGAACATGATGGTTATCGTGT |
| | TGTTCAGCGTCTGCGTGCAGCAAGCATGGGTCTGCCGTTTATTCCGGCACCGGAT |
| | GCAGATGTTAGCGGTCTGGCACGTACCGAACCGCCTCCGACCGTTGAAGATCCGT |
| | TTACCGGTCTGCGTGTTGCAGTTGAACCGGCATTTTATCCGGATGTTGCACTGCT |
| | GCACGCACGTGCAGCCGATGAACGTGGTAATCTGTATATGGAAGATCCGACCACC |
| | GATCTGCTGGTTGCGGGTGCAGCAAAACGTGTTATTGCAACCGTTGAAGAACGTG |
| | TTGCAAAACTGCCTCGTGCAACCCTGCCTGGTTTTCAGGTTGATCGTATTGTTCT |
| | GGCACCGGGTGGTGCACTGCCGACCGGTTGTGCAGGTCTGTATCCGCATGATGAT |
| | GAAATGCTGGCACGTTATCTGAGCCTGGCAGAAACCGGTCGTGAAGCCGAATTTC |
| | TGGAAACCCTGCTGACCCGTCGTGCAGCATAATGAggatccGAAGGAGATATACA |
| | TAtgAGCGCAACCCTGGATATTACACCGGCAGAAACCGTTGTTAGCCTGCTGGCA |
| | CGTCAGATTGATGATGGTGGTGTTGTTGCAACCGGTGTTGCAAGTCCGCTGGCAA |
| | TTCTGGCCATTGCAGTTGCACGTGCCACCCATGCACCGGATCTGACCTATCTGGC |
| | ATGTGTTGGTAGCCTGGACCCGGAAATTCCGACCCTGCTGCCGAGCAGCGAAGAC |
| | CTGGGTTATCTGGATGGTCGTAGCGCAGAAATTACCATTCCGGACCTGTTTGATC |
| | ATGCACGTCGTGGTCGTGTTGATACCGTTTTTTTTGGTGCAGCCGAAGTTGATGC |
| | CGAAGGTCGTACCAATATGACCGCAAGCGGTAGTCTGGATAAACCGCGTACCAAA |
| | TTTCCGGGTGTTGCCGGTGCAGCCACCCTGCGTCAGTGGGTTCGTCGTCCGGTTC |
| | TGCTGGTTCCGCGTCAGAGCCGTCGTAATCTGGTTCCGGAAGTTCAGGTTGCAAC |
| | CACCCGTGATCCGCGTCGTCCGGTGACCCTGATTAGCGATCTGGGTGTTTTTGAA |
| | CTGGGTGCAAGCGGTGCACGTCTGCTGGCACGCCATCCGTGGGCAAGCGAAGAAC |
| | ATATTGCAGAACGTACCGGTTTTGCATTTCAGGTTAGCGAAGCACTGAGCGTTAC |
| | CAGCCTGCCGGATGCACGTACCGTTGCAGCAATTCGTGCAATTGATCCGCATGGC |
| | TATCGTGATGCACTGGTTGGTGCATAATTAgtcagaaggagatataCATATGAGC |
| | CTGCCGCATTGTGAAACCCTGCTGCTGGAACCGATTGAAGGTGTTCTGCGTATTA |
| | CCCTGAATCGTCCGCAGAGCCGTAATGCAATGAGCCTGGCAATGGTTGGTGAACT |
| | GCGTGCAGTTCTGGCAGCAGTTCGTGATGATCGTAGCGTTCGTGCACTGGTTCTG |
| | CGTGGTGCAGATGGTCATTTTTGTGCCGGTGGTGATATTAAAGATATGGCAGGCG |
| | CACGTGCAGCCGGTGCAGAAGCATATCGTACACTGAATCGTGCATTTGGTAGCCT |
| | GCTGGAAGAAGCACAGGCAGCACCGCAGCTGCTGGTTGCACTGGTTGAAGGTGCC |
| | GTTCTGGGTGGTGGTTTTGGTCTGGCATGTGTTAGTGATGTTGCAATTGCAGCAG |
| | CAGATGCACAGTTTGGTCTGCCGGAAACCAGCCTGGGTATTCTGCCTGCACAGAT |
| | TGCACCGTTTGTTGTTCGTCGTATTGGTCTGACCCAGGCACGTCGTCTGGCACTG |
| | ACCGCAGCACGTTTTGATGGTCGTGAAGCACTGCGTCTGGGTCTGGTTCATTTTT |
| | GTGAAGCAGATGCAGATGCACTGGAACAGCGTCTGGAAGAAACCCTGGAACAGCT |
| | GCGTCGTTGTGCACCGAATGCAAATGCAGCAACCAAAGCACTGCTGCTGGCAAGC |
| | GAAAGCGGTGAACTGGGTGCACTGCTGGATGATGCAGCACGTCAGTTTGCCGAAG |
| | CAGTTGGTGGTGCAGAAGGTAGCGAAGGCACCCTGGCATTTGTTCAGAAACGTAA |
| | ACCGGTTTGGGCACAGTAATAAtgaaagagaccagcctgatacagattaaatcag |
| | aacgcagaagcggtctgataaaacagaatttgcctggcggcagtagcgcggtggt |
| | cccacctgaccccatgccgaactcagaagtgaaacgccgtagcgccgatggtagt |
| | gtggggtcaccccatgcgagagtagggaactgccaggcatcaaataaaacgaaag |
| | gctcagtcgaaagactgggcctttcgttttatctgttgtttgtcggtgaactACT |
| | AGAatttaaatagtcaaaagcctccgaccggaggcttttgactgACCTATTGACA |
| | ATTAAAGGCTAAAATGCTATAATTCCACtaatagaaataattttgtttaacttta |
| | ggtctctatcgaccataaTTAATTAActttaagaaggagatataCATatgAGCAG |
| | CACCACCTATAAAAGCGAAGCATTTGATCCGGAACCGCCTCATCTGAGCTTTCGT |
| | AGCTTTGTTGAAGCACTGCGTCAGGATAATGATCTGGTGGATATTAATGAACCGG |
| | TTGATCCGGATCTGGAAGCAGCAGCAATTACCCGTCTGGTTTGTGAAACCGATGA |
| | TAAAGCACCGCTGTTTAATAACGTGATTGGTGCAAAAGATGGTCTGTGGCGTATT |
| | CTGGGTGCACCGGCAAGCCTGCGTAGCAGCCCGAAAGAACGTTTTGGTCGTCTGG |
| | CACGTCATCTGGCACTGCCTCCGACCGCAAGCGCAAAAGATATTCTGGATAAAAT |
| | GCTGAGCGCCAATAGCATTCCGCCTATTGAACCGGTTATTGTTCCGACCGGTCCG |
| | GTTAAAGAAAATAGCATTGAAGGCGAAAACATTGATCTGGAAGCCCTGCCTGCAC |
| | CGATGGTTCATCAGAGTGATGGTGGCAAGTATATCCAGACCTATGGTATGCATGT |
| | TATCCAGAGTCCGGATGGTTGTTGGACCAATTGGAGCATTGCCCGTGCAATGGTT |
| | AGCGGTAAACGTACCCTGGCAGGTCTGGTTATTAGTCCGCAGCATATTCGTAAAA |
| | TTCAGGATCAGTGGCGTGCAATTGGTCAAGAAGAAATTCCTTGGGCACTGGCATT |
| | TGGTGTTCCGCCTACCGCAATTATGGCAAGCAGTATGCCGATTCCGGATGGTGTT |
| | AGCGAAGCAGGTTATGTTGGTGCAATTGCCGGTGAACCGATTAAACTGGTTAAAT |
| | GCGATACCAACAATCTGTATGTTCCGGCAAATAGCGAAATTGTTCTGGAAGGCAC |
| | CCTGAGCACCACCAAAATGGCACCGGAAGGTCCGTTTGGTGAAATGCATGGTTAT |
| | GTTTATCCGGGTGAAAGCCATCCGGGTCCGGTTTATACCGTTAACAAAATTACCT |
| | ATCGCAACAATGCAATTCTGCCGATGAGCGCATGTGGTCGTCTGACCGATGAAAC |
| | CCAGACCATGATTGGCACCCTGGCAGCAGCAGAAATTCGTCAGCTGTGTCAGGAT |
| | GCAGGTCTGCCGATTACCGATGCATTTGCACCGTTTGTTGGTCAGGCAACCTGGG |
| | TTGCACTGAAAGTTGATACCAAACGTCTGCGTGCAATGAAAACCAATGGTAAAGC |
| | ATTTGCAAAACGTGTTGGTGATGTTGTGTTTACCCAGAAACCGGGTTTTACCATT |
| | CATCGTCTGATTCTGGTTGGTGATGATATTGATGTGTATGACGATAAAGATGTGA |
| | TGTGGGCATTTACCACCCGTTGTCGTCCGGGTACAGATGAAGTTTTTTTTGATGA |
| | TGTTGTGGGCTTTCAGCTGATCCCGTATATGAGTCATGGTAATGCCGAAGCAATT |
| | AAAGGTGGTAAAGTTGTTAGTGATGCACTGCTGACCGCAGAATATACCACCGGTA |
| | AAGATTGGGAAAGCGCAGATTTCAAAAACAGCTATCCGAAAAGCATCCAGGATAA |
| | AGTTCTGAATAGCTGGGAACGCCTGGGTTTCAAAAAACTGGATTAATAACCATGG |
| | ttataagagagaccagcctGACTCCTGTTGATAGATCCAGTAATGACCTCAGAAC |
| | TCCATCTGGATTTGTTCAGAACGCTCGGTTGCCGCCGGGCGTTTTTTATTGGTGA |
| | GAATaactACTAGTtggcggGCGGCCGCttagctCTGCAGatgagaaattcttga |
| | agacgaaagggcctcgtgatacgcctatttttataggttaatgtcatgataataa |
| | tggtttAAGCTTcttagaataGCTCTTCTATGaggtggcacttttcggggaaaGA |
| | TATCcgcatatatggtgcactctcagtacaatctgctctgatgccgcatagttaa |
| | gccagtatacactccgctatcgctacgtgactgggtcatggctgcgccccgacac |
| | ccgccaacacccgctgacgcgccctgacgggcttgtctgctcccggcatccgctt |
| | acagacaagctgtgaccgtctccgggagctgcatgtgtcagaggttttcaccgtc |
| | atcaccgaaacgcgcgaggcagctgcggtaaagctcatcagcgtggtcgtgaagc |
| | gattcacagatgtctgcctgttcatcGGTACCtttcatgatatatctcccaattt |
| | gtgtagggcttattatgcacgcttaaaaataataaaagcagacttgacctgatag |
| | tttggctgtgagcaattatgtgcttagtgcatctaacgcttgagttaagccgcgc |
| | cgcgaagcggcgtcggcttgaacgaattgttagacattatttgccgactaccttg |
| | gtgatctcgcctttcacgtagtggacaaattcttccaactgatctgcgcgcgagg |
| | ccaagcgatcttcttcttgtccaagataagcctgtctagcttcaagtatgacggg |
| | ctgatactgggccggcaggcgctccattgcccagtcggcagcgacatccttcggc |
| | gcgattttgccggttactgcgctgtaccaaatgcgggacaacgtaagcactacat |
| | ttcgctcatcgccagcccagtcgggcggcgagttccatagcgttaaggtttcatt |
| | tagcgcctcaaatagatcctgttcaggaaccggatcaaagagttcctccgccgct |
| | ggacctaccaaggcaacgctatgttctcttgcttttgtcagcaagatagccagat |
| | caatgtcgatcgtggctggctcgaagatacctgcaagaatgtcattgcgctgcca |
| | ttctccaaattgcagttcgcgcttagctggataacgccacggaatgatgtcgtcg |
| | tgcacaacaatggtgacttctacagcgcggagaatctcgctctctccaggggaag |
| | ccgaagtttccaaaaggtcgttgatcaaagctcgccgcgttgtttcatcaagcct |
| | tacggtcaccgtaaccagcaaatcaatatcactgtgtggcttcaggccgccatcc |
| | actgcggagccgtacaaatgtacggccagcaacgtcggttcgagatggcgctcga |
| | tgacgccaactacctctgatagttgagtcgatacttcggcgatcaccgcttccct |
| | catgatgtttaactttgttttagggcgactgccctgctgcgtaacatcgttgctg |
| | ctccataacatcaaacatcgacccacggcgtaacgcgcttgctgcttggatgccc |
| | gaggcatagactgtaccccaaaaaaacagtcataacaagccatgaaaaccgccac |
| | GAGCTCctgtcagaccaagtttacgagctcgcttggactcctgttgatagatcca |
| | gtaatgacctcagaactccatctggatttgttcagaacgctcggttgccgccggg |
| | cgttttttattggtgagaatccaagcactagggacagtaagacgggtaagcctgt |
| | tgatgataccgctgccttactgggtgcattagccagtctgaatgacctgtcacgg |
| | gataatccgaagtggtcagactggaaaatcagagggcaggaactgctgaacagca |
| | aaaagtcagatagcaccacatagcagacccgccataaaacgccctgagaagcccg |
| | tgacgggcttttcttgtattatgggtagtttccttgcatgaatccataaaaggcg |
| | cctgtagtgccatttacccccattcactgccagagccgtgagcgcagcgaactga |
| | atgtcacgaaaaagacagcgactcaggtgcctgatggtcggagacaaaaggaata |
| | ttcagcgatttgcccgagcttgcgagggtgctacttaagcctttagggttttaag |
| | gtctgttttgtagaggagcaaacagcgtttgcgacatccttttgtaatactgcgg |
| | aactgactaaagtagtgagttatacacagggctgggatctattctttttatcttt |
| | ttttattctttctttattctataaattataaccacttgaatataaacaaaaaaaa |
| | cacacaaaggtctagcggaatttacagagggtctagcagaatttacaagttttcc |
| | agcaaaggtctagcagaatttacagatacccacaactcaaaggaaaaggacatgt |
| | aattatcattgactagcccatctcaattggtatagtgattaaaatcacctagacc |
| | aattgagatgtatgtctgaattagttgttttcaaagcaaatgaactagcgattag |
| | tcgctatgacttaacggagcatgaaaccaagctaattttatgctgtgtggcacta |
| | ctcaaccccacgattgaaaaccctacaaggaaagaacggacggtatcgttcactt |
| | ataaccaatacgctcagatgatgaacatcagtagggaaaatgcttatggtgtatt |
| | agctaaagcaaccagagagctgatgacgagaactgtggaaatcaggaatcctttg |
| | gttaaaggctttgagattttccagtggacaaactatgccaagttctcaagcgaaa |
| | aattagaattagtttttagtgaagagatattgccttatcttttccagttaaaaaa |
| | attcataaaatataatctggaacatgttaagtcttttgaaaacaaatactctatg |
| | aggatttatgagtggttattaaaagaactaacacaaaagaaaactcacaaggcaa |
| | atatagagattagccttgatgaatttaagttcatgttaatgcttgaaaataacta |
| | ccatgagtttaaaaggcttaaccaatgggttttgaaaccaataagtaaagattta |
| | aacacttacagcaatatgaaattggtggttgataagcgaggccgcccgactgata |
| | cgttgattttccaagttgaactagatagacaaatggatctcgtaaccgaacttga |
| | gaacaaccagataaaaatgaatggtgacaaaataccaacaaccattacatcagat |
| | tcctacctacgtaacggactaagaaaaacactacacgatgctttaactgcaaaaa |
| | ttcagctcaccagttttgaggcaaaatttttgagtgacatgcaaagtaagcatga |
| | tctcaatggttcgttctcatggctcacgcaaaaacaacgaaccacactagagaac |
| | atactggctaaatacggaaggatctgaggttcttatggctcttgtatctatcagt |
| | gaagcatcaagactaacaaacaaaagtagaacaactgttcaccgttagatatcaa |
| | agggaaaactgtccataagcacagatgaaaacggtgtaaaaaagatagatacatc |
| | agagcttttacgagtttttggtgcatttaaagctgttcaccatgaacagatcgac |
| | aatgtaacGCATGCaccgagcgcagcgagtcagtgagcgaggaagcggaacagcg |
| | cctg (SEQ ID NO: 94) |

These recombinant pGBE 5771 and pGBE5796 plasmids were verified by sequencing.

MG1655 E. coli strain was made electrocompetent and was transformed with pGBE5771 and pGBE5796 or with the corresponding empty vectors (pUC18 MGS and pGB2021) in order to create negative controls. The strains thus produced are summarized in Table M.

TABLE M

| Strain number | Vectors |
|---|---|
| Strain 1 (metabolic | pUC18_MCS + pGB 2021 |
| pathway-free control), | |
| containing the empty | |
| vectors. | |
| Strain 2, expressing only | pGB 5796 + pGB 2021 |
| UbiX protein | |
| Strain 3, expressing the | pUC18_MCS + PGB 5771 |
| whole metabolic pathway, | |
| without overexpression of | |
| UbiX protein on plasmid. | |
| Strain 4, expressing the | pGB 5796 + pGB 5771 |
| whole metabolic pathway, | |
| comprising | |
| overexpression of UbiX | |
| protein on plasmid. | |

The transformed cells were then plated on LB plates, supplied with ampicillin (100 μg/ml) and spectinomycin (100 μg/ml). Plates were incubated overnight at 30° C. Isolated colonies were used to inoculate 1.4 ml of ZYM-5052 auto-inducing media (Studier FW, Prot. Exp. Pur. 41, (2005), 207-234) supplemented with ampicillin, spectinomycin and 0.5 mM flavin mononucleotide. These cultures were grown for 16 h at 30° C. and 700 rpm shaking in 96 deep-well microplates. Then the cultures were centrifuged and the pellets were resuspended in 0.4 ml of MS medium (Richaud C., Mengin-Leucreulx D., Pochet S., Johnson EJ., Cohen G N. and Marliere P, The Journal of Biological Chemistry, 268, (1993), 26827-26835) containing glucose (45 g/L), and MgSO₄ (1 mM). The cultures were further incubated in 96 deep-well sealed microplates at 30° C., 700 rpm shaking for 24 hours. The production of isobutene was stopped by incubating the microplates for 5 min at 80° C. and the isobutene formed in the reaction headspace was analysed by Gas Chromatography (GC) equipped with Flame Ionization Detector (FID). 100 μL of headspace gases from each enzymatic reaction are injected in a Brucker GC-450 system equipped with a Flame Ionization Detector (FID). Compounds present in samples were separated by chromatography using a GS-alumina column (30 m×0.53 mm) (Agilent) using isothermal mode at 130° C. Nitrogen was used as carrier gas with a flow rate of 6 ml/min. Upon injection, peak areas of isobutene were calculated; Table N.

TABLE N

| | | IBN production, |
|---|---|---|
| Strain number | Vectors | arbitrary units |
| Strain 1 (metabolic | pUC18_MCS + pGB 2021 | 950 |
| pathway-free control), | | |
| containing the empty | | |
| vectors | | |
| Strain 2, expressing only | pGB 5796 + pGB 2021 | 710 |
| UbiX proteine | | |
| Strain 3, expressing the | pUC18_MCS + PGB 5771 | 625 |
| whole metabolic pathway, | | |
| without overexpression of | | |
| UbiX protein on plasmid | | |
| Strain 4, expressing the | pGB 5796 + pGB 5771 | 15192 |
| whole metabolic pathway, | | |
| comprising overexpression | | |
| of UbiX protein on plasmid | | |

1-36. (canceled)

37. A recombinant microorganism capable of producing isobutene, wherein said microorganism expresses polypeptides comprising:
a) i) a thioester hydrolase (EC 3.1.2.-); or
ii) a phosphate butyryltransferase (EC 2.3.1.19) and a phosphotransferase with a carboxy group as acceptor (EC 2.7.2.-); and
b) an FMN-dependent decarboxylase associated with an FMN prenyl transferase (EC 2.5.1.-), an aconitate decarboxylase (EC 4.1.1.6), a geranoyl-CoA carboxylase (EC 6.4.1.5), or a protocatechuate (PCA) decarboxylase (EC 4.1.1.63).

38. The recombinant organism or microorganism of claim 37, wherein the thioester hydrolase (EC 3.1.2.-) is selected from an acetyl-CoA hydrolase (EC 3.1.2.1), an ADP-dependent short-chain-acyl-CoA hydrolase (EC 3.1.2.18) or an acyl-CoA hydrolase (EC 3.1.2.20).

39. The recombinant organism or microorganism of claim 37, wherein the phosphotransferase with a carboxy group as acceptor (EC 2.7.2.-) is selected from a propionate kinase (EC 2.7.2.15), an acetate kinase (EC 2.7.2.1), a butyrate kinase (EC 2.7.2.7) or a branched-chain-fatty-acid kinase (EC 2.7.2.14).

40. The recombinant organism or microorganism of claim 37, wherein the recombinant organism or microorganism comprises a FMN-dependent decarboxylase associated with an FMN prenyl transferase (EC 2.5.1.-).

41. The recombinant organism or microorganism of claim 38, wherein the recombinant organism or microorganism comprises a FMN-dependent decarboxylase associated with an FMN prenyl transferase (EC 2.5.1.-).

42. The recombinant organism or microorganism of claim 39, wherein the recombinant organism or microorganism comprises a FMN-dependent decarboxylase associated with an FMN prenyl transferase (EC 2.5.1.-).

43. The recombinant organism or microorganism of claim 37, wherein the recombinant organism or microorganism further expresses a polypeptide selected from a methylcrotonyl-CoA carboxylase (EC 6.4.1.4) or a geranoyl-CoA carboxylase (EC 6.4.1.5).

44. The recombinant organism or microorganism of claim 43, wherein the thioester hydrolase (EC 3.1.2.-) is selected from an acetyl-CoA hydrolase (EC 3.1.2.1), an ADP-dependent short-chain-acyl-CoA hydrolase (EC 3.1.2.18) or an acyl-CoA hydrolase (EC 3.1.2.20).

45. The recombinant organism or microorganism of claim 43, wherein the phosphotransferase with a carboxy group as acceptor (EC 2.7.2.-) is selected from a propionate kinase (EC 2.7.2.15), an acetate kinase (EC 2.7.2.1), a butyrate kinase (EC 2.7.2.7) or a branched-chain-fatty-acid kinase (EC 2.7.2.14).

46. The recombinant organism or microorganism of claim 43, wherein the recombinant organism or microorganism comprises a FMN-dependent decarboxylase associated with an FMN prenyl transferase (EC 2.5.1.-).

47. The recombinant organism or microorganism of claim 45, wherein the recombinant organism or microorganism comprises a FMN-dependent decarboxylase associated with an FMN prenyl transferase (EC 2.5.1.-).

48. The recombinant organism or microorganism of claim 44, wherein the recombinant organism or microorganism comprises a FMN-dependent decarboxylase associated with an FMN prenyl transferase (EC 2.5.1.-).

49. The recombinant organism or microorganism of claim 37, wherein the recombinant organism or microorganism further expresses a polypeptide selected from a 3-methylglutaconyl-coenzyme A hydratase (EC 4.2.1.18), a 3-hydroxyacyl-CoA dehydratase (EC 4.2.1.-) or an enoyl-CoA hydratase (EC 4.2.1.-).

50. The recombinant organism or microorganism of claim 49, wherein the thioester hydrolase (EC 3.1.2.-) is selected from an acetyl-CoA hydrolase (EC 3.1.2.1), an ADP-dependent short-chain-acyl-CoA hydrolase (EC 3.1.2.18) or an acyl-CoA hydrolase (EC 3.1.2.20).

51. The recombinant organism or microorganism of claim 49, wherein the phosphotransferase with a carboxy group as acceptor (EC 2.7.2.-) is selected from a propionate kinase (EC 2.7.2.15), an acetate kinase (EC 2.7.2.1), a butyrate kinase (EC 2.7.2.7) or a branched-chain-fatty-acid kinase (EC 2.7.2.14).

52. The recombinant organism or microorganism of claim 49, wherein the recombinant organism or microorganism comprises a FMN-dependent decarboxylase associated with an FMN prenyl transferase (EC 2.5.1.-).

53. The recombinant organism or microorganism of claim 51, wherein the recombinant organism or microorganism comprises a FMN-dependent decarboxylase associated with an FMN prenyl transferase (EC 2.5.1.-).

54. The recombinant organism or microorganism of claim 50, wherein the recombinant organism or microorganism comprises a FMN-dependent decarboxylase associated with an FMN prenyl transferase (EC 2.5.1.-).

55. The recombinant organism or microorganism of claim 37, wherein the recombinant organism or microorganism further expresses a 3-hydroxy-3-methylglutaryl-CoA synthase.

56. The recombinant organism or microorganism of claim 55, wherein the thioester hydrolase (EC 3.1.2.-) is selected from an acetyl-CoA hydrolase (EC 3.1.2.1), an ADP-dependent short-chain-acyl-CoA hydrolase (EC 3.1.2.18) or an acyl-CoA hydrolase (EC 3.1.2.20).

57. The recombinant organism or microorganism of claim 55, wherein the phosphotransferase with a carboxy group as acceptor (EC 2.7.2.-) is selected from a propionate kinase (EC 2.7.2.15), an acetate kinase (EC 2.7.2.1), a butyrate kinase (EC 2.7.2.7) or a branched-chain-fatty-acid kinase (EC 2.7.2.14).

58. The recombinant organism or microorganism of claim 55, wherein the recombinant organism or microorganism comprises a FMN-dependent decarboxylase associated with an FMN prenyl transferase (EC 2.5.1.-).

59. The recombinant organism or microorganism of claim 57, wherein the recombinant organism or microorganism comprises a FMN-dependent decarboxylase associated with an FMN prenyl transferase (EC 2.5.1.-).

60. The recombinant organism or microorganism of claim 56, wherein the recombinant organism or microorganism comprises a FMN-dependent decarboxylase associated with an FMN prenyl transferase (EC 2.5.1.-).
